(19) United States
(12) Patent Application Publication
(10) Pub. No.: US 2024/0409643 A1
(43) Pub. Date: Dec. 12, 2024

(54) ANTI-NMDAR2B ANTIBODIES, ANTIBODY-DRUG CONJUGATES, AND CHIMERIC ANTIGEN RECEPTORS, AND COMPOSITIONS AND METHODS OF USE

(71) Applicant: THE TRUSTEES OF DARTMOUTH COLLEGE, HANOVER, NH (US)

(72) Inventor: William G. NORTH, Lebanon, NH (US)

(21) Appl. No.: 18/621,312

(22) Filed: Mar. 29, 2024

Related U.S. Application Data
(63) Continuation of application No. PCT/US2022/077362, filed on Sep. 30, 2022.
(60) Provisional application No. 63/250,694, filed on Sep. 30, 2021.

(51) Int. Cl. C07K 16/28; A61K 39/00; A61P 35/00

(52) U.S. Cl. CPC C07K 16/286; A61P 35/00; A61K 2039/505; C07K 2317/55

(57) ABSTRACT

The disclosure provides anti-NMDAR2B agents such as anti-NMDAR2B antibodies (Abs), antigen-binding Ab fragments, multi-specific Abs and antigen-binding Ab fragments, antibody-drug conjugates (ADCs), and chimeric antigen receptors (CARs). The disclosure also provides polynucleotides and vectors encoding, cells and pharmaceutical compositions comprising such anti-NMDAR2B agents and/or polynucleotides. The present disclosure further relates to methods of treating a subject using such anti-NMDAR2B agents and compositions, and to methods of treating, preventing, or diagnosing a disease such as cancer and methods of stimulating an immune response. Also provided are methods of producing such anti-NMDAR2B agents and cells.

### CROSS-REFERENCE TO RELATED APPLICATIONS

This application is a continuation of PCT/US2022/077362 filed Sep. 30, 2022, which claims priority to U.S. Provisional Application No. 63/250,694, filed on Sep. 30, 2021, the contents of all of which are each incorporated by reference in their entirety herein.

### STATEMENT REGARDING FEDERALLY SPONSORED RESEARCH OR DEVELOPMENT

This invention was made with government support under R21 CA167329 awarded by the National Institutes of Health. The government has certain rights in the invention.

### SEQUENCE DISCLOSURE

The contents of the electronic sequence listing (11432520005801.xml; Size: 525,290 bytes; and Date of Creation: Mar. 26, 2024) is herein incorporated by reference in its entirety.

### FIELD OF INVENTION

The present disclosure relates to anti-NMDAR2B agents such as anti-NMDAR2B antibodies (Abs), antigen-binding Ab fragments, multi-specific Abs and antigen-binding Ab fragments, antibody-drug conjugates (ADCs), chimeric antigen receptors (CARs), cells expressing such Abs, Ab fragments, and CARs. The disclosure also relates to polynucleotides, polynucleotide combination, vectors, and vector combinations encoding, cells comprising such polynucleotide(s) and/or vector(s), pharmaceutical compositions comprising such anti-NMDAR2B agents, and methods of making such anti-NMDAR2B agents. The present disclosure further relates to methods of treating a subject or a disease and to methods of diagnosing a disease such as cancer, methods of determining NMDAR2B expression, and methods of stimulating an immune response, using such anti-NMDAR2B agents and compositions

### BACKGROUND OF THE INVENTION

Immunotherapy is a growing field, allowing for treatment of a range of diseases that did not have an effective treatment option before. Many examples of immunotherapy have been employed in oncology, including antibody therapies targeting CD20 (Non-Hodgkin's lymphoma), HER2 (HER2-positive breast cancer), and immune checkpoints such as PD-1, PD-L1, and CTLA-4 (various cancers), and chimeric antigen receptor (CAR)-based cell therapies targeting cancer antigens such as CD19 (various cancers of B cells) (Leyfman Y, et al., Cancer Cell Int. 2018 Nov. 14; 18:182). Immunotherapies are also being developed, tested, and/or marketed for non-cancer disease indications, such as autoimmune diseases (Wraith D C et al, Front Immunol. 2017 Nov. 28; 8:1668).

N-methyl D-aspartate (NMDA) receptors (NMDARs) are glutamate-gated ionotropic receptor with high calcium permeability (Blanke M L and VanDongen AMJ. Activation Mechanisms of the NMDA Receptor. In: Van Dongen A M, editor. Biology of the NMDA Receptor. Boca Raton (FL): CRC Press/Taylor & Francis; 2009. Chapter 13), and are involved in neuronal development, synaptic plasticity, learning, and memory (Blanke M L and VanDongen AMJ. Activation Mechanisms of the NMDA Receptor. In: Van Dongen A M, editor. Biology of the NMDA Receptor. Boca Raton (FL): CRC Press/Taylor & Francis; 2009. Chapter 13). NMDARs are generally expressed as heterotetramers of two glycin-binding NR1 subunits and two glutamate-binding NR2 subunits assembled around a central permeation pathway, and binding of both ligands, glycine and glutamate, is required for NMDAR activation.

NMDA receptor subtype 2B (NMDAR2B), is one of the four subtypes of NR2 (Paoletti P, et al. Nat Rev Neurosci. 2013 June; 14(6):383-400) and is also referred to as NMDA receptor subunit epsilon-2, NR2B, hNR3, glutamate receptor subunit epsilon-2, GluN2B, glutamate ionotropic receptor NMDA type subunit 2B, GRIN2B, MRD6, EIEE27NR3, DEE27. In healthy humans, the protein expression of NMDAR2B is limited to the brain (https://www.proteinatlas.org/ENSG00000273079-GRIN2B/tissue).

### SUMMARY OF THE INVENTION

The present disclosure provides anti-NMDAR2B agents.

In some aspects, the anti-NMDAR2B agent is an antibody (Ab) or antigen-binding Ab fragment. NMDAR2B optionally comprises the amino acid sequence of SEQ ID NO: 1.

In some embodiments, the Ab or antigen-binding Ab fragment may comprise: (a) a heavy chain variable region (VH) comprising a VH complementarity determining region (CDR) 1 (also referred to as CDR-H1), a VH CDR 2 (also referred to as CDR-H2), and a VH CDR 3 (also referred to as CDR-H3); and (b) a light chain variable region (VL) comprising a VL CDR 1 (CDR-L1), a VL CDR 2 (CDR-L2), and a VL CDR 3 (CDR-L3).

In some embodiments, the CDR-H1 may comprise or consist of the CDR-H1 sequence contained in the VH of Ab8, Ab9, Ab4, Ab5, Ab6, Ab7, or Ab10. In some embodiments, the CDR-H2 may comprise or consist of the CDR-H2 sequence contained in the VH of Ab8, Ab9, Ab4, Ab5, Ab6, Ab7, or Ab10. In some embodiments, the CDR-H3 may comprise or consist of the CDR-H3 sequence contained in the VH of Ab8, Ab9, Ab4, Ab5, Ab6, Ab7, or Ab10.

In some embodiments, the CDR-L1 may comprise or consist of the CDR-L1 sequence contained in the VL of Ab8, Ab9, Ab4, Ab5, Ab6, Ab7, Ab10, or Ab3. In some embodiments, the CDR-L2 may comprise or consist of the CDR-L2 sequence contained in the VL of Ab8, Ab9, Ab4, Ab5, Ab6, Ab7, Ab10, or Ab3. In some embodiments, the CDR-L3 may comprise or consist of the CDR-L3 sequence contained in the VL of Ab8, Ab9, Ab4, Ab5, Ab6, Ab7, Ab10, or Ab3.

In some embodiments, the CDR-H1 may comprise or consist of the CDR-H1 sequence contained in SEQ ID NO: 81, 91, 41, 51, 61, 71, or 101 or may comprise or consist of the amino acid sequence of SEQ ID NO: 82, 92, 42, 52, 62, 72, 102, or 32.

In some embodiments, the CDR-H2 may comprise or consist of the CDR-H2 sequence contained in SEQ ID NO: 81, 91, 41, 51, 61, 71, or 101 or may comprise or consist of the amino acid sequence of SEQ ID NO: 83, 93, 43, 53, 63, 73, or 103.

In some embodiments, the CDR-H3 may comprise or consist of the CDR-H3 sequence contained in SEQ ID NO: 81, 91, 41, 51, 61, 71, or 101 or may comprise or consist of the amino acid sequence of SEQ ID NO: 84, 94, 44, 54, 64, 74, 104, or 34.

In some embodiments, the CDR-L1 may comprise or consist of the CDR-L1 sequence contained in SEQ ID NO: 86, 96, 46, 56, 66, 76, 106, or 36 may comprise or consist of the amino acid sequence of SEQ ID NO: 87, 97, 47, 57, 67, 77, 107, or 37.

In some embodiments, the CDR-L2 may comprise or consist of the CDR-L2 sequence contained in SEQ ID NO: 86, 96, 46, 56, 66, 76, 106, or 36 may comprise or consist of the amino acid sequence of SEQ ID NO: 88, 98, 48, 58, 68, 78, 108, or 38.

In some embodiments, the CDR-L3 may comprise or consist of the CDR-L3 sequence contained in SEQ ID NO: 86, 96, 46, 56, 66, 76, 106, or 36 or may comprise or consist of the amino acid sequence of SEQ ID NO: 89, 99, 49, 59, 69, 79, 109, or 39.

In certain embodiments, some but not all of the six CDRs of an Ab or Ab fragment according to the present invention may comprise the same sequence(s) as the corresponding CDR(s) of a first anti-NMDAR2B antibody, optionally selected from Ab8, Ab9, Ab4, Ab5, Ab6, Ab7, and Ab10. In certain embodiments, the remainder of the CDRs of such an Ab or Ab fragment may comprise the same sequence(s) as the corresponding CDR(s) of a second anti-NMDAR2B antibody, optionally selected from Ab8, Ab9, Ab4, Ab5, Ab6, Ab7, and Ab10. In some cases, the first anti-NMDAR2B antibody and the second anti-NMDAR2B antibody may be the same antibody, e.g., the combination of Ab8 and Ab8 or the combination of Ab9 and Ab9. In some cases, the first anti-NMDAR2B antibody may differ from the second anti-NMDAR2B antibody, e.g., the combination of Ab8 and Ab9, the combination of Ab8 and Ab3, or the combination of Ab9 and Ab3.

In certain embodiments, the VH of an Ab or Ab fragment according to the present invention may comprise the three CDRs contained in the VH of a first anti-NMDAR2B antibody, optionally selected from Ab8, Ab9, Ab4, Ab5, Ab6, Ab7, and Ab10.

In some embodiments, the VL of such an Ab or Ab fragment according to the present invention may comprise the three CDRs contained in the VL of a second anti-NMDAR2B, optionally antibody selected from Ab8, Ab9, Ab4, Ab5, Ab6, Ab7, or Ab10. In some cases, the first anti-NMDAR2B antibody and the second anti-NMDAR2B antibody may be the same antibody, e.g., the combination of Ab8 and Ab8 or the combination of Ab9 and Ab9.

In some cases, the first anti-NMDAR2B antibody may differ from the second anti-NMDAR2B antibody, e.g., the combination of Ab8 and Ab9, the combination of Ab8 and Ab3, or the combination of Ab9 and Ab3.

In some embodiments, the CDR-H1 and CDR-H3 may comprise or consist of the amino acid sequences set forth in SEQ ID NOs: 32 and 34, respectively, and/or the CDR-L1, CDR-L2, and CDR-L3 may comprise or consist of the amino acid sequence set forth in SEQ ID NOs: 37, 38, and 39, respectively. Optionally, in such an embodiment, the CDR-H2 may comprise or consist of the amino acid sequence of SEQ ID NO: 83, 93, 43, 53, 63, 73, or 103.

In some embodiments, the CDR-H1, CDR-H2, and CDR-H3 may comprise or consist of the amino acid sequences set forth in SEQ ID NOs: 42, 43, and 44, respectively, and/or the CDR-L1, CDR-L2, and CDR-L3 may comprise or consist of the amino acid sequence set forth in SEQ ID NOs: 47, 48, and 49, respectively.

In some embodiments, the CDR-H1, CDR-H2, and CDR-H3 may comprise or consist of the amino acid sequences set forth in SEQ ID NOs: 52, 53, and 54, respectively, and/or the CDR-L1, CDR-L2, and CDR-L3 may comprise or consist of the amino acid sequence set forth in SEQ ID NOs: 57, 58, and 59, respectively.

In some embodiments, the CDR-H1, CDR-H2, and CDR-H3 may comprise or consist of the amino acid sequences set forth in SEQ ID NOs: 62, 63, and 64, respectively, and/or the CDR-L1, CDR-L2, and CDR-L3 may comprise or consist of the amino acid sequence set forth in SEQ ID NOs: 67, 68, and 69, respectively.

In some embodiments, the CDR-H1, CDR-H2, and CDR-H3 may comprise or consist of the amino acid sequences set forth in SEQ ID NOs: 72, 73, and 74, respectively, and/or the CDR-L1, CDR-L2, and CDR-L3 may comprise or consist of the amino acid sequence set forth in SEQ ID NOs: 77, 78, and 79, respectively.

In some embodiments, the CDR-H1, CDR-H2, and CDR-H3 comprise or consist of the amino acid sequences set forth in SEQ ID NOs: 82, 83, and 84, respectively, and/or the CDR-L1, CDR-L2, and CDR-L3 may comprise or consist of the amino acid sequence set forth in SEQ ID NOs: 87, 88, and 89, respectively.

In some embodiments, the CDR-H1, CDR-H2, and CDR-H3 may comprise or consist of the amino acid sequences set forth in SEQ ID NOs: 92, 93, and 94, respectively, and/or the CDR-L1, CDR-L2, and CDR-L3 may comprise or consist of the amino acid sequence set forth in SEQ ID NOs: 97, 98, and 99, respectively.

In some embodiments, the CDR-H1, CDR-H2, and CDR-H3 may comprise or consist of the amino acid sequences set forth in SEQ ID NOs: 102, 103, and 104, respectively, and/or the CDR-L1, CDR-L2, and CDR-L3 may comprise or consist of the amino acid sequence set forth in SEQ ID NOs: 107, 108, and 109, respectively.

In some embodiments, the Ab or Ab fragment may be an affinity matured variant of any of the foregoing Abs and antigen-binding Ab fragments.

In particular embodiments, the Ab or antigen-binding Ab fragment comprises the CDR-H1, CDR-H2, and CDR-H3 of SEQ ID NOs: 82, 83, and 84, respectively and the CDR-L1, CDR-L2, and CDR-L3 of SEQ ID NOs: 87, 88, and 89, respectively, or the CDR-H1, CDR-H2, and CDR-H3 of SEQ ID NOs: 92, 93, and 94, respectively and the CDR-L1, CDR-L2, and CDR-L3 of SEQ ID NOs: 97, 98, and 99, respectively.

In some embodiments, the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 36 and may comprise the same CDRs as those contained in SEQ ID NO: 36.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 41 and may comprise the same CDRs as those contained in SEQ ID NO:41, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 46 and may comprise the same CDRs as those contained in SEQ ID NO:46.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 51 and may comprise the same CDRs as those contained in SEQ ID NO: 51, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 56 and may comprise the same CDRs as those contained in SEQ ID NO:56.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 61 and may comprise the same CDRs as those contained in SEQ ID NO: 61, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 66 and may comprise the same CDRs as those contained in SEQ ID NO: 66.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 71 and may comprise the same CDRs as those contained in SEQ ID NO: 71, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 76 and may comprise the same CDRs as those contained in SEQ ID NO: 76.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 81 and may comprise the same CDRs as those contained in SEQ ID NO: 81, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 86 and may comprise the same CDRs as those contained in SEQ ID NO: 86.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 91 and may comprise the same CDRs as those contained in SEQ ID NO: 91, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 96 and may comprise the same CDRs as those contained in SEQ ID NO: 96.

In some embodiments, the VH may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 101 and may comprise the same CDRs as those contained in SEQ ID NO: 101, and/or the VL may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 106 and may comprise the same CDRs as those contained in SEQ ID NO: 106.

In certain embodiments, (a) the VH may comprise an amino acid sequence which is at least 90%, at least 92%, at least 94%, at least 95%, at least 96%, at least 97%, at least 98%, or at least 99% identical to SEQ ID NO: 81 and may comprise the same CDRs as those contained in SEQ ID NO: 81 and (b) the VL may comprise an amino acid sequence which is at least 90%, at least 92%, at least 94%, at least 95%, at least 96%, at least 97%, at least 98%, or at least 99% identical to SEQ ID NO: 86 and may comprise the same CDRs as those contained in SEQ ID NO: 86.

In certain embodiments, (a) the VH may comprise an amino acid sequence which is at least 90%, at least 92%, at least 94%, at least 95%, at least 96%, at least 97%, at least 98%, or at least 99% identical to SEQ ID NO: 91 and may comprise the same CDRs as those contained in SEQ ID NO: 91 and (b) the VL may comprise an amino acid sequence which is at least 90%, at least 92%, at least 94%, at least 95%, at least 96%, at least 97%, at least 98%, or at least 99% identical to SEQ ID NO: 96 and may comprise the same CDRs as those contained in SEQ ID NO: 96.

In some embodiments, the VH of an Ab or Ab fragment according to the present invention may comprise the VH sequence of a first anti-NMDAR2B antibody, optionally selected from Ab8, Ab9, Ab4, Ab5, Ab6, Ab7, and Ab10. In some embodiments, VL of such an Ab or Ab fragment may comprise the VL sequence of a second anti-NMDAR2B antibody, optionally selected from Ab8, Ab9, Ab4, Ab5, Ab6, Ab7, and Ab10. In some cases, the first anti-NMDAR2B antibody and the second anti-NMDAR2B antibody may be the same antibody, e.g., the combination of Ab8 and Ab8 or the combination of Ab9 and Ab9. In some cases, the first anti-NMDAR2B antibody may differ from the second anti-NMDAR2B antibody, e.g., the combination of Ab8 and Ab9, the combination of Ab8 and Ab3, or the combination of Ab9 and Ab3.

In particular embodiments, the Ab or antigen-binding Ab fragment comprises the VH and VL of SEQ ID NOS: 81 and 86, respectively, or the VH and VL of SEQ ID NOS: 91 and 96, respectively.

The Ab or antigen-binding Ab fragment according to the present disclosure may be a human, humanized, or chimeric Ab or Ab fragment; may be monospecific, bispecific, or multispecific; may be monovalent, bivalent, trivalent, tetravalent, pentavalent, hexavalent, or multivalent; may be monomeric, dimeric, trimeric, tetrameric, pentameric, or multimeric; may be recombinant or synthetic; may be or may comprise an IgM, IgD, IgG, IgE, or IgA; may be or may comprise an IgG1, IgG2, IgG3, IgG4, IgA1, or IgA2; and/or may be or may comprise a single chain Ab, a domain-deleted Ab, a hybrid Ab, CDR-grafted Ab, a fragment antigen-binding (Fab), a F(ab′)2, a Fab′ fragment, a variable fragment (Fv), a single-chain Fv (scFv), an Fd fragment, a scFv-Fc, an scFc fusion protein, a diabody, and/or a minibody.

The Ab or antigen-binding Ab fragment according to the present disclosure may be or comprises an IgG, an IgM, an IgD, an IgE, or an IgA, which comprises a heavy chain comprising any of the VHs described above and a light chain comprising any of the VLs described above.

In some embodiments, the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 35 and comprises the same CDRs as those contained in SEQ ID NO: 35.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 40 and comprises the same CDRs as those contained in SEQ ID NO: 40, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 45 and comprises the same CDRs as those contained in SEQ ID NO: 45.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 50 and comprises the same CDRs as those contained in SEQ ID NO:50, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 55 and comprises the same CDRs as those contained in SEQ ID NO: 55.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 60 and comprises the same CDRs as those contained in SEQ ID NO: 60, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 65 and comprises the same CDRs as those contained in SEQ ID NO: 65.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 70 and comprises the same CDRs as those contained in SEQ ID NO: 70, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 75 and comprises the same CDRs as those contained in SEQ ID NO: 75.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 80 and comprises the same CDRs as those contained in SEQ ID NO: 80, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 85 and comprises the same CDRs as those contained in SEQ ID NO: 85.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 90 and comprises the same CDRs as those contained in SEQ ID NO: 90, and/or the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 95 and comprises the same CDRs as those contained in SEQ ID NO: 95.

In some embodiments, the heavy chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 100 and may comprise the same CDRs as those contained in SEQ ID NO: 100, and/or the light chain comprises an amino acid sequence which is at least 80%, at least 85%, at least 90%, at least 92%, at least 94%, at least 95%, at least 96%, at least 97%, at least 98%, at least 99%, or 100% identical to SEQ ID NO: 105 and comprises the same CDRs as those contained in SEQ ID NO: 105.

In certain embodiments, the Ab is an IgG consisting of any of the heavy and light chain combinations described above.

In certain embodiments, (a) the heavy chain may comprise an amino acid sequence which is at least 90%, at least 92%, at least 94%, at least 95%, at least 96%, at least 97%, at least 98%, or at least 99% identical to SEQ ID NO: 80 and comprises the same CDRs as those contained in SEQ ID NO: 80, and (b) the light chain may comprise an amino acid sequence which is at least 90%, at least 92%, at least 94%, at least 95%, at least 96%, at least 97%, at least 98%, or at least 99% identical to SEQ ID NO: 85 and comprises the same CDRs as those contained in SEQ ID NO: 85.

In certain embodiments, (a) the heavy chain may comprise an amino acid sequence which is at least 90%, at least 92%, at least 94%, at least 95%, at least 96%, at least 97%, at least 98%, at least 99% identical to SEQ ID NO: 90 and comprises the same CDRs as those contained in SEQ ID NO: 90, and (b) the light chain may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 95 and comprises the same CDRs as those contained in SEQ ID NO: 95.

In some embodiments, the heavy chain of an Ab or Ab fragment according to the present invention may comprise the heavy chain sequence of a first anti-NMDAR2B antibody, optionally selected from Ab8, Ab9, Ab4, Ab5, Ab6, Ab7, and Ab10. In some embodiments, light chain of such an Ab or Ab fragment may comprise the light chain sequence of a second anti-NMDAR2B antibody, optionally selected from Ab8, Ab9, Ab4, Ab5, Ab6, Ab7, and Ab10. In some cases, the first anti-NMDAR2B antibody and the second anti-NMDAR2B antibody may be the same antibody, e.g., the combination of Ab8 and Ab8 or the combination of Ab9 and Ab9. In some cases, the first anti-NMDAR2B antibody may differ from the second anti-NMDAR2B antibody, e.g., the combination of Ab8 and Ab9, the combination of Ab8 and Ab3, or the combination of Ab9 and Ab3.

In particular embodiments, the Ab is an IgG consisting of the heavy and light chains of SEQ ID NOS: 80 and 85, respectively, or the heavy and light chains of SEQ ID NOS: 90 and 95, respectively.

The Ab or Ab fragment according to the present disclosure may be or may comprise a scFv comprising, in the direction from the N-terminus to the C-terminus: the VH, a linker, and the VL; or the VL, a linker, and the VH. The linker may be any appropriate linker that allows for the VH and VL to form a binding site for NMDAR2B.

In some embodiments, the linker may comprise the amino acid sequence of SEQ ID NO: 110 or 111 or a multiple repeat of SEQ ID NO: 110.

In some embodiments, the scFv may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 240, 245, 250, 255, 260, 265, 270, 275, 280, 285, 290, 295, 300, or 305 and comprises the same CDRs as those contained in said respective SEQ ID NOS.

In particular embodiments, the scFv comprise the amino acid sequence of any one of SEQ ID NOS: 280, 285, 290, and 295.

In some embodiments, the scFv may compete for binding to NMDAR2B with a scFv comprising an amino acid sequence which is at least 80%, at least 85%, at least 90%, at least 92%, at least 94%, at least 95%, at least 96%, at least 97%, at least 98%, at least 99%, or 100% identical to SEQ ID NOs: 240, 245, 250, 255, 260, 265, 270, 275, 280, 285, 290, 295, 300, or 305.

The Ab or Ab fragment according to the present disclosure may comprise at least a first binding specificity and a second binding specificity, and the first specificity is to an epitope in NMDAR2B, and optionally, the second specificity is to another epitope in NMDAR2B or to an epitope in a second antigen other than NMDAR2B.

In some embodiments, the second specificity is to a molecule expressed on an immune cell, such as a T cell, B cell, NK cells, NKT cells, macrophages, monocytes, dendritic cells, neutrophils, eosinophils, basophils, or mast cells, and in some cases the second specificity may provide, facilitate, or support an effector function of the Ab or Ab fragment, such as Ab-dependent cellular cytotoxicity (ADCC) and Ab-dependent cellular phagocytosis (ADCP).

In some embodiments, the second specificity is to CD3, NKG2D, 4-1BB, or Fc receptor (FcR), optionally wherein the FcR is selected from the group consisting of Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn).

The Ab or Ab fragment according to the present disclosure may comprise one or more of a human heavy chain constant region domain 1 (CH1) or a variant thereof, a human hinge, a human heavy chain constant region domain 2 (CH2) or a variant thereof, a human heavy chain constant region domain 3 (CH3) or a variant thereof, a human kappa light chain constant region domain (CLκ) or a variant thereof; and/or a human lambda light chain constant region domain (CLλ) or a variant thereof.

In certain embodiments, the human CH1 may comprise or consist of the amino acid sequence of: SEQ ID NO: 310 or is at least 80%, at least 85%, at least 90%, at least 92%, at least 94%, at least 95%, at least 96%, at least 97%, at least 98%, or at least 99% identical thereto.

In certain embodiments, the human hinge may comprise or consist of the amino acid sequence of: SEQ ID NO: 311 or is at least 80%, at least 85%, at least 90%, at least 92%, at least 94%, at least 95%, at least 96%, at least 97%, at least 98%, or at least 99% identical thereto.

In certain embodiments, the human CH2 may comprise or consist of the amino acid sequence of: SEQ ID NO: 312 or is at least 80%, at least 85%, at least 90%, at least 92%, at least 94%, at least 95%, at least 96%, at least 97%, at least 98%, or at least 99% identical thereto.

In certain embodiments, the human CH3 may comprise or consist of the amino acid sequence of: SEQ ID NO: 313, 314, 315, or 316 or is at least 80%, at least 85%, at least 90%, at least 92%, at least 94%, at least 95%, at least 96%, at least 97%, at least 98%, or at least 99% identical thereto.

In certain embodiments, the human CLκ may comprise or consist of the amino acid sequence of: SEQ ID NO: 325 or is at least 80%, at least 85%, at least 90%, at least 92%, at least 94%, at least 95%, at least 96%, at least 97%, at least 98%, or at least 99% identical thereto.

In certain embodiments, the human CLλ may comprise or consist of the amino acid sequence of: SEQ ID NO: 326 or is at least 80%, at least 85%, at least 90%, at least 92%, at least 94%, at least 95%, at least 96%, at least 97%, at least 98%, or at least 99% identical thereto.

The Ab or Ab fragment according to the present disclosure may comprise a fragment crystallizable (Fc) region.

In some embodiments, the Fc region may or may be derived from a human Fc region.

In some embodiments, the Fc region may or may be derived from the Fc region of an IgM, an IgD, an IgG, an IgE, or an IgA, optionally of an IgG1, an IgG2, an IgG3, or an IgG4.

In some embodiments, the Fc region binds to an FcR selected from the group consisting of Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn).

In certain embodiments, the Fc region comprises or consists of the amino acid sequence of SEQ ID NO: 317, 318, 319, or 320 or is at least 80%, at least 85%, at least 90%, at least 92%, at least 94%, at least 95%, at least 96%, at least 97%, at least 98%, at least 99%, or 100% identical thereto.

In some embodiments, the Fc region is modified to alter at least one of effector function, half-life, proteolysis, or glycosylation.

In some embodiments, Fc region contains one or more amino acid substitutions or modifications that alters or eliminates N- and/or O-glycosylation.

The Ab or Ab fragment according to the present disclosure may comprise a heavy chain constant region.

In some embodiments, the heavy chain constant region may be or may be derived from a human heavy chain constant region.

In some embodiments, the heavy chain constant region may be or may be derived from the heavy chain constant region of an IgM, an IgD, an IgG, an IgE, or an IgA, optionally of an IgG1, an IgG2, an IgG3, or an IgG4.

In some embodiments, the heavy chain constant region may bind to an FcR selected from the group consisting of Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn).

In certain embodiments, the heavy chain constant region may comprise or consist of the amino acid sequence of SEQ ID NO: 321, 322, 323, or 324 or is at least 80%, at least 85%, at least 90%, at least 92%, at least 94%, at least 95%, at least 96%, at least 97%, at least 98%, at least 99%, or 100% identical thereto.

In some embodiments, the heavy chain constant region is modified to alter at least one of effector function, half-life, proteolysis, or glycosylation.

In some embodiments, the heavy chain constant region contains one or more amino acid substitutions or modifications that alters or eliminates N- and/or O-glycosylation.

In some embodiments, the Ab or Ab fragment according to the present disclosure may bind to human NMDAR2B with a dissociation constant (Kd) of less than 10 nM.

In some embodiments, the Ab or Ab fragment according to the present disclosure may bind to human NMDAR2B peptide comprising the amino acid sequence of SEQ ID NO: 2 with a dissociation constant (Kd) of less than 10 nM, optionally with a Kd of about 5 nM, further optionally wherein human NMDAR2B peptide is conjugated to a protein, further optionally wherein the Kd is determined by ELISA.

In some embodiments, the Ab or Ab fragment according to the present disclosure may bind to human NMDAR2B expressed on the surface of cancer cells, and some instances the cancer cells may be cells of small-cell lung cancer (SCLC), pancreatic cancer, ovarian cancer, breast cancer, or prostate cancer.

In some embodiments, the Ab or Ab fragment according to the present disclosure may be capable of reducing viability of cells expressing human NMDAR2B, and in some instances the cells may be cells of SCLC, pancreatic cancer, ovarian cancer, breast cancer, or prostate cancer.

In some embodiments, the Ab or Ab fragment according to the present disclosure may be capable of inhibiting cancer growth in a subject having cancer, and in some instances the cancer may be SCLC, pancreatic cancer, ovarian cancer, breast cancer, or prostate cancer.

In some embodiments, the Ab or Ab fragment according to the present disclosure may work synergistically with another anti-cancer agent in inhibiting cancer growth in a subject having cancer, and in some instances the cancer may be SCLC, pancreatic cancer, ovarian cancer, breast cancer, or prostate cancer, and in certain instances the other anti-cancer agent may be an alkylating agent, such as cyclophosphamide.

In some embodiments, the Ab or Ab fragment according to the present disclosure may be attached to at least one effector moiety, chemical inker, detectable moiety, or functional moiety, and in some instances the detectable moiety may be a fluorescent dye, an enzyme, a substrate, a bioluminescent material, a radioactive material, a chemiluminescent moiety, or a combination thereof.

In some aspects, the anti-NMDAR2B agents according to the present disclosure encompass antibody-drug conjugates (ADCs) which comprises any of the Abs or Ab fragments described above and a drug conjugated to the Ab or Ab fragment.

In some embodiments, the drug in the ADC may be selected from the group consisting of an anti-cancer drug, an anti-proliferative drug, a cytotoxic drug, an alkylating agent, a platinum agent, an anthracycline, a taxane, an anti-angiogenic drug, an apoptotic drug, an immunostimulatory drug, an anti-microbial drug, an antibiotic drug, an antiviral drug, an anti-inflammatory drug, an NMDA receptor antagonist, an NMDA receptor signaling inhibitor, an NMDAR1 inhibitor, an NMDAR2B inhibitor, an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, and an imaging drug.

In some embodiments, the drug in the ADC may be one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU) doxorubicin, daunorubicin, cucurbitacin, chaetocin, chaetoglobosin, chlamydocin, calicheamicin, epirubicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, dactinomycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone, or a derivative thereof.

In some aspects, the anti-NMDAR2B agents according to the present disclosure encompass chimeric antigen receptors (CARs) comprising: an antigen-binding domain that binds to human NMDAR2B; a transmembrane (TM) domain; an intracellular signaling (ICS) domain; optionally a hinge that joins said antigen-binding domain and said TM domain; and optionally one or more costimulatory (CS) domains.

In some embodiments, the antigen-binding domain of the CAR may be any of the Abs or Ab fragments described above.

In some embodiments, the antigen-binding domain of the CAR may be any of the scFvs described above.

In some embodiments, the antigen-binding domain of the CAR may comprise an amino acid sequence which is at least 80%, at least 85%, at least 90%, at least 92%, at least 94%, at least 95%, at least 96%, at least 97%, at least 98%, at least 99%, or 100% identical to SEQ ID NOs: 240, 245, 250, 255, 260, 265, 270, 275, 280, 285, 290, 295, 300, or 305 and comprises the same CDRs as those contained in said respective SEQ ID NO.

In particular embodiments, the antigen-binding domain of the CAR may comprise an amino acid sequence which is at least 80%, at least 85%, at least 90%, at least 92%, at least 94%, at least 95%, at least 96%, at least 97%, at least 98%, at least 99%, or 100% identical to SEQ ID NOs: 280, 285, 290, or 295 and comprises the same CDRs as those contained in said respective SEQ ID NO.

In some embodiments, the antigen-binding domain of the CAR may compete for binding to NMDAR2B with a scFv comprising an amino acid sequence which is at least 80%, at least 85%, at least 90%, at least 92%, at least 94%, at least 95%, at least 96%, at least 97%, at least 98%, at least 99%, or 100% identical to SEQ ID NOs: 240, 245, 250, 255, 260, 265, 270, 275, 280, 285, 290, 295, 300, or 305.

In some embodiments, the TM domain of the CAR may be derived from the TM region, or a membrane-spanning portion thereof, of a protein selected from the group consisting of CD28, CD3e, CD4, CDS, CD8, CD9, CD16, CD22, CD33, CD37, CD45, CD64, CD80, CD86, CD134, CD137, CD154, TCRa, TCRb, and CD3z.

In some embodiments, the TM domain of the CAR may be derived from the TM region of CD28, or a membrane-spanning portion thereof.

In particular embodiments, the TM domain may comprise an amino acid sequence which is at least 80%, at least 85%, at least 90%, at least 95%, at least 98% at least 99%, or 100% identical to the amino acid sequence of SEQ ID NO: 114.

In some embodiments, the ICS domain of the CAR may be derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CDS, CD22, CD66d, CD79a, CD79b, CD278 (ICOS), FceRI, DAP10, and DAP12.

In some embodiments, the ICS domain may be derived from a cytoplasmic signaling sequence of CD3z, or a functional fragment thereof.

In particular embodiments, the ICS domain may comprise an amino acid sequence which is at least 80%, at least 85%, at least 90%, at least 95%, at least 98% at least 99%, or 100% identical to the amino acid sequence of SEQ ID NO: 118.

In some embodiments, the hinge of the CAR may be derived from CD28.

In particular embodiments, the hinge may comprise an amino sequence which is at least 80%, at least 85%, at least 90%, at least 95%, at least 98% at least 99%, or 100% identical to the amino acid sequence of SEQ ID NO: 113.

In some embodiments, the CAR may comprise at least one of the one or more CS domains, which may be derived from a cytoplasmic signaling sequence, or functional fragment thereof, of a protein selected from the group consisting of CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

In some embodiments, the CS domain may comprise a cytoplasmic signaling sequence of CD28, 4-1BB, or DAP10, or functional fragment thereof.

In particular embodiments, the CS domain may comprise an amino sequence which is at least 80%, at least 85%, at least 90%, at least 95%, at least 98% at least 99%, or 100% identical to the amino acid sequence of SEQ ID NO: 115, 116, or 117.

In some embodiments, the CAR may comprise an amino acid sequence which is at least 80%, at least 85%, at least 90%, at least 95%, at least 98% at least 99%, or 100% identical to the amino acid sequence of Ab4scFvHL-CD28H-CD28TM-CD28CS-CD3zICS (SEQ ID NO: 241), Ab4scFvHL-CD28H-CD28TM-41BBCS-CD3zICS (SEQ ID NO: 242), Ab4scFvHL-CD28H-CD28TM-DAP10CS-CD3zICS (SEQ ID NO: 243), Ab4scFvLH-CD28H-CD28TM-CD28CS-CD3zICS (SEQ ID NO: 246), Ab4scFvLH-CD28H-CD28TM-41BBCS-CD3zICS (SEQ ID NO: 247), Ab4scFvLH-CD28H-CD28TM-DAP10CS-CD3zICS (SEQ ID NO: 248), Ab5scFvHL-CD28H-CD28TM-CD28CS-CD3zICS (SEQ ID NO: 251), Ab5scFvHL-CD28H-CD28TM-41BBCS-CD3zICS (SEQ ID NO: 252), Ab5scFvHL-CD28H-CD28TM-DAP10CS-CD3zICS (SEQ ID NO: 253), Ab5scFvLH-CD28H-CD28TM-CD28CS-CD3zICS (SEQ ID NO: 256), Ab5scFvLH-CD28H-CD28TM-41BBCS-CD3zICS (SEQ ID NO: 257), Ab5scFvLH-CD28H-CD28TM-DAP10CS-CD3zICS (SEQ ID NO: 258), Ab6scFvHL-CD28H-CD28TM-CD28CS-CD3zICS (SEQ ID NO: 261), Ab6scFvHL-CD28H-CD28TM-41BBCS-CD3zICS (SEQ ID NO: 262), Ab6scFvHL-CD28H-CD28TM-DAP10CS-CD3zICS (SEQ ID NO: 263), Ab6scFvLH-CD28H-CD28TM-CD28CS-CD3zICS (SEQ ID NO: 266), Ab6scFvLH-CD28H-CD28TM-41BBCS-CD3zICS (SEQ ID NO: 267), Ab6scFvLH-CD28H-CD28TM-DAP10CS-CD3zICS (SEQ ID NO: 268), Ab7scFvHL-CD28H-CD28TM-CD28CS-CD3zICS (SEQ ID NO: 271), Ab7scFvHL-CD28H-CD28TM-41BBCS-CD3zICS (SEQ ID NO: 272), Ab7scFvHL-CD28H-CD28TM-DAP10CS-CD3zICS (SEQ ID NO: 273), Ab7scFvLH-CD28H-CD28TM-CD28CS-CD3zICS (SEQ ID NO: 276), Ab7scFvLH-CD28H-CD28TM-41BBCS-CD3zICS (SEQ ID NO: 277), Ab7scFvLH-CD28H-CD28TM-DAP10CS-CD3zICS (SEQ ID NO: 278), Ab8scFvHL-CD28H-CD28TM-CD28CS-CD3zICS (SEQ ID NO: 281), Ab8scFvHL-CD28H-CD28TM-41BBCS-CD3zICS (SEQ ID NO: 282), Ab8scFvHL-CD28H-CD28TM-DAP10CS-CD3zICS (SEQ ID NO: 283), Ab8scFvLH-CD28H-CD28TM-CD28CS-CD3zICS (SEQ ID NO: 286), Ab8scFvLH-CD28H-CD28TM-41BBCS-CD3zICS (SEQ ID NO: 287), Ab8scFvLH-CD28H-CD28TM-DAP10CS-CD3zICS (SEQ ID NO: 288), Ab9scFvHL-CD28H-CD28TM-CD28CS-CD3zICS (SEQ ID NO: 291), Ab9scFvHL-CD28H-CD28TM-41BBCS-CD3zICS (SEQ ID NO: 292), Ab9scFvHL-CD28H-CD28TM-DAP10CS-CD3zICS (SEQ ID NO: 293), Ab9scFvLH-CD28H-CD28TM-CD28CS-CD3zICS (SEQ ID NO: 296), Ab9scFvLH-CD28H-CD28TM-41BBCS-CD3zICS (SEQ ID NO: 297), Ab9scFvLH-CD28H-CD28TM-DAP10CS-CD3zICS (SEQ ID NO: 298), Ab10scFvHL-CD28H-CD28TM-CD28CS-CD3zICS (SEQ ID NO: 301), Ab10scFvHL-CD28H-CD28TM-41BBCS-CD3zICS (SEQ ID NO: 302), Ab10scFvHL-CD28H-CD28TM-DAP10CS-CD3zICS (SEQ ID NO: 303), Ab10scFvLH-CD28H-CD28TM-CD28CS-CD3zICS (SEQ ID NO: 306), Ab10scFvLH-CD28H-CD28TM-41BBCS-CD3zICS (SEQ ID NO: 307), or Ab10scFvLH-CD28H-CD28TM-DAP10CS-CD3zICS (SEQ ID NO: 308).

In particular embodiments, the CAR may comprise an amino acid sequence which is at least 80%, at least 85%, at least 90%, at least 95%, at least 98% at least 99%, or 100% identical to the amino acid sequence of Ab8scFvHL-CD28H-CD28TM-CD28CS-CD3zICS (SEQ ID NO: 281), Ab8scFvHL-CD28H-CD28TM-41BBCS-CD3zICS (SEQ ID NO: 282), Ab8scFvHL-CD28H-CD28TM-DAP10CS-CD3zICS (SEQ ID NO: 283), Ab8scFvLH-CD28H-CD28TM-CD28CS-CD3zICS (SEQ ID NO: 286), Ab8scFvLH-CD28H-CD28TM-41BBCS-CD3zICS (SEQ ID NO: 287), Ab8scFvLH-CD28H-CD28TM-DAP10CS-CD3zICS (SEQ ID NO: 288), Ab9scFvHL-CD28H-CD28TM-CD28CS-CD3zICS (SEQ ID NO: 291), Ab9scFvHL-CD28H-CD28TM-41BBCS-CD3zICS (SEQ ID NO: 292), Ab9scFvHL-CD28H-CD28TM-DAP10CS-CD3zICS (SEQ ID NO: 293), Ab9scFvLH-CD28H-CD28TM-CD28CS-CD3zICS (SEQ ID NO: 296), Ab9scFvLH-CD28H-CD28TM-41BBCS-CD3zICS (SEQ ID NO: 297), or Ab9scFvLH-CD28H-CD28TM-DAP10CS-CD3zICS (SEQ ID NO: 298).

In specific embodiments, the CAR may comprise an amino acid sequence which is at least 80%, at least 85%, at least 90%, at least 95%, at least 98% at least 99%, or 100% identical to the amino acid sequence of Ab8scFvHL-CD28H-CD28TM-CD28CS-CD3zICS (SEQ ID NO: 281), Ab8scFvLH-CD28H-CD28TM-CD28CS-CD3zICS (SEQ ID NO: 286), Ab9scFvHL-CD28H-CD28TM-CD28CS-CD3zICS (SEQ ID NO: 291), ir Ab9scFvLH-CD28H-CD28TM-CD28CS-CD3zICS (SEQ ID NO: 296).

In some embodiments, the CAR according to the present disclosure may further comprise a drug conjugated to the antigen-binding domain.

In some embodiments, the drug is one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU) doxorubicin, daunorubicin, cucurbitacin, chaetocin, chaetoglobosin, chlamydocin, calicheamicin, epirubicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, dactinomycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone, or a derivative thereof.

The present disclosure also provides isolated polynucleotides or combinations of isolated polynucleotides encoding any of the Abs or Ab fragments described above.

In some embodiments, the VH-encoding polynucleotide may comprise the CDR-H1- and CDR-H3-encoding nucleic acid sequences of SEQ ID NOs: 132 and 134, respectively, and/or the VL-encoding polynucleotide may comprise the CDR-L1-, CDR-L2-, and CDR-L3-encoding nucleic acid sequences of SEQ ID NOs: 137, 138, and 139, respectively.

In some embodiments, the VH-encoding polynucleotide may comprise the CDR-H1-, CDR-H2-, and CDR-H3-encoding nucleic acid sequences of SEQ ID NOs: 142, 143, and 144, respectively, and/or the VL-encoding polynucleotide may comprise the CDR-L1-, CDR-L2-, and CDR-L3-encoding nucleic acid sequences of SEQ ID NOs: 147, 148, and 149, respectively.

In some embodiments, the VH-encoding polynucleotide may comprise the CDR-H1-, CDR-H2-, and CDR-H3-encoding nucleic acid sequences of SEQ ID NOs: 152, 153, and 154, respectively, and/or the VL-encoding polynucleotide may comprise the CDR-L1-, CDR-L2-, and CDR-L3-encoding nucleic acid sequences of SEQ ID NOs: 157, 158, and 159, respectively.

In some embodiments, the VH-encoding polynucleotide may comprise the CDR-H1-, CDR-H2-, and CDR-H3-encoding nucleic acid sequences of SEQ ID NOs: 162, 163, and 164, respectively, and/or the VL-encoding polynucleotide may comprise the CDR-L1-, CDR-L2-, and CDR-L3-encoding nucleic acid sequences of SEQ ID NOs: 167, 168, and 169, respectively.

In some embodiments, the VH-encoding polynucleotide may comprise the CDR-H1-, CDR-H2-, and CDR-H3-encoding nucleic acid sequences of SEQ ID NOs: 172, 173, and 174, respectively, and/or the VL-encoding polynucleotide may comprise the CDR-L1-, CDR-L2-, and CDR-L3-encoding nucleic acid sequences of SEQ ID NOs: 177, 178, and 179, respectively.

In some embodiments, the VH-encoding polynucleotide may comprise the CDR-H1-, CDR-H2-, and CDR-H3-encoding nucleic acid sequences of SEQ ID NOs: 182, 183, and 184, respectively, and/or the VL-encoding polynucleotide may comprise the CDR-L1-, CDR-L2-, and CDR-L3-encoding nucleic acid sequences of SEQ ID NOs: 187, 188, and 189, respectively.

In some embodiments, the VH-encoding polynucleotide may comprise the CDR-H1-, CDR-H2-, and CDR-H3-encoding nucleic acid sequences of SEQ ID NOs: 192, 193, and 194, respectively, and/or the VL-encoding polynucleotide may comprise the CDR-L1-, CDR-L2-, and CDR-L3-encoding nucleic acid sequences of SEQ ID NOs: 197, 198, and 199, respectively.

In some embodiments, the VH-encoding polynucleotide may comprise the CDR-H1-, CDR-H2-, and CDR-H3-encoding nucleic acid sequences of SEQ ID NOs: 202, 203, and 204, respectively, and/or the VL-encoding polynucleotide may comprise the CDR-L1-, CDR-L2-, and CDR-L3-encoding nucleic acid sequences of SEQ ID NOs: 207, 208, and 209, respectively.

In some embodiments, the VL-encoding nucleic acid sequence may be at least 85%, at least 90%, at least 92%, at least 94%, at least 95%, at least 96%, at least 97%, at least 98%, at least 99%, or 100% identical to SEQ ID NO: 136 and encode the same CDRs as those contained in SEQ ID NO: 36.

In some embodiments, the the VH-encoding nucleic acid sequence may be at least 85%, at least 90%, at least 92%, at least 94%, at least 95%, at least 96%, at least 97%, at least 98%, at least 99%, or 100% identical to SEQ ID NO: 141 and encode the same CDRs as those contained in SEQ ID NO: 41, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 146 and encode the same CDRs as those contained in SEQ ID NO: 46.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 151 and encode the same CDRs as those contained in SEQ ID NO: 51, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 156 and encode the same CDRs as those contained in SEQ ID NO: 56.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 161 and encode the same CDRs as those contained in SEQ ID NO: 61, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 166 and encode the same CDRs as those contained in SEQ ID NO: 66.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 171 and encode the same CDRs as those contained in SEQ ID NO: 71, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 176 and encode the same CDRs as those contained in SEQ ID NO: 76.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 181 and encode the same CDRs as those contained in SEQ ID NO: 81, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 186 and encode the same CDRs as those contained in SEQ ID NO: 86.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 191 and encode the same CDRs as those contained in SEQ ID NO: 91, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 196 and encode the same CDRs as those contained in SEQ ID NO: 96.

In some embodiments, the VH-encoding nucleic acid sequence may be at least 85%, at least 90%, at least 92%, at least 94%, at least 95%, at least 96%, at least 97%, at least 98%, at least 99%, or 100% identical to SEQ ID NO: 201 and encode the same CDRs as those contained in SEQ ID NO: 101, and/or the VL-encoding nucleic acid sequence may be at least 85%, at least 90%, at least 92%, at least 94%, at least 95%, at least 96%, at least 97%, at least 98%, at least 99%, or 100% identical to SEQ ID NO: 206 and encode the same CDRs as those contained in SEQ ID NO: 106. In some embodiments, the isolated polynucleotide or the combination of isolated polynucleotides may encode a heavy chain comprising a VH and a light chain comprising a VL, and the VH and VL combination may be any of the above-described.

In certain embodiments, the VH-encoding nucleic acid sequence may comprise the nucleic acid sequence of SEQ ID NO: 181, and the VL-encoding nucleic acid sequence may comprise the nucleic acid sequence of SEQ ID NO: 186.

In certain embodiments, the VH-encoding nucleic acid sequence may comprise the nucleic acid sequence of SEQ ID NO: 191, and the VL-encoding nucleic acid sequence may comprise the nucleic acid sequence of SEQ ID NO: 196.

In some embodiments, the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 135 and encode the same CDRs as those contained in SEQ ID NO: 35.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 140 and encode the same CDRs as those contained in SEQ ID NO: 40, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 145 and encode the same CDRs as those contained in SEQ ID NO: 45.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 150 and encode the same CDRs as those contained in SEQ ID NO: 50, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 155 and encode the same CDRs as those contained in SEQ ID NO: 55.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 160 and encode the same CDRs as those contained in SEQ ID NO: 60, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 165 and encode the same CDRs as those contained in SEQ ID NO: 65.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 170 and encode the same CDRs as those contained in SEQ ID NO: 70, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 175 and encode the same CDRs as those contained in SEQ ID NO: 75.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 180 and encode the same CDRs as those contained in SEQ ID NO: 80, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 185 and encode the same CDRs as those contained in SEQ ID NO: 85.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 190 and encode the same CDRs as those contained in SEQ ID NO: 90, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 195 and encode the same CDRs as those contained in SEQ ID NO: 95.

In some embodiments, the heavy chain-encoding nucleic acid sequence may be at least 85%, at least 90%, at least 92%, at least 94%, at least 95%, at least 96%, at least 97%, at least 98%, at least 99%, or 100% identical to SEQ ID NO: 200 and encode the same CDRs as those contained in SEQ ID NO: 100, and/or the light chain-encoding nucleic acid sequence may be at least 85%, at least 90%, at least 92%, at least 94%, at least 95%, at least 96%, at least 97%, at least 98%, at least 99%, or 100% identical to SEQ ID NO: 205 and encode the same CDRs as those contained in SEQ ID NO: 105.

In certain embodiments, the heavy chain-encoding nucleic acid sequence may comprise the nucleic acid sequence of SEQ ID NO: 180, and the light chain-encoding nucleic acid sequence may comprise the nucleic acid sequence of SEQ ID NO: 185.

In certain embodiments, the heavy chain-encoding nucleic acid sequence may comprise the nucleic acid sequence of SEQ ID NO: 190 and the light chain-encoding nucleic acid sequence may comprise the nucleic acid sequence of SEQ ID NO: 195.

In the isolated polynucleotide or the combination of isolated polynucleotides according to the present disclosure, in some embodiments, the VH may be contained in a first polypeptide and encoded under a first promoter, and the VL may be contained in a second polypeptide and encoded under a second promoter, wherein the first and second polypeptides are separate polypeptides and the first and second promoters are separate promoters (which may or may not be a same kind of promoters).

In the isolated polynucleotide or the combination of isolated polynucleotides according to the present disclosure, in some embodiments, the VH may be contained in a first polypeptide and encoded under a first promoter, and the VL may be contained in a second polypeptide and encoded under the first promoter, wherein the first and second polypeptides are separate polypeptides. In such an embodiment, a ribosome skip sequence (e.g., T2A) may be inserted between the first polypeptide-encoding sequence and the second polypeptide-encoding sequence, which allows generating two separate polypeptides (first and second polypeptides) once translated.

In the isolated polynucleotide or the combination of isolated polynucleotides according to the present disclosure, in some embodiments, the VH is contained in a first polypeptide and encoded under a first promoter, and the VL is contained in a first polypeptide and encoded under the first promoter. For example, an scFv may be encoded in this manner.

The present disclosure also provides isolated polynucleotides or combinations of isolated polynucleotides encoding any of the scFvs described above.

In some embodiments, the linker-encoding nucleic acid sequence may comprise or consist of SEQ ID NO: 210 or 211.

In some embodiments, the scFv-encoding polynucleotide may comprise a nucleic acid sequence which is at least 80%, at least 85%, at least 90%, at least 92%, at least 94%, at least 95%, at least 96%, at least 97%, at least 98%, at least 99%, or 100% identical to SEQ ID NO: 340, 345, 350, 355, 360, 365, 370, 375, 380, 385, 390, 395, 400, or 405 and encode the same CDRs as those contained in SEQ ID NO: 240, 245, 250, 255, 260, 265, 270, 275, 280, 285, 290, 295, 300, or 305, respectively.

In the isolated polynucleotide or the combination of isolated polynucleotides encoding the Ab or Ab fragment according to the present disclosure, the Ab or Ab fragment may comprise: a human CH1 or a variant thereof; a human hinge; a human CH2 or a variant thereof; a human CH3 or a variant thereof; a human CLκ or a variant thereof; and/or a human CLλ or a variant thereof.

In certain embodiments, the human CH1-encoding sequence may comprise or consist of the nucleic acid sequence of: SEQ ID NO: 410 or is at least 80%, at least 85%, at least 90%, at least 92%, at least 94%, at least 95%, at least 96%, at least 97%, at least 98%, or at least 99% identical thereto.

In certain embodiments, the human hinge-encoding sequence may comprise or consist of the nucleic acid sequence of: SEQ ID NO: 411 or is at least 80%, at least 85%, at least 90%, at least 92%, at least 94%, at least 95%, at least 96%, at least 97%, at least 98%, or at least 99% identical thereto.

In certain embodiments, the human CH2-encoding sequence may comprise or consist of the nucleic acid sequence of: SEQ ID NO: 412 or is at least 80%, at least 85%, at least 90%, at least 92%, at least 94%, at least 95%, at least 96%, at least 97%, at least 98%, or at least 99% identical thereto.

In certain embodiments, the human CH3-encoding sequence may comprise or consist of the nucleic acid sequence of: SEQ ID NO: 413, 414, 415, or 416 or is at least 80%, at least 85%, at least 90%, at least 92%, at least 94%, at least 95%, at least 96%, at least 97%, at least 98%, or at least 99% identical thereto.

In certain embodiments, the human CLκ-encoding sequence may comprise or consist of the nucleic acid sequence of: SEQ ID NO: 425 or is at least 80%, at least 85%, at least 90%, at least 92%, at least 94%, at least 95%, at least 96%, at least 97%, at least 98%, or at least 99% identical thereto.

In certain embodiments, the human CLλ-encoding sequence may comprise or consist of the nucleic acid sequence of: SEQ ID NO: 426 or is at least 80%, at least 85%, at least 90%, at least 92%, at least 94%, at least 95%, at least 96%, at least 97%, at least 98%, or at least 99% identical thereto.

In the isolated polynucleotide or the combination of isolated polynucleotides encoding the Ab or Ab fragment according to the present disclosure, the Ab or Ab fragment may comprise a Fc region.

In certain embodiments, the Fc region-encoding sequence may comprise or consist of the nucleic acid sequence of: SEQ ID NO: 417, 418, 419, or 420 or is at least 80%, at least 85%, at least 90%, at least 92%, at least 94%, at least 95%, at least 96%, at least 97%, at least 98%, or at least 99% identical thereto.

In the isolated polynucleotide or the combination of isolated polynucleotides encoding the Ab or Ab fragment according to the present disclosure, the Ab or Ab fragment may comprise a heavy chain constant region.

In certain embodiments, the heavy chain constant region-encoding sequence may comprise or consist of the nucleic acid sequence of: SEQ ID NO: 421, 422, 423, or 424 or is at least 80%, at least 85%, at least 90%, at least 92%, at least 94%, at least 95%, at least 96%, at least 97%, at least 98%, or at least 99% identical thereto.

The present disclosure also provides isolated polynucleotides or combinations of isolated polynucleotides encoding any of the CARs described herein.

In some embodiments, the antigen-binding domain-encoding sequence may be according to the sequence of the isolated polynucleotide or the combination of isolated polynucleotides encoding an Ab or Ab fragment (which may be a scFv) as described above.

In certain embodiments, the antigen-binding domain-encoding sequence may comprise or consist of the nucleic acid sequence of SEQ ID NO: 340, 345, 350, 355, 360, 365, 370, 375, 380, 385, 390, 395, 400, or 405, or may be at least 80%, at least 85%, at least 90%, at least 92%, at least 94%, at least 95%, at least 96%, at least 97%, at least 98%, at least 99%, or 100% identical thereto and encode the same CDRs as those contained in SEQ ID NO: 240, 245, 250, 255, 260, 265, 270, 275, 280, 285, 290, 295, 300, or 305, respectively.

In certain embodiments, the TM domain-encoding sequence may comprise or consist of the nucleic acid sequence of SEQ ID NO: 214 or is at least 80%, at least 85%, at least 90%, at least 92%, at least 94%, at least 95%, at least 96%, at least 97%, at least 98%, at least 99%, or 100% identical thereto.

In certain embodiments, ICS domain-encoding sequence may comprise or consist of the nucleic acid sequence of SEQ ID NO: 218 or is at least 80%, at least 85%, at least 90%, at least 92%, at least 94%, at least 95%, at least 96%, at least 97%, at least 98%, at least 99%, or 100% identical thereto.

In certain embodiments, the hinge-encoding sequence may comprise or consist of the nucleic acid sequence of SEQ ID NO: 213 or is at least 80%, at least 85%, at least 90%, at least 92%, at least 94%, at least 95%, at least 96%, at least 97%, at least 98%, at least 99%, or 100% identical thereto.

In certain embodiments, the CS domain-encoding sequence may comprise or consist of the nucleic acid sequence of SEQ ID NO: 215, 216, or 217 or is at least 80%, at least 85%, at least 90%, at least 92%, at least 94%, at least 95%, at least 96%, at least 97%, at least 98%, at least 99%, or 100% identical thereto.

In particular embodiments, the CAR-encoding nucleic acid sequence may comprise or consist of the nucleic acid sequence of any one of SEQ ID NOS: 341, 342, 343, 346, 347, 348, 351, 352, 353, 356, 357, 358, 361, 362, 363, 366, 367, 368, 371, 372, 373, 376, 377, 378, 381, 382, 383, 386, 387, 388, 391, 392, 393, 396, 397, 398, 401, 402, 403, 406, 407, and 408.

In certain embodiments, the CAR-encoding nucleic acid sequence may comprise or consist of a nucleic acid sequence at least 80%, at least 85%, at least 90%, at least 92%, at least 94%, at least 95%, at least 96%, at least 97%, at least 98%, at least 99%, or 100% identical to anyone of SEQ ID NOS: 341, 342, 343, 346, 347, 348, 351, 352, 353, 356, 357, 358, 361, 362, 363, 366, 367, 368, 371, 372, 373, 376, 377, 378, 381, 382, 383, 386, 387, 388, 391, 392, 393, 396, 397, 398, 401, 402, 403, 406, 407, and 408, and encode the same CDRs as those contained in SEQ ID NOS: 241, 242, 243, 246, 247, 248, 251, 252, 253, 256, 257, 258, 261, 262, 263, 266, 267, 268, 271, 272, 273, 276, 277, 278, 281, 282, 283, 286, 287, 288, 291, 292, 293, 296, 297, 298, 301, 302, 303, 306, 307, and 308, respectively.

In some embodiments, the CAR-encoding isolated polynucleotide or the combination of isolated polynucleotides may further comprise a leader sequence (LS).

In particular embodiments, the LS may comprise or consist of the nucleic acid sequence of SEQ ID NO: 212 or may be at least 80%, at least 85%, at least 90%, at least 92%, at least 94%, at least 95%, at least 96%, at least 97%, at least 98%, at least 99%, or 100% identical thereto.

In some embodiments, the CAR-encoding isolated polynucleotide or the combination of isolated polynucleotides may further comprise a ribosome skip sequence and/or a sequence encoding truncated CD19 (trCD19).

In particular embodiments, the ribosome skip sequence may be a T2A sequence, optionally comprising or consisting of a nucleic acid sequence which is at least 80%, at least 85%, at least 90%, at least 95%, at least 98% at least 99%, or 100% identical to SEQ ID NO: 219.

In particular embodiments, the trCD19-encoding sequence may comprise or consist of a nucleic acid sequence which is at least 80%, at least 85%, at least 90%, at least 95%, at least 98% at least 99%, or 100% identical to SEQ ID NO: 220.

In particular embodiments, the CAR-encoding polynucleotide may may comprise or consist of the nucleic acid sequence of SEQ ID NO: 384, 389, 394, or 399.

The present disclosure also provides vectors or combination of vectors encoding any of the Abs or Ab fragments disclosed herein or any of the CARs disclosed herein.

In some embodiments, the vector or the combination of vectors may comprise a polynucleotide or a combination of polynucleotides as disclosed herein.

In some embodiments, the combination of vectors encoding an Ab or Ab fragment may comprise a first vector encoding a VH and a second vector encoding a VL.

In some embodiments, the first vector may encode a heavy chain comprising the VH and the second vector may encode a light chain comprising the VL.

In some embodiments, in the vector or a combination of vectors according to the present disclosure, the vector or each of the vectors may be individually selected from a DNA, an RNA, a plasmid, a cosmid, a viral replicon, a viral vector, a lentiviral vector, an adenoviral vector, or a retroviral vector.

The present disclosure also provides recombinant or isolated cells comprising: any of the Abs or Ab fragments described above; any of the ADCs described above; any of the CARs described above; any of the polynucleotides or combinations of polynucleotides described above; and/or any of the vectors or combinations of vectors described above.

In some embodiments, when the cells comprise an Ab or Ab fragments, the cell may be for producing or manufacturing the Ab or Ab fragments.

In some embodiments, when the cells comprise a CAR, the cell may be for use in therapy.

In some embodiments, the recombinant or isolated cell according to the present disclosure may be a non-mammalian cell, such as but not limited to a plant cell, a bacterial cell, a fungal cell, a yeast cell, a protozoa cell, or an insect cell.

In some embodiments, the recombinant or isolated cell according to the present disclosure may be a non-mammalian cell, such as but not limited to a mammalian cell, optionally a human cell, a monkey cell, a rabbit cell, a rat cell, or a mouse cell, further optionally a Chinese Hamster Ovary (CHO) cell, a Human Embryonic Kidney (HEK) cell, a HEK293 cell, or a HEK293T cell.

In some embodiments, the recombinant or isolated cell according to the present disclosure may be a stem cell or an immune cell.

In some embodiments, the recombinant or isolated cell according to the present disclosure may be a primary cell, optionally a human primary cell or derived therefrom.

In some embodiments, the recombinant or isolated cell according to the present disclosure may be a cell line, optionally a hybridoma cell line or a T cell line.

In some embodiments, the recombinant or isolated cell according to the present disclosure may be MHC+ or MHC.

In some embodiments, the recombinant or isolated cell according to the present disclosure may be a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer (NK) cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, or a dendritic cell.

In some embodiments, for example the cell encodes and/or express the Ab or Ab fragment or the ADC according to the present disclosure, the recombinant or isolated cell may be a mammalian cell (e.g., NS0 murine myeloma cells, PER.C6® human cells, HEK cells, HEK293 cells, HEK293T cells, CHO cells), a yest cell (e.g., Saccharomyces cerevisiae, Pichia pastoris,), an insect cell (e.g., Spodoptera frugiperda Sf9, Trichoplusia ni BTI-TN5B1-4 “High Five”, Spodoptera frugiperda SfSWT-1 “Mimic™” insect cells), a plant cell (e.g., N. tabacum (such as BY2 or NT1 cells), Oryza sativa (such as Bengal, Donjin, or Taipie cells), Hordeum vulgare), or a bacteria cell (e.g., Escherichia coli).

In some embodiments, for example the cell encodes and/or express the CAR according to the present disclosure, the recombinant or isolated cell may be a T cell, and in certain embodiments, the T cell may be modified such that its endogenous T cell receptor (TCR) is not expressed, not functionally expressed, or expressed at reduced levels compared to a wild-type T cell.

In certain embodiments, the TCR expression may be eliminated or reduced via genome editing (e.g., CRISPR/Cas, transcription activator-like effector nuclease (TALEN), or zinc-finger nucleases (ZFN)), siRNA, miRNA, or introducing a dominant negative mutation(s), each targeting the TCR or an element of a TCR component

In some embodiments, when the Ab or Ab fragment, the ADC, or the CAR binds to its target molecule, the recombinant or isolated cell may be activated or stimulated to proliferate.

In some embodiments, when the Ab or Ab fragment, the ADC, or the CAR binds to its target molecule, the cell may exhibit cytotoxicity against cells expressing the target molecule.

In some embodiments, when the Ab or Ab fragment, the ADC, or the CAR binds to its target molecule, the cell may increase expression of a cytokine (e.g., an immunostimulatory cytokines such as IFN-g) and/or a chemokine.

In some embodiments, when the Ab or Ab fragment, the ADC, or the CAR binds to its target molecule, the cell may decrease expression of a cytokine (e.g., an immunosuppressive cytokines such as TGF-b and/or IL-10) and/or a chemokine.

In some embodiments, when the Ab or Ab fragment, the ADC, or the CAR binds to its target molecule, administration of the cell to a subject may ameliorate a disease, optionally cancer, in the subject.

The present disclosure also provides populations of any of the cells described above.

The present disclosure also provides pharmaceutical compositions comprising: any of the Abs or Ab fragments described above, any of the ADCs described above; any of the CARs described above; any of the polynucleotides or combinations of polynucleotides described above; any of the vectors or combinations of vectors described above; any of the cells described above; and/or any of the populations of cells described above.

In some embodiments, the pharmaceutical composition may further comprise a pharmaceutically acceptable excipient or carrier.

In some embodiments, the pharmaceutical composition may further comprise another therapeutic agent.

In some embodiments the other therapeutic agent may be an anti-cancer agent, an anti-proliferative drug, a cytotoxic drug, an anti-angiogenic drug, an apoptotic drug, an immunostimulatory drug, an NMDA receptor antagonist, an NMDA receptor signaling inhibitor, an NMDAR1 inhibitor, an NMDAR2B inhibitor.

In some embodiments the other therapeutic agent may be an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, or an oligonucleotide.

In some embodiments the other therapeutic agent may be a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof.

In some embodiments the other therapeutic agent may be an immunotherapeutic agent, optionally an immune checkpoint inhibitor or a growth factor or growth factor receptor inhibitor, further optionally an inhibitor of PD-L1, PD-1, CTLA-4, VISTA, EGF, EGFR, VEGF, and/or VEGFR, or an antibody or antibody fragment against PD-L1, PD-1, CTLA-4, VISTA, EGF, EGFR, VEGF, and/or VEGFR, or an antibody or antibody fragment against a cancer antigen other than NMDAR2B.

In some embodiments the other therapeutic agent may be an anti-emetic agent, optionally one or more selected from a neurokinin-1 receptor antagonist (NK1 RA), serotonin receptor antagonist (5-HT3 RA), dexamethasone, olanzapine, and palonosetron.

The present disclosure also provides methods of treating a subject.

In some embodiments, the method may comprise administering to a subject in need thereof a therapeutically effective amount of: any of the Abs or Ab fragments described above, any of the ADCs described above; any of the CARs described above; any of the polynucleotides or combinations of polynucleotides described above; any of the vectors or combinations of vectors described above; any of the cells described above; any of the populations of cells described above; and/or any of the pharmaceutical compositions described above.

In some embodiments, the method may be for the treatment of cancer.

In some embodiments, the method may be for the treatment of pancreatic cancer, prostate cancer, ovarian cancer, small cell lung cancer, or breast cancer.

The present disclosure also provides methods for stimulating an immune response in a subject.

In some embodiments, the method may comprise administering to the subject a therapeutically effective amount of: any of the Abs or Ab fragments described above, any of the ADCs described above; any of the CARs described above; any of the polynucleotides or combinations of polynucleotides described above; any of the vectors or combinations of vectors described above; any of the cells described above; any of the populations of cells described above; and/or any of the pharmaceutical compositions described above.

In some embodiments, the method may be for stimulating an immune response against NMDAR2B-expressing cancer cells and/or for the treatment of cancer.

In some embodiments, the cancer may be pancreatic cancer, prostate cancer, ovarian cancer, small cell lung cancer, or breast cancer.

The present disclosure also provides methods of treating a disease in a subject.

In some embodiments, the method may comprise administering to the subject in need thereof a therapeutically effective amount of: any of the Abs or Ab fragments described above, any of the ADCs described above; any of the CARs described above; any of the polynucleotides or combinations of polynucleotides described above; any of the vectors or combinations of vectors described above; any of the cells described above; any of the populations of cells described above; and/or any of the pharmaceutical compositions described above.

In some embodiments, the disease may comprise cancer, which may be e.g., pancreatic cancer, prostate cancer, ovarian cancer, small cell lung cancer, or breast cancer.

The present disclosure also provides methods of treating cancer in a subject.

In some embodiments, the method may comprise the steps of: (a) obtaining or having obtained a biological sample from the subject, (b) measuring the expression level of NMDAR2B in the biological sample, (c) determining whether the biological sample overexpresses NMDAR2B, and (d) if NMDAR2B is overexpressed, administering to the subject a therapeutically effective amount of: any of the Abs or Ab fragments described above, any of the ADCs described above; any of the CARs described above; any of the polynucleotides or combinations of polynucleotides described above; any of the vectors or combinations of vectors described above; any of the cells described above; any of the populations of cells described above; and/or any of the pharmaceutical compositions described above.

In some embodiments, the cancer may be pancreatic cancer, prostate cancer, ovarian cancer, small cell lung cancer, or breast cancer.

In some embodiments, the methods described above may further comprise administering another agent.

In some embodiments, the other agent may be an anti-cancer agent, an anti-proliferative drug, a cytotoxic drug, an anti-angiogenic drug, an apoptotic drug, an immunostimulatory drug, an NMDA receptor antagonist, an NMDA receptor signaling inhibitor, an NMDAR1 inhibitor, an NMDAR2B inhibitor.

In some embodiments, the other agent may be an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, or an oligonucleotide.

In some embodiments, the other agent may be a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof.

In some embodiments, the other agent may be an immunotherapeutic agent, optionally an immune checkpoint inhibitor or a growth factor or growth factor receptor inhibitor, further optionally an inhibitor of PD-L1, PD-1, CTLA-4, VISTA, EGF, EGFR, VEGF, and/or VEGFR, or an antibody or antibody fragment against PD-L1, PD-1, CTLA-4, VISTA, EGF, EGFR, VEGF, and/or VEGFR, or an antibody or antibody fragment against a cancer antigen other than NMDAR2B.

In some embodiments, the other agent may be an anti-emetic agent, optionally one or more selected from a neurokinin-1 receptor antagonist (NK1 RA), serotonin receptor antagonist (5-HT3 RA), dexamethasone, olanzapine, and palonosetron

The present disclosure also provides methods of determining the expression level of NMDAR2B in a biological sample of a subject.

In some embodiments, the method comprises: (a) obtaining or having obtained a biological sample from the subject, and (b) contacting the biological sample with any of the Abs or Ab fragments described above, thereby determining the expression level of NMDAR2B in the biological sample.

In some embodiments, the Ab or Ab fragment may be attached to at least one detectable moiety, optionally selected from a fluorescent dye, an enzyme, a substrate, a bioluminescent material, a radioactive material, a chemiluminescent moiety, or a combination thereof.

In some embodiments, the biological sample may contain or may be suspected to contain cancer cells, optionally cells of pancreatic cancer, prostate cancer, ovarian cancer, small cell lung cancer, or breast cancer.

The present disclosure also provides methods of determining the expression level of NMDAR2B in a subject.

In some embodiments, the method may comprise administering to the subject any of the Abs or Ab fragments described herein.

In some embodiments, the Ab or Ab fragment may optionally be attached to at least one detectable moiety, which may be, e.g., a fluorescent dye, an enzyme, a substrate, a bioluminescent material, a radioactive material, a chemiluminescent moiety, or a combination thereof.

In some embodiments, the subject may have or may be suspected to have cancer, optionally pancreatic cancer, prostate cancer, ovarian cancer, small cell lung cancer, or breast cancer.

In some embodiments, the expression level determined by the determination method described herein may provide guidance as to whether the subject has cancer or a specific type of cancer and/or the subject may be treated with a particular therapy, which may optionally include the use of any of the Abs, Ab fragments, ADCs, and/or CAR-based cell therapy described herein.

The present disclosure also provides methods of diagnosing cancer in a subject.

In some embodiments, the method may comprising: (a) obtaining or having obtained a biological sample from the subject; and (b) contacting the biological sample with any of the Abs or Ab fragments described herein, thereby determining the expression level of NMDAR2B in the biological sample; and (c) diagnosing that the subject has cancer, if the expression level of NMDAR2B is higher than a standard level or the level in a biological sample from a healthy subject

In some embodiments, the biological sample may be suspected to contain cancer cells, such as but not limited to cells of pancreatic cancer, prostate cancer, ovarian cancer, small cell lung cancer, or breast cancer.

In some embodiments, the method may comprise: (a) administering to the subject any of the Abs or Ab fragments described above, (b) determining the expression level of NMDAR2B in the subject; and (c) diagnosing that the subject has cancer, if the expression level of NMDAR2B is higher than a standard level or the level in healthy subject

In some embodiments, the Ab or Ab fragment may be attached to at least one detectable moiety, such as but not limited to a fluorescent dye, an enzyme, a substrate, a bioluminescent material, a radioactive material, a chemiluminescent moiety, or a combination thereof.

In some embodiments, the subject may be suspected to have cancer, optionally pancreatic cancer, prostate cancer, ovarian cancer, small cell lung cancer, or breast cancer.

The present disclosure also provides methods of making any of the Abs or Ab fragments described herein.

In some embodiments, the method comprises: (a) culturing a cell comprising a polynucleotide or a combination of polynucleotides encoding the Ab or Ab fragment and/or comprising a vector or a combination of vectors encoding the Ab or Ab fragment in a condition that allows for expression of the Ab or Ab fragment; and optionally (b) purifying the Ab or Ab fragment from the culture.

In some embodiments, the polynucleotide or the combination of polynucleotides may be any of the Ab or Ab fragment-encoding polynucleotides or the Ab or Ab fragment-encoding combinations of polynucleotides described above.

In some embodiments, the vector or the combination of vectors may be any of the Ab or Ab fragment-encoding vectors or the Ab or Ab fragment-encoding combinations of vectors described above.

In some embodiments, the cell may be any of the Ab or Ab fragment-encoding cells described above.

In some embodiments, the method comprises: (a) in vitro translating or expressing a polynucleotide or a combination of polynucleotides encoding the Ab or Ab fragment; and optionally (b) purifying the Ab or Ab fragment.

In some embodiments, the polynucleotide or the combination of polynucleotides may be any of the Ab or Ab fragment-encoding polynucleotides or the Ab or Ab fragment-encoding combinations of polynucleotides described above.

The present disclosure also provides methods of making one or more cells expressing a CAR according to the present disclosure.

In some embodiments, the method comprises: (a) introducing into cells a polynucleotide or a combination of polynucleotides encoding the CAR and/or a vector or a combination of vectors encoding the CAR; (b) culturing the cells in a condition that allows for expression of the CAR; and optionally (c) purifying cells that express the CAR.

In some embodiments, the polynucleotide or the combination of polynucleotides may be any of the CAR-encoding polynucleotide or the CAR-encoding combination of polynucleotides described herein.

In some embodiments, the vector or the combination of vectors may be any of the CAR-encoding vector or the CAR-encoding combination of vectors described above.

In some embodiments, the cells obtained from step (a) may comprise at least one CAR-encoding cell described above.

In some embodiments, the CAR-encoding polynucleotide or the CAR-encoding combination of polynucleotides and/or a CAR-encoding vector or a CAR-encoding combination of vectors encodes a selection marker for expression of the CAR, and the purifying of step (c) is based on the selection marker.

In certain embodiments, the selection marker may be trCD19.

The present disclosure also provides methods of identifying an improved Ab or antigen-binding Ab fragment thereof which binds to NMDAR2B.

In some embodiments, the method may comprise: (a) modifying the amino acid sequence of the Ab or Ab fragment of any one of claims 1-12; (b) optionally expressing the Ab or Ab fragment modified in (a) and assessing a biochemical and/or biological property of interest of the expressed Ab or Ab fragment; and (c) optionally selecting an improved Ab or Ab fragment when an improved biochemical and/or biological property is observed in (b).

In certain embodiments, in (a), the modifying may comprise amino acid substitution, amino acid insertion, and/or deletion.

In certain embodiments, in (a), the VH and/or VL sequence(s) may be modified.

In certain embodiments, in (a), one or more of the CDR sequences may be modified. In certain embodiments, in (a) one or more of the framework region (FR) sequences are modified. In certain embodiments, in (a), the constant region sequence(s) may be modified.

In certain embodiments, in (b), the biochemical and/or biological property of interest may comprise any one or more of the following: (i) affinity to a NMDAR2B peptide, optionally a human or cynomolgus NMDAR2B peptide; (ii) biding to a NMDAR2B-expressing cell, optionally a cancer cell; (iii) expression level; (iv) stability or shelf-life of the Ab or Ab fragment; (v) in vitro or ex vivo function and/or activity, optionally inhibition of ligand binding and/or cytotoxicity against a NMDAR2B-expressing cell; (vi) in vitro or in vivo toxicity, optionally in a mammal such as a rodent, rabbit, human, or non-human primate; (vii) in vivo half-life of the Ab or Ab fragment, optionally in a mammal such as a rodent, rabbit, human, or non-human primate; (viii) biodistribution, optionally the ability of the Ab or Ab fragment to reach a desired site (e.g., disease site) and/or the inability of the Ab or Ab fragment to reach an undesired site (e.g., brain), optionally in a mammal such as a rodent, rabbit, human, or non-human primate; (ix) in vivo function and/or activity, optionally inhibition of ligand binding and/or cytotoxicity against a NMDAR2B-expressing cell, optionally in a mammal such as a rodent, rabbit, human, or non-human primate; and/or (x) in vivo efficacy, optionally based on an anti-cancer effect or survival, optionally in a mammal such as a rodent, rabbit, human, or non-human primate.

In certain embodiments, in (c), the improved may comprise any one or more of the following: (i) increased affinity to a NMDAR2B peptide (e.g., in case of using an Ab or Ab fragment describe herein to perform affinity maturation), optionally a human or cynomolgus NMDAR2B peptide; (ii) increased biding to a NMDAR2B-expressing cell, optionally a cancer cell; (iii) increased expression level; (iv) increased stability or shelf-life of the Ab or Ab fragment; (v) increased in vitro or ex vivo function and/or activity, optionally increased inhibition of ligand binding and/or cytotoxicity against a NMDAR2B-expressing cell; (vi) increased in vitro or in vivo toxicity, optionally in a mammal such as a rodent, rabbit, human, or non-human primate; (vii) increased in vivo half-life of the Ab or Ab fragment, optionally in a mammal such as a rodent, rabbit, human, or non-human primate; (viii) improved biodistribution, optionally increased ability of the Ab or Ab fragment to reach a desired site (e.g., disease site) and/or decreased ability of the Ab or Ab fragment to reach an undesired site (e.g., brain), optionally in a mammal such as a rodent, rabbit, human, or non-human primate; (ix) increased in vivo function and/or activity, optionally increased inhibition of ligand binding and/or cytotoxicity against a NMDAR2B-expressing cell, optionally in a mammal such as a rodent, rabbit, human, or non-human primate; and/or (x) increased in vivo efficacy, optionally based on an enhanced anti-cancer effect or prolonged survival, optionally in a mammal such as a rodent, rabbit, human, or non-human primate.

### DETAILED DESCRIPTION OF THE SEVERAL VIEWS OF THE DRAWINGS

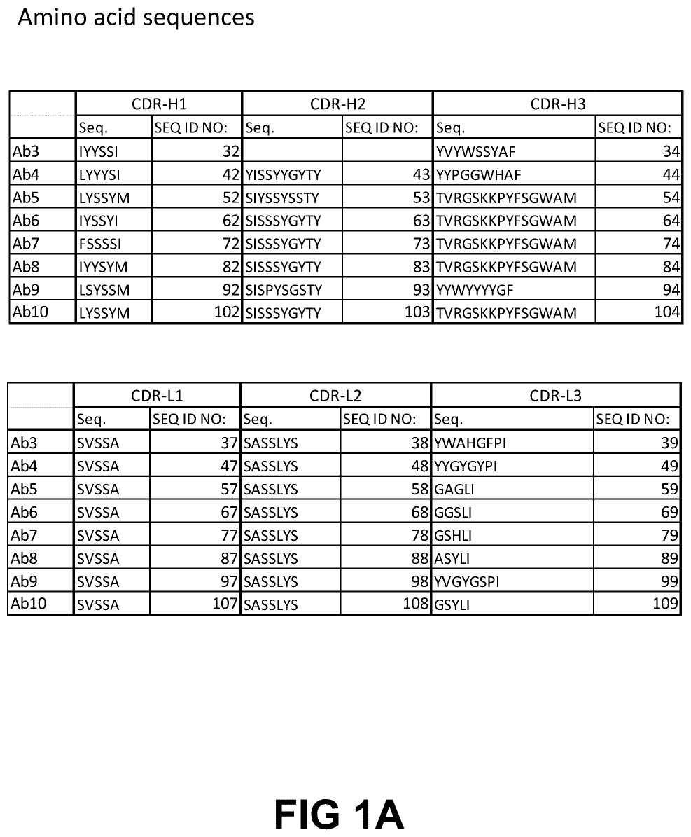

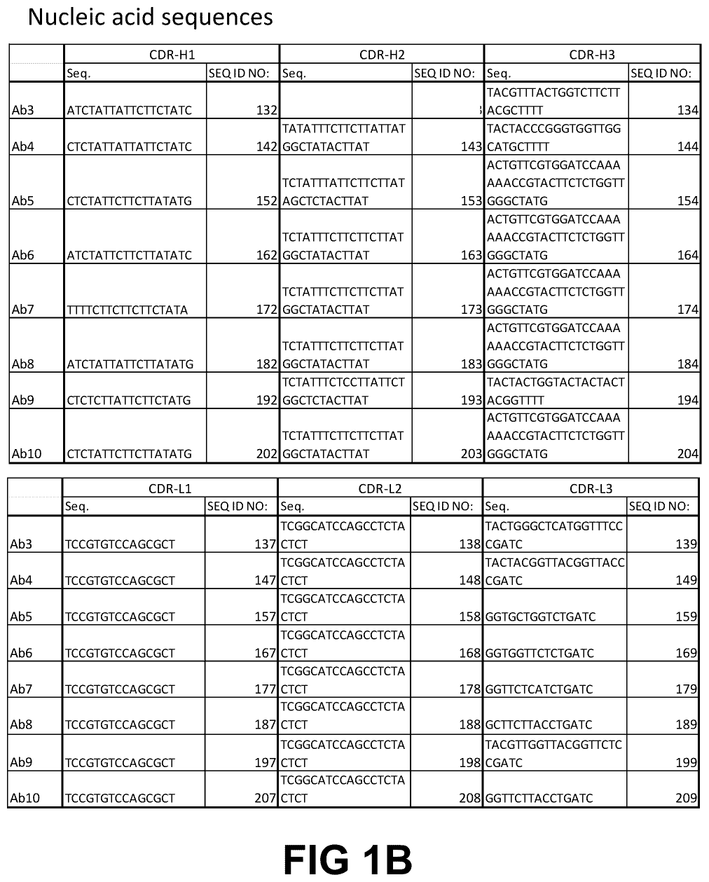

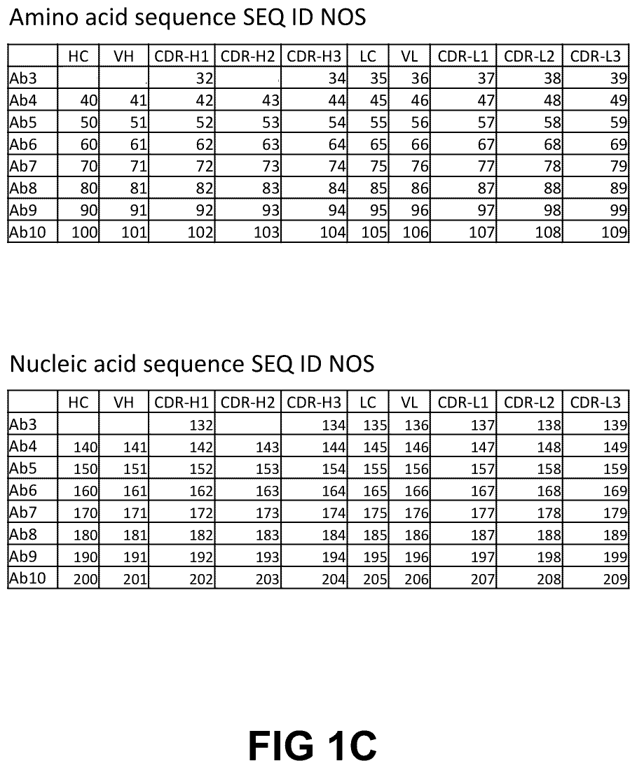

FIGS. 1A-1C provide amino acid sequences of various polypeptides of exemplary anti-NMDAR2B antibodies and exemplary nucleic acid sequences encoding such polypeptides, and SEQ ID NOS assigned thereto. FIG. 1A provides the amino acid sequences of the CDRs of the VH (top) and VL (bottom) of exemplary anti-NMDAR2B antibodies, Ab3 through Ab10, and SEQ ID NOS assigned thereto. FIG. 1B provides exemplary nucleic acid sequences encoding the CDRs of the VH (top) and VL (bottom) of Ab3 through Ab10 and SEQ ID NOS assigned thereto. FIG. 1C provides SEQ ID NOS assigned to the amino acid sequences of the HC, VH, VH CDRs, LC, VL, and VL CDRs of Ab3 through Ab10 (top) and SEQ ID NOS assigned to exemplary nucleic acid sequences encoding the HC, VH, VH CDRs, LC, VL, and VL CDRs of Ab3 through Ab10 (bottom).

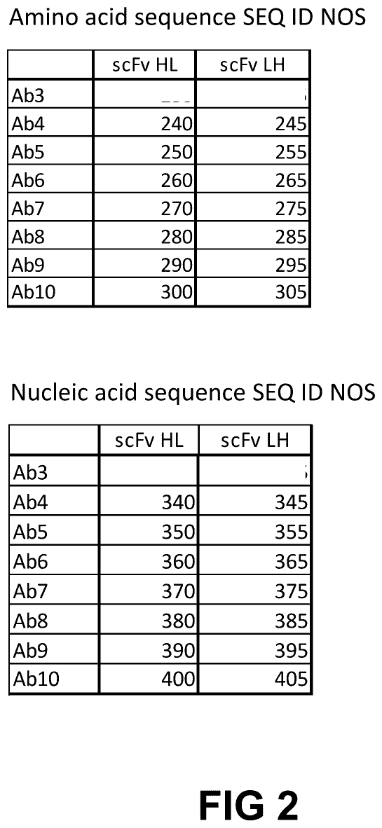

FIG. 2 provides SEQ ID NOS assigned to the amino acid sequences of exemplary scFvs comprising the VH and VL set of Ab4 through Ab10 (top) and SEQ ID NOS assigned to exemplary nucleic acid sequences encoding such scFvs (bottom). “scFv HL” in FIG. 2 comprises the structure of VH-GS4 linker-VL from the N-terminus to the C-terminus, and “scFv LH” in FIG. 2 comprises the structure of VL-GS4X3 linker-VH from the N-terminus to the C-terminus. “scFv HL” under “Ab8”, for example, which is also referred to herein as “Ab8 scFv HL” or “Ab8scFvHL” comprises the VH and VL of Ab8 and comprises the amino acid sequence of SEQ ID NO: 280, which may be encoded by the nucleic acid sequence of SEQ ID NO: 380.

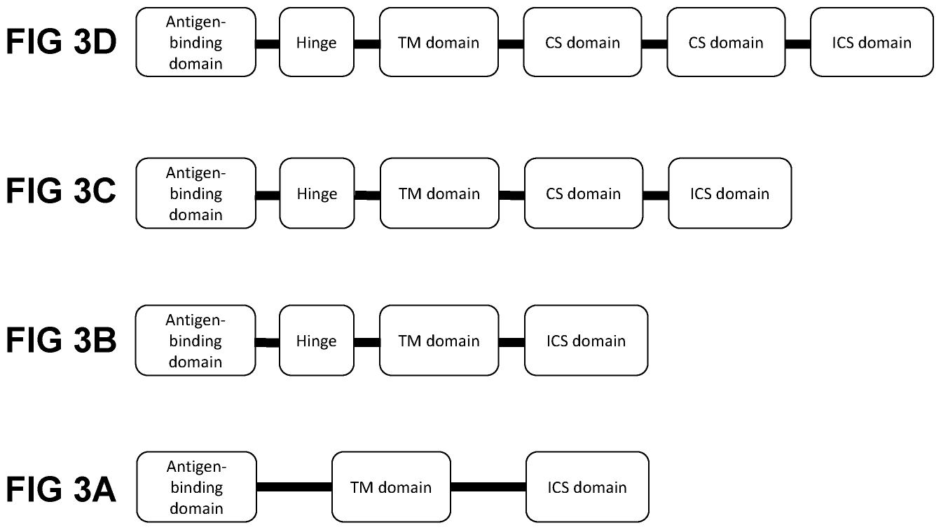

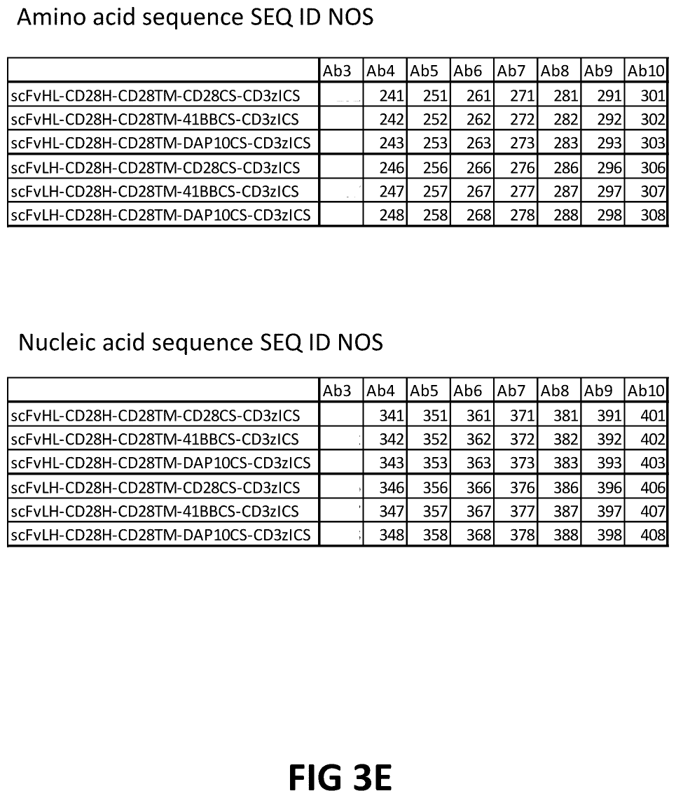

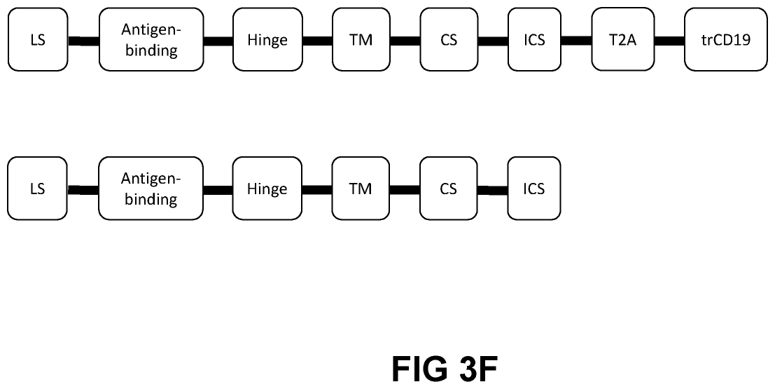

FIGS. 3A-3F provide exemplary schematics of chimeric antigen receptors (CARs) according to the present disclosure and exemplary anti-NMDAR2B CAR sequences. FIG. 3A shows a general schematic of CARs of the present disclosure, which comprises an antigen-binding domain, transmembrane (TM) domain, and an intracellular signaling (ICS) domain. FIGS. 3B-3D show further, exemplary schematics of a CAR construct according to the present disclosure, which comprises an antigen-binding domain, a TM domain, and an ICS domain, and further comprises a hinge that joins the antigen-binding and TM domains (FIG. 3B) along with one (FIG. 3C) or two (FIG. 3D) costimulatory (CS) domains. FIG. 3E provides SEQ ID NOS assigned to the amino acid sequences of exemplary anti-NMDAR2B CARs which comprise a scFv comprising the VH and VL set of Ab4 through Ab10 (top) and SEQ ID NOS assigned to exemplary nucleic acid sequences encoding such CARs (bottom). The CAR named “scFvHL-CD28H-CD28TM-CD28CS-CD3zICS” under “Ab8”, for example, comprises, from the N-terminus to the C-terminus, the scFv HL based on Ab8 as shown in FIG. 2, the hinge derived from the hinge sequence of CD28 (SEQ ID NO: 113), the TM domain derived the transmembrane sequence of CD28 (SEQ ID NO: 114), the CS domain derived from the cytoplasmic signaling sequence CD28 (SEQ ID NO: 115), and the ICS domain derived from an intracellular signaling sequence of CD3ζ (SEQ ID NO: 118), and comprises the amino acid sequence of SEQ ID NO: 281, which may be encoded by the nucleic acid sequence of SEQ ID NO: 381. “41BBCS” means the CS domain derived from the cytoplasmic signaling sequence of 4-1BB (SEQ ID NO: 116), and “DAP10CS” means the CS domain derived from the cytoplasmic signaling sequence of DAP10 (SEQ ID NO: 117). FIG. 3F shows exemplary schematics of a CAR-encoding construct that may be, e.g., included in a vector and/or used to express a CAR. Such a construct comprises a leader sequence (LS) upstream of a CAR construct (left) and optionally further comprises a ribosomal skip sequence (T2A is shown as an example) and an expression/purification marker (truncated CD19 (trCD19) is shown as an example). The CAR construct may be according to, e.g., any of FIGS. 3A-3E. In FIGS. 3A-D and F, different domains (e.g., antigen-binding domain, TM domain, etc) are connected via a black line, each representing a potential additional sequence that may or may not be included between domains.

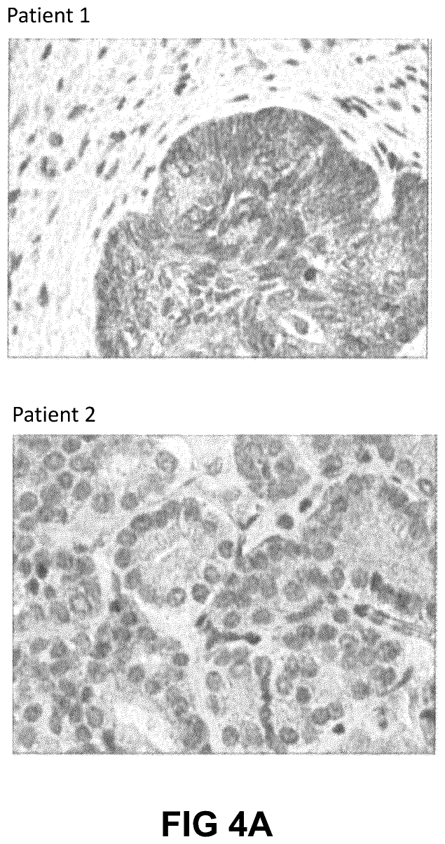

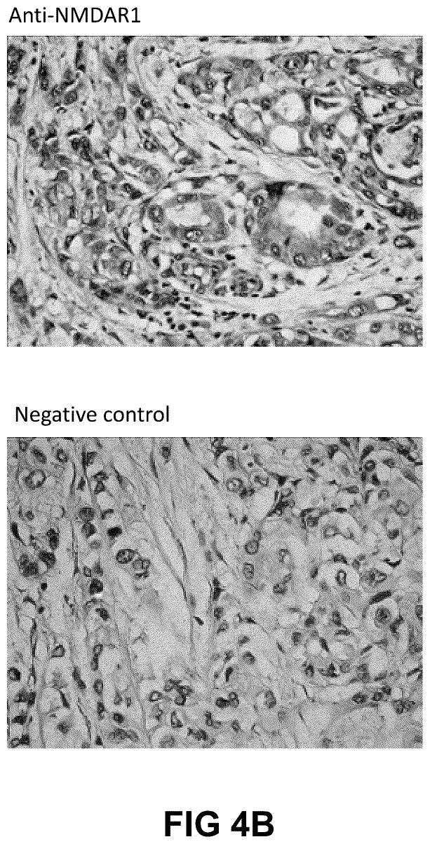

FIGS. 4A-4B show exemplary NMDAR1 staining by immunohistochemistry on pancreatic cancer sections. Sections shown in FIG. 4A are from two pancreatic cancer patients, as described in Example 1. FIG. 4B provides pancreatic adenocarcinoma immunohistochemistry stained with antibodies to NMDAR1 (top) compared with antibody negative control (bottom).

FIG. 5 shows exemplary NMDAR1 staining by confocal microscopy on LNCAP cells in Example 2.

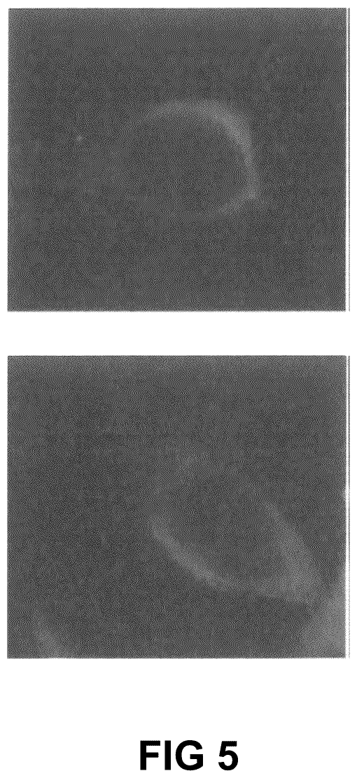

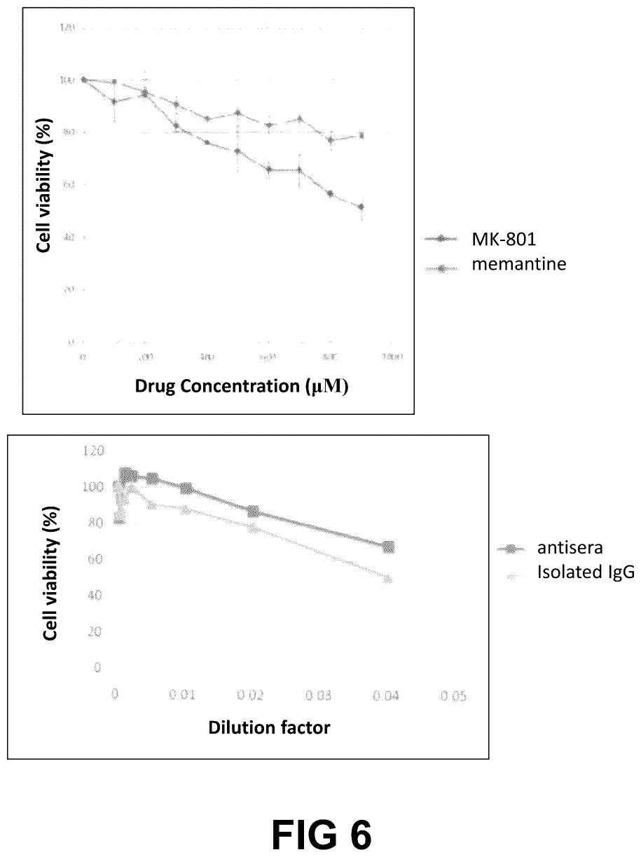

FIG. 6 shows exemplary results of the cell viability assay in Example 3. Effects of different amounts of NMDA receptor antagonists, MK-801 and memantine, and anti-NMDAR1 antisera and IgG on LNCAP cell viability are shown.

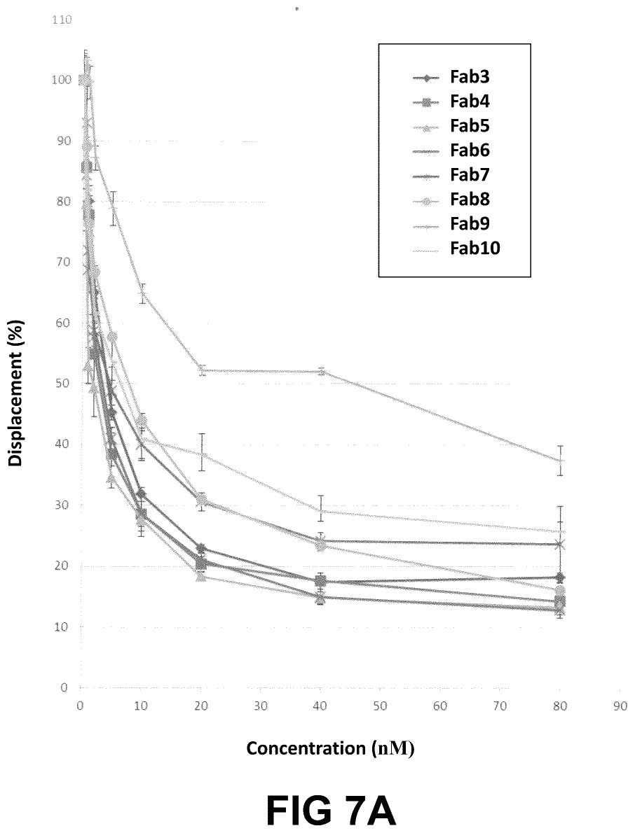

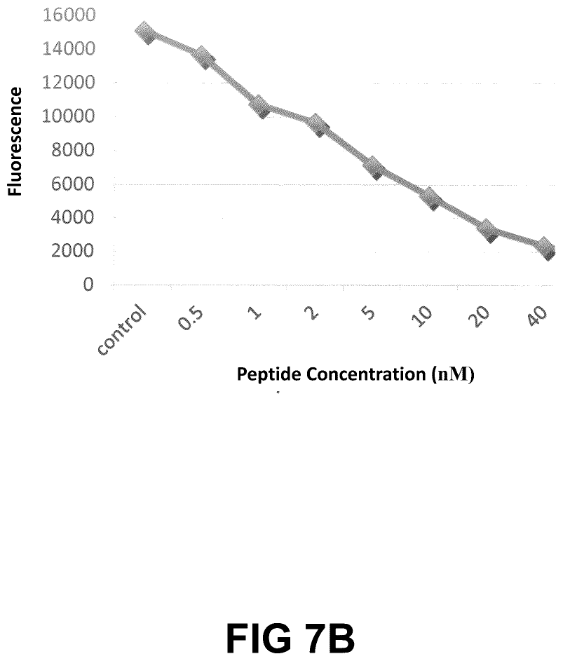

FIGS. 7A-7B contain shows exemplary results of the competition displacement assay in Example 8. Labeled BSA-coupled NMDAR2B immunogen peptides bound to Fabs were displaced by increasing amounts of unlabeled BSA-coupled NMDAR2B immunogen peptides. FIG. 7B shows exemplary results comparing displacement on Fab3 through Fab10. FIG. 7B shows exemplary results on Fab9.

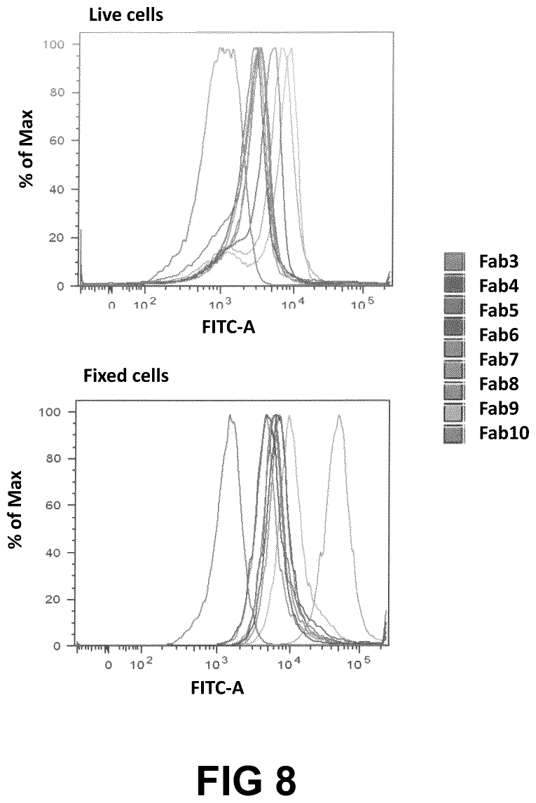

FIG. 8 provides exemplary flow cytometry results in Example 9. Histograms showing binding of Fab3 through Fab10 to live (top) and paraformaldehyde-fixed (bottom) NCI-H82 cells are shown.

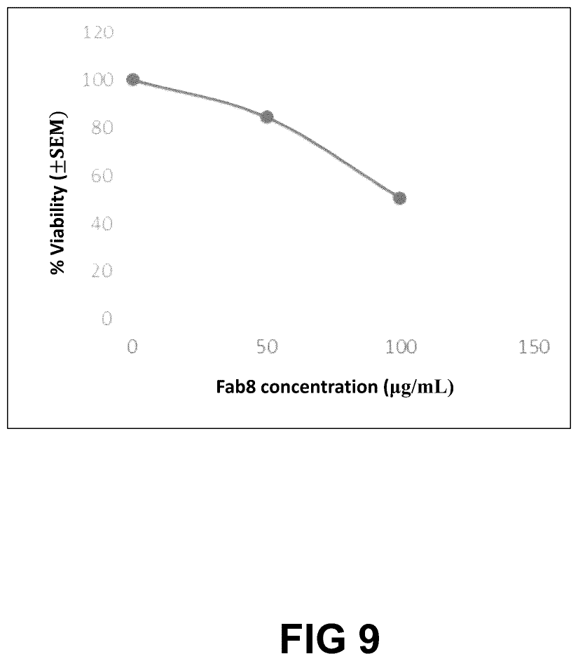

FIG. 9 provides exemplary results of the cell viability assay in Example 10. Effects of different concentrations of Fab8 on NCI-H82 cell viability are shown.

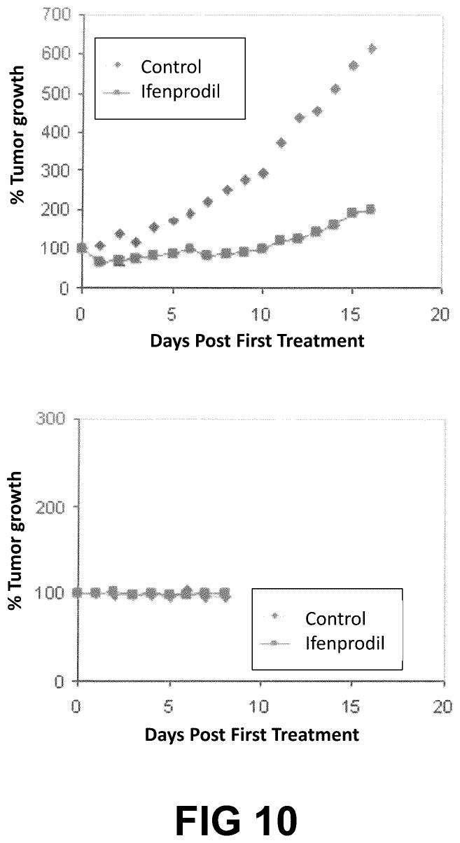

FIG. 10 provides exemplary results of in vivo treatment in Example 11. Changes in the tumor size (top) and body weight (bottom) in mice treated with ifenprodil (diamond) or saline control (square) are shown.

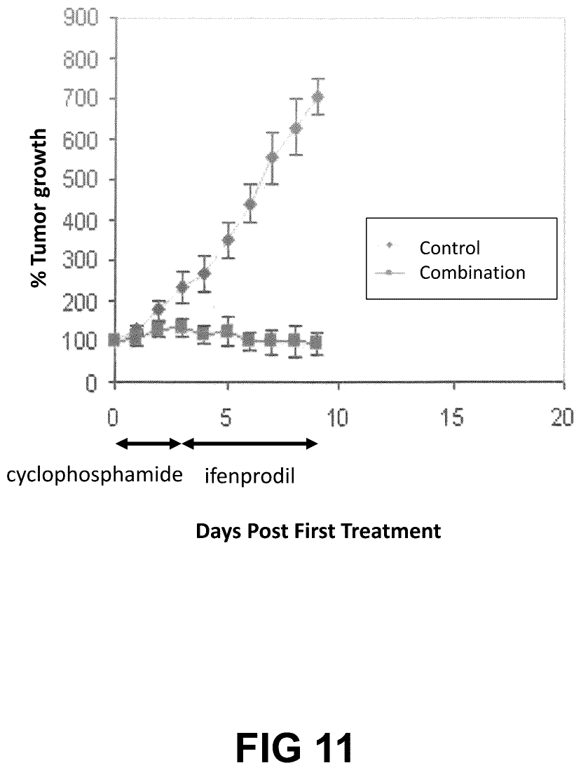

FIG. 11 provides exemplary results of in vivo combination treatment in Example 12. Changes in the tumor size in mice treated with saline control (diamond) and mice that received the combination therapy, which is cyclophosphamide followed by ifenprodil (square) are compared. The time periods of cyclophosphamide treatment and ifenprodil treatment are respectively shown with arrows.

### DETAILED DESCRIPTION

An aspect of the invention in general relates to novel NMDAR2B-binding agents and use thereof.

In one aspect, the anti-NMDAR2B agent is, for example, but not limited to, anti-NMDAR2B antibodies (Abs), antigen-binding Ab fragments, multi-specific Abs, multi-specific antigen-binding Ab fragments, antibody-drug conjugates (ADCs), chimeric antigen receptors (CARs), and cells expressing anti-NMDAR2B CARs.

The present disclosure also provides polynucleotides and combinations of polynucleotides encoding such an Ab, antigen-binding Ab fragment, multi-specific Ab, multi-specific antigen-binding Ab fragment, ADC, or CAR that binds to NMDAR2B, vectors and combinations of vectors comprising such polynucleotides and combinations of polynucleotides, and cells comprising such an Ab, antigen-binding Ab fragment, multi-specific Ab, multi-specific antigen-binding Ab fragment, ADC, or CAR, such a polynucleotide or a combination of polynucleotides, or such a vector or a combination of vectors. The present disclosure also provides compositions comprising such an Ab, antigen-binding Ab fragment, multi-specific Ab, multi-specific antigen-binding Ab fragment, ADC, CAR, such a polynucleotide or a combination of polynucleotides, or such a vector or a combination of vectors, or such a cell.

The present disclosure further provides methods of making and using an NMDAR2B-binding Ab, antigen-binding Ab fragment, multi-specific Ab, multi-specific antigen-binding Ab fragment, ADC, or CAR, or cells expressing an NMDAR2B-binding Ab, antigen-binding Ab fragment, multi-specific Ab, multi-specific antigen-binding Ab fragment, ADC, or CAR. The invention also provides methods for treating a subject or treating or diagnosing a disease such as one associated with NMDAR2B expression in a subject, such as cancer, using anti-NMDAR2B Abs, antigen-binding Ab fragments, multi-specific Abs, multi-specific antigen-binding Ab fragments, ADCs, CARs, and cells comprising or comprising a polynucleotide(s) encoding such an NMDAR2B-binding Ab, antigen-binding Ab fragment, multi-specific Ab, multi-specific antigen-binding Ab fragment, ADC, or CAR.

### Binding Target

In one aspect, the anti-NMDAR2B agents of the present invention may bind to a NMDA receptor.

NMDARs are glutamate-gated ionotropic receptor with high calcium permeability (Blanke M L and VanDongen AMJ. Activation Mechanisms of the NMDA Receptor. In: Van Dongen A M, editor. Biology of the NMDA Receptor. Boca Raton (FL): CRC Press/Taylor & Francis; 2009. Chapter 13), and are involved in neuronal development, synaptic plasticity, learning, and memory (Blanke M L and VanDongen AMJ. Activation Mechanisms of the NMDA Receptor. In: Van Dongen A M, editor. Biology of the NMDA Receptor. Boca Raton (FL): CRC Press/Taylor & Francis; 2009. Chapter 13). NMDARs are generally expressed as heterotetramers of two glycin-binding NR1 subunits and two glutamate-binding NR2 subunits assembled around a central permeation pathway, and binding of both ligands, glycine and glutamate, is required for NMDAR activation.

NR1 and NR2 share the basic structure comprising an N-terminal domain (NTD), two ligand-binding domains (S1 and S2) forming a ligand binding site (for glycine or glutamate), four hydrophobic domains (M1, M2, M3, and M4), and a C-terminal domain (CTD). The domains within the NR1 or NR2 subunit protein are in the order of NTD-S1-M1-M2-M3-S2-M4-CTD, from the N-terminus to the C-terminus. The NTD, S1, and S2 are extracellular, M1, M3, and M4 are predicted to cross the membrane, and M2 is predicted to be cytoplasmic, and CTD localizes to the cytoplasm. S1 and S2. There is one NR1 subunit-encoding gene and four NR2 subunit-encoding genes (A, B, C, and D).

NMDA receptor subtype 2B (NMDAR2B), is one of the four subtypes of NR2 (Paoletti P, et al. Nat Rev Neurosci. 2013 June; 14(6):383-400) and is also referred to as NMDA receptor subunit epsilon-2, NR2B, hNR3, glutamate receptor subunit epsilon-2, GluN2B, glutamate ionotropic receptor NMDA type subunit 2B, GRIN2B, MRD6, EIEE27NR3, DEE27. In humans, NMDAR2B is encoded by the GRIN2B gene on chromosome 12, with gene location 12p13.1 (NCBI). Human NMDAR2B may have an amino acid sequence provided as NCBI Reference Sequence: NP_000825.2. In one aspect, human NMDAR2B has the amino acid sequence provided as SEQ ID NO: 1 (which may be encoded by the nucleic acid sequence of SEQ ID NO: 11) or the equivalent residues from a non-human species, e.g., mouse, rodent, monkey, ape and the like.

In one aspect, the anti-NMDAR2B agents of the present invention may specifically bind to NMDAR2B.

In one aspect, the target, or the binding target, of the anti-NMDAR2B agents of the present invention is NMDAR2B.

In one aspect, the anti-NMDAR2B Ab, anti-NMDAR2B antigen-binding Ab fragment, anti-NMDAR2B multi-specific Ab, anti-NMDAR2B multi-specific antigen-binding Ab fragment, anti-NMDAR2B ADC, and anti-NMDAR2B CAR of the present disclosure individually comprise an antigen-binding domain which binds to NMDAR2B.

In healthy humans, the protein expression of NMDAR2B is limited to the brain, especially, the cerebellum (https://www.proteinatlas.org/ENSG00000273079-GRIN2B/tissue). However, NMDAR2B is upregulated in different types of cancer such as, but not limited to, small-cell lung cancer (SCLC) (North W. et al., Clinical Pharmacology: Advances and Applications. 2010:2 31-40), pancreatic cancer (North W. et al., Clinical Pharmacology: Advances and Applications. 2017:9 79-86), ovarian cancer (North W. et al., Clinical Pharmacology: Advances and Applications. 2015:7 111-117), and breast cancer (North W. et al., Breast Cancer Res Treat. 2010 July; 122(2): 307-314.), and glioma (Gao X et al., Neurosurg Focus. 2014 December; 37(6):E17.), and also likely prostate cancer. Studies further report that NMDAR2B is also expressed in, for example, hepatoma (Yamaguchi F., BMC Cancer. 2013 Oct. 10; 13:468.), colon cancer (Stepulak A. et al., Histochem Cell Biol. 2009 October; 132(4):435-45.), thyroid cancer (Stepulak A. et al., Histochem Cell Biol. 2009 October; 132(4):435-45.), laryngeal cancer (Stepulak A. et al., Anticancer Res. 2011 February; 31(2):565-73.).

NMDAR2B-containing NMDA receptors seem to have roles in cancer progression and pathology. For example, Li and Hanahan (Li L. and Hanahan D. Cell. 2013 Mar. 28; 153(1):86-100.) demonstrated using a mouse model of pancreatic ductal adenocarcinoma that NMDAR2B expression is elevated and NMDAR2B phosphorylation at Y1252, which enhances NMDAR activity, is more pronounced toward the periphery of tumors, particularly at invasion fronts, and NMDAR2B expression is positively correlated with tumor size. The study describes a similar observation with a human breast cancer sample. Li and Hanahan further found that, in human glioblastoma (grade 4 glioma) and in human ovarian cancer, patients with low expression of NMDAR2B and vesicular glutamate transporter 2 (vGlut2) has much longer survival compared to patients with high NMDAR2B and vGlut2 expression. Therefore, NMDAR2B is predictive of poor prognosis for cancer patients. Furthermore, overactivation of NMDAR2B is implicated in peritumoral seizures, an early symptom of glioma (Gao X et al., Neurosurg Focus. 2014 December; 37(6):E17.).

Therefore, in some embodiments, the anti-NMDAR2B agents of the present invention may bind to or target NMDAR2B on cancer cells, such as cells of any of the above-mentioned cancer types.

In some embodiments, the anti-NMDAR2B agents disclosed herein may bind SCLC cancer cells. In some embodiments, the anti-NMDAR2B agents disclosed herein may bind pancreatic cancer cells. In some embodiments, the anti-NMDAR2B agents disclosed herein may bind breast cancer cells. In some embodiments, the anti-NMDAR2B agents disclosed herein may bind ovarian cancer cells. In some embodiments, the anti-NMDAR2B agents disclosed herein may bind glioma cells. In some embodiments, the anti-NMDAR2B agents disclosed herein may bind prostate cancer cells.

NMDAR2B is also implicated in diseases other than cancer. For example, cerebral ischemia causes brain damage by recruiting death-associated protein kinase 1 (DAPK1) to and phosphorylating the C-terminus of NMDAR2B, thereby activating the NMDAR receptor signaling (Tu W. et al., Cell. 2010 Jan. 22; 140(2):222-34.). The role of NMDAR2B in neurodegenerative diseases have been also studied. NMDAR2B expression is elevated in mouse (Maher A. et al., Brain Res Bull. 2014 October; 109:109-16.) and rat (Liu Z. et al., Neurochem Res. 2012 July; 37(7):1420-7.) models of Alzheimer's disease, and the excitotoxicity in Alzheimer's disease is associated with extrasynaptic NMDAR2B-containing NMDARs (Liu J. et al., Front Neurosci. 2019 Feb. 8; 13:43.). In Huntington disease, NMDAR2B is elevated in striatal medium-sized spiny projection neurons (MSNs), the neurons most severely undergo neurodegeneration, and the NMDAR2B-containing NMDA receptor signaling is potentiated by the disease-causing mutant huntingtin (Li L. et al., Neurobiol Aging. 2003 December; 24(8):1113-21.).

Therefore, in some embodiments, the anti-NMDAR2B agents of the present invention may bind to or target NMDAR2B on neural cells, such as those associated with the above-mentioned neural diseases.

### Anti-NMDAR2B Antibody, Antigen-Binding Fragment, Multi-Specific Antibody, Multi-Specific Antigen-Binding Fragment, and Antibody-Drug Conjugate

In some embodiments, the anti-NMDAR2B antibody (Ab), anti-NMDAR2B antigen-binding (AB) fragment, anti-NMDAR2B multi-specific Ab, anti-NMDAR2B multi-specific antigen-binding Ab fragment, and anti-NMDAR2B antibody-drug conjugate (ADC) of the present invention, individually comprise at least one antigen-binding domain that binds to NMDAR2B.

Eight novel human anti-NMDAR2B antibodies, named Ab3, Ab4, Ab5, Ab6, Ab7, Ab8, Ab9, and Ab10 were identified as described in Example 4 and FIGS. 1A-1C.

Ab3 comprises: (a) a VH and (b) a VL sequence as set forth in SEQ ID NO: 36, encoded by SEQ ID NO: 136. The CDR 1 and CDR 3 of the VH (i.e., CDR-H1 and CDR-H3) comprise the amino acid sequence of SEQ ID NOs: 32, and 34, respectively, encoded by SEQ ID NOs: 132 and 134, respectively. The CDR1, CDR 2, and CDR 3 of the VL (i.e., CDR-L1, CDR-L2, and CDR-L3) comprise the amino acid sequence of SEQ ID NOs: 37, 38, and 39, respectively, encoded by SEQ ID NOs: 137, 138, and 139, respectively. The light chain amino acid sequence is SEQ ID NO: 35, encoded by SEQ ID NO: 135.

Ab4 comprises: (a) a VH sequence as set forth in SEQ ID NO: 41, encoded by SEQ ID NO: 141; and (b) a VL sequence as set forth in SEQ ID NO: 46, encoded by SEQ ID NO: 146. The CDR 1, CDR 2, and CDR 3 of the VH (i.e., CDR-H1, CDR-H2, and CDR-H3) comprise the amino acid sequence of SEQ ID NOs: 42, 43, and 44, respectively, encoded by SEQ ID NOs: 142, 143, and 144, respectively. The CDR1, CDR 2, and CDR 3 of the VL (i.e., CDR-L1, CDR-L2, and CDR-L3) comprise the amino acid sequence of SEQ ID NOs: 47, 48, and 49, respectively, encoded by SEQ ID NOs: 147, 148, and 149, respectively. The heavy and light chain amino acid sequences are SEQ ID NOS: 40 and 45, respectively, encoded by SEQ ID NOS: 140 and 145, respectively.

Ab5 comprises: (a) a VH sequence as set forth in SEQ ID NO: 51, encoded by SEQ ID NO: 151; and (b) a VL sequence as set forth in SEQ ID NO: 56, encoded by SEQ ID NO: 156. The CDR 1, CDR 2, and CDR 3 of the VH (i.e., CDR-H1, CDR-H2, and CDR-H3) comprise the amino acid sequence of SEQ ID NOs: 52, 53, and 54, respectively, encoded by SEQ ID NOs: 152, 153, and 154, respectively. The CDR1, CDR 2, and CDR 3 of the VL (i.e., CDR-L1, CDR-L2, and CDR-L3) comprise the amino acid sequence of SEQ ID NOs: 57, 58, and 59, respectively, encoded by SEQ ID NOs: 157, 158, and 159, respectively. The heavy and light chain amino acid sequences are SEQ ID NOS: 50 and 55, respectively, encoded by SEQ ID NOS: 150 and 155, respectively.

Ab6 comprises: (a) a VH sequence as set forth in SEQ ID NO: 61, encoded by SEQ ID NO: 161; and (b) a VL sequence as set forth in SEQ ID NO: 66, encoded by SEQ ID NO: 166. The CDR 1, CDR 2, and CDR 3 of the VH (i.e., CDR-H1, CDR-H2, and CDR-H3) comprise the amino acid sequence of SEQ ID NOs: 62, 63, and 64, respectively, encoded by SEQ ID NOs: 162, 163, and 164, respectively. The CDR1, CDR 2, and CDR 3 of the VL (i.e., CDR-L1, CDR-L2, and CDR-L3) comprise the amino acid sequence of SEQ ID NOs: 67, 68, and 69, respectively, encoded by SEQ ID NOs: 167, 168, and 169, respectively. The heavy and light chain amino acid sequences are SEQ ID NOS: 60 and 65, respectively, encoded by SEQ ID NOS: 160 and 165, respectively.

Ab7 comprises: (a) a VH sequence as set forth in SEQ ID NO: 71, encoded by SEQ ID NO: 171; and (b) a VL sequence as set forth in SEQ ID NO: 76, encoded by SEQ ID NO: 176. The CDR 1, CDR 2, and CDR 3 of the VH (i.e., CDR-H1, CDR-H2, and CDR-H3) comprise the amino acid sequence of SEQ ID NOs: 72, 73, and 74, respectively, encoded by SEQ ID NOs: 172, 173, and 174, respectively. The CDR1, CDR 2, and CDR 3 of the VL (i.e., CDR-L1, CDR-L2, and CDR-L3) comprise the amino acid sequence of SEQ ID NOs: 77, 78, and 79, respectively, encoded by SEQ ID NOs: 177, 178, and 179, respectively. The heavy and light chain amino acid sequences are SEQ ID NOS: 70 and 75, respectively, encoded by SEQ ID NOS: 170 and 175, respectively.

Ab8 comprises: (a) a VH sequence as set forth in SEQ ID NO: 81, encoded by SEQ ID NO: 181; and (b) a VL sequence as set forth in SEQ ID NO: 86, encoded by SEQ ID NO: 186. The CDR 1, CDR 2, and CDR 3 of the VH (i.e., CDR-H1, CDR-H2, and CDR-H3) comprise the amino acid sequence of SEQ ID NOs: 82, 83, and 84, respectively, encoded by SEQ ID NOs: 182, 183, and 184, respectively. The CDR1, CDR 2, and CDR 3 of the VL (i.e., CDR-L1, CDR-L2, and CDR-L3) comprise the amino acid sequence of SEQ ID NOs: 87, 88, and 89, respectively, encoded by SEQ ID NOs: 187, 188, and 189, respectively. The heavy and light chain amino acid sequences are SEQ ID NOS: 80 and 85, respectively, encoded by SEQ ID NOS: 180 and 185, respectively.

Ab9 comprises: (a) a VH sequence as set forth in SEQ ID NO: 91, encoded by SEQ ID NO: 191; and (b) a VL sequence as set forth in SEQ ID NO: 96, encoded by SEQ ID NO: 196. The CDR 1, CDR 2, and CDR 3 of the VH (i.e., CDR-H1, CDR-H2, and CDR-H3) comprise the amino acid sequence of SEQ ID NOs: 92, 93, and 94, respectively, encoded by SEQ ID NOs: 192, 193, and 194, respectively. The CDR1, CDR 2, and CDR 3 of the VL (i.e., CDR-L1, CDR-L2, and CDR-L3) comprise the amino acid sequence of SEQ ID NOs: 97, 98, and 99, respectively, encoded by SEQ ID NOs: 197, 198, and 199, respectively. The heavy and light chain amino acid sequences are SEQ ID NOS: 90 and 95, respectively, encoded by SEQ ID NOS: 190 and 195, respectively.

Ab10 comprises: (a) a VH sequence as set forth in SEQ ID NO: 101, encoded by SEQ ID NO: 201; and (b) a VL sequence as set forth in SEQ ID NO: 106, encoded by SEQ ID NO: 206. The CDR 1, CDR 2, and CDR 3 of the VH (i.e., CDR-H1, CDR-H2, and CDR-H3) comprise the amino acid sequence of SEQ ID NOs: 102, 103, and 104, respectively, encoded by SEQ ID NOs: 202, 203, and 204, respectively. The CDR1, CDR 2, and CDR 3 of the VL (i.e., CDR-L1, CDR-L2, and CDR-L3) comprise the amino acid sequence of SEQ ID NOs: 107, 108, and 109, respectively, encoded by SEQ ID NOs: 207, 208, and 209, respectively. The heavy and light chain amino acid sequences are SEQ ID NOS: 100 and 105, respectively, encoded by SEQ ID NOS: 200 and 205, respectively.

In some embodiments, the NMDAR2B-binding domain of the anti-NMDAR2B Abs or antigen-binding Ab fragments according to the present disclosure may comprise a VH and a VL, which respectively comprise the three CDRs of the VH and the three CDRs of the VL of the anti-NMDAR2B monoclonal antibody, Ab3, Ab4, Ab5, Ab6, Ab7, Ab8, Ab9, or Ab10, as disclosed herein.

In some particular embodiments, the VH and VL of the anti-NMDAR2B Abs or antigen-binding Ab fragments according to the present disclosure may comprise the three CDRs of the VH and the three CDRs of the VL of the anti-NMDAR2B monoclonal antibody Ab8 or Ab9.

In some embodiments, the NMDAR2B-binding domain of the anti-NMDAR2B Abs or antigen-binding Ab fragments according to the present disclosure may comprise a VH and a VL, which respectively comprise the CDR-H1, CDR-H2, and CDR-H3 of the VH and the CDR-L1, a CDR-L2, and CDR-L3 of the VL, of the anti-NMDAR2B monoclonal antibody, Ab3, Ab4, Ab5, Ab6, Ab7, Ab8, Ab9, or Ab10, as disclosed herein.

In some particular embodiments, the VH and VL of the anti-NMDAR2B Abs or antigen-binding Ab fragments according to the present disclosure may comprise the CDR-H1, CDR-H2, and CDR-H3 of the VH and the CDR-L1, a CDR-L2, and CDR-L3 of the VL, of the anti-NMDAR2B monoclonal antibody Ab8 or Ab9.

Antibody binding specificity is determined by the CDR sequences. Therefore, Abs and Ab fragments having the combination of the six CDR amino acid sequences of any one of Ab3 through Ab10 but having the framework amino acid sequences varied from those of the VH and VL of Ab3 through Ab10 are also provided herein.

Therefore, in some embodiments, the NMDAR2B-binding domain of the anti-NMDAR2B Abs or antigen-binding Ab fragments according to the present disclosure may comprise: (a) a VH comprising (a-1) the three CDRs of the VH of Ab3, Ab4, Ab5, Ab6, Ab7, Ab8, Ab9, or Ab10 and (a-2) at least 80%, at least 85%, at least 90%, at least 92%, at least 94%, at least 95%, at least 96%, at least 97%, at least 98%, at least 99%, or 100% identical to the VH of Ab3, Ab4, Ab5, Ab6, Ab7, Ab8, Ab9, or Ab10, respectively; and (b) a VL comprising (b-1) the three CDRs of the VL of Ab3, Ab4, Ab5, Ab6, Ab7, Ab8, Ab9, or Ab10 and (b-2) at least 80%, at least 85%, at least 90%, at least 92%, at least 94%, at least 95%, at least 96%, at least 97%, at least 98%, at least 99%, or 100% identical to the VL of Ab3, Ab4, Ab5, Ab6, Ab7, Ab8, Ab9, or Ab10, respectively.

In some particular embodiments, the NMDAR2B-binding domain of the anti-NMDAR2B Abs or antigen-binding Ab fragments according to the present disclosure may comprise: (a) a VH comprising (a-1) the three CDRs of the VH of Ab8 or Ab9 and (a-2) at least 80%, at least 85%, at least 90%, at least 92%, at least 94%, at least 95%, at least 96%, at least 97%, at least 98%, at least 99%, or 100% identical to the VH of Ab8 or Ab9, respectively; and (b) a VL comprising (b-1) the three CDRs of the VL of Ab8 or Ab9, respectively, and (b-2) at least 80%, at least 85%, at least 90%, at least 92%, at least 94%, at least 95%, at least 96%, at least 97%, at least 98%, at least 99%, or 100% identical to the VL of Ab8 or Ab9, respectively.

In some embodiments, the variable domain of an anti-NMDAR2B agent disclosed herein may be altered without inhibiting NMDAR2B binding. As long as the antigen-binding domain sufficiently binds to NMDAR2B, the sequence of the variable domain may be altered. Antigen-Ab interactions are largely determined by six CDRs, while a person of skilled in the art will appreciate that some deviation from the exact CDR sequences may be possible. Any suitable techniques such as affinity maturation can be used to alter the CDR sequence. Among the six CDRs, CDR-H3 and CDR-L3 are generally believed as the key determinant of specificity in antigen recognition. Particularly, diversity in the CDR 3 of VH (i.e., CDR-H3) may be particularly important for providing most antibody specificities (Xu J. L., Immunity. 2000 July; 13(1):37-45). Therefore, one or more mutations may be incorporated in the CDR 1 and/or CDR 2 without greatly decreasing the binding affinity while achieving a more desired property of an Ab.

Therefore, Abs or antigen-binding Ab fragments comprising CDR-H1, CDR-H2, CDR-L1, CDR-L2, and/or CDR-L3 with at least 80%, at least 85%, at least 90%, at least 92%, at least 94%, at least 95%, at least 96%, at least 97%, at least 98%, at least 99%, or 100% sequence identity to the CDR-H1, CDR-H2, CDR-L1, CDR-L2, and/or CDR-L3 disclosed herein, respectively, are also within the scope of the present invention. Furthermore, one or more mutations in CDR-H3 may be incorporated to modify, increase, or fine tune the binding or any other properties of the antigen-binding domain. Alternatively, since any one mutation may alter biochemical properties such as thermodynamic stability or immunogenicity in addition to the affinity, all possible mutations in any of the six CDRs, and/or any combination thereof, and/or even in the framework sequence, may be tested to see if the sequence modification provides an improved or more desired overall property (Rajpal A. et al., Proc Natl Acad Sci USA. 2005 Jun. 14; 102(24): 8466-8471. doi: 10.1073/pnas.0503543102; Julian M. C. et al., Sci Rep. 2017; 7: 45259. Published online 2017 Mar. 28. doi: 10.1038/srep45259). To test the binding of a modified Ab to the target, any appropriate technique such as, but not limited to, ELISA, RIA, FACS, bioassay, or Western Blot assay may be used.

In some embodiments, the anti-NMDAR2B Ab or Ab fragment of the present disclosure may be: a human, humanized, or chimeric Ab or Ab fragment; monospecific, bispecific, or multispecific; monovalent, bivalent, trivalent, tetravalent, pentavalent, hexavalent, or multivalent; monomeric, dimeric, trimeric, tetrameric, pentameric, or multimeric; recombinant or synthetic.

The anti-NMDAR2B Ab or Ab fragment of the present disclosure may be or may comprise a full-size or intact immunoglobulin (Ig) molecule or any appropriate antigen-binding Ab fragments thereof.

Therefore, in some embodiments, the anti-NMDAR2B Ab or Ab fragment of the present disclosure may be or may comprise: an IgM, IgD, IgG, IgE, or IgA; an IgG1, IgG2, IgG3, IgG4, IgA1, or IgA2; a single chain Ab, a domain-deleted Ab, a hybrid Ab, CDR-grafted Ab, a fragment antigen-binding (Fab), a F(ab′)2, a Fab′ fragment, a variable fragment (Fv), a single-chain Fv (scFv), an Fd fragment, a scFv-Fc, an scFc fusion protein, a diabody, or a minibody; or any combination thereof.

In some embodiments, the anti-NMDAR2B Ab or Ab fragment of the present disclosure may be or may comprise, an IgM, IgD, IgG, IgE, or IgA, which comprise a heavy chain and a light chain. The heavy and light chains may comprise any of the VH and VL combinations described above.

Antibody binding specificity is determined by the CDR sequences. Therefore, Abs and Ab fragments having the combination of the six CDR amino acid sequences of any one of Ab3 through Ab10 but having the framework amino acid sequences and constant region sequences varied from those of the VH and VL of Ab3 through Ab10 are also provided herein.

Therefore, in some embodiments, the NMDAR2B-binding domain of the anti-NMDAR2B Abs or antigen-binding Ab fragments according to the present disclosure may comprise: (a) a heavy chain comprising (a-1) the three CDRs of Ab3, Ab4, Ab5, Ab6, Ab7, Ab8, Ab9, or Ab10 and (a-2) at least 80%, at least 85%, at least 90%, at least 92%, at least 94%, at least 95%, at least 96%, at least 97%, at least 98%, at least 99%, or 100% identical to the heavy chain of Ab3, Ab4, Ab5, Ab6, Ab7, Ab8, Ab9, or Ab10, respectively; and (b) a light chain comprising (b-1) the three CDRs of Ab3, Ab4, Ab5, Ab6, Ab7, Ab8, Ab9, or Ab10 and (b-2) at least 80%, at least 85%, at least 90%, at least 92%, at least 94%, at least 95%, at least 96%, at least 97%, at least 98%, at least 99%, or 100% identical to the light chain of Ab3, Ab4, Ab5, Ab6, Ab7, Ab8, Ab9, or Ab10, respectively.

In some particular embodiments, the NMDAR2B-binding domain of the anti-NMDAR2B Abs or antigen-binding Ab fragments according to the present disclosure may comprise: (a) a heavy chain comprising (a-1) the three CDRs of Ab8 or Ab9 and (a-2) at least 80%, at least 85%, at least 90%, at least 92%, at least 94%, at least 95%, at least 96%, at least 97%, at least 98%, at least 99%, or 100% identical to the heavy chain of Ab8 or Ab9, respectively; and (b) a light chain comprising (b-1) the three CDRs of Ab8 or Ab9 and (b-2) at least 80%, at least 85%, at least 90%, at least 92%, at least 94%, at least 95%, at least 96%, at least 97%, at least 98%, at least 99%, or 100% identical to the light chain of Ab8 or Ab9, respectively.

scFv

In some embodiments the anti-NMDAR2B agent is a scFv comprising a VH, a linker, and a VL, where the VH and VL combinations are any of the combinations described above. The scFv according to the present disclosure may comprise: a VH, a linker, and a VL, or a VL, a linker, and a VH, in the direction from the N-terminus to the C-terminus. Any appropriate linker that allows the VH and VL to form an antigen-binding site that binds to NMDAR2B may be used.

In some embodiments, the linker may be a GS linker, which is one or more repeats of a sequence unit containing G (glycine) and S (serine), such as the GS linker unit of SEQ ID NO: 110. For example, the G4SX3 linker of SEQ ID NO: 111 may be used.

In some embodiments, the anti-NMDAR2B scFv according to the present disclosure may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 240, 245, 250, 255, 260, 265, 270, 275, 280, 285, 290, 295, 300, or 305 and comprises the same CDRs as those contained in said respective SEQ ID NOS.

In particular embodiments, the anti-NMDAR2B scFv according to the present disclosure may comprise an amino acid sequence which is at least 80%, at least 85%, at least 90%, at least 92%, at least 94%, at least 95%, at least 96%, at least 97%, at least 98%, at least 99%, or 100% identical to SEQ ID NO: 270, 275, 280, or 285 and comprises the same six CDRs as those contained in said respective SEQ ID NOS.

SEQ ID NO:240, 245, 250, 255, 260, 265, 270, 275, 280, 285, 290, 295, 300,or 305 may be encoded by the nucleic acid sequence of SEQ ID NO: 340, 345, 350, 355, 360, 365, 370, 375, 380, 385, 390, 395, 400, or 405, respectively.

### Multiple Specificity

In some embodiments, the anti-NMDAR2B Abs or Ab fragments of the present invention may comprise two or more binding specificities (i.e., bispecific, trispecific, or multispecific generally). The first specificity is to an epitope in NMDAR2B (first NMDAR2B epitope).

In one aspect, the anti-NMDAR2B Abs or Ab fragments of the present disclosure may have a second binding specificity to another epitope (i.e., second NMDAR2B epitope) in NMDAR2B. The second NMDAR2B epitope may or may not be overlapped with the first NMDAR2B epitope.

In another aspect, the second specificity may be to an epitope in a second antigen other than NMDAR2B. A multi-specific anti-NMDAR2B Ab or Ab fragment according to the present disclosure may bind to NMDAR2B and one or more other targets. In some embodiments, a multi-specific anti-NMDAR2B Ab or Ab fragment binds to NMDAR2B and a protein on an effector cell. In some embodiments, a multi-specific anti-NMDAR2B Ab or Ab fragment binds to NMDAR2B and a protein on a target (e.g., cancer) cell. In some embodiments, binding to a second antigen may improve functional characteristics of the anti-NMDAR2B Ab or Ab fragment, e.g., recruitment, effector functions, lysis of target cells.

In certain embodiments, the second antigen may be, for example without limitation, CD3, NKG2D, 4-1BB, or an Fc receptor (FcR), such as Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn). For anti-NMDAR2B Abs and antigen-binding Ab fragments, having a specificity to an FcR allows FcR-mediated effects such as antibody-dependent cellular phagocytosis (ADCP) or antibody-dependent cellular cytotoxicity (ADCC) of NMDAR2B-expressing cells or cytotoxic mediator release by Fc-expressing cells.

When the anti-NMDAR2B agent has two specificities, the agent may be called bispecific. Bispecific anti-NMDAR2B agents include bispecific anti-NMDAR2B Abs or antigen-binding Ab fragments. When the anti-NMDAR2B agent has two or more specificities, the agent may be called multispecific. Multispecific anti-NMDAR2B agents include multispecific anti-NMDAR2B Abs or antigen-binding Ab fragments.

The present invention encompasses any types of bispecific Ab-like molecules (Abs or antigen-binding Ab fragments) such as reviewed in Brinkmann U. et al., MAbs. 2017 February-March; 9(2): 182-212. Published online 2017 Jan. 10. doi: 10.1080/19420862.2016.1268307; Klein C. et al., MAbs. 2016 August-September; 8(6):1010-20. doi: 10.1080/19420862.2016.1197457. In a bispecific embodiment according to the present disclosure, one of the antigen-binding domains is an anti-NMDAR2B binding domain. General methods for designing and construction of bispecific or multispecific Abs or antigen-binding Ab fragments are known in the art (Brinkmann U. et al., MAbs. 2017 February-March; 9(2): 182-212. Published online 2017 Jan. 10. doi: 10.1080/19420862.2016.1268307; Dimasi N. et al. Methods. 2018 Aug. 11. pii: S1046-2023(18)30149-X. doi: 10.1016/j.ymeth.2018.08.004; Sedykh S. E. et al., Drug Des Devel Ther. 2018; 12: 195-208). Such methods include chemical conjugation, covalent attachment of fragments, and genetic engineering. For example, full-length bispecific Abs or antigen-binding Ab fragments may be generated by co-expressing two pairs of heavy and light chains, each pair having different specificities. The two pairs may be encoded in one vector, or encoded in separate vectors but expressed in the same host cell. Alternatively, antigen-binding Ab fragments or the antigen-binding domains having different specificities may be generated separately and then conjugated to one another, for example using sulfhydryl bonding (of, for example, the VH C-terminus hinge regions) and/or an appropriate coupling or crosslinking agent. Bispecific antigen-binding Ab fragments may also be generated, for example, by using leucine zippers or by using scFv dimers (see for example, Kosteln et al., J Immunol. 1992 Mar. 1; 148(5):1547-53). Binding of the bispecific agent of the present invention may be confirmed using any appropriate method, such as but not limited to, enzyme-linked immunosorbent assay (ELISA), radioimmunoassay (RIA), flow cytometry, bioassay, or Western blot.

Constant Domains. Fc Region, and Constant Region

In some embodiments, the anti-NMDAR2B agents of the present invention may comprise one or more of the following domains: a CH1 or a variant thereof; a hinge; a CH2 or a variant thereof; a CH3 or a variant thereof; a CLκ or a variant thereof; and/or a CLλ or a variant thereof. Any of the domains may be derived from a corresponding human Ig domain.

In some embodiments, the CH1 or a variant thereof may comprise or consist of the amino acid sequence of: SEQ ID NO: 310 or is at least 80%, at least 85%, at least 90%, at least 92%, at least 94%, at least 95%, at least 96%, at least 97%, at least 98%, or at least 99% identical thereto. SEQ ID NO: 310 may be encoded by the nucleic acid sequence of SEQ ID NO: 410.

In some embodiments, the human hinge may comprise or consist of the amino acid sequence of: SEQ ID NO: 311 or is at least 80%, at least 85%, at least 90%, at least 92%, at least 94%, at least 95%, at least 96%, at least 97%, at least 98%, or at least 99% identical thereto. SEQ ID NO: 311 may be encoded by the nucleic acid sequence of SEQ ID NO: 411.

In some embodiments, the human CH2 or a variant thereof may comprise or consist of the amino acid sequence of: SEQ ID NO: 312 or is at least 80%, at least 85%, at least 90%, at least 92%, at least 94%, at least 95%, at least 96%, at least 97%, at least 98%, or at least 99% identical thereto. SEQ ID NO: 312 may be encoded by the nucleic acid sequence of SEQ ID NO: 412.

In some embodiments, the human CH3 or a variant thereof may comprise or consist of the amino acid sequence of: SEQ ID NO: 313, 314, 315, or 316 or is at least 80%, at least 85%, at least 90%, at least 92%, at least 94%, at least 95%, at least 96%, at least 97%, at least 98%, or at least 99% identical thereto. SEQ ID NO: 313 and 314 have E (glutamate) and M (methionine) at positions 356 and 358, respectively, while SEQ ID NO: 315 and 316 have D (aspartate) and L (leucine) at positions 356 and 358, respectively, and both are allotypes frequently found in humans and show similar FcR binding profiles (Shields et al., J Biol Chem. 2001 Mar. 2; 276(9):6591-604). SEQ ID NO: 313 and 315 have one K (lysine) at the N-terminus, while SEQ ID NO: 314 and 316 do not the N-terminal K. Studies have shown that IgG antibodies often do not have the C-terminal lysine residue due to “C-terminal lysine clipping” (van den Bremer et al. MAbs. 2015; 7(4):672-80.). SEQ ID NO: 313, 314, 315, or 316 may be encoded by the nucleic acid sequence of SEQ ID NO: 413, 414, 415, or 416, respectively.

In some embodiments, the human CLκ or a variant thereof may comprise or consist of the amino acid sequence of: SEQ ID NO: 325 or is at least 80%, at least 85%, at least 90%, at least 92%, at least 94%, at least 95%, at least 96%, at least 97%, at least 98%, or at least 99% identical thereto. SEQ ID NO: 325 may be encoded by the nucleic acid sequence of SEQ ID NO: 425.

In some embodiments, the human CLλ or a variant thereof may comprise or consist of the amino acid sequence of: SEQ ID NO: 326 or is at least 80%, at least 85%, at least 90%, at least 92%, at least 94%, at least 95%, at least 96%, at least 97%, at least 98%, or at least 99% identical thereto. SEQ ID NO: 326 may be encoded by the nucleic acid sequence of SEQ ID NO: 426.

In some embodiments, the anti-NMDAR2B Abs and Ab fragments of the present invention may comprise a Fc region.

In some embodiments, the Fc region may be or may be derived from: a human Fc region; an IgM, an IgD, an IgG, an IgE, or an IgA; or an IgG1, an IgG2, an IgG3, or an IgG4.

In some embodiments, the Fc region may bind to an Fc receptor (FcR). The FcR may be, but is not limited to, Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn).

In some embodiments, the Fc region may comprise or consist of the amino acid sequence of SEQ ID NO: 317, 318, 319, or 320 or is at least 80%, at least 85%, at least 90%, at least 92%, at least 94%, at least 95%, at least 96%, at least 97%, at least 98%, at least 99%, or 100% identical thereto. SEQ ID NOS: 317 and 318 have E (glutamate) and M (methionine) at positions 356 and 358, respectively, and SEQ ID NOS: 319 and 320 have D (aspartate) and L (leucine) at positions 356 and 358, respectively. SEQ ID NO: 317 and 319 have one K (lysine) at the N-terminus, while SEQ ID NO: 318 and 320 do not the N-terminal K. SEQ ID NO: 317, 318, 319, or 320 may be encoded by the nucleic acid sequence of SEQ ID NO: 417, 418, 419, or 420, respectively.

In some embodiments, the anti-NMDAR2B Abs and Ab fragments of the present invention may comprise a heavy chain constant region.

In some embodiments, the heavy chain constant region may be or may be derived from: a human Fc region; an IgM, an IgD, an IgG, an IgE, or an IgA; or an IgG1, an IgG2, an IgG3, or an IgG4.

In some embodiments, the heavy chain constant region may bind to an Fc receptor (FcR). The FcR may be, but is not limited to, Fc gamma receptor (FcγR), FcγRI, FcγRIIA, FcγRIIB1, FcγRIIB2, FcγRIIIA, FcγRIIIB, Fc epsilon receptor (FcεR), FcεRI, FcεRII, Fc alpha receptor (FcαR), FcαRI, Fc alpha/mu receptor (Fcα/μR), and neonatal Fc receptor (FcRn).

In some embodiments, the heavy chain constant region may comprise or consist of the amino acid sequence of SEQ ID NO: 321, 322, 323, or 324 or is at least 80%, at least 85%, at least 90%, at least 92%, at least 94%, at least 95%, at least 96%, at least 97%, at least 98%, at least 99%, or 100% identical thereto. SEQ ID NO: 321, 322, 323, or 324 may be encoded by the nucleic acid sequence of SEQ ID NO: 421, 422, 423, or 424 respectively.

### Constant Region/Fc Region Modifications

In some embodiments, the Fc region and/or the heavy chain constant region is modified to alter at least one of effector function, half-life, proteolysis, or glycosylation.

In some embodiments, the Fc region and/or the heavy chain constant region contains one or more amino acid substitutions or modifications that alters or eliminates N- and/or O-glycosylation.

Certain amino acid modifications in the Fc region and/or the heavy chain constant region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

When the Ab is an IgG1, the Fc region may comprise one or more amino acid substitutions. The substitution may be, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, and/or any combination thereof (the residue numbering is according to EU or Kabat numbering) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73). The Fc region may further comprise one or more additional amino acid substitutions. The substitution may be, for example, but is not limited to, A330L, L234F, L23SE, P3318, and/or any combination thereof (the residue numbering is according to EU or Kabat numbering).

When the Ab is an IgG2, the Fc region may comprise one or more amino acid substitutions. The substitution may be, for example, but is not limited to, P238S, V234A, G237A, H268A, H268Q, H268E, V309L, N297A, N297Q, A330S, P331S, C232S, C233S, M252Y, S254T, T256E, and/or any combination thereof (the residue numbering is according to EU or Kabat numbering). The Fc region may further comprise one or more additional amino acid substitutions. The substitution may be, for example, but is not limited to, M252Y, S254T, T256E, and/or any combination thereof (the residue numbering is according to EU or Kabat numbering).

When the Ab is an IgG3, the Fc region may comprise one or more amino acid substitutions. The substitution may be, for example, but is not limited to, E235Y (the residue numbering is according to EU or Kabat numbering).

When the Ab is an IgG4, the Fc region may comprise one or more amino acid substitutions. The substitution may be, for example, but is not limited to, E233P, F234V, L235A, G237A, E318A, S228P, L236E, S241P, L248E, T394D, M252Y, S254T, T256E, N297A, N297Q, and/or any combination thereof (the residue numbering is according to EU or Kabat numbering). The substitution may be, for example, S228P (the residue numbering is according to EU or Kabat numbering).

In some embodiments, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

### Binding Affinity

The Ab or Ab fragments according to the present disclosure may bind to NMDAR2B, or the human NMDAR2B peptide comprising the amino acid sequence of SEQ ID NO: 2 with a dissociation constant (Kd) of, for example, less than 10 nM.

In some embodiments, the Kd may be about 5 nM.

In some embodiments, the Ab or Ab fragments according to the present disclosure may bind to the human NMDAR2B peptide of SEQ ID NO: 2 or 3 conjugated to a protein.

The Kd values may be determined by any appropriate methods, such as but not limited to surface plasmon resonance (SPR), surface plasmon resonance imaging (SPRi), ELISA, AlphaLISA, radioimmunoassay (RIA), or biolayer interferometry (BLI).

In some embodiments, the Kd is determined by ELISA.

### Target Cell and Effects Thereon

The Ab or Ab fragments according to the present disclosure may bind to NMDAR2B expressed on the surface of cancer cells.

In some particular embodiments, the cancer cells are cells of SCLC, pancreatic cancer, ovarian cancer, breast cancer, prostate cancer, or glioma.

In some embodiments, the Ab or Ab fragments according to the present disclosure is capable of reducing viability of cells expressing human NMDAR2B.

In some particular embodiments, the Ab or Ab fragments may reduce viability of cells of SCLC, pancreatic cancer, ovarian cancer, breast cancer, prostate cancer, or glioma.

In some embodiments, the Ab or Ab fragments according to the present disclosure is capable of inhibiting cancer growth in a subject having cancer, and in some particular embodiments, the cancer may be SCLC, pancreatic cancer, ovarian cancer, breast cancer, or prostate cancer.

In some embodiments, the Ab or Ab fragments according to the present disclosure may work synergistically with another anti-cancer agent in inhibiting cancer growth in a subject having cancer, and in some particular embodiments, the cancer may be SCLC, pancreatic cancer, ovarian cancer, breast cancer, or prostate cancer, and in some particular embodiments, the other anti-cancer agent may be an alkylating agent, optionally cyclophosphamide.

### Ab Conjugation

Any of the Abs or Ab fragments described herein may be attached to at least one effector moiety, chemical inker, detectable moiety, or functional moiety.

In some embodiments, the detectable moiety is a fluorescent dye, an enzyme, a substrate, a bioluminescent material, a radioactive material, a chemiluminescent moiety, or a combination thereof.

In some aspects, the anti-NMDAR2B agent of the present invention may be an antibody-drug conjugate (ADC). The ADC may comprise: (a) any Ab or antigen-binding Ab fragment described herein; and (b) a drug conjugated to the Ab or antigen-binding Ab fragment

### Drugs/Agents to be Conjugated

In some embodiments, the drug may be, but not limited to, an anti-cancer drug, an anti-proliferative drug, a cytotoxic drug, an alkylating agent, a platinum agent, an anthracycline, a taxane, an anti-angiogenic drug, an apoptotic drug, an immunostimulatory drug, an anti-microbial drug, an antibiotic drug, an antiviral drug, an anti-inflammatory drug, an NMDA receptor antagonist, an NMDA receptor signaling inhibitor, an NMDAR1 inhibitor, an NMDAR2B inhibitor, an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, and an imaging drug.

The toxin may be a bacterial, fungal, plant, or animal toxin, or a fragment thereof. Examples include, but are not limited to, diphtheria A chain, diphtheria toxin, exotoxin A chain, ricin A chain, abrin A chain, modeccin A chain, alpha sarcin, Aleurites fordii protein, a dianthin protein, or a Phytolacca Americana protein.

In the clinic, treatment of breast cancer may involve the use of Anthracyclines, such as doxorubicin (Adriamycin) and epirubicin; Taxanes, such as paclitaxel (Taxol) and docetaxel; 5-fluorouracil (5-FU) or capecitabine; Cyclophosphamide; Carboplatin; Taxanes, such as paclitaxel (Taxol), docetaxel, and albumin-bound paclitaxel; Anthracyclines (Doxorubicin, pegylated liposomal doxorubicin, and Epirubicin); Platinum agents (cisplatin, carboplatin); Vinorelbine; Capecitabine; Gemcitabine; and/or Ixabepilone.

In the clinic, treatment of ovarian cancer may involve the use of Albumin bound paclitaxel; Altretamine; Capecitabine; Cyclophosphamide; Etoposide; Gemcitabine; Ifosfamide; Irinotecan; Liposomal doxorubicin; Melphalan; Pemetrexed; Topotecan; Vinorelbine; TIP (paclitaxel/Taxol, ifosfamide, and cisplatin/Platinol); VeIP (vinblastine, ifosfamide, and cisplatin/Platinol); VIP (etoposide/VP-16, ifosfamide, and cisplatin/Platinol); and/or VAC (vincristine, dactinomycin, and cyclophosphamide).

In the clinic, treatment of pancreatic cancer may involve the use of Gemcitabine; 5-fluorouracil (5-FU); Oxaliplatin; Albumin-bound paclitaxel; Paclitaxel; Docetaxel Capecitabine; Cisplatin; Irinotecan; and/or Liposomal Irinotecan.

In the clinic, treatment of prostate cancer may involve the use of Docetaxel; Cabazitaxel; Mitoxantrone; and/or Estramustine.

In the clinic, treatment of SCLC may involve the use of cisplatin, etoposide, carboplatin, and/or irinotecan.

The anti-cancer or anti-proliferative drug in the ADC according to the disclosure may be, for example, but is not limited to, cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU) doxorubicin, daunorubicin, cucurbitacin, chaetocin, chaetoglobosin, chlamydocin, calicheamicin, epirubicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, dactinomycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone, or a derivative thereof (for example, see Polakis P. et al., Pharmacol Rev. 2016 January; 68(1):3-19. doi: 10.1124/pr.114.009373) (the drugs may be obtained from many vendors, including Creative Biolabs®).

The radioisotope may be for example, but is not limited to, At211, I131, In131, I125, Y90, Re186, Re188, Sm153, Bi212, P32, Pb212 and radioactive isotopes of Lu.

In certain embodiments, the drug may be, but is not limited to, MMAE or MMAF.

In some embodiments, the Ab or antigen-binding Ab fragment is directly conjugated to the drug to form an ADC.

In some embodiments, the Ab or antigen-binding Ab fragment is indirectly conjugated to the drug to form an ADC.

Any appropriate conjugation method may be used to generate an ADC (for example, Nolting B. Methods Mol Biol. 2013; 1045:71-100. doi: 10.1007/978-1-62703-541-5_5; Jain N. et al., Pharm Res. 2015 November; 32(11):3526-40. doi: 10.1007/s11095-015-1657-7. Epub 2015 Mar. 11; Tsuchikama K. et al., Protein Cell. 2018 January; 9(1):33-46. doi: 10.1007/s13238-016-0323-0. Epub 2016 Oct. 14; Polakis P. et al., Pharmacol Rev. 2016 January; 68(1):3-19. doi: 10.1124/pr.114.009373). Examples of methods that may be used to perform conjugation include, but are not limited to, chemical conjugation and enzymatic conjugation.

### Conjugation Methods

Chemical conjugation may utilize, for example, but is not limited to, lysine amide coupling, cysteine coupling, and/or non-natural amino acid incorporation by genetic engineering. Enzymatic conjugation may utilize, for example, but is not limited to, transpeptidation using sortase, transpeptidation using microbial transglutaminase, and/or N-Glycan engineering.

In certain embodiments, one or more of cleavable linkers may be used for conjugation. The cleavable linker may enable cleavage of the drug upon responding to, for example, but not limited to, an environmental difference between the extracellular and intracellular environments (pH, redox potential, etc.) or by specific lysosomal enzymes.

Examples of the cleavable linker include, but are not limited to, hydrazone linkers, peptide linkers including cathepsin B-responsive linkers, such as valin-citrulline (vc) linker, disulfide linkers such as

N-succinimidyl-4-(2-pyridyldithio) (SPP) linker or N-succinimidyl-4-(2-pyridyldithio)butanoate (SPDB) linker, and pyrophosphate diester linkers.

Alternatively or simultaneously, one or more of non-cleavable linkers may be used. Examples of non-cleavable linkers include thioether linkers, such as N-succinimidyl 4-(N-maleimidomethyl) cyclohexane-1-carboxylate (SMCC), and maleimidocaproyl (mc) linkers. Generally, non-cleavable linkers are more resistant to proteolytic degradation and more stable compared to cleavable linkers.

### Anti-NMDAR2B Chimeric Antigen Receptors (CAR)

In some embodiments, an anti-NMDAR2B agent according to the present disclosure may be a chimeric antigen receptor (CAR). In particular, the CARs of the present invention comprise an antigen binding domain that binds to NMDAR2B, a transmembrane (TM) domain, and an intracellular signaling (ICS) domain.

A schematic showing a generic CAR construct of the present invention is depicted in FIG. 3A.

The CAR may optionally comprise a hinge that joins the antigen-binding domain and said TM domain. The CAR may optionally comprise one or more costimulatory (CS) domains. Schematics showing three more generic CAR constructs of the present invention are depicted in FIGS. 3B-3D.

### Antigen-Binding Domain

The CAR of the present disclosure comprises an antigen-binding domain which binds to NMDAR2B.

In some embodiments, the antigen-binding domain of the CAR may comprise any of the anti-NMDAR2B Abs, Ab fragments, or drug conjugates thereof disclosed herein.

In some embodiments, the antigen-binding domain of the CAR may comprise any of the antigen-binding domain of any of the anti-NMDAR2B Abs, Ab fragments disclosed herein.

In some particular embodiments, the antigen-binding domain of the CAR may comprise an anti-NMDAR2B scFv, which may be any of the anti-NMDAR2B scFv disclosed herein.

In some embodiments, the antigen-binding domain may comprise an amino acid sequence which is at least 80%, at least 85%, at least 90%, at least 92%, at least 94%, at least 95%, at least 96%, at least 97%, at least 98%, at least 99%, or 100% identical to SEQ ID NOs: 240, 245, 250, 255, 260, 265, 270, 275, 280, 285, 290, 295, 300, or 305 and comprises the same six CDRs as those contained in said respective SEQ ID NOS.

In some embodiments, the antigen-binding domain may comprise an amino acid sequence of SEQ ID NOs: 280, 285, 290, or 295.

In some embodiments, the antigen-binding domain may compete for binding to NMDAR2B with a scFv comprising an amino acid sequence which is at least 80%, at least 85%, at least 90%, at least 92%, at least 94%, at least 95%, at least 96%, at least 97%, at least 98%, at least 99%, or 100% identical to SEQ ID NOs: 240, 245, 250, 255, 260, 265, 270, 275, 280, 285, 290, 295, 300, or 305 and comprises the same six CDRs as those contained in said respective SEQ ID NOS. Such scFvs may be encoded by a nucleic acid sequence which is at least 80%, at least 85%, at least 90%, at least 92%, at least 94%, at least 95%, at least 96%, at least 97%, at least 98%, at least 99%, or 100% identical to SEQ ID NOs: 340, 345, 350, 355, 360, 365, 370, 375, 380, 385, 390, 395, 400, or 405, respectively.

### Hinge

In some embodiments, the CAR may comprise a hinge sequence between the antigen-binding domain and the TM domain. One of the ordinary skill in the art will appreciate that a hinge sequence is a short sequence of amino acids that facilitates flexibility (see, e.g. Woof J. M. et al., Nat Rev. Immunol., 4(2): 89-99 (2004)). The hinge sequence can be any suitable sequence derived or obtained from any suitable molecule.

In some embodiments, the length of the hinge sequence may be optimized based on the desired length of the extracellular portion of the CAR, which may be based on the location of the epitope within the target molecule. For example, if the epitope is in the membrane proximal region within the target molecule, longer hinges may be optimal.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge includes at least a portion of an IgG1, an IgG2, an IgG3, an IgG4, an IgE, an IgM, or an IgA immunoglobulin Fc region that falls within its CH2 and CH3 domains. In some embodiments, the hinge may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge to an Fc receptor (FcR). In some embodiments, the modified immunoglobulin Fc region may be designed with one or more mutations which result in one or more amino acid substitutions, modifications, or deletions that cause impaired binding of the hinge to one or more FcR including, but not limited to, FcγRI, FcγR2A, FcγR2B1, Fcγ2B2, Fcγ 3A, Fcγ 3B, FcεRI, FcεR2, FcαRI, Fcα/μR, or FcRn.

In some embodiments, a portion of the immunoglobulin constant region serves as a hinge between the antigen-binding domain, for example scFv or nanobody, and the TM domain. The hinge can be of a length that provides for increased responsiveness of the CAR-expressing cell following antigen binding, as compared to in the absence of the hinge. In some examples, the hinge is at or about 12 amino acids in length or is no more than 12 amino acids in length. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. Exemplary hinges include a CD28 hinge, IgG4 hinge alone, IgG4 hinge linked to CH2 and CH3 domains, or IgG4 hinge linked to the CH3 domain. Exemplary hinges include, but are not limited to, those described in Hudecek M. et al. (2013) Clin. Cancer Res., 19:3153, international patent application publication number WO2014031687, U.S. Pat. No. 8,822,647 or published App. No. US2014/0271635.

In some embodiments, the hinge sequence is derived from CD8α molecule or a CD28 molecule. In a preferred embodiment, the hinge sequence is derived from CD28. In one embodiment, the hinge comprises the amino acid sequence of human CD28 hinge (SEQ ID NO: 113). In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 113. Such a hinge sequence may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 213.

### Transmembrane (TM) Domain

With respect to the TM domain, the CAR can be designed to comprise a TM domain that is fused to the antigen-binding domain of the CAR. A hinge sequence may be inserted between the antigen-binding domain and the TM domain. In one embodiment, the TM domain that naturally is associated with one of the domains in the CAR is used. In some instances, the TM domain can be selected or modified by amino acid substitution to avoid binding of such domains to the transmembrane domains of the same or different surface membrane proteins to minimize interactions with other members of the receptor complex.

The TM domain may be derived either from a natural or from a synthetic source. Where the source is natural, the domain may be derived from any membrane-bound or transmembrane protein. Typically, the TM domain denotes a single transmembrane a helix of a transmembrane protein, also known as an integral protein. TM domains of particular use in this invention may be derived from (i.e. comprise at least the transmembrane region(s) of) CD28, CD3 ε, CD4, CD5, CD8, CD9, CD16, CD22, CD33, CD37, CD45, CD64, CD80, CD86, CD134, CD137, CD154, TCR α, TCR β, or CD3 zeta and/or TM domains containing functional variants thereof such as those retaining a substantial portion of the structural, e.g., transmembrane, properties thereof.

Alternatively the TM domain may be synthetic, in which case the TM domain will comprise predominantly hydrophobic residues such as leucine and valine. Preferably a triplet of phenylalanine, tryptophan and valine will be found at each end of a synthetic TM domain. A TM domain of the invention is thermodynamically stable in a membrane. It may be a single a helix, a transmembrane p barrel, a 3-helix of gramicidin A, or any other structure. Transmembrane helices are usually about 20 amino acids in length.

Preferably, the TM domain in the CAR of the invention is derived from the TM region of CD28. In one embodiment, the TM domain comprises the amino acid sequence of human CD28 TM (SEQ ID NO: 161) or the sequence encoded by SEQ ID NO: 261. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 114. Such a TM domain sequence may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 214.

Optionally, a short oligo- or polypeptide spacer, preferably between 2 and 10 amino acids in length may form the linkage between the TM domain and the ICS domain(s) of the CAR. A glycine-serine doublet may provide a suitable spacer.

### Intracellular Signaling (ICS) Domain and Costimulatory (CS) Domain

The ICS domain or otherwise the cytoplasmic domain of the CAR of the invention triggers or elicits activation of at least one of the normal effector functions of the cell in which the CAR has been placed. The term “effector function” refers to a specialized function of a cell. Effector function of a T cell, for example, may be cytolytic activity or helper activity including the secretion of cytokines. Thus, the term “intracellular signaling domain” or “ICS domain” refers to the portion of a protein which transduces the effector function signal and directs the cell to perform a specialized function. While usually the entire ICS domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling domain” or “ICS domain” is thus meant to include any truncated portion of the ICS domain sufficient to transduce the effector function signal.

Preferred examples of ICS domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one ICS domain alone may be insufficient for full activation of a cell, and a secondary or costimulatory signal may also be required. In such cases, a costimulatory domain (CS domain) may be included in the cytoplasmic portion of a CAR. A CS domain is a domain that transduces such a secondary or costimulatory signal. Optionally, the CAR of the present invention may comprise two or more CS domains. The CS domain(s) may be placed upstream of the ICS domain or downstream of the ICS domain. Two exemplary schematics of CAR constructs of the present invention containing at least one CS domain are illustrated in FIG. 3C and FIG. 3D.

In some embodiments, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or costimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Such a cytoplasmic signaling sequence may be contained in the ICS or the CS domain of the CAR of the present invention.

Examples of ITAM-containing primary cytoplasmic signaling sequences that are of particular use in the invention include those derived from an ICS domain of a lymphocyte receptor chain, a TCR/CD3 complex protein, an Fc receptor subunit, an IL-2 receptor subunit, CD3 ζ, FcRγ, FcR β, CD3γ, CD3 δ, CD3 ε, CD5, CD22, CD66d, CD79a, CD79b, CD278 (ICOS), Fc ε RI, DAP10, and DAP12.

It is particularly preferred that the ICS domain in the CAR of the invention comprises a cytoplasmic signaling sequence derived from CD3 zeta. In one embodiment, the ICS domain comprises the amino acid sequence of human CD3 ζ ICS (SEQ ID NO: 118). In some embodiments, the ICS domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 118. Such a sequence of CD3 zeta may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 218.

In a preferred embodiment, the cytoplasmic domain of the CAR may be designed to comprise the CD3 ζ ICS domain by itself. In another preferred embodiment, the CD3 ζ ICS domain may be combined with one or more of any other desired cytoplasmic domain(s) useful in the context of the CAR of the invention. For example, the cytoplasmic domain of the CAR can comprise a CD3 ζ ICS domain and a costimulatory (CS) domain. The CS region refers to a portion of the CAR comprising the intracellular domain of a costimulatory molecule. A costimulatory molecule is a cell surface molecule other than an antigen receptor or their ligands that is required for an efficient response of lymphocytes to an antigen.

Various CS domains have been reported to confer differing properties. For example, the 4-1BB CS domain showed enhanced persistence in in vivo xenograph models (Milone M. C. et al. Mol Ther 2009; 17:1453-1464; Song D. G. et al. Cancer Res 2011; 71:4617-4627). Additionally, these different CS domains produce different cytokine profiles, which in turn, may produce effects on target cell-mediated cytotoxicity and the disease microenvironment. Indeed, DAP10 signaling in NK cells has been associated with an increase in Th1 and inhibition of Th2 type cytokine production in CD8+ T cells (Barber A. et al. Blood 2011; 117:6571-6581).

Examples of co-stimulatory molecules include an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R P, IL2Ry, IL7R a, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like. Thus, while the invention is exemplified primarily with regions of CD28, DAP10, and/or 4-1BB as the CS domain, other costimulatory elements are within the scope of the invention.

The ICS domain and the CS domain(s) of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR is designed to comprise a cytoplasmic signaling sequence of CD3 ζ as the ICS domain and comprise a cytoplasmic signaling sequence of CD28 as the CS domain. In another embodiment, the CAR is designed to comprise a cytoplasmic signaling sequence of CD3 ζ as the ICS domain and comprise a cytoplasmic signaling sequence of DAP10 as the CS domain. In yet another embodiment, the CAR is designed to comprise a cytoplasmic signaling sequence of CD3 ζ as the ICS domain and comprise a cytoplasmic signaling sequence of 4-1BB as the CS domain. Such a cytoplasmic signaling sequence of CD3 ζ may be at least 85%, 86%, 87%, 88%, 89%, 90%, 91%, 92%, 93%, 94%, 95%, 96%, 97%, 98%, 99%, or 100% identical to the CD3 ζ ICS domain comprising the amino acid sequence of human CD3z ICS (SEQ ID NO: 118). Such a cytoplasmic signaling sequence of CD3 zeta may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 218.

Such a cytoplasmic signaling sequence of CD28 may be at least 85%, 86%, 87%, 88%, 89%, 90%, 91%, 92%, 93%, 94%, 95%, 96%, 97%, 98%, 99%, or 100% identical to the sequence of human CD28 CS domain (SEQ ID NO: 115). Such a cytoplasmic signaling sequence of CD28 may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 215. Such a cytoplasmic signaling sequence of DAP10 may be at least 85%, 86%, 87%, 88%, 89%, 90%, 91%, 92%, 93%, 94%, 95%, 96%, 97%, 98%, 99%, or 100% identical to the sequence of human 4-1BB CS domain (SEQ ID NO: 116). Such a cytoplasmic signaling sequence of 4-1BB may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 216. Such a cytoplasmic signaling sequence of DAP10 may be at least 85%, 86%, 87%, 88%, 89%, 90%, 91%, 92%, 93%, 94%, 95%, 96%, 97%, 98%, 99%, or 100% identical to the sequence of human DAP10 CS domain (SEQ ID NO: 117). Such a cytoplasmic signaling sequence of DAP10 may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 217.

Alternatively, when the antigen-binding domain comprises the NMDAR2B-binding portion of a molecule that binds to NMDAR2B as described above, the TM domain of the CAR may be derived from the transmembrane portion of the molecule.

### Exemplary CAR Constructs

In the following CAR examples, the CAR construct is described as “Antigen-binding domain-hinge-TM domain-CS domain-ICS domain.”

The CARs of the present invention may comprise an amino acid sequence at least 80%, at least 85%, at least 90%, at least 95%, at least 98% at least 99%, or 100% identical to any of the exemplary constructs below and comprise the six CDR sequences contained in the respective constructs:
(i) Ab8scFvHL-CD28H-CD28TM-CD28CS-CD3zICS (SEQ ID NO: 281), which may be encoded by the nucleic acid sequence of SEQ ID NO: 381;
(ii) Ab8scFvHL-CD28H-CD28TM-41BBCS-CD3zICS (SEQ ID NO: 282), which may be encoded by the nucleic acid sequence of SEQ ID NO: 382;
(iii) Ab8scFvHL-CD28H-CD28TM-DAP10CS-CD3zICS (SEQ ID NO: 283), which may be encoded by the nucleic acid sequence of SEQ ID NO: 383;
(iv) Ab8scFvLH-CD28H-CD28TM-CD28CS-CD3zICS (SEQ ID NO: 286), which may be encoded by the nucleic acid sequence of SEQ ID NO: 386;
(v) Ab8scFvLH-CD28H-CD28TM-41BBCS-CD3zICS (SEQ ID NO: 287), which may be encoded by the nucleic acid sequence of SEQ ID NO: 387;
(vi) Ab8scFvLH-CD28H-CD28TM-DAP10CS-CD3zICS (SEQ ID NO: 288), which may be encoded by the nucleic acid sequence of SEQ ID NO: 388;
(vii) Ab9scFvHL-CD28H-CD28TM-CD28CS-CD3zICS (SEQ ID NO: 291), which may be encoded by the nucleic acid sequence of SEQ ID NO: 391;
(viii) Ab9scFvHL-CD28H-CD28TM-41BBCS-CD3zICS (SEQ ID NO: 292), which may be encoded by the nucleic acid sequence of SEQ ID NO: 392;
(ix) Ab9scFvHL-CD28H-CD28TM-DAP10CS-CD3zICS (SEQ ID NO: 293), which may be encoded by the nucleic acid sequence of SEQ ID NO: 393;
(x) Ab9scFvLH-CD28H-CD28TM-CD28CS-CD3zICS (SEQ ID NO: 296), which may be encoded by the nucleic acid sequence of SEQ ID NO: 396;
(xi) Ab9scFvLH-CD28H-CD28TM-41BBCS-CD3zICS (SEQ ID NO: 297), which may be encoded by the nucleic acid sequence of SEQ ID NO: 397;
(xii) Ab9scFvLH-CD28H-CD28TM-DAP10CS-CD3zICS (SEQ ID NO: 298), which may be encoded by the nucleic acid sequence of SEQ ID NO: 398;
(xiii) Ab4scFvHL-CD28H-CD28TM-CD28CS-CD3zICS (SEQ ID NO: 241), which may be encoded by the nucleic acid sequence of SEQ ID NO: 341;
(xiv) Ab4scFvHL-CD28H-CD28TM-41BBCS-CD3zICS (SEQ ID NO: 242), which may be encoded by the nucleic acid sequence of SEQ ID NO: 342;
(xv) Ab4scFvHL-CD28H-CD28TM-DAP10CS-CD3zICS (SEQ ID NO: 243), which may be encoded by the nucleic acid sequence of SEQ ID NO: 343;
(xvi) Ab4scFvLH-CD28H-CD28TM-CD28CS-CD3zICS (SEQ ID NO: 246), which may be encoded by the nucleic acid sequence of SEQ ID NO: 346;
(xvii) Ab4scFvLH-CD28H-CD28TM-41BBCS-CD3zICS (SEQ ID NO: 247), which may be encoded by the nucleic acid sequence of SEQ ID NO: 347;
(xviii) Ab4scFvLH-CD28H-CD28TM-DAP10CS-CD3zICS (SEQ ID NO: 248), which may be encoded by the nucleic acid sequence of SEQ ID NO: 348;
(xix) Ab5scFvHL-CD28H-CD28TM-CD28CS-CD3zICS (SEQ ID NO: 251), which may be encoded by the nucleic acid sequence of SEQ ID NO: 351;
(xx) Ab5scFvHL-CD28H-CD28TM-41BBCS-CD3zICS (SEQ ID NO: 252), which may be encoded by the nucleic acid sequence of SEQ ID NO: 352;
(xxi) Ab5scFvHL-CD28H-CD28TM-DAP10CS-CD3zICS (SEQ ID NO: 253), which may be encoded by the nucleic acid sequence of SEQ ID NO: 353;
(xxii) Ab5scFvLH-CD28H-CD28TM-CD28CS-CD3zICS (SEQ ID NO: 256), which may be encoded by the nucleic acid sequence of SEQ ID NO: 356;
(xxiii) Ab5scFvLH-CD28H-CD28TM-41BBCS-CD3zICS (SEQ ID NO: 257), which may be encoded by the nucleic acid sequence of SEQ ID NO: 357;
(xxiv) Ab5scFvLH-CD28H-CD28TM-DAP10CS-CD3zICS (SEQ ID NO: 258), which may be encoded by the nucleic acid sequence of SEQ ID NO: 358;
(xxv) Ab6scFvHL-CD28H-CD28TM-CD28CS-CD3zICS (SEQ ID NO: 261), which may be encoded by the nucleic acid sequence of SEQ ID NO: 361;
(xxvi) Ab6scFvHL-CD28H-CD28TM-41BBCS-CD3zICS (SEQ ID NO: 262), which may be encoded by the nucleic acid sequence of SEQ ID NO: 362;
(xxvii) Ab6scFvHL-CD28H-CD28TM-DAP10CS-CD3zICS (SEQ ID NO: 263), which may be encoded by the nucleic acid sequence of SEQ ID NO: 363;
(xxviii) Ab6scFvLH-CD28H-CD28TM-CD28CS-CD3zICS (SEQ ID NO: 266), which may be encoded by the nucleic acid sequence of SEQ ID NO: 366;
(xxix) Ab6scFvLH-CD28H-CD28TM-41BBCS-CD3zICS (SEQ ID NO: 267), which may be encoded by the nucleic acid sequence of SEQ ID NO: 367;
(xxx) Ab6scFvLH-CD28H-CD28TM-DAP10CS-CD3zICS (SEQ ID NO: 268), which may be encoded by the nucleic acid sequence of SEQ ID NO: 368;
(xxxi) Ab7scFvHL-CD28H-CD28TM-CD28CS-CD3zICS (SEQ ID NO: 271), which may be encoded by the nucleic acid sequence of SEQ ID NO: 371;
(xxxii) Ab7scFvHL-CD28H-CD28TM-41BBCS-CD3zICS (SEQ ID NO: 272), which may be encoded by the nucleic acid sequence of SEQ ID NO: 372;
(xxxiii) Ab7scFvHL-CD28H-CD28TM-DAP10CS-CD3zICS (SEQ ID NO: 273), which may be encoded by the nucleic acid sequence of SEQ ID NO: 373;
(xxxiv) Ab7scFvLH-CD28H-CD28TM-CD28CS-CD3zICS (SEQ ID NO: 276), which may be encoded by the nucleic acid sequence of SEQ ID NO: 376;
(xxxv) Ab7scFvLH-CD28H-CD28TM-41BBCS-CD3zICS (SEQ ID NO: 277), which may be encoded by the nucleic acid sequence of SEQ ID NO: 377;
(xxxvi) Ab7scFvLH-CD28H-CD28TM-DAP10CS-CD3zICS (SEQ ID NO: 278), which may be encoded by the nucleic acid sequence of SEQ ID NO: 378;
(xxxvii) Ab10scFvHL-CD28H-CD28TM-CD28CS-CD3zICS (SEQ ID NO: 301), which may be encoded by the nucleic acid sequence of SEQ ID NO: 401;
(xxxviii) Ab10scFvHL-CD28H-CD28TM-41BBCS-CD3zICS (SEQ ID NO: 302), which may be encoded by the nucleic acid sequence of SEQ ID NO: 402;
(xxxix) Ab10scFvHL-CD28H-CD28TM-DAP10CS-CD3zICS (SEQ ID NO: 303), which may be encoded by the nucleic acid sequence of SEQ ID NO: 403;
(xl) Ab10scFvLH-CD28H-CD28TM-CD28CS-CD3zICS (SEQ ID NO: 306), which may be encoded by the nucleic acid sequence of SEQ ID NO: 406;
(xli) Ab10scFvLH-CD28H-CD28TM-41BBCS-CD3zICS (SEQ ID NO: 307), which may be encoded by the nucleic acid sequence of SEQ ID NO: 407; or
(xlii) Ab10scFvLH-CD28H-CD28TM-DAP10CS-CD3zICS (SEQ ID NO: 308) which may be encoded by the nucleic acid sequence of SEQ ID NO: 408.

The CARs having an amino acid sequence at least 80%, at least 85%, at least 90%, at least 95%, at least 98% at least 99%, or 100% to the respective CAR sequences above and comprising the six CDRs contained in the respective CAR sequences above may be encoded by a nucleic acid sequence at least 80%, at least 85%, at least 90%, at least 95%, at least 98% at least 99%, or 100% to SEQ ID NOS: SEQ ID NO: 381, 382, 383, 386, 387, 388, 391, 392, 393, 396, 397, 398, 341, 342, 343, 346, 347, 348, 351, 352, 353, 356, 357, 358, 361, 362, 363, 366, 367, 368, 371, 372, 373, 376, 377, 378, 401, 402, 403, 406, 407, and 408, respectively.

### Further Modification

The CARs of the present invention, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-target effect, reduced subject toxicity, improved target cytotoxicity, improved attraction of disease alleviating immune cells, detection, selection, targeting, and the like. For example, the cells may be engineered to express another CAR, or to have a suicide mechanism, and may be modified to remove or modify expression of an endogenous receptor or molecule such as a TCR and/or MHC molecule.

In some embodiments, a leader sequence (LS) may be placed upstream of the polynucleotide sequences encoding the foregoing exemplary CARs. The leader sequence facilitates the expression of the CAR on the cell surface. The polynucleotide sequence of such a lead sequence may be as set forth in SEQ ID NO: 212, which encodes the amino acid sequence as set forth in SEQ ID NO: 112. Any other sequences that facilitate the expression of the CAR on the cell surface may be used.

A general exemplary schematic of a construct for a LS-containing CAR of the present invention is shown in FIG. 3F (left).

In some embodiments, the vector or polynucleotide encoding the CAR further encodes other genes. The vector or polynucleotide may be constructed to allow for the co-expression of multiple genes using a multitude of techniques including co-transfection of two or more plasmids, the use of multiple or bidirectional promoters, or the creation of bicistronic or multicistronic vectors.

The construction of multicistronic vectors may include the encoding of IRES elements or 2A peptides, such as T2A, P2A, E2A, or F2A (for example, see Kim, J. H., et al., “High cleavage efficiency of a 2A peptide derived from porcine teschovirus-1 in human cell lines, zebrafish and mice”, PLoS One. 2011; 6(4)).

In some embodiments, the polynucleotide sequences for expressing the foregoing exemplary CARs further comprise a ribosomal skip sequence (or also referred to as T2A) and/or a marker that indicates successful CAR expression, such as but not limited to a sequence encoding truncated CD19 (or also referred to as trCD19). The nucleic acid sequence for T2A may be as provided by SEQ ID NO: 219, which encodes the amino acid sequence provided by SEQ ID NO: 119. trCD19 may have the sequence as provided by SEQ ID NO: 120, which may be encoded by, for example, SEQ ID NO: 220.

A schematic showing such polynucleotide constructs are shown in FIG. 3F (right).

When a ribosomal skip sequence and a marker-encoding sequence are placed downstream of the CAR sequence, the translation will be interrupted by the ribosomal skip sequence, resulting in two separate translation products, CAR protein and the marker protein. In some embodiments, cells expressing the CAR may be purified based on the presence of the marker protein.

In a particular embodiment, the polynucleotide or vector encoding the CAR further encodes a LS and trCD19 with the use of a T2A ribosomal skip sequence.

In some particular embodiments, exemplary constructs for expressing such a CAR may be provided by, for examples, SEQ ID NOS: 384, 389, 394, and 399.

The CAR expressing cell may further comprise a disruption to one or more endogenous genes. In some embodiments, the endogenous gene encodes TCRα, TCRβ, CD52, glucocorticoid receptor (GR), deoxycytidine kinase (dCK), or an immune checkpoint protein such as, for example, programmed death-1 (PD-1).

In some embodiments, the antigen-binding domain of the CAR according to the present disclosure may be conjugated to a drug.

In some embodiments, the drug may be any of the drugs that may be used in the anti-NMDAR2B ADC described above.

### Efficacy

The CARs of the present invention and cells expressing these CARs may be further modified to improve efficacy against cells expressing the target molecule. The cells may be cells expressing NMDAR2B. The cells expressing NMDAR2B may be cancer cells, vascular cells, or any other target disease-associated cells. In some embodiments, the improved efficacy may be measured by increased cytotoxicity against cells expressing the target molecule, for example cytotoxicity against cancer cells. In some embodiments, the improved efficacy may also be measured by increased production of cytotoxic mediators such as, but not limited to, IFN γ, perforin, and granzyme B. In some embodiments, the improved efficacy may be shown by reduction in the signature cytokines of the diseases, or alleviated symptoms of the disease when the CAR expressing cells are administered to a subject Other cytokines that may be reduced include TGF-beta, IL-6, IL-4, IL-10, and/or IL-13. the improved efficacy may be shown by NMDAR2B-specific immune cell responses, such as T cell cytotoxicity. In case of cancer, improved efficacy may be shown by better tumor cytotoxicity, better infiltration into the tumor, reduction of immunosuppressive mediators, reduction in weight decrease, reduction in ascites, reduction in tumor burden, and/or increased lifespan. In some embodiments, gene expression profiles may be also investigated to evaluate the efficacy of the CAR.

In one aspect, the CAR expressing cells are further modified to evade or neutralize the activity of immunosuppressive mediators, including, but not limited to prostaglandin E2 (PGE2) and adenosine. In some embodiments, this evasion or neutralization is direct In other embodiments, this evasion or neutralization is mediated via the inhibition of protein kinase A (PKA) with one or more binding partners, for example ezrin. In a specific embodiment, the CAR-expressing cells further express the peptide “regulatory subunit I anchoring disruptor” (RIAD). RIAD is thought to inhibit the association of protein kinase A (PKA) with ezrin, which thus prevents PKA's inhibition of TCR activation (Newick K. et al. Cancer Immunol Res. 2016 June; 4(6):541-51. doi: 10.1158/2326-6066.CIR-15-0263. Epub 2016 Apr. 4).

In some embodiments, the CAR expressing cells of the invention may induce a broad immune response, consistent with epitope spreading.

In some embodiments, the CAR expressing cells of the invention further comprise a homing mechanism. For example, the cell may transgenically express one or more stimulatory chemokines or cytokines or receptors thereof. In particular embodiments, the cells are genetically modified to express one or more stimulatory cytokines. In certain embodiments, one or more homing mechanisms are used to assist the inventive cells to accumulate more effectively to the disease site. In some embodiments, the CAR expressing cells are further modified to release inducible cytokines upon CAR activation, e.g., to attract or activate innate immune cells to a targeted cell (so-called fourth generation CARs or TRUCKS). In some embodiments, CARs may co-express homing molecules, e.g., CCR4 or CCR2b, to increase trafficking to the disease site.

### Controlling CAR Expression

In some instances, it may be advantageous to regulate the activity of the CAR or CAR expressing cells CAR. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (AD CC). For example, CAR-expressing cells described herein may also express an antigen that is recognized by molecules capable of inducing cell death, e.g., AD CC or compliment-induced cell death. For example, CAR expressing cells described herein may also express a receptor capable of being targeted by an antibody or antibody fragment Examples of such receptors include EpCAM, VEGFR, integrins (e.g., integrins αvβ, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing AD CC, e.g., cetuximab (ERBITUX®), such that administration of cetuximab induces AD CC and subsequent depletion of the CAR-expressing cells (see, e.g., WO2011/056894, and Jonnalagadda et al., “Gene Ther. 2013; 20(8)853-860).

In some embodiments, the CAR cell comprises a polynucleotide encoding a suicide polypeptide, such as for example RQR8. See, e.g., WO2013153391A, which is hereby incorporated by reference in its entirety. In CAR cells comprising the polynucleotide, the suicide polypeptide may be expressed at the surface of a CAR cell. The suicide polypeptide may also comprise a signal peptide at the amino terminus. Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., “Blood. 2014; 124(8)1277-1287). Other methods for depleting CAR-expressing cells described herein include administration of CAMPATH®, a monoclonal anti-CD52 antibody that selectively binds and targets mature lymphocytes, e.g., CAR-expressing cells, for destruction, e.g., by inducing AD CC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In some embodiments, a regulatable CAR (RCAR) where the CAR activity can be controlled is desirable to optimize the safety and efficacy of a CAR therapy. In some embodiments, a RCAR comprises a set of polypeptides, typically two in the simplest embodiments, in which the components of a standard CAR described herein, e.g., an antigen-binding domain and an ICS domain, are partitioned on separate polypeptides or members. In some embodiments, the set of polypeptides include a dimerization switch that, upon the presence of a dimerization molecule, can couple the polypeptides to one another, e.g., can couple an antigen-binding domain to an ICS domain. Additional description and exemplary configurations of such regulatable CARs are provided herein and in International Publication No. WO 2015/090229, hereby incorporated by reference in its entirety.

In an aspect, an RCAR comprises two polypeptides or members: 1) an intracellular signaling member comprising an ICS domain, e.g., a primary ICS domain described herein, and a first switch domain; 2) an antigen binding member comprising an antigen-binding domain, e.g., that specifically binds a target molecule described herein, as described herein and a second switch domain. Optionally, the RCAR comprises a TM domain described herein. In an embodiment, a TM domain can be disposed on the intracellular signaling member, on the antigen binding member, or on both. Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to an ICS domain can be different, e.g., reversed.

In some embodiments, the CAR expressing immune cell may only transiently express a CAR. For example, the cells of the invention may be transduced with mRNA comprising a nucleic acid sequence encoding an inventive CAR. In this vein, the present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequences (“UTRs”), a 5′ cap and/or Internal Ribosome Entry Site (IRES), the nucleic acid to be expressed, and a polyA tail, typically 50-2000 bases in length. RNA so produced can efficiently transfect different kinds of cells. In one embodiment, the template includes sequences for the CAR. In an embodiment, an RNA CAR vector is transduced into a cell by electroporation.

### Target Specificity

The CAR expressing cells of the present invention may further comprise one or more CARs, in addition to the first CAR. These additional CARs may or may not be specific for the target molecule of the first CAR. In some embodiments, the one or more additional CARs may act as inhibitory or activating CARs. In some embodiments, the CAR of some embodiments is the stimulatory or activating CAR; in other embodiments, it is the costimulatory CAR. In some embodiments, the cells further include inhibitory CARs (iCARs, see Fedorov et al., Sci. Transl. Medicine, 2013 December; 5(215): 215ra172), such as a CAR recognizing an antigen other than the target molecule of the first CAR, whereby an activating signal delivered through the first CAR is diminished or inhibited by binding of the inhibitory CAR to its ligand, e.g., to reduce off-target effects.

In some embodiments, the antigen-binding domain of the CAR is or is part of an immunoconjugate, in which the antigen-binding domain is conjugated to one or more heterologous molecule(s), such as, but not limited to, a cytotoxic agent, an imaging agent, a detectable moiety, a multimerization domain, or other heterologous molecule. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; antibiotics; toxins such as small molecule toxins or enzymatically active toxins. In some embodiments, the antigen-binding domain is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, to enhance persistence, the cells of the invention may be further modified to overexpress pro-survival signals, reverse anti-survival signals, overexpress Bcl-xL, overexpress hTERT, lack Fas, or express a TGF-β dominant negative receptor. Persistence may also be facilitated by the administration of cytokines, e.g., IL-2, IL-7, and IL-15.

### Polynucleotides

The present disclosure also provides polynucleotides and combinations of polynucleotides encoding the anti-NMDAR2B Abs, Ab fragments, or CARs of the present invention.

The polynucleotide or combination of polynucleotides according to the disclosure may comprise (a) a VH-encoding polynucleotide comprising CDR-H1-, CDR-H2-, and CDR-H3-encoding nucleic acid sequences, and (b) a VL-encoding polynucleotide comprising CDR-L1-, CDR-L2-, and CDR-L3-encoding nucleic acid sequences.

In some embodiments, the VH may be contained in a first polypeptide and encoded under a first promoter, and the VL may be contained in a second polypeptide and encoded under a second promoter, wherein the first and second polypeptides are separate polypeptides and the first and second promoters are separate promoters.

In some embodiments, the VH may be contained in a first polypeptide and encoded under a first promoter, and the VL may be contained in a second polypeptide and encoded under the first promoter, wherein the first and second polypeptides are separate polypeptides. In certain embodiments, an IRES or a ribosomal skip sequence may be inserted between the first polypeptide-encoding and the second polypeptide-encoding sequences, which allow for production of two separate polypeptides, the first and the second polypeptides.

In some embodiments, the VH may be contained in a first polypeptide and encoded under a first promoter, and the VL may be contained in a first polypeptide and encoded under the first promoter. In certain embodiments, a scFv may be encoded in this manner.

The CDR-H1-, CDR-H2-, CDR-H3-, CDR-L1-, CDR-L2-, and CDR-L3-encoding nucleic acid sequences may be the CDR-H1-, CDR-H2-, CDR-H3-, CDR-L1-, CDR-L2-, and CDR-L3-encoding nucleic acid sequences of any one of Ab3 through Ab10.

In some particular embodiments, the CDR-H1-, CDR-H2-, CDR-H3-, CDR-L1-, CDR-L2-, and CDR-L3-encoding nucleic acid sequences may be the CDR-H1-, CDR-H2-, CDR-H3-, CDR-L1-, CDR-L2-, and CDR-L3-encoding nucleic acid sequences of Ab8 or Ab9.

The VH- and VL-encoding nucleic acid sequences may be at least 85%, at least 90%, at least 92%, at least 94%, at least 95%, at least 96%, at least 97%, at least 98%, at least 99%, or 100% identical to the VH- and VL-encoding nucleic acid sequences of any one of Ab3, Ab4, Ab5, Ab6, Ab7, Ab8, Ab9, or Ab10 and encode the six CDRs of the respective Abs.

In some particular embodiments, the VH- and VL-encoding nucleic acid sequences may be at least 85%, at least 90%, at least 92%, at least 94%, at least 95%, at least 96%, at least 97%, at least 98%, at least 99%, or 100% identical to the VH- and VL-encoding nucleic acid sequences of Ab8 or Ab9 and encode the six CDRs of the respective Abs.

In some embodiments, the polynucleotide or combination of polynucleotides according to the disclosure may comprise (a) a heavy chain-encoding polynucleotide which encodes CDR-H1, CDR-H2, and CDR-H3, and (b) a light chain-encoding polynucleotide which encodes CDR-L1, CDR-L2, and CDR-L3.

In some embodiments, the heavy chain- and light chain-encoding nucleic acid sequences may be at least 85%, at least 90%, at least 92%, at least 94%, at least 95%, at least 96%, at least 97%, at least 98%, at least 99%, or 100% identical to the heavy chain- and light chain-encoding nucleic acid sequences of any one of Ab3, Ab4, Ab5, Ab6, Ab7, Ab8, Ab9, or Ab10 and encode the six CDRs of the respective Abs.

In some particular embodiments, the heavy chain- and light chain-encoding nucleic acid sequences may be at least 85%, at least 90%, at least 92%, at least 94%, at least 95%, at least 96%, at least 97%, at least 98%, at least 99%, or 100% identical to the heavy chain- and light chain-encoding nucleic acid sequences of Ab8 or Ab9 and encode the six CDRs of the respective Abs.

In some embodiments, the polynucleotide or combination of polynucleotides according to the disclosure may encode a scFv according to the present disclosure.

In some embodiments, the the linker-encoding nucleic acid sequence may comprise or consist of SEQ ID NO: 210 or 211.

In some embodiments, the scFv-encoding polynucleotide may comprise a nucleic acid sequence which is at least 80%, at least 85%, at least 90%, at least 92%, at least 94%, at least 95%, at least 96%, at least 97%, at least 98%, at least 99%, or 100% identical to SEQ ID NO: 340, 345, 350, 355, 360, 365, 370, 375, 380, 385, 390, 395, 400, or 405 and encodes the same CDRs as those contained in SEQ ID NO: 240, 245, 250, 255, 260, 265, 270, 275, 280, 285, 290, 295, 300, or 305, respectively.

In some embodiments, the polynucleotide or combination of polynucleotides according to the disclosure may encode: (i) a human CH1 or a variant thereof; (ii) a human hinge; (iii) a human CH2 or a variant thereof; (iv) a human CH3 or a variant thereof; (v) a human CLκ or a variant thereof; and/or (vi) a human CLλ or a variant thereof.

In some embodiments, the human CH1-, hinge, and CH2-encoding sequences may comprise or consist of the nucleic acid sequence of: SEQ ID NO: 410, 411, and 412, respectively, or may be at least 80%, at least 85%, at least 90%, at least 92%, at least 94%, at least 95%, at least 96%, at least 97%, at least 98%, or at least 99% identical thereto;

In some embodiments, the the human CH3-encoding sequence may comprise or consist of the nucleic acid sequence of: SEQ ID NO: 413, 414, 415, or 416 or may be at least 80%, at least 85%, at least 90%, at least 92%, at least 94%, at least 95%, at least 96%, at least 97%, at least 98%, or at least 99% identical thereto.

In some embodiments, the human CLκ- and CLλ-encoding sequences may comprise or consist of the nucleic acid sequence of: SEQ ID NO: 425 and 426, respectively or is at least 80%, at least 85%, at least 90%, at least 92%, at least 94%, at least 95%, at least 96%, at least 97%, at least 98%, or at least 99% identical thereto.

In some embodiments, the polynucleotide or combination of polynucleotides according to the disclosure may encode a Fc region.

In some embodiments, the Fc region-encoding sequence may comprise or consist of the nucleic acid sequence of: SEQ ID NO: 417, 418, 419, or 420 or may be at least 80%, at least 85%, at least 90%, at least 92%, at least 94%, at least 95%, at least 96%, at least 97%, at least 98%, or at least 99% identical thereto.

In some embodiments, the polynucleotide or combination of polynucleotides according to the disclosure may encode a heavy chain constant region.

In some embodiments, the heavy chain constant region-encoding sequence may comprise or consist of the nucleic acid sequence of: SEQ ID NO: 421, 422, 423, or 424 or may be at least 80%, at least 85%, at least 90%, at least 92%, at least 94%, at least 95%, at least 96%, at least 97%, at least 98%, or at least 99% identical thereto.

In some embodiments, the polynucleotide or combination of polynucleotides according to the disclosure may encode a CAR according to the present disclosure.

In some embodiments, the sequence encoding the antigen-binding domain of the CAR may comprise or consist of the nucleic acid sequence of: SEQ ID NO: 340, 345, 350, 355, 360, 365, 370, 375, 380, 385, 390, 395, 400, or 405 or may be at least 80%, at least 85%, at least 90%, at least 92%, at least 94%, at least 95%, at least 96%, at least 97%, at least 98%, or at least 99% identical thereto, and encodes the six CDRs contained in SEQ ID NO: 240, 245, 250, 255, 260, 265, 270, 275, 280, 285, 290, 295, 300, or 305, respectively.

In some embodiments, the sequence encoding the TM domain of the CAR may comprise or consist of the nucleic acid sequence of: SEQ ID NO: 214 or may be at least 80%, at least 85%, at least 90%, at least 92%, at least 94%, at least 95%, at least 96%, at least 97%, at least 98%, or at least 99% identical thereto.

In some embodiments, the sequence encoding the ICS domain of the CAR may comprise or consist of the nucleic acid sequence of: SEQ ID NO: 218 or may be at least 80%, at least 85%, at least 90%, at least 92%, at least 94%, at least 95%, at least 96%, at least 97%, at least 98%, or at least 99% identical thereto.

In some embodiments, the sequence encoding the hinge of the CAR may comprise or consist of the nucleic acid sequence of: SEQ ID NO: 213 or may be at least 80%, at least 85%, at least 90%, at least 92%, at least 94%, at least 95%, at least 96%, at least 97%, at least 98%, or at least 99% identical thereto.

In some embodiments, the sequence encoding the CS domain of the CAR may comprise or consist of the nucleic acid sequence of: SEQ ID NO: 215, 216, or 217 or may be at least 80%, at least 85%, at least 90%, at least 92%, at least 94%, at least 95%, at least 96%, at least 97%, at least 98%, or at least 99% identical thereto.

In some embodiments, the CAR-encoding sequence may comprise or consist of the nucleic acid sequence of: any one of SEQ ID NOS: 341, 342, 343, 346, 347, 348, 351, 352, 353, 356, 357, 358, 361, 362, 363, 366, 367, 368, 371, 372, 373, 376, 377, 378, 381, 382, 383, 386, 387, 388, 391, 392, 393, 396, 397, 398, 401, 402, 403, 406, 407, and 408 or may be at least 80%, at least 85%, at least 90%, at least 92%, at least 94%, at least 95%, at least 96%, at least 97%, at least 98%, or at least 99% identical thereto. In some embodiments, the CAR-encoding sequence may at least 80%, at least 85%, at least 90%, at least 92%, at least 94%, at least 95%, at least 96%, at least 97%, at least 98%, at least 99%, or 100% identical to the said respective SEQ ID NOS and encode the six corresponding CDRs contained in SEQ ID NOS: 241, 242, 243, 246, 247, 248, 251, 252, 253, 256, 257, 258, 261, 262, 263, 266, 267, 268, 271, 272, 273, 276, 277, 278, 281, 282, 283, 286, 287, 288, 291, 292, 293, 296, 297, 298, 301, 302, 303, 306, 307, and 308, respectively.

In some embodiments, the CAR-encoding sequence may comprise a LS sequence.

In some embodiments, the LS sequence may comprise or consist of the nucleic acid sequence of SEQ ID NO: 212, or is at least 80%, at least 85%, at least 90%, at least 92%, at least 94%, at least 95%, at least 96%, at least 97%, at least 98%, at least 99%, or 100% identical thereto.

In some embodiments, the CAR-encoding sequence may comprise a ribosome skip sequence.

In some embodiments, the ribosome skip sequence may be a T2A sequence and may comprise or consist of the nucleic acid sequence of SEQ ID NO: 219, or is at least 80%, at least 85%, at least 90%, at least 92%, at least 94%, at least 95%, at least 96%, at least 97%, at least 98%, at least 99%, or 100% identical thereto.

In some embodiments, the CAR-encoding sequence may encode a marker sequence, such as trCD19. When a marker such as trCD19, which can be expressed on the cell surface is used, the expression of the marker may be determined via any available technique including, but not limited to, flow cytometry or immunofluorescence assays. Expression of such a marker typically indicates successful introduction and expression of the transgene(s) introduced together with the marker gene. Therefore, cells expressing the anti-NMDAR2B CAR of the invention may be, for example, selected based on the expression of the marker.

In some embodiments, the trCD19-encoding sequence and may comprise or consist of the nucleic acid sequence of SEQ ID NO: 220, or is at least 80%, at least 85%, at least 90%, at least 92%, at least 94%, at least 95%, at least 96%, at least 97%, at least 98%, at least 99%, or 100% identical thereto.

In some embodiments, the CAR-encoding sequence may comprise a LS, a T2A, and trCD10-encoding sequences.

In particular embodiments, such a CAR-encoding sequence may comprise or consist of the nucleic acid sequence of SEQ ID NO: 384, 389, 394, or 399.

### Vectors

The present invention also provides vectors and combinations of vectors. in which a polynucleotide encoding an anti-NMDAR2B Ab, Ab fragment, or CAR of the present invention is inserted.

In some embodiments, an anti-NMDAR2B Ab, Ab fragment, or CAR may be encoded in one vector.

In some embodiments, an anti-NMDAR2B Ab, Ab fragment, or CAR may be encoded in a combination of vectors.

In some embodiments, the combination of vectors may comprise: (a) a first vector encoding the VH; and (b) a second vector encoding the VL,

In some embodiments, the combination of vectors may comprise: (a) the first vector encodes a heavy chain; and (b) the second vector encodes a light chain.

The vector may be, for example, a DNA vector or a RNA vector. The vector may be, for example, but not limited to, a plasmid, a cosmid, a viral replicon, or a viral vector.

The viral vector may be a vector of a DNA virus, which may be an adenovirus, or an RNA virus, which may be a retrovirus. Types of vectors suite for Abs, antigen-binding Ab fragments, and/or CARs are well known in the art (for example, see Rita Costa A. et al., Eur J Pharm Biopharm. 2010 February; 74(2):127-38. doi: 10.1016/j.ejpb.2009.10.002. Epub 2009 Oct. 22; Frenzel A. et al. Front Immunol. 2013; 4: 217. Published online 2013 Jul. 29. doi: 10.3389/fimmu.2013.00217).

When the host cells are insect cells, such as for producing Abs or antigen-binding Ab fragments, insect-specific viruses may be used. Examples of the insect-specific viruses include, but are not limited to, the family of Baculoviridae, particularly the Autographa californica nuclear polyhedrosis virus (AcNPV). When the host cells are plant cells, plant-specific viruses and bacteria, such as Agrobacterium tumefaciens, may be used.

For expressing vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. This would be particularly beneficial for expressing CAR constructs.

In brief summary, the expression of nucleic acids encoding anti-NMDAR2B agents is typically achieved by operably linking a nucleic acid encoding the anti-NMDAR2B agent polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired polynucleotide.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, y-retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art In some embodiments, adenovirus vectors are used. A number of adenovirus vectors are known in the art In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, the CMV-actin-globin hybrid (CAG) promotor, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfection/Transduction

Methods of introducing and expressing genes into a cell are known in the art In the context of an expression vector, the vector can be readily introduced into a host cell, e.g., mammalian, bacterial, yeast, or insect cell by any method in the art. For example, the expression vector can be transferred into a host cell by physical, chemical, or biological means.

For transduction of CAR constructs to obtain CAR-expressing cells, any appropriate methods may be used.

For example, on day 1, cells (e.g., T cells, cell populations including T cells) may be harvested and stimulated (e.g., using an anti-CD3 antibody such as an OKT3 and IL-2); on day 2, cells may be transduced with a CAR-encoding virus (e.g., by spinfection, using an appropriate condition (e.g., 1500 g for 1 hr)) and rested overnight; on day 3, another transduction may be performed; on days 4-7, cells may be split to allow growth and/or remove dead cells and/or enrich successfully transduced cells; on day 8, cells may be harvested for use or storage for later use. Culture conditions and duration may be adjusted as appropriate.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., “1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Cells

Also provided are cells, cell populations, and compositions containing the cells, e.g., cells comprising a polynucleotide encoding an anti-NMDAR2B Ab, Ab fragment, or CAR of the present invention. Cells expressing anti-NMDAR2B Abs or antigen-binding Ab fragments may be used to harvest the Abs or antigen-binding Ab fragments. Cells expressing anti-NMDAR2B CARs may be administered to a subject or may be incorporated in a composition to be administered to a subject Among the compositions are pharmaceutical compositions and formulations for administration, such as for adoptive cell therapy.

Also provided are therapeutic methods for administering the Abs or Ab fragments or the cells and compositions to subjects, e.g., patients.

### Cell Types

Thus, also provided are cells expressing the anti-NMDAR2B Abs, Ab fragments, or CARs of the present invention.

For expressing an anti-NMDAR2B Ab or antigen-binding Ab fragment, any appropriate cells may be used. For example, cells may be: (i) prokaryotic cells, such as gram-negative bacteria and gram-positive bacteria; or (ii) eukaryotic cells, such as yeast, filamentous fungi, protozoa, insect cells, plant cells, and mammalian cells (reviewed in Frenzel A. et al. Front Immunol. 2013; 4: 217. Published online 2013 Jul. 29. doi: 10.3389/fimmu.2013.00217).

Specific examples of gram-negative bacteria that are suited for production of Ab or antigen-binding Ab fragments include, but are not limited to, E. coli, Proteus mirabilis, and Pseudomonas putidas. Specific examples of gram-positive bacteria include, but are not limited to, Bacillus brevis, Bacillus subtilis, Bacillus megaterium, Lacto-bacilluszeae/casei, and Lactobacillus paracasei. Specific examples of yeast bacteria that are suited for production of Ab or antigen-binding Ab fragments include, but are not limited to, Pichia pastoris, Saccharomyces cerevisiae, Hansenula polymorpha, Schizosaccharomyces pombe, Schwanniomyces occidentalis, Kluyveromyces lactis, and Yarrowia lipolytica. Specific examples of filamentous fungi that are suited for production of Ab or antigen-binding Ab fragments include, but are not limited to, the genera Trichoderma and Aspergillus, A. niger (subgenus A. awamori), Aspergillus oryzae, and Chrysosporium lucknowense. Specific examples of protozoa that are suited for production of Ab or antigen-binding Ab fragments include, but are not limited to, Leishmania tarentolae. Specific examples of insect cells that are suited for production of Ab or antigen-binding Ab fragments include, but are not limited to, insect cell lines like Sf-9 and Sf-21 or SfSWT-1 “Mimic™” cells of Spodoptera frugiperda, DS2 cells of Drosophila melanogaster, High Five cells (BTI-TN-5B1-4) of Trichopulsia ni, or Schneider2 (S2) cells of D. melanogaster., They can be efficiently transfected with insect-specific viruses from the family of Baculoviridae, particularly the Autographa californica nuclear polyhedrosis virus (AcNPV). Specific examples of mammalian cells that are suited for production of Ab or antigen-binding Ab fragments include, but are not limited to, Chinese hamster ovary (CHO) cells, the human embryonic retinal cell line Per.C6 [Crucell, Leiden, Netherlands], CHO-derived cell lines such as K1-, DukXB11-, Lec13, and DG44-cell lines, mouse myeloma cells such as SP 2/0, YB 2/0, and NS0 cells, GS-NSO, hybridoma cells, baby hamster kidney (BHK) cells, and the human embryonic kidney cell line HEK293, HEK293T, HEK293E, and human neuronal precursor cell line AGE1.HN (Probiogen, Berlin, Germany). Specific examples of plant cells that are suited for production of Ab or antigen-binding Ab fragments include, but are not limited to BY2 or NT1 cells of N. tabacum, Bengal, Donjin, or Taipie cells of Oryza sativa, and cells of Hordeum vulgare.

Alternatively, genetically modified organisms such as transgenic plants and transgenic animals may be used. Exemplary plants that may be used include, but are not limited to, tobacco, maize, duckweed, Chlamydomonas reinhardtii, Nicotiana tabacum, Nicotianaben thamiana, and Nicotiana benthamiana. Exemplary animals that may be used include, but are not limited to mouse, rat, and chicken.

For expressing an anti-NMDAR2B CAR, the cells generally are eukaryotic cells, such as mammalian cells, and typically are human cells, more typically primary human cells, e.g., allogeneic or autologous donor cells. The cells for introduction of the CAR may be isolated from a sample, such as a biological sample, e.g., one obtained from or derived from a subject In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immunity, e.g., myeloid cells, including monocytes, macrophages, dendritic cells, neutrophils, eosinophils, basophils, or mast cells, or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Alternatively, an immortalized cell or a cell line may be used for expressing a CAR of the present disclosure. Such examples include, but are not limited to, a T cell line, a CD4+ T cell line, a CD8+ T cell line, a regulatory T cell line, an NK-T cell line, an NK cell line (e.g., NK-92), a monocyte line, a macrophage line, a dendritic cell line, and a mast cell line. Furthermore, a desired cell type for CAR expression, for example T cells or NK cells may be generated from a stem cell, such as an embryonic stem cell, iPSCs, or hematopoietic stem cell.

With reference to the subject to be treated with cells expressing an anti-NMDAR2B CAR, the cells may be allogeneic and/or autologous. Among the methods include off-the-shelf methods. In some embodiments, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

In some embodiments, the cells are T cells. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells.

In some embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. In some embodiments, the cells are monocytes or granulocytes, e.g., myeloid cells, macrophages, neutrophils, dendritic cells, mast cells, eosinophils, and/or basophils. CAR-expressing phagocytic cells expressing may be able to bind to and phagocytose or nibble target cells (Morrissey M. A. et al., Elife. 2018 Jun. 4; 7. pii: e36688. doi: 10.7554/eLife.36688).

In some embodiments, the cells are derived from cell lines, e.g., T cell lines. The cells in some embodiments are obtained from a xenogeneic source, for example, from mouse, rat, non-human primate, and pig.

In some embodiments, the T cells may have been modified such that its endogenous T cell receptor (TCR) is not expressed, not functionally expressed, or expressed at reduced levels compared to a wild-type T cell. Such T cells may be useful for preventing or alleviating unintended reactions mediated by the TCRs encoded by the cells, such as the graft-versus-host disease.

In some embodiments, the recombinant or isolated cell may be activated or stimulated to proliferate; exhibit cytotoxicity against cells expressing the target molecule; increase expression of a cytokine, optionally IFN-g, and/or a chemokine; and/or decrease expression of a cytokine, optionally TGF-b and/or IL-10, and/or a chemokine, when the Ab or Ab fragment, the ADC, or the CAR binds to its target molecule.

In some embodiments, the administration of the cell to a subject may ameliorate a disease, optionally cancer, in the subject, when the Ab or Ab fragment, the ADC, or the CAR binds to its target molecule.

### Cell Acquisition for Anti-NMDAR2B CAR Expression

For cells for expressing anti-NMDAR2B CARs, prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and disease sites such as tumors. In some embodiments, any number of T cell lines available and known to those skilled in the art, may be used. In some embodiments, cells can be derived from a healthy donor, from a patient diagnosed with cancer or from a patient diagnosed with an infection. In some embodiments, cells can be part of a mixed population of cells which present different phenotypic characteristics.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some embodiments, the sample from which the cells are derived or isolated is blood or a blood-derived sample, or is or is derived from a leukapheresis product Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, tongue, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some embodiments, contain lymphocytes, including T cells, monocytes, granulocytes, B cells, other nucleated white blood cells, red blood cells, and/or platelets, and in some embodiments contains cells other than red blood cells and platelets.

Also provided herein are cell lines obtained from a transformed cell according to any of the above-described methods. Also provided herein are modified cells resistant to an immunosuppressive treatment In some embodiments, an isolated cell according to the invention comprises a polynucleotide encoding a CAR.

### Cell Purification

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some embodiments, a washing step is accomplished a semi-automated “flow-through” centrifuge (for example, the Cobe 2991 cell processor, Baxter) according to the manufacturer's instructions. In some embodiments, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. This would be particularly useful for isolating CAR-expressing cells. In a specific embodiment, the surface maker is trCD19. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some embodiments, negative selection can be particularly useful where no antibody is available that specifically identifies a cell type in a heterogeneous population, such that separation is best carried out based on markers expressed by cells other than the desired population.

In some embodiments, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+ T cells can be positively selected using CD3 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker high) on the positively or negatively selected cells, respectively.

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some embodiments is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy. In embodiments, memory T cells are present in both CD62L+ and CD62L-subsets of CD8+ peripheral blood lymphocytes. PBMC can be enriched for or depleted of CD62L-CD8+ and/or CD62L+CD8 fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some embodiments, it is based on negative selection for cells expressing or highly expressing CD45RA and/or granzyme B. In some embodiments, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some embodiments are carried out simultaneously and in other embodiments are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In some embodiments, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some embodiments, the particles are left attached to the cells for administration to a patient In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In certain embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the methods include density-based cell separation methods, such as the preparation of white blood cells from peripheral blood by lysing the red blood cells and centrifugation through a Percoll or Ficoll gradient.

In any of the aforementioned separation steps, the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

### Cell Preparation and Expansion

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation.

In some embodiments, the compositions or cells are incubated in the presence of stimulating conditions or a stimulatory agent Such conditions include those designed to induce proliferation, expansion, activation, and/or survival of cells in the population, to mimic antigen exposure, and/or to prime the cells for genetic engineering, such as for the introduction of a recombinant antigen receptor. The cells of the invention can be activated and expanded, either prior to or after genetic modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Particularly in relation to CAR-expressing cells, T cells can be expanded in vitro or in vivo. Generally, the T cells of the invention can be expanded, for example, by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. In some embodiments, the T cell populations may be stimulated in vitro by contact with Muromonab-CD3 ζ OKT3). For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640® or, X-vivo 5®, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, IL-21, TGF-β, and TNF, or any other additives for the growth of cells known to the skilled artisan. In a preferred embodiment, T cells are stimulated in vitro by exposure to OKT3 and IL-2. Other additives for the growth of cells include, but are not limited to, surfactant, Plasmanate, and reducing agents such as N-acetyl-cysteine and 2-mercaptoethanol. Media can include RPMI 1640®, A1M-V, DMEM, MEM, a-MEM, F-12, X-Vivo 1®, and X-Vivo 20®, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° Celsius) and atmosphere (e.g., air plus 5% C02). T cells that have been exposed to varied stimulation times may exhibit different characteristics.

In some embodiments, the isolated cells of the invention can be expanded by co-culturing with tissue or cells. The cells can also be expanded in vivo, for example in the subject's blood after administrating the cell into the subject.

In some embodiments, when cells are expanded in vivo, at least one cell of the invention may be administered to a subject, and the administration may lead to an expansion of the cell in the subject, resulting in a population of cells. Alternatively, a polynucleotide or vector of the invention may be administered to a subject Once the polynucleotide or vector is taken up by a cell within the subject and the cell proliferate or expand in the subject, this may result in a population of cells of the invention within the subject.

In certain embodiments, the resulting population of cells persists in the subject for at least three months, at least four months, at least five months, at least six months, at least seven months, at least eight months, at least nine months, at least ten months, at least eleven months, at least twelve months, at least eighteen months, at least two years, or at least three years after administration.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some embodiments, the non-dividing feeder cells can comprise y-irradiated PBMC feeder cells. In some embodiments, the PBMC are irradiated with γ rays in the range of about 3000 to 3600 rads to prevent cell division. In some embodiments, the feeder cells are added to culture medium prior to the addition of the populations of T cells.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some embodiments may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° Celsius at a rate of 1 degree per minute and stored in the vapor phase of a liquid nitrogen storage tank.

Isolation of Ab or Antigen-Binding Ab Fragment from Cell Culture

Cells, such as hybridomas, bacterial cells, yeast cells, insect cells, plant cells, or mammalian cells such as CHO and HEK cells that are producing Abs or antigen-binding Ab fragments of the present invention may be grown using standard methods, in suitable culture medium for this purpose (such as D-MEM or RPMI-1640), or in vivo as ascites. Abs or antigen-binding Ab fragments secreted by the cells can be separated from the culture medium, ascites fluid, or serum using conventional immunoglobulin purification procedures, such as, but not limited to, protein A-Sepharose, hydroxyapatite chromatography, gel electrophoresis, dialysis, or affinity chromatography (Ma H. et al., Methods. 2017 Mar. 1; 116:23-33. doi: 10.1016/j.ymeth.2016.11.008. Epub 2016 Nov. 18; Shukla A. A. et al. Trends Biotechnol. 2010 May; 28(5):253-61. doi: 10.1016/j.tibtech.2010.02.001. Epub 2010 Mar. 19; Arora S. et al., Methods. 2017 Mar. 1; 116:84-94. doi: 10.1016/j.ymeth.2016.12.010. Epub 2016 Dec. 22).

Methods for expressing, isolating, and evaluating multispecific and bispecific Abs and antigen-binding Ab fragments are also known in the art (for example, see Brinkmann U. et al., MAbs. 2017 February-March; 9(2): 182-212. Published online 2017 Jan. 10. doi: 10.1080/19420862.2016.1268307; Dimasi N. et al. Methods. 2018 Aug. 11. pii: S1046-2023(18)30149-X. doi: 10.1016/j.ymeth.2018.08.004).

### Therapeutic Applications

Anti-NMDAR2B agents of the present invention (Abs, antigen-binding Ab fragments, multi-specific Abs, multi-specific antigen-binding Ab fragments, ADCs, or CARs that binds to NMDAR2B, and cells encoding and/or expressing any of the foregoing), nucleic acids encoding such an agent, vectors encoding such an agent, isolated cells obtained by the methods described above, or cell lines derived from such isolated cells, and/or pharmaceutical compositions comprising thereof can be used as a medicament in the treatment of a disease, disorder, or condition in a subject In some embodiments, such a medicament can be used for treating an NMDAR2B-associated disease or condition.

### Target Diseases and Conditions

In some embodiments, anti-NMDAR2B agents according to the present disclosure may be used to treat a disease or condition associated with NMDAR2B or elevated NMDAR2B expression.

The NMDAR2B-associated condition may be, for example, but not limited to, cancer and cancer-associated diseases and conditions.

In particular embodiments, the anti-NMDAR2B agents of the present invention may be used to treat a cancer. NMDAR2B is upregulated in different types of cancer such as, but not limited to, small-cell lung cancer (SCLC) (North W. et al., Clinical Pharmacology: Advances and Applications. 2010:2 31-40), pancreatic cancer (North W. et al., Clinical Pharmacology: Advances and Applications. 2017:9 79-86), ovarian cancer (North W. et al., Clinical Pharmacology: Advances and Applications. 2015:7 111-117), and breast cancer (North W. et al., Breast Cancer Res Treat. 2010 July; 122(2): 307-314.), and glioma (Gao X et al., Neurosurg Focus. 2014 December; 37(6):E17.), and also likely prostate cancer. Studies further report that NMDAR2B is also expressed in, for example, hepatoma (Yamaguchi F., BMC Cancer. 2013 Oct. 10; 13:468.), colon cancer (Stepulak A. et al., Histochem Cell Biol. 2009 October; 132(4):435-45.), thyroid cancer (Stepulak A. et al., Histochem Cell Biol. 2009 October; 132(4):435-45.), laryngeal cancer (Stepulak A. et al., Anticancer Res. 2011 February; 31(2):565-73.).

Therefore, preferred target diseases include these cancers. In some embodiments, pancreatic cancer is a preferred target disease. In some embodiments, prostate cancer is a preferred target disease. In some embodiments, SCLC is a preferred target disease. In some embodiments, breast cancer is a preferred target disease. In some embodiments, ovarian cancer is a preferred target disease. In some embodiments, glioma is a preferred target disease. The anti-NMDAR2B agents according to the present invention may also be used to treat any other cancers in which NMDAR2B is upregulated or has a pathological role.

Other exemplary target diseases may include cerebral ischemia and neurodegenerative diseases such as Alzheimer's disease and Huntington's disease.

### Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Lagomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

In some embodiments, the subject, to whom the Abs, antigen-binding Ab fragments, ADCs, CAR expressing cells, cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes such as cytokine release syndrome (CRS).

In some embodiments, the subject has persistent or relapsed disease, e.g., following treatment with another immunotherapy and/or other therapy. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the methods include administration of an Ab, Ab fragments, ADC, or CAR expressing cell or a composition containing such an anti-NMDAR2B agent to a subject, tissue, or cell, such as one having, at risk for, or suspected of having a disease, condition or disorder associated with NMDAR2B, such as cancer. In some embodiments, the anti-NMDAR2B agents and/or compositions are administered to a subject having the particular disease or condition to be treated, e.g., via adoptive cell therapy, such as adoptive T cell therapy. In some embodiments, the anti-NMDAR2B agents or compositions are administered to the subject, such as a subject having or at risk for the disease or condition. In some embodiments, the methods thereby treat, e.g., ameliorate one or more symptom of the disease or condition, for example, by reducing, inhibiting, or inactivating NMDAR2B and/or NMDAR2B-expressing cells.

### Cell Origin

For purposes of the methods of anti-NMDAR2B CAR therapy, wherein host cells or populations of cells are administered, the cells can be cells that are xenogeneic, allogeneic or autologous to the subject.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject Thus, in some embodiments, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject

In certain embodiments, where cells are T cells and are not autologous to the subject, the expression of the cell's endogenous T cell receptors (TCRs) may be suppressed or disrupted. The TCR expression may be suppressed via any appropriate technique, for example, by silencing any compartment of the endogenous TCRs using tools such as, but not limited to, an siRNA, shRNA, micro RNA, or artificial microRNA. Alternatively, TCR gene may be disrupted or deleted via any appropriate technique, for example using the CRISPR/Cas system, transcription activator-like effector nucleases (e.g., TALEN®), or Zinc finger nucleases (ZFNs). The suppression or disruption of TCR may allow for reduction or prevention of undesired effects in which the TCRs recognize antigens in the subject as foreign and cause immune responses against the subject, an immune attack often called as graft-versus-host disease (GVHD).

In certain embodiments, where cells (donor cells) are not autologous to the subject, the expression of endogenous MHC or HLA gene(s) may be suppressed or disrupted, which may be achieved via any appropriate technique, such as but not limited to, an siRNA, shRNA, micro RNA, artificial microRNA, or gene editing using the CRISPR/Cas system, transcription activator-like effector nucleases (e.g., TALEN®), or Zinc finger nucleases (ZFNs). The suppression or disruption of MHC or HLA gene(s) may allow for reduction or prevention of undesired effects in which the subject's endogenous T cells recognize the donor cell's antigens presented on donor cell's MHC molecules as foreign and cause immune responses against the donor cells and increase the persistence of the administered cells within the subject. Cells expressing anti-NMDAR2B Abs or antigen-binding Ab fragments or a composition comprising such may also be administered to a subject. In some embodiments, B cells or plasma cells expressing anti-NMDAR2B Abs or antigen-binding Ab fragments may be adoptively transferred.

### Functional Activity

In one embodiment, the present invention includes a type of cellular therapy where isolated cells are genetically modified to express a CAR against NMDAR2B, and the CAR cell is infused into a subject in need thereof. Such administration can promote activation of the cells (e.g., T cell activation) in a target molecule specific manner, such that the cells of the disease or disorder are targeted for destruction. In the case where the cell is a T cell, cells, such as CAR T cells, are able to replicate in vivo resulting in long-term persistence that may lead to sustained control of diseases, disorders, or conditions associated with NMDAR2B, such as cancer.

In one embodiment, the isolated cells of the invention can undergo in vivo expansion and can persist for an extended amount of time. In another embodiment, where the isolated cell is a T cell, the isolated T cells of the invention evolve into specific memory T cells that can be reactivated to inhibit growth of any additional target molecule expressing cells. T cells may differentiate in vivo into a central memory-like state upon encounter and subsequent elimination of target cells expressing the surrogate antigen. Similarly, in certain embodiments, where the isolated cells are B cells, the isolated B cells may evolve into memory B cells that can be reactivated to inhibit the growth of any additional target molecule expressing cells.

Without wishing to be bound by any particular theory, the immune response elicited by the isolated anti-NMDAR2B Ab, Ab fragment, or CAR-modified immune cells may be an active or a passive immune response. In addition, the anti-NMDAR2B agent-mediated immune response may be part of an adoptive immunotherapy approach in which anti-NMDAR2B Ab, Ab fragment, or CAR-modified immune cells induce an immune response specific to the antigen-binding domain of the anti-NMDAR2B agent.

In certain embodiments, anti-NMDAR2B Ab, Ab fragment, or CAR-expressing cells are modified in any number of ways, such that their therapeutic or prophylactic efficacy is increased. For example, the anti-NMDAR2B Ab, Ab fragment, or CAR may be conjugated either directly or indirectly through a linker to a targeting moiety. The practice of conjugating compounds, e.g., the CAR, to targeting moieties is known in the art. See, for instance, Wadwa et al., J. Drug Targeting 3: 1 1 1 (1995), and U.S. Pat. No. 5,087,616.

Once the cells are administered to a subject (e.g., a human), the biological activity of the engineered cell populations and/or antibodies in some embodiments is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain mediators, such as GM-CSF, IL-6, RANTES (CCL5), TNF-α, IL-4, IL-10, IL-13, IFN-γ, granzyme B, perforin, CD 107a, or IL-2.

In some embodiments the biological activity is measured by assessing clinical outcome, such as the reduction in disease symptoms. In case of cancer, improved efficacy may be shown by better infiltration of disease-resolving immune cells into the tumor, reduced tumor sizes, or reduced ascites. In some embodiments, gene expression profiles may be also investigated to evaluate the activity.

### Target Cells

Cells that may be targeted by any anti-NMDAR2B agents of present invention include any NMDAR2B-expressing cells. The target cell may be present in any part of the body of a subject, including blood or lymphatic circulation, and disease-affected tissues. For example, when the target disease is solid cancer, the disease-affected tissues include, but are not limited to, pancreas, prostate, breast, lung, or ovarian tissue, or draining lymph nodes thereof. Alternatively, target cells may blood cells or hematopoietic cells.

Preferably, the anti-NMDAR2B agent-expressing cells of the invention are used to treat cancer, wherein NMDAR2B is upregulated. In particular, the cells of the invention may be used to treat pancreatic cancer, prostate cancer, ovarian cancer, breast cancer, lung cancer, especially SCLC, or glioma.

In general, cells that are positive for NMDAR2B may be identified via known methods, for example, immunofluorescence or flow cytometry using specific antibodies, or alternatively, through cytotoxicity against target cells. Methods of testing an anti-NMDAR2B agent for the ability to recognize target cells and for antigen specificity are known in the art. For instance, Clay et al., J. Immunol., 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, CAR function can be evaluated by measurement of cellular cytotoxicity, as described in Zhao et al., J. Immunol., 174: 4415-4423 (2005).

A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer. With respect to detecting the presence of cells expressing an anti-NMDAR2B Ab, Ab fragment, or CAR in a host, the sample comprising cells of the host can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the host, e.g., the cells of any organ or tissue, including blood cells or endothelial cells.

### Pharmaceutical Compositions

Also provided herein are pharmaceutical compositions.

The pharmaceutical compositions according to the present disclosure may comprise: (a) (i) the Ab or Ab fragment, (ii) the ADC, (iii) the CAR, (iv) the polynucleotide or combination of polynucleotides, (v) the vector or combination of vectors, (vi) the cell, and/or (vii) the population of cells, according to the present disclosure; and optionally (b) a pharmaceutically acceptable excipient or carrier.

The compositions of the present invention may be administered in a number of ways depending upon whether local or systemic treatment is desired.

In general, administration may be topical, parenteral, or enteral.

As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

Compositions of the present invention may be suited for topical, parenteral, or enteral administration.

Preferably, formulated compositions comprising Abs, antigen-binding Ab fragments, ADCs, or CARs, polynucleotides or vectors encoding such, or cells expressing thereof are suitable for administration via parenteral administration for example, subcutaneous, intramuscular, intraperitoneal or intravenous injection.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release. Such formulation may be, for example, made of a biodegradable, biocompatible polymer, such as, but not limited to, ethylene vinyl acetate, poly(alkyl cyanoacrylates), poly(anhydrides), poly(amides), poly(ester), poly(ester amides), poly(phosphoesters), polyglycolic acid (PGA), collagen, polyorthoester, polylactic acid (PLA), poly(lactic-co-glycolidic acid) (PLAGA), or naturally occurring biodegradable polymers such as chitosan and hyaluronic acid-based polymers (Kamaly N. et al, Chem Rev. Author manuscript; available in PMC 2017 Jul. 13).

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated to provide appropriate in vivo distribution of the active ingredient In many cases, concentrating the distribution of an anti-tumor drug in the tumor site is challenging, and it can be so even when a drug has a specificity to a molecule expressed by cancer cells. Various strategies have been developed to address the issue and any appropriate strategies may be applied for the current invention (for example, reviewed in Rosenblum D. et al., Nat Commun. 2018 Apr. 12; 9(1):1410. doi: 10.1038/s41467-018-03705-y). For delivering a drug to the brain, the drug needs to cross the blood-brain barrier (BBB). Any appropriate strategies to enable BBB crossing may be utilized to for the delivery of any of the anti-NMDAR2B agents of the agents (see for example, Dong X. et al., Theranostics. 2018; 8(6): 1481-1493, for exemplary strategies).

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising any of the anti-NMDAR2B agents of the present invention or populations of cells expressing any of the anti-NMDAR2B agents such as cells expressing anti-NMDAR2B CARs of the present invention may include pharmaceutically acceptable excipient(s). Excipients included in the formulations will have different purposes depending, for example, on the CAR construct, the subpopulation of cells used, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising populations of the CAR-expressing cells of the present invention will typically have been prepared and cultured in the absence of any non-human components, such as animal serum (e.g., bovine serum albumin).

The formulation or composition may also contain more than one active ingredient, such as therapeutic agents, useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs.

Such agents or drugs may be, but are not limited to: (i) an anti-cancer agent, an anti-proliferative drug, a cytotoxic drug, an anti-angiogenic drug, an apoptotic drug, an immunostimulatory drug, an NMDA receptor antagonist, an NMDA receptor signaling inhibitor, an NMDAR1 inhibitor, an NMDAR2B inhibitor; (ii) an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, or an oligonucleotide; (iii) a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof; (iv) an immunotherapeutic agent, optionally an immune checkpoint inhibitor or a growth factor or growth factor receptor inhibitor, further optionally an inhibitor of PD-L1, PD-1, CTLA-4, VISTA, EGF, EGFR, VEGF, and/or VEGFR, or an antibody or antibody fragment against PD-L1, PD-1, CTLA-4, VISTA, EGF, EGFR, VEGF, and/or VEGFR, or an antibody or antibody fragment against a cancer antigen other than NMDAR2B; and/or (v) an anti-emetic agent, optionally one or more selected from a neurokinin-1 receptor antagonist (NK1 RA), serotonin receptor antagonist (5-HT3 RA), dexamethasone, olanzapine, and palonosetron.

The pharmaceutical composition in some embodiments can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

### Kits

Also provided herein are kits comprising (a) one or more of anti-NMDAR2B Abs, antigen-binding Ab fragments, ADCs, polynucleotides encoding such, vectors encoding such, cells expressing such; and (b) for example an instruction for use in treating or diagnosing a disease or condition associated with NMDAR2B. The kit may include a label indicating the intended use of the contents of the kit. The term “label” as used herein includes any written materials, marketing materials, or recorded materials supplied on, with, in, or appended to the kit.

In some embodiments, a kit may be a diagnosis kit or a therapeutic kit

### Treatment Methods

Also provided herein are treatment methods using any of the anti-NMDAR2B agents and/or composition disclosed herein.

The methods encompass methods of treating a subject, methods of treating a disease in a subject, and methods of stimulating an immune response in a subject.

In some embodiment, administering to the subject in need thereof a therapeutically effective amount of: (i) the Ab or Ab fragment, (ii) the ADC, (iii) the CAR, (iv) the polynucleotide or combination of polynucleotides, (v) the vector or combination of vectors, (vi) the cell, (vii) the population of cells, or (viii) the pharmaceutical according to the present disclosure.

In some embodiments, the method may for stimulating an immune response against NMDAR2B-expressing cancer cells and/or for the treatment of cancer.

In some particular embodiments, the cancer may be pancreatic cancer, prostate cancer, ovarian cancer, small cell lung cancer, or breast cancer, or glioma.

In some embodiments, the method may further comprise administering another agent.

In some embodiments, the other agent may be: (i) an anti-cancer agent, an anti-proliferative drug, a cytotoxic drug, an anti-angiogenic drug, an apoptotic drug, an immunostimulatory drug, an NMDA receptor antagonist, an NMDA receptor signaling inhibitor, an NMDAR1 inhibitor, an NMDAR2B inhibitor; (ii) an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, or an oligonucleotide; (iii) a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof; (iv) an immunotherapeutic agent, optionally an immune checkpoint inhibitor or a growth factor or growth factor receptor inhibitor, further optionally an inhibitor of PD-L1, PD-1, CTLA-4, VISTA, EGF, EGFR, VEGF, and/or VEGFR, or an antibody or antibody fragment against PD-L1, PD-1, CTLA-4, VISTA, EGF, EGFR, VEGF, and/or VEGFR, or an antibody or antibody fragment against a cancer antigen other than NMDAR2B; and/or (v) an anti-emetic agent, optionally one or more selected from a neurokinin-1 receptor antagonist (NK1 RA), serotonin receptor antagonist (5-HT3 RA), dexamethasone, olanzapine, and palonosetron.

### Method of Administration

The administration route used in the method of the present invention may be any appropriate route, which depends upon whether local or systemic treatment is desired.

In general, administration may be topical, parenteral, or enteral.

Preferably, formulated compositions comprising Abs, antigen-binding Ab fragments, ADCs, or CARs, polynucleotides or vectors encoding such, cells expressing such may be administered parenterally, for example, via subcutaneous, intramuscular, intraperitoneal or intravenous injection.

In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the composition of the present invention may be administered using any appropriate medical devices (for example, reviewed in Richter B. B., J. BioDrugs (2018) 32: 425).

### Dosing

For administration of any of the anti-NMDAR2B agents and compositions of the present invention, the dosage will vary and depend on, for example, the target disease, the severity of the disease, the route of administration, and pharmacokinetic factors. Dosing may be modified based on the response observed in the subject.

For administration of any of the anti-NMDAR2B Abs, antigen-binding Ab fragments, or ADCs, or compositions comprising such, appropriate dosage regimen may be determined using any appropriate methodology (for example, Bai S. et al., Clin Pharmacokinet. 2012 Feb. 1; 51(2):119-35. doi: 10.2165/11596370-000000000-00000).

In some embodiments, the dosage may be from about 1 ng/kg to about 1 g/kg (of the body weight of a subject) per day. In some embodiments, the dose may be from about 10 ng/kg/day to about 900 mg/kg/day, from about 20 ng/kg/day to about 800 mg/kg/day, from about 30 ng/kg/day to about 800 mg/kg/day, from about 40 ng/kg/day to about 700 mg/kg/day, from about 50 ng/kg/day to about 600 mg/kg/day, from about 60 ng/kg/day to about 500 mg/kg/day, from about 70 ng/kg/day to about 400 mg/kg/day, from about 80 ng/kg/day to about 300 mg/kg/day, from about 90 ng/kg/day to about 200 mg/kg/day, or from about 100 ng/kg/day to about 100 mg/kg/day. The treatment may be repeated or periodically given to a subject for days, months, or years, or until the desired effect is achieved. An exemplary dosing regimen include administering an initial dose of an anti-NMDAR2B Abs, antigen-binding Ab fragments, or ADCs of about 2 mg/kg, followed by a weekly maintenance dose of about 1 mg/kg.

Dosing frequency may be, for example, three times per day, twice per day, once per day, every other day, once per week, every other week, once per three weeks, once per four weeks, once per five weeks, once per six weeks, once per seven weeks, once per eight weeks, once per nine weeks, once per ten weeks, once per three months, once per four months, once per six months, once per year, or even less frequent.

The pharmaceutical composition in some embodiments contains cells expressing the CAR of the present invention in amounts effective to treat or prevent the disease or condition, such as a therapeutically effective or prophylactically effective amount Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells expressing an anti-NMDAR2B agent such as a CAR-encoding and/or expressing cells, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 103 to about 109 cells per kg body weight including all integer values of cell numbers within those ranges.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection into the disease site.

For purposes of the invention, the amount or dose of the inventive anti-NMDAR2B agent administered should be sufficient to effect a therapeutic or prophylactic response in the subject or animal over a reasonable time frame. For example, the dose of the inventive anti-NMDAR2B agent should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular inventive anti-NMDAR2B agent and the condition of the animal (e.g., human), as well as the body weight of the animal (e.g., human) to be treated.

For purposes of the invention, an assay, which comprises, for example, comparing the extent to which target cells are lysed or, in the context of CARs, IFN-γ is secreted by T cells expressing the inventive CAR, polypeptide, or protein upon administration of a given dose of such T cells to a mammal, among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

In some embodiments, two or more of the anti-NMDAR2B agents or compositions of the present invention may be administered to a subject in combination or separately.

In some embodiments, the anti-NMDAR2B agents or compositions of the present invention are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In some contexts, the anti-NMDAR2B agents or compositions are co-administered with another therapy sufficiently close in time such that the anti-NMDAR2B agents or compositions enhance the effect of one or more additional therapeutic agents, or vice versa. In some embodiments, the cells or antibodies are administered prior to the one or more additional therapeutic agents. In some embodiments, the anti-NMDAR2B agents, such as anti-NMDAR2B CAR T cells or antibodies are administered after the one or more additional therapeutic agents. Furthermore, the compositions of the present invention may be given to a subject along with one or more of other therapies, which may be surgery, or a radiotherapy.

In some embodiments, in CART therapy, a lymphodepleting chemotherapy is administered to the subject prior to, concurrently with, or after administration (e.g., infusion) of CAR cells. In an example, the lymphodepleting chemotherapy is administered to the subject prior to administration of the cells. For example, the lymphodepleting chemotherapy ends 1-4 days (e.g., 1, 2, 3, or 4 days) prior to CAR cell infusion. In embodiments, multiple doses of CAR cells are administered, e.g., as described herein. In embodiments, a lymphodepleting chemotherapy is administered to the subject prior to, concurrently with, or after administration (e.g., infusion) of a CAR-expressing cell described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, anti-thymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination.

### Use as a Diagnostic Tool

The anti-NMDAR2B agents of the present invention, for example, anti-NMDAR2B Abs and antigen-binding Ab fragments, can be also useful as a diagnostic tool that may be used in vivo, ex vivo, or in vitro.

For example, an anti-NMDAR2B Abs or antigen-binding Ab fragment conjugated to an imaging agent may be administered to a subject or a patient to test if a diseased cell or tissue in the patient expresses NMDAR2B. The diagnoses may be done using any imaging tools that can detect the imaging agent. Alternatively, a biological sample such as, but is not limited to, blood or biopsy sample (e.g., cells or tissues suspected to contain cancer cells), may be obtained, and an anti-NMDAR2B Abs or antigen-binding Ab fragment may be applied to the sample to test the expression of NMDAR2B. In some embodiments, the Ab or Ab fragment may be attached to at least one detectable moiety, optionally selected from a fluorescent dye, an enzyme, a substrate, a bioluminescent material, a radioactive material, a chemiluminescent moiety, or a combination thereof.

A disease diagnosed by such a method may be cancer. In some embodiments, the cancer may be pancreatic cancer, prostate cancer, ovarian cancer, small cell lung cancer, breast cancer, or glioma.

These tests may determine whether the subject, or the cell or tissue of the subject, expresses NMDAR2B or not In some embodiments, the test may determine whether the subject, or the cell or tissue of the subject, expresses sufficient amount of NMDAR2B to be targeted by the anti-NMDAR2B therapeutic agent of the present invention. In some embodiments, the test may classify patients into different levels of NMDAR2B expression. In one aspect, a subject may be classified as an expressor or a non-expressor. In another aspect, a subject may be classified as an over-expressor, mid-expressor, or low-expressor.

In some embodiments, an appropriate therapeutic approach may be determined depending on the NMDAR2B expression. The expression may be determined using an anti-NMDAR2B agent of the present invention as described herein, or alternatively using any other appropriate method, such as, but not limited to, by measuring RNA expression levels or by quantifying NMDAR2B protein levels using an appropriate tool and/or technique. In one aspect, the anti-NMDAR2B agent of the present invention may be given to an expressor but not to a non-expressor. In another aspect the anti-NMDAR2B agent of the present invention may be given to an over-expressor but not to a mid-expressor or a low-expressor. In another aspect, the anti-NMDAR2B agent of the present invention may be given to an over-expressor or a mid-expressor but not to a low-expressor. In yet another aspect, the anti-NMDAR2B agent of the present invention may be given to a mid-expressor but not to a high-expressor or a low-expressor.

### Methods of Making

Anti-NMDAR2B agents of the present invention may be manufactured using any appropriate method used in the field.

The methods of making the Ab or Ab fragments according to the present disclosure may comprise: (a) culturing a cell comprising a polynucleotide or a combination of polynucleotides encoding the Ab or Ab fragment and/or comprising a vector or a combination of vectors encoding the Ab or Ab fragment in a condition that allows for expression of the Ab or Ab fragment; and optionally (b) purifying the Ab or Ab fragment from the culture.

The polynucleotide or the combination of polynucleotides may be according to any one of the Ab- or Ab fragment-encoding polynucleotides or the Ab- or Ab fragment-encoding combinations of polynucleotides disclosed herein. The vector or the combination of vectors may be according to any of the Ab- or Ab fragment-encoding vectors or the Ab- or Ab fragment-encoding combinations of vectors disclosed herein.

The cell may be according to any of the cells encoding the Ab or Ab fragment disclosed herein.

In certain embodiments, the methods of making the Ab or Ab fragments according to the present disclosure may comprise (a) in vitro translating or expressing a polynucleotide or a combination of polynucleotides encoding the Ab or Ab fragment; and optionally (b) purifying the Ab or Ab fragment.

The polynucleotide or the combination of polynucleotides may be according to any one of the Ab- or Ab fragment-encoding polynucleotides or the Ab- or Ab fragment-encoding combinations of polynucleotides disclosed herein.

The methods of making the cells encoding and/or expressing the CAR according to the present disclosure may comprise: (a) introducing into cells a polynucleotide or a combination of polynucleotides encoding the CAR and/or a vector or a combination of vectors encoding the CAR; (b) culturing the cells in a condition that allows for expression of the CAR; and optionally (c) purifying cells that express the CAR.

The polynucleotide or the combination of polynucleotides Ab- or Ab fragment-encoding according to any one of the CAR-encoding polynucleotides or the CAR-encoding combinations of polynucleotides disclosed herein. The vector or the combination of vectors Ab- or Ab fragment-encoding according to any of the CAR-encoding vectors or the CAR-encoding combinations of vectors disclosed herein. The cells obtained in step (c) may comprise at least one CAR-encoding and/or expressing cells according to the present disclosure.

In some embodiments, the CAR-encoding polynucleotide or the CAR-encoding combination of polynucleotides and/or a CAR-encoding vector or a CAR-encoding combination of vectors may encode a selection marker (e.g., trCD19) for expression of the CAR, and the purifying of step (c) may be based on the selection marker.

### Variations

Included in the scope of the invention are functional portions of the inventive anti-NMDAR2B agents described herein. The term “functional portion,” when used in reference to an Ab, antigen-binding Ab fragment, ADC, or CAR, refers to any part or fragment of the Ab, antigen-binding Ab fragment, ADC, or CAR of the invention, which part or fragment retains the biological activity of the Ab, antigen-binding Ab fragment, ADC, or CAR of which it is a part (the parent). Functional portions encompass, for example, those parts of an Ab, antigen-binding Ab fragment, ADC, or CAR that retain the ability to recognize target cells, or detect, treat, or prevent a disease, to a similar extent, the same extent, or to a higher extent, as the parent In reference to the parent Ab, antigen-binding Ab fragment, ADC, or CAR, the functional portion can comprise, for instance, about 10%, 25%, 30%, 50%, 68%, 80%, 90%, 95%, or more, of the parent.

The functional portion can comprise additional amino acids at the amino or carboxy terminus of the portion, or at both termini, which additional amino acids are not found in the amino acid sequence of the parent Ab, antigen-binding Ab fragment, ADC, or CAR. Desirably, the additional amino acids do not interfere with the biological function of the functional portion, e.g., recognize target cells, detect, treat, or prevent a target disease and/or condition, etc. More desirably, the additional amino acids enhance the biological activity, as compared to the biological activity of the parent Ab, antigen-binding Ab fragment, ADC, or CAR.

Included in the scope of the invention are functional variants of the inventive Abs, antigen-binding Ab fragments, ADCs, or CARs described herein. The term “functional variant” as used herein refers to an Ab, antigen-binding Ab fragment, ADC, or CAR polypeptide, or protein having substantial or significant sequence identity or similarity to a parent, which functional variant retains the biological activity of the Ab, antigen-binding Ab fragment, ADC, or CAR of which it is a variant Functional variants encompass, for example, those variants of the Ab, antigen-binding Ab fragment, ADC, or CAR described herein (the parent) that retain the ability to recognize target cells to a similar extent, the same extent, or to a higher extent, as the parent In reference to the parent Ab, antigen-binding Ab fragment, ADC, or CAR, the functional variant can, for instance, be at least about 30%, 50%, 75%, 80%, 90%, 98% or more identical in amino acid sequence to the parent.

A functional variant can, for example, comprise the amino acid sequence of the parent with at least one conservative amino acid substitution. Alternatively, or additionally, the functional variants can comprise the amino acid sequence of the parent with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent.

Amino acid substitutions of the inventive anti-NMDAR2B agents are preferably conservative amino acid substitutions. Conservative amino acid substitutions are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design.

The anti-NMDAR2B agents can consist essentially of the specified amino acid sequence or sequences described herein, such that other components, e.g., other amino acids, do not materially change the biological activity of the functional variant.

The Abs, antigen-binding Ab fragments, ADCs, or CARs of embodiments of the invention (including functional portions and functional variants) can be of any length, i.e., can comprise any number of amino acids, provided that the Abs, antigen-binding Ab fragments, ADCs, or CARs (or functional portions or functional variants thereof) retain their biological activity, e.g., the ability to specifically bind to antigen, detect diseased cells in a mammal, or treat or prevent disease in a mammal, etc. For example, the Ab, antigen-binding Ab fragment, ADC, or CAR can be about 50 to about 5000 amino acids long, such as 50, 70, 75, 100, 125, 150, 175, 200, 300, 400, 500, 600, 700, 800, 900, 1000 or more amino acids in length.

The Abs, antigen-binding Ab fragments, ADCs, or CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The Abs, antigen-binding Ab fragments, ADCs, or CARs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The Abs, antigen-binding Ab fragments, ADCs, or CARs of embodiments of the invention (including functional portions and functional variants thereof) can be obtained by methods known in the art. The Abs, antigen-binding Ab fragments, ADCs, and CARs may be made by any suitable method of making polypeptides or proteins. Suitable methods of de novo synthesizing polypeptides and proteins are described in references, such as Chan et al., “Fmoc Solid Phase Peptide Synthesis”, Oxford University Press, Oxford, United Kingdom, 2000; “Peptide and Protein Drug Analysis”, ed. Reid, R., Marcel Dekker, Inc., 2000; “Epitope Mapping”, ed. Westwood et al., “Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., “Molecular Cloning: A Laboratory Manual”, 3rd ed., Cold Spring Harbor Press, Cold Spring Harbor, N.Y. 2001; and Ausubel et al., “Current Protocols in Molecular Biology”, Greene Publishing Associates and John Wiley & Sons, N Y, 1994. Further, some of the Abs, antigen-binding Ab fragments, or CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the Abs, antigen-binding Ab fragments, ADCs, or CARs described herein (including functional portions and functional variants thereof) can be commercially synthesized by companies. In this respect, the inventive Abs, antigen-binding Ab fragments, ADCs, or CARs can be synthetic, recombinant, isolated, and/or purified.

### Definitions

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

Unless defined otherwise, all technical and scientific terms used herein have the same meaning as commonly understood by one of ordinary skill in the art to which this disclosure belongs.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “4-1BB,” “41BB,” or “BB” as used herein refers to a member of the TNFR superfamily with an amino acid sequence provided as GenBank Acc. No. AAA53133.1, or the equivalent residues from a non-human species, e.g., mouse, rodent, monkey, ape and the like. In one aspect, the “4-1BB costimulatory domain,” also referred to as “4-1BB CS domain” or “41BBCS,” may be derived from the cytoplasmic domain of 4-1BB. In some embodiments, “41BBCS” comprises the sequence provided as SEQ ID NO: 116 or the equivalent residues from a non-human species, e.g., mouse, rodent, monkey, ape and the like. In some embodiments, “41BBCS” may be encoded by a nucleic acid sequence provided as SEQ ID NO: 216.

The term “about” or “approximately” as used herein when referring to a numerical value, such as of weight, mass, volume, concentration, or time, should not be limited to the recited numerical value but rather encompasses variations of +/−10% of a given value.

The term “allogeneic” as used herein refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “antibody” or “Ab” is used herein in the broadest sense and encompasses various antibody structures, including but not limited to full-length or full-size immunoglobulins, monoclonal antibodies, polyclonal antibodies, multispecific antibodies (e.g., bispecific antibodies), and/or antibody fragments (preferably those fragments that exhibit the desired antigen-binding activity, which is also referred to as “antigen-binding antibody fragments”). Typically, a full-size Ab comprises two pairs (in case of IgD, IgE, IgG), four pairs (in case of IgA), or 10 pairs (in case of IgM) of chains, each pair comprising a heavy chain (HC) and a light chain (LC) interconnected by disulfide bonds. A HC typically comprises a variable region and a constant region. A LC also typically comprises a variable region and constant region.

The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively), respectively located between FR1 and FR2, between FR2 and FR3, and between FR3 and FR4. The constant region of a HC typically comprises a CH1 domain, hinge, a CH2 domain, and a CH3 domain. CH2 and CH3 domains form a fragment crystallizable region (Fc region), which dictates the isotype of the Ab (IgA (further divided into IgA1 and IgA2 subclasses), IgD, IgG (further divided into IgG1, IgG2, IgG3, and IgG4 subclasses), IgE, and IgM), the type of Fc receptor the Ab binds to, and therefore the effector function of the Ab. Fc receptor types include, but are not limited to, FcaR (such as FcaRI), Fca/mR, FceR (such as FceRI, FceRII),and FcgR (such as FcgRI, FcgRIIA, FcgRIIB1, FcgRIIB2, FcgRIIIA, FcgRIIIB) and their associated downstream effects are well known in the art. The “Fc region” of an Ab typically comprises a CH2 domain, and a CH3 domain.

The variable region of a light chain (VL) also typically comprises CDRs, which are CDR 1, CDR 2, and CDR 3 (or referred to as CDR-L1, CDR-L2, CDR-L3, respectively), respectively located between FR1 and FR2, between FR2 and FR3, and between FR3 and FR4. The constant region of a LC typically comprises a CL domain (kappa or lambda type).

According to IMGT, the CH1 domain is the amino acid positions (or simply referred to as “positions” herein) 118-215 (EU numbering). A human IgG1 CH1 domain reference sequence, corresponding to the amino acid positions 118-215 according to EU numbering, is provided herein as SEQ ID NO: 310, which is an exemplary amino acid sequence of a wild-type (WT) CH1 domain.

Human IgG1 CH1 domain reference sequence:

(SEQ ID NO: 310)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV.
(positions 118-215 according to EU numbering)

According to IMGT, the hinge is the amino acid positions 216-230 (EU numbering). A human IgG1 hinge reference sequence, corresponding to the amino acid positions 216-230 according to EU numbering, is provided herein as SEQ ID NO: 311, which is an exemplary amino acid sequence of a wild-type (WT) hinge.

Human IgG1 hinge reference sequence:

(SEQ ID NO: 311)
EPKSCDKTHTCPPCP.

According to IMGT, the CH2 domain is the amino acid positions 231-340 (EU numbering). A human IgG1 CH2 domain reference sequence, corresponding to the amino acid positions 231-340 according to EU numbering, is provided herein as SEQ ID NO: 312, which is an exemplary amino acid sequence of a wild-type (WT) CH2 domain.

Human IgG1 CH2 domain reference sequence:

(SEQ ID NO: 312)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK.

According to IMGT, the CH3 domain is the amino acid positions 341-446 (EU numbering). A CH3 domain reference sequence, corresponding to the amino acid positions 341-446 according to EU numbering, is provided herein as SEQ ID NO: 313, which is an exemplary amino acid sequence of a wild-type (WT) CH3 domain.

Human IgG1 CH3 domain reference sequence:

(SEQ ID NO: 313)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK.

Another commonly existing human IgG1 CH3 domain sequence, which differ from SEQ ID NO: 313 by two amino acid substitutions (E356D and M358L, relative to SEQ ID NO: 313), is further provided herein as SEQ ID NO: 315.

Human IgG1 CH3 domain alternative reference sequence (E356D and M358L)

(SEQ ID NO: 315)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK.

IgG antibodies often do not have the C-terminal lysine residue due to “C-terminal lysine clipping” and in some cases IgGs without the C-terminal lysine may have increased effector functions such as complement activation (van den Bremer et al. MAbs. 2015; 7(4):672-80.). Human IgG1 CH3 domain sequences without the C-terminal lysine are further provided herein as SEQ ID NO: 314 and 316.

Human IgG1 CH3 domain sequence (356E and 358M; without C-terminal K):

(SEQ ID NO: 314)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG.

Human IgG1 CH3 domain sequence (356D and 358L; without C-terminal K):

(SEQ ID NO: 316)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG.

There are two major CL isotypes, kappa (“κ”) and lambda (“λ”), and such CL domains are referred to herein as kappa CL domain (“CLκ” domain) and lambda CL domain (“CLλ” domain).

According to IMGT, the CLκ domain is the amino acid positions 108-214 (EU numbering). A CLκ domain reference sequence, corresponding to the amino acid positions 108-214 (EU numbering), is provided herein as SEQ ID NO: 325, which is an exemplary amino acid sequence of a wild-type (WT) CLκ domain.

CLκ domain reference sequence:

(SEQ ID NO: 325)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.
(positions 108 to 214 according to EU numbering)

According to IMGT, the CLλ domain is the amino acid positions 107-215 (EU numbering). A CLλ domain reference sequence, corresponding to the amino acid positions 107-215 (EU numbering), is provided herein as SEQ ID NO: 326, which is an exemplary amino acid sequence of a wild-type (WT) CLλ domain.

CLλ domain reference sequence:

(SEQ ID NO: 326)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS.
(positions 107 to 215 according to EU numbering)

The term “antibody-drug conjugate” “Ab-drug conjugate”, or “ADC” as used herein refers to a conjugate of an Ab or antigen-binding Ab fragment and a drug. The drug may be attached to any part of the Ab or the antigen-binding Ab fragment via a direct or indirect attachment, such as via a linker. In some embodiments, an ADC may comprise an antibody (or antibody fragment such as a single-chain variable fragment (scFv)) linked to a payload drug (often cytotoxic). The antibody causes the ADC to bind to the target cancer cells. In some embodiments, the ADC is then internalized by the cell and the drug is released into the cell. Because of the targeting, the side effects may be lower and may provide a wider therapeutic window. Hydrophilic linkers (e.g., PEG4Mal) may prevent the drug being pumped out of resistant cancer cells through MDR (multiple drug resistance) transporters. The present disclosure is also related to immunoconjugates comprising an anti-NMDAR2B binding agent conjugated to a therapeutic agent, such as a cytotoxin, a drug (e.g., an immunosuppressant) or a radiotoxin. Such conjugates may be referred to as “immunoconjugates”. Immunoconjugates that include one or more cytotoxins may also be referred to as “immunotoxins.” A cytotoxin or cytotoxic agent includes any agent that is detrimental to (e.g., kills) cells. Cytotoxins can be conjugated to antibodies according to at least some embodiments of the invention using linker technology available in the art Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55: 199-215; Trail, P. A. et al. (2003) Cancer Immunol. Immunother. 52:328-337; Payne, G. (2003) Cancer Cell 3:207-212; Allen, T. M. (2002) Nat Rev. Cancer 2:750-763; Pastan, I. and Kreitman, R. J. (2002) Curr. Opin. Investig. Drugs 3: 1089-1091; Senter, P. D. and Springer, C. J. (2001) Adv. Drug Deliv. Rev. 53:247-264. Antibodies of the present invention also can be conjugated to a radioactive isotope to generate cytotoxic radiopharmaceuticals, also referred to as radioimmunoconjugates.

The term “antibody fragment” or “Ab fragment” as used herein refers to any portion or fragment of an Ab, including intact or full-length Abs that may be of any class or sub-class, including IgG and sub-classes thereof, IgM, IgE, IgA and sub-classes thereof, and IgD. The term encompasses molecules constructed using one or more portions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv.

Unless otherwise stated, the term “Ab fragment” should be understood to encompass functional antibodies and fragments thereof. A portion of an Ab fragment that comprises a structure that enables specific binding to an antigen may be referred to as “antigen-binding Ab fragment,” “antigen-binding domain,” or “antigen-binding region” of the Ab fragment.

A “heavy chain” or “HC” of an Ab, as used herein, refers to the larger of the two types of polypeptide chains present in all Ab molecules in their naturally occurring conformations.

A “light chain” or “LC” of an Ab, as used herein, refers to the smaller of the two types of polypeptide chains present in all Ab molecules in their naturally occurring conformations. Kappa and lambda light chains refer to the two major antibody light chain isotypes.

An “anti-NMDAR2B agent” or “anti-NMDAR2B material” as used herein refers to any agents that are able to target NMDAR2B directly or indirectly. Anti-NMDAR2B agents of the present invention include, but are not limited to, anti-NMDAR2B Abs, anti-NMDAR2B antigen-binding Ab fragments, anti-NMDAR2B multi-specific Abs, anti-NMDAR2B multi-specific antigen-binding Ab fragments, anti-NMDAR2B ADCs, and anti-NMDAR2B CARs, and polynucleotides and vectors encoding the same, and cells encoding and/or expressing the same. In a broad sense, anti-NMDAR2B agents may also encompass pharmaceutical compositions comprising any of the above-mentioned anti-NMDAR2B agents.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one genes and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a cancer tissue sample, a tumor tissue sample, a leukemic cell sample, an inflamed tissue sample, and a cell or a fluid with other biological components. In some embodiments, the antigen is NMDAR2B.

The term “antigen-binding domain” refers to a portion of the anti-NMDAR2B agents, such as anti-NMDAR2B chimeric antigen receptors, of the present invention and the portion comprises a structure that allows for specific binding of the anti-NMDAR2B agents to NMDAR2B. When the anti-NMDAR2B agent is an Ab, the antigen-binding domain may comprise the variable region of the Ab or a portion of the variable region, such as the CDRs. When the anti-NMDAR2B agent is an antigen-binding Ab fragment or an antibody-drug conjugate, the antigen-binding domain may comprise the variable region or a portion of the variable region, such as the CDRs, of the Ab that the anti-NMDAR2B agent is derived from. When the anti-NMDAR2B agent is a chimeric antigen receptor (CAR), the antigen-binding domain may be one or more extracellular domains of the CAR which have specificity for NMDAR2B. When the antigen-binding domain is derived from an Ab or antigen-binding Ab fragment, the antigen-binding domain may comprise the antigen-binding domain, such as the variable region or a portion of the variable region, such as the CDRs, of the Ab or antigen-binding Ab fragment that it is derived from. In some embodiments, the antigen-binding domain of an anti-NMDAR2B agent of the present invention is scFv.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” or “donor-derived” as used herein refers to any material derived from the same individual to whom it is later to be re-introduced.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “cancer” refers to a disease characterized by the uncontrolled growth of aberrant cells. Cancer cells can spread locally or through the bloodstream and lymphatic system to other parts of the body. Examples of various cancers relevant to the present invention include, but are not limited, pancreatic cancer, testicular cancer, cervical cancer, endometrial cancer, ovarian cancer, stomach cancer, colorectal cancer, lung cancer, mesothelioma, and tongue cancer.

The term “bispecific” as used herein refers to having two binding specificities. An anti-NMDAR2B bispecific Ab or a bispecific antigen-binding Ab fragment, for example, of the present invention has at least one specificity for NMDAR2B. When the first specificity is for an epitope for NMDAR2B, the second specificity may be for another non-overlapping or non-competing epitope for NMDAR2B or may be for a molecule other than NMDAR2B, such as CD3. The term “bispecific” is also used in the same manner for any other anti-NMDAR2B agents of the present invention, such as anti-NMDAR2B CARs.

The term “CD28” refers to the protein Cluster of Differentiation 28, one of the proteins expressed on T cells that provide co-stimulatory signals required for T cell activation and survival. Human CD28 protein may have at least 85, 90, 95, 96, 97, 98, 99 or 100% identity to NCBI Reference No: NP_006130 or a fragment thereof that has stimulatory activity. The term “CD28 transmembrane domain,” also referred to as “CD28 TM domain” or “CD28TM” refers to the amino acid residues derived from the transmembrane domain of CD28. In some embodiments, “CD28TM” comprises the sequence provided as SEQ ID NO: 114 or the equivalent residues from a non-human species, e.g., mouse, rodent, monkey, ape and the like. In some embodiments, “CD28 TM domain” may be encoded by the nucleic acid sequence provided as SEQ ID NO: 141. The term “CD28 hinge” as used herein refers to amino acid residues that may be used to join two domains or two portions within a domain in CARs of some of the embodiments. In some embodiments, “CD28 hinge” comprises the sequence provided as SEQ ID NO: 113 or the equivalent residues from a non-human species, e.g., mouse, rodent, monkey, ape and the like. In some embodiments, “CD28 hinge” may be encoded by the nucleic acid sequence provided as SEQ ID NO: 213. The term “CD28 costimulatory domain,” also referred to as “CD28 CS domain” or “CD28CS,” refers to the amino acid residues derived from the cytoplasmic domain of CD28. In some embodiments, “CD28CS” comprises the sequence provided as SEQ ID NO: 115 or the equivalent residues from a non-human species, e.g., mouse, rodent, monkey, ape and the like. In some embodiments, “CD28 CS domain” may be encoded by the nucleic acid sequence provided as SEQ ID NO: 215.

The term “CD3 zeta,” or alternatively, “zeta,” “zeta chain,” “CD3-zeta,” “CD3z,” “TCR-zeta,” or “CD247,” is a protein encoded by the CD247 gene on chromosome 1, with gene location 1q24.2, in humans. CD3 zeta, together with T cell receptor (TCR) and CD3 (a protein complex composed of a CD3 gamma, a CD3 delta, and two CD3 epsilon), forms the TCR complex. Human CD3 zeta may have an amino acid sequence provided as NP_000725 or NP_932170, or the equivalent residues from a non-human species, e.g., mouse, rodent, monkey, ape and the like. The term “CD3 zeta intracellular signaling domain,” or alternatively “CD3 zeta ICS domain” or a “CD3zICS,” is defined as the amino acid residues from the cytoplasmic domain of the CD3 zeta chain, or functional derivatives thereof, that are sufficient to functionally transmit an initial signal necessary for T cell activation. In one aspect, “CD3 zeta ICS domain” is the sequence provided as SEQ ID NO: 118. In one aspect, “CD3 zeta ICS domain” is encoded by the nucleic acid sequence provided as SEQ ID NO: 218.

The term “Chimeric Antigen Receptor” or alternatively a “CAR” refers to a set of polypeptides, typically two in the simplest embodiments, which when in an immune effector cell, provides the cell with specificity for a target cell, and with intracellular signal generation. In some embodiments, a CAR comprises at least an extracellular antigen-binding domain (AB domain), a transmembrane domain (TM domain) and a cytoplasmic signaling domain (also referred to herein as “an intracellular signaling domain (ICS domain)”) comprising a functional signaling domain derived from a stimulatory molecule and/or costimulatory molecule as defined below. In some embodiments, the set of polypeptides are contiguous with each other. In some embodiments, the set of polypeptides include a dimerization switch that, upon the presence of a dimerization molecule, can couple the polypeptides to one another, e.g., can couple an antigen-binding domain to an ICS domain. In one aspect, the stimulatory molecule is the zeta chain associated with the T cell receptor complex. In one aspect, the cytoplasmic portion of a CAR further comprises a costimulatory domain (CS domain) comprising one or more functional signaling domains derived from at least one costimulatory molecule as defined below. In one aspect, the costimulatory molecule is chosen from the costimulatory molecules described herein, e.g., 4-1BB (i.e., CD137), DAP10 and/or CD28. In one aspect, the CAR comprises a chimeric fusion protein comprising an extracellular antigen-binding domain, a TM domain and an ICS domain comprising a functional signaling domain derived from a stimulatory molecule. In one aspect, the CAR comprises a chimeric fusion protein comprising an extracellular antigen-binding domain, a TM domain, an ICS domain comprising a functional signaling domain derived from a stimulatory molecule, and a CS domain comprising a functional signaling domain derived from a costimulatory molecule. In one aspect, the CAR comprises a chimeric fusion protein comprising an extracellular antigen-binding domain, a TM domain, an ICS domain comprising a functional signaling domain derived from a stimulatory molecule, and two CS domains each of the two comprising a functional signaling domain derived from a costimulatory molecule(s) that is/are same with or different from each other. In one aspect, the CAR comprises a chimeric fusion protein comprising an extracellular antigen-binding domain, a TM domain, an ICS domain comprising a functional signaling domain derived from a stimulatory molecule, and at least two CS domains each comprising a functional signaling domain derived from a costimulatory molecule(s) that is/are same with or different from each other. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In some embodiments, the leader sequence (LS) comprises the amino acid sequence provided as SEQ ID NO: 112. In some embodiments, the LS may be encoded by a nucleic acid sequence provided as SEQ ID NO: 212.

The term “compete”, as used herein with regard to an Ab, antigen-binding Ab fragment, of antigen-binding domain of any of the anti-NMDAR2B agents of the present invention, means that a first Ab, antigen-binding Ab fragment, or antigen-binding domain, binds to an epitope in a manner sufficiently similar to the binding of a second Ab, antigen-binding Ab fragment, or antigen-binding domain, such that the result of binding of the first Ab, antigen-binding Ab fragment, or antigen-binding domain with its cognate epitope is detectably decreased in the presence of the second Ab, antigen-binding Ab fragment, or antigen-binding domain compared to the binding of the first Ab, antigen-binding Ab fragment, or antigen-binding domain in the absence of the second Ab, antigen-binding Ab fragment, or antigen-binding domain. The alternative, where the binding of the second Ab, antigen-binding Ab fragment, or antigen-binding domain to its epitope is also detectably decreased in the presence of the first antibody, can, but need not be the case. That is, a first Ab, antigen-binding Ab fragment, or antigen-binding domain can inhibit the binding of a second Ab, antigen-binding Ab fragment, or antigen-binding domain to its epitope without that second Ab, antigen-binding Ab fragment, or antigen-binding domain inhibiting the binding of the first Ab, antigen-binding Ab fragment, or antigen-binding domain to its respective epitope. However, where each Ab, antigen-binding Ab fragment, or antigen-binding domain detectably inhibits the binding of the other Ab, antigen-binding Ab fragment, or antigen-binding domain with its cognate epitope or ligand, whether to the same, greater, or lesser extent, the two (Ab, antigen-binding Ab fragment, or antigen-binding domain) are said to “cross-compete” with each other for binding of their respective epitope(s). Both competing and cross-competing Abs, antigen-binding Ab fragments, or antigen-binding domains are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing Abs, antigen-binding Ab fragments, or antigen-binding domains are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

The term “conservative amino acid substitutions” herein are as commonly used in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc. Non-conservative amino acid substitutions are amino acid substitutions that are not conservative amino acid substitutions.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83. In embodiments wherein a CAR comprises one or more CS domain, each CS domain comprises a functional signaling domain derived from a costimulatory molecule. In some embodiments, the encoded CS domain comprises 4-1BB, CD28, or DAP10. In one embodiment, the CS domain comprises the amino acid sequence of CD28CS, 41BBCS, or DAP10CS (SEQ ID NO: 115, 116, or 117), or is encoded by the nucleotide sequence encoding provided as SEQ ID NOs: 215, 216, or 217.

The term “cytokines” as used herein refers to a broad category of small proteins that are involved in cell signaling. Generally, their release has some effect on the behavior of cells around them. Cytokines may be involved in autocrine signaling, paracrine signaling and/or endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, epithelial cells, and various stromal cells. “Chemokines” are a family of cytokines generally involved in mediating chemotaxis.

The term “cytotoxicity” generally refers to any cytocidal activity resulting from the exposure of the anti-NMDAR2B agents of the invention or cells comprising the same to cells expressing NMDAR2B. This activity may be measured by known cytotoxicity assays, including IFN-γ production assays. When the target cell is a cancer or tumor cell, the term “anti-cancer cytotoxicity” or “anti-tumor cytotoxicity” may be used.

The term “DAP10” refers to a protein, which in humans is encoded by the HSCT gene. It may also be referred to as HSCT, KAP10, PIK3AP, or hematopoietic cell signal transducer. In some embodiments, DAP10 may have the sequence provided in Genbank Accession No.: Q9UBK5.1. The term “DAP10 costimulatory domain,” also referred to as “DAP10 CS domain” or “DAP10CS,” refers to the amino acid residues derived from the cytoplasmic domain of DAP10. In some embodiments, “DAP10CS” comprises the sequence provided as SEQ ID NO: 117 or the equivalent residues from a non-human species, e.g., mouse, rodent, monkey, ape and the like. In some embodiments, “DAP10 CS domain” may be encoded by the nucleic acid sequence provided as SEQ ID NO: 217.

The phrase “disease associated with expression of NMDAR2B” or “NMDAR2B-associated disease” includes, but is not limited to, a disease associated with expression of NMDAR2B or condition associated with cells which express NMDAR2B including, e.g., proliferative diseases such as a cancer or malignancy or a precancerous condition; or a noncancer-related indication associated with cells which express NMDAR2B. Examples of various cancers that express NMDAR2B include but are not limited to, pancreatic cancer, testicular cancer, cervical cancer, endometrial cancer, ovarian cancer, stomach cancer, colorectal cancer, lung cancer, mesothelioma, and tongue cancer, and the like.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, another pre-existing condition, and the judgment of the prescribing physician. The size of the dose will also be determined by the active ingredient selected, method of administration, timing and frequency of administration, the existence, nature, and extent of any adverse side effects that might accompany the administration of a particular active ingredient, and the desired physiological effect. It will be appreciated by one of skill in the art that various diseases or disorders could require prolonged treatment involving multiple administrations, perhaps using the inventive anti-NMDAR2B agents, nucleic acids, vectors, cells, or compositions in each or various rounds of administration.

The terms “enteral,” “enterally,” “oral,” “orally,” “non-parenteral,” “non-parenterally,” and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “framework” as used herein refers to the non-CDR portions of the variable region of an Ab, or in some embodiments, Antigen-binding Ab fragment or an antigen-binding domain of a CAR. “Heavy chain (HC) framework” and “VH framework” are used interchangeably herein and refer to the non-CDR portion of a HC variable region, and in general, there are four framework regions (FRs) in each full-length heavy chain variable region (FR-H1, FR-H2, FR-H3, and FR-H4). “Light chain (LC) framework” and “VL framework” are used interchangeably herein and refer to the non-CDR portion of a LC variable region, and in general, there are four FRs in each full-length light chain variable region (FR-L1, FR-L2, FR-L3, and FR-L4). In some embodiments, “human HC framework”, “human VH framework”, “human-like HC framework”, or “human-like VH framework” is at least 80%, at least 85%, at least 90%, at least 92%, at least 94%, at least 95%, at least 96%, at least 97%, at least 98%, at least 99%, or 100% identical to a human HC framework. In some embodiments, “human LC framework”, “human VL framework”, “human-like LC framework”, or “human-like VL framework” is at least 80%, at least 85%, at least 90%, at least 92%, at least 94%, at least 95%, at least 96%, at least 97%, at least 98%, at least 99%, or 100% identical to a human LC framework.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “hinge”, “spacer”, or “linker” refers to an amino acid sequence of variable length typically encoded between two or more domains or portions of a polypeptide construct to confer flexibility, improved spatial organization, proximity, etc.

As used herein, “human antibody” means an antibody having an amino acid sequence corresponding to that of an antibody produced by a human and/or which has been made using any of the techniques for making human antibodies known to those skilled in the art or disclosed herein. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “humanization” of an Ab refers to modification of an Ab of a non-human origin to increase the sequence similarity to an Ab naturally produced in humans. The term “humanized antibody” as used herein refers to Abs generated via humanization of an Ab. Generally, a humanized or engineered antibody has one or more amino acid residues from a source which is non-human, e.g., but not limited to mouse, rat, rabbit, non-human primate or another mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html, each entirely incorporated herein by reference. Such imported sequences can be used to reduce immunogenicity or reduce, enhance or modify binding, affinity, avidity, specificity, half-life, or any other suitable characteristic, as known in the art Generally, part or all of the non-human or human CDR sequences are maintained while part or all of the non-human sequences of the framework and/or constant regions are replaced with human or other amino acids. Antibodies can also optionally be humanized with retention of high affinity for the antigen and other favorable biological properties using three-dimensional immunoglobulin models that are known to those skilled in the art Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, for example, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

The term “iCAR” is a chimeric antigen receptor which contains inhibitory receptor signaling domains. These domains may be based, for example, on protectin D1 (PD1) or CTLA-4 (CD152). In some embodiments, the CAR expressing cells of the invention are further transduced to express an iCAR. In one aspect, this iCAR is added to restrict the CAR expressing cell's functional activity to tumor cells.

The term “immune cell” refers to a cell of hematopoietic origin functionally involved in the initiation and/or execution of innate and/or adaptive immune response.

The term “intracellular signaling domain” or “ICS domain” as used herein, refers to an intracellular portion of a molecule. The intracellular signaling domain generates a signal that promotes an immune effector function of the cell transduced with a polynucleotide comprising a CAR, e.g., a CAR T cell. Examples of immune effector function, e.g., in a CAR T cell, include cytolytic activity and helper activity, including the secretion of cytokines. ICS domains include an ICS domain of a lymphocyte receptor chain, a TCR/CD3 complex protein, an Fc receptor subunit, an IL-2 receptor subunit, CD3 zeta, FcR gamma, FcR beta, CD3 gamma, CD3 delta, CD3 epsilon, CD5, CD22, CD79a, CD79b, CD66d, CD278(ICOS), Fc epsilon RI, DAP10, or DAP12.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

A “leader sequence” or “LS” as used herein, also referred to as “signal peptide,” “signal sequence,” “targeting signal,” “localization signal,” “localization sequence,” “transit peptide,” or “leader peptide” in the art, is a short peptide present at the N-terminus of the majority of newly synthesized proteins that are destined towards the secretary pathway. The core of the signal peptide may contain a long stretch of hydrophobic amino acids. The signal peptide may or may not be cleaved from the mature polypeptide.

The term “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises one or more repeats of the amino acid sequence unit Gly-Gly-Gly-Gly-Ser (SEQ ID NO: 110), which may be encoded by SEQ ID NO: 210. In one embodiment, the flexible polypeptide linker includes, but is not limited to, (Gly4Ser)3, which is also referred to as G4S X3 (SEQ ID NO: 111). Such a linker may be encoded for example, by the nucleic acid sequence as set forth in SEQ ID NO: 211.

The term “mammal” refers to any mammal, including, but not limited to, mammals of the order Rodentia, such as mice, rats, and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “masked CAR” refers to a CAR expressing cell that further comprises a masking peptide. This masking peptide may prevent off-target cell killing. The masking peptide is often N-terminal to the CAR construct and may block the cell's ability to bind to unintended targets. The masking peptide may be cleaved from the CAR expressing cell when it encounters a tumor thereby allowing the CAR expressing cell to attack its target without killing off-target cells. An anti-NMDAR2B CARs of the present invention may be constructed to be a masked CAR.

The term “multispecific” as used herein refers to having two or more binding specificities. An anti-NMDAR2B multispecific Ab or a multispecific antigen-binding Ab fragment, for example, of the present invention has at least one specificity for NMDAR2B. When the first specificity is for an epitope for NMDAR2B, the second (or third, fourth, and so forth) specificity may be for another epitope for NMDAR2B or may be for a molecule other than NMDAR2B. The term “multispecific” is also used in the same manner for any other anti-NMDAR2B agents of the present invention, such as anti-NMDAR2B CARs.

The term “NMDAR2B” as used herein, is the subtype B of the NR2 subunit protein of NMDA receptors (Paoletti P, et al. Nat Rev Neurosci. 2013 June; 14(6):383-400). NMDAR2B is also known as NMDA receptor subtype 2B, NMDA receptor subunit epsilon-2, NR2B, hNR3, glutamate receptor subunit epsilon-2, GluN2B, glutamate ionotropic receptor NMDA type subunit 2B, GRIN2B, MRD6, EIEE27NR3, DEE27. In humans, NMDAR2B is encoded by the GRIN2B gene on chromosome 12, with gene location 12p13.1 (NCBI). Human NMDAR2B may have an amino acid sequence provided as NCBI Reference Sequence: NP_000825.2. In one aspect, human NMDAR2B-L has the amino acid sequence provided as SEQ ID NO: 1 or the equivalent residues from a non-human species, e.g., mouse, rodent, monkey, ape and the like. SEQ ID NO: 1 may encoded by, e.g., SEQ ID NO: 11.

The term “NMDA receptor antagonist” refers to a molecule or a substance which is capable of inhibiting, blocking, deactivating, and/or antagonizing the NMDA receptor function such as but not limited to the receptor signaling. NMDA receptor antagonists may be classified into four categories based on the inhibiting, blocking, deactivating, and/or antagonizing mechanism. “Competitive antagonists” bind to and/or block the glutamate-binding site of NMDA receptors. “Uncompetitive antagonists” blocks the ion channel and prevents ions to path through NMDA receptors. “Non-competitive antagonists” binds to a site of NMDA receptors other than the ligand-binding site (i.e., an allosteric site) and prevents the ion channel from fully opening. “Glycine antagonist” bind to and/or block the glycine-binding site of NMDA receptors. For example, ifenprodil is a noncompetitive NMDA antagonist, which specifically antagonizes NMDARB2-containing NMDA receptors (Williams K. Mol Pharmacol. 1993 October; 44(4):851-9.).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “OKT3” or “Muromonab-CD3” or “Orthoclone OKT3” refers to a monoclonal anti-CD3 Ab.

The term “parenteral” or “parenterally” as used herein includes any route of administration of a compound or composition, characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage.

The term “promoter”, as used herein, is defined as a DNA sequence recognized by the synthetic machinery of the cell, or introduced synthetic machinery, required to initiate the specific transcription of a polynucleotide sequence.

The “ribosome skip sequence” refers to an amino acid sequence that, when translated, causes cleavage of a nascent polyprotein on the ribosome, allowing for co-expression of multiple genes. In one aspect, the ribosome skip sequence may be the T2A sequence such as SEQ ID NO: 119, which may be encoded by SEQ ID NO: 219. Alternatively, any other 2A sequences may be used. Examples of other sequences may be found elsewhere in the literature of the relevant art (for example, see Kim, J. H., et al., High cleavage efficiency of a 2A peptide derived from porcine teschovirus-1 in human cell lines, zebrafish and mice. PLoS One. 2011; 6(4)).

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). In this case, when the scFv is for example derived from Ab9, the construct may be referred to as Ab9scFvHL, Ab9HL, Ab9scFvVHVL, or Ab9VHVL. Alternatively, the heavy chain variable domain may be placed downstream of the light chain variable domain, and the two domains may optionally be linked via a linker (for example, the G4S X3 linker). In this case, when the scFv is for example derived from Ab9, the construct may be referred to as Ab9scFvLH, Ab9LH, Ab9scFvVLVH, or Ab9VLVH. The same naming rules apply to other similar constructs herein.

The term “signaling domain” refers to the functional portion of a protein which acts by transmitting information within the cell to regulate cellular activity via defined signaling pathways by generating second messengers or functioning as effectors by responding to such messengers.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence that are of particular use in the invention include, but are not limited to, those derived from CD3 zeta, common FcR gamma (FCER1G), Fc gamma RIIa, FcR beta (Fc epsilon R1b), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3 zeta. In a specific CAR of the invention, the primary signaling sequence of human CD3 zeta, referred to as “CD3zICS” herein, is the amino acid sequence provided as SEQ ID NO: 118, and may be encoded by the nucleotide sequence SEQ ID NO: 218. Alternatively, equivalent residues from a non-human or mouse species, e.g., rodent, monkey, ape and the like, may be utilized.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “suicide mechanism” as used herein refers to a mechanism by which anti-NMDAR2B agent-expressing cells of present invention may be eradicated from a subject administered with such cells. The suicide mechanism may be driven by, for example, inducible caspase 9 (Budde L. E. et al., PLoS One. 2013 Dec. 17; 8(12):e82742. doi: 10.1371/journal.pone.0082742. eCollection 2013), codon-optimized CD20 (Martin V. et al., Hum Gene Ther Methods. 2012 December; 23(6): 376-386), CD34, or polypeptide RQR8 (Philip et al, and WO2013153391A, which is hereby incorporated herein by reference). In some embodiments, the suicide mechanism may be included and utilized in CAR-expressing cells of present invention to optimize the length for the CAR-expressing cells to stay in the system of a subject or the amount of the CAR-expressing cells, to reduce or minimize the toxicity and/or to maximize the benefit of CAR-expressing cells.

The term “synthetic Ab” or “synthetic antigen-binding Ab fragment” as used herein, refers to an Ab or antigen-binding Ab fragment which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “target” as used herein refers to the molecule that an anti-NMDAR2B agents of the present invention specifically binds to. The term also encompasses cells and tissues expressing the target molecule and also diseases that are associated with expression of the target.

The term “target cell” as used herein refers to a cell expressing the target molecule (such as NMDAR2B) of the anti-NMDAR2B agents of the present invention on the cell surface. In some embodiments, the target cell is a cancer cell or tumor cell. In some embodiments, the target cell is a vascular cell. In some embodiments, the target cell is an epithelial cell. In some embodiments, the target cell is a cell type that has a particular role in the pathology of cancer or inflammation. In some embodiments, the target cell is a cell type that has a particular role in the pathology of a disease such as but not limited to cancer.

The term “target molecule” as used herein refers to a molecule that is targeted by the anti-NMDAR2B agents of the present invention. The antigen-binding domain of the anti-NMDAR2B agents of the present invention has a binding affinity for the target molecule. In some embodiments, the target molecule is NMDAR2B.

The term “trCD19” refers to a truncated version of the CD19 protein, B-lymphocyte antigen CD19, also known as CD19 (Cluster of Differentiation 19), which is a protein that is encoded by the CD19 gene in humans and is found on the surface of B-cells. The trCD19 construct is any truncated version of said protein, such that a nucleic acid sequence encoding this construct may be transduced into a host cell and expressed on the surface of this cell for the purposes of detection, selection, and/or targeting. In one aspect, human trCD19 may comprise the amino acid sequence of SEQ ID NO: 120, which may be encoded by e.g., the nucleotide sequence SEQ ID NO: 220.

The term “transfected,” “transformed,” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “transmembrane domain” or “TM domain”, what is implied is any three-dimensional protein structure which is thermodynamically stable in a membrane. This may be a single alpha helix, a transmembrane beta barrel, a beta-helix of gramicidin A, or any other structure. Transmembrane helices are usually about 20 amino acids in length. Typically, the transmembrane domain denotes a single transmembrane alpha helix of a transmembrane protein, also known as an integral protein.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The type of disease or condition to be treated may be, for example, but are not limited to, cancer and cancer-associated diseases and conditions. Examples of cancer include, but are not limited to, pancreatic cancer, testicular cancer, cervical cancer, endometrial cancer, ovarian cancer, stomach cancer, colorectal cancer, lung cancer, mesothelioma, and tongue cancer. In specific embodiments, the effect of the “treatment” may be evaluated by the amelioration of at least one measurable physical parameter of a disease, resulting from the administration of one or more therapies (e.g., an anti-NMDAR2B Ab or antigen-binding Ab fragment, anti-NMDAR2B ADC, or anti-NMDAR2B CAR expressing cell). The parameter may be, for example, gene expression profiles, the mass of disease-affected tissues, inflammation-associated markers, cancer-associated markers, the number or frequency of disease-associated cells, tumor burden, the presence or absence of certain cytokines or chemokines or other disease-associated molecules, and may not necessarily discernible by the patient In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

The term “xenogeneic” or “xeno-” refers to a graft derived from an animal of a different species.

Examples are provided below to illustrate the present invention. These examples are not meant to constrain the present invention to any particular application or theory of operation.

### EXAMPLES

### Example 1: NMDA Receptor Expression in Aggressive Prostate Cancer

### <Methods>

Generation of anti-NMDAR1 polyclonal Ab: An amidated extracellular N-terminal segment (SEQ ID NO: 5) of human NMDAR1 was coupled to bovine thyroglobulin and used as an immunogen. Rabbits were immunized with the immunogen and antisera were collected. The active IgG2b fraction was isolated using protein A Sepharose and used for experiments.

### Immunohistochemistry:

A tissue array of 12 invasive prostate adenocarcinoma cancers and a series of normal human tissues (ovary, kidney, breast, and lung). 4 m sections were generated and immunohistochemistry was performed using the Streptavidin-Biotin Complex (SABC) technique with citrate-based antigen recovery (BioGenex). Controls included successfully blocking primary Ab with excess of peptide antigen.

### <Results>

NMDAR1 expression was confirmed in seemingly all neoplastic cells of all tumor microdots and all cancer sections tested. FIG. 4A shows exemplary NMDAR1 staining on sections from two pancreatic cancer patients. FIG. 4B shows exemplary comparison between anti-NMDAR1 staining and negative control staining. No staining was observed in any of the normal tissues, and this was further confirmed by no detection of NMDAR1 protein by Western blot.

### Example 2: NMDA Receptor Expression on Prostate Cancer Cell Lines

### <Methods>

NMDAR1 expression by LNCAP and PC3 cell lines, both of which represent aggressive adenocarcinoma subtype of prostate cancer, were analyzed by Western blot using the polyclonal Ab described in Example 1 and cell staining with an antibody against the extracellular N-terminal segment (SEQ ID NO: 5) of human NMDAR1 followed by confocal microscopy.

### <Results>

NMDAR1 expression was confirmed by both Western blot and confocal microscopy. FIG. 5 shows exemplary results from the confocal microscopy analysis.

### Example 3: Effects of NMDA Receptor Inhibition on Cancer Cell Viability

### <Methods>

LNCAP cells were plated in 96 well plates at 1-2×10⁴ cells/well. The cells were incubated with PBS with glycine containing albumin (1.25 mg/ml) in the presence of different concentrations of a NMDAR receptor antagonist, MK-801 (also known as dizocilpine) or memantine, dilutions of the anti-NMDAR1 antisera described in Example 1 or anti-NMDAR1 IgG isolated from such antisera, or vehicle only. Each group was run in quadruplicate. AlamarBlue® was later added to each well following an incubation period of 48 hours, and fluorescent readings were taken with excitation at 530-560 nm and emission at 590 nm and above on a SYNERGY™ HT Multi Detection Reader to (BioTek®). Cell viability (%) was calculated as % of fluorescence of vehicle treated cells at 48 hours.

### <Results>

FIG. 6 shows exemplary results of the cell viability assay. Both MK-801 and memantine, along with anti-NMDAR1 antisera and IgG, reduced cell viability in a does-dependent manner. The reduction was statistically significant (p<0.01) for example for >300 pM concentrations of MK-801 and nanogram quantities of anti-NMDAR Abs.

### Example 4: Identification of Anti-NMDAR2B Antibodies

### <Methods>

Immunogen preparation: An amidated extracellular N-terminal peptide (KDAHEKDDF (SEQ ID NO: 2)) of human NMDAR2B, amidated and further added with a N-terminal tyrosine (YKDAHEKDDF-amide (SEQ ID NO: 3)) (NMDAR2B immunogen peptide) was coupled to bovine albumin (BSA) at a 10:1 peptide to BSA ratio using glutaraldehyde as the coupling agent.

Anti-NMDAR2B antibody screening: Fab antibody fragments that bind to human NMDA2B were identified by performing subtractive selection on a naïve human Fab phage library. Specifically, plates were coated with the BSA-coupled NMDAR2B immunogen peptide at concentrations of 5-100 μg/mL. Separate plates were coated with the same concentrations (as the BSA-coupled NMDAR2B immunogen peptide) of BSA or BSA that had undergone the glutaraldehyde coupling reaction without the peptide. As the pre-screening step, subtractive selection was performed using the BSA-coated plates to remove all phages expressing Fabs that bind to BSA or BSA that had undergone the glutaraldehyde coupling reaction. The remaining phages were then subjected to the plates coated with the BSA-coupled NMDAR2B immunogen peptide to select phages expressing Fabs that bind to the peptide. The antibody variable region sequences of the Fabs of the selected phages were sequenced.

### <Results>

Eight Fabs, named Fab3 through Fab10, each with unique variable region (VH and VL) sequences were identified, and exemplary IgG antibodies having the VH and VL sequences of Fab3 through Fab10 were named Ab3 through Ab10, respectively. FIGS. 1A and 1B show the CDR sequences of Ab3 through Ab10 and the nucleic acid sequences encoding such CDRs, respectively. FIG. 1C shows SEQ ID NOS assigned to the amino acid sequences of HC, VH, CDR-H1, CDR-H2, CDR-H3, LC, VL, CDR-L1, CDR-L2, and CDR-L3 of Ab3 through Ab10, and nucleic acid sequences encoding such.

### Example 5: Fab Binding to NMDAR2B Immunogen Peptide

### <Methods>

Fab production: Fab3 through Fab10 were obtained via culturing of recombinant cells encoding such Fabs followed by purification. SEQ ID NOS assigned to the sequences used for Fab production are shown in FIG. 2.

Radioimmunoassay: Radioimmunoassay on Fab3 through Fab10 was performed using ¹²⁵I-labeled (radiolabel on the N-terminal tyrosine) and free (no radiolabel or additional conjugation) NMDAR2B immunogen peptides.

### <Results>

No measurable radioactivity by Fab binding was observed.

### Example 6: Fab Binding to Biotinylated NMDAR2B Immunogen Peptide

### <Methods>

Fab production: Fab3 through Fab10 were obtained via culturing of recombinant cells encoding such Fabs followed by purification. SEQ ID NOS assigned to the sequences used for Fab production are shown in FIG. 2.

Peptide biotinylation; Biotinylation of the NMDAR2B immunogen peptide was performed using Biotin (Long Arm) NHS (Vector Laboratories Inc. (Burlingame, CA); product SP 1210) following the manufacturer recommendations and blockade of unused reagent with excess glycinamide.

ELISA assay: 96 well plates (ELISA Microlon 66 (Greiner Bio-one); #655061) were coated with Affinipure anti-human IgG antibodies (which bind to Fabs) (Jackson Immunoresearch Laboratories; #109-005-0060). Each well was added with a 50 μl volume containing 5 ng or 10 ng of an appropriate Fab and another 50 l volume containing the NMDAR2B immunogen peptide in a range of 10 μg to 10 ng, followed by incubation at 4° C. for 24-48 hours. Plates were washed, and each well was incubated with the biotinylated peptide at 100 ng/ml for 30 minutes to one hour and then with HRP-conjugated streptavidin (Thermo Industries; Product #N100) was added at 1:10,000 dilution for 30 minutes. Plates were washed and incubated with a mixture of 0.007% H₂O₂ and Amplex® Ultrared Reagent (Molecular Probes) for one hour, and fluorescence was read at excitation at 550 nm and fluorescence at 590 nm on a SYNERGY™ HT Multi Detection Reader to (BioTek®).

### <Results>

No measurable fluorescence was observed.

### Example 7: Fab Binding to NMDAR2B Immunogen Peptide Coupled to Biotinylated BSA

### <Methods>

Fab production: Fab3 through Fab10 were obtained via culturing of recombinant cells encoding such Fabs followed by purification. SEQ ID NOS assigned to the sequences used for Fab production are shown in FIG. 2.

Coupling to biotinylated BSA: BSA was biotinylated using the biotinylation method in Example 6 with glycinamide blocking, and biproducts were removed by passage through a Sephadex® G50 column. The biotinylated BSA was coupled to the NMDAR2B immunogen peptide at a 1:10 molar ratio using glutaraldehyde at pH 8.0 and stored at 10 μg/ml peptide in PBS with 3% BSA.

ELISA assay: ELISA as in Example 6 was performed using the NMDAR2B immunogen peptide coupled to biotinylated BSA and then, to test competition, the free NMDAR2B immunogen peptide.

### <Results>

Intense fluorescence was observed with all Fabs in the presence of the BSA-coupled peptide, indicating strong binding of the Fabs to the BSA-coupled peptide. However, the binding signal was not competed off the Fab used by increasing amounts of the free NMDAR2B immunogen peptide, indicating no binding of the Fabs to the free peptide.

### Example 8: Fab Binding Affinity to BSA-Conjugated NMDAR2B Immunogen Peptide

### <Methods>

Fab production: Fab3 through Fab10 were obtained via culturing of recombinant cells encoding such Fabs followed by purification. SEQ ID NOS assigned to the sequences used for Fab production are shown in FIG. 2.

ELISA assay: ELISA as in Example 6 was performed using the NMDAR2B immunogen peptide coupled to BSA (as prepared in Example 4) and the NMDAR2B immunogen peptide coupled to biotinylated BSA (as prepared in Example 7). Competition displacement curves were generated as repeated points in quadruplicate, and dissociation constants for respective Fabs to the BSA-coupled NMDAR2B immunogen peptide were calculated.

### <Results>

FIG. 7A provides exemplary competition displacement curves for all tested Fabs.

Dissociation constants (Kd) for the Fabs were between 4×10⁹ M and 2×10⁸ M. Although the binding of Fab9 seem to differ slightly from the rest of the Fabs in FIG. 7A, additional ELISA studies indicated that the binding affinity of Fab9 is similar to other Fabs and the Kd was about 5×10⁹ M, as shown in FIG. 7B.

### Example 9: Fab Binding to NMDAR2B on Cancer Cells

### <Materials>

NCI-H82 small-cell lung cancer (SCLC) cell line: NCI-H82 cells are representative of SCLC, and of pancreatic cancer cells, ovarian cancer cells, breast cancer cells, and prostate cancer cells all of which have been demonstrated to express NMDAR2B protein on their surface.

### <Methods>

Fab production: Fab3 through Fab10 were obtained via culturing of recombinant cells encoding such Fabs followed by purification. SEQ ID NOS assigned to the sequences used for Fab production are shown in FIG. 2.

Cell binding assay: Fabs at 5 μg/mL were incubated for one hour at room temperature (21° C.) with live NCI-H82 cells or NCI-H82 cells that had been fixed with 4% paraformaldehyde. Cells were washed and incubated for one hour with FITC-anti-human IgG (whole molecule) goat antibody (Sigma; F3512) at 1:500. Cells were washed and analyzed using the 8-Laser MACSQuant® flow cytometer and fluorescence was compared to control samples.

### <Results>

Representative histograms for each Fab on live and fixed cells are provided in the top and bottom panels, respectively, of FIG. 8. As shown in FIG. 8, binding was confirmed for all Fabs on both live and fixed cells. Fab8 and Fab9 showed the highest binding on live cells, and Fab8 showed the highest binding on fixed cells.

### Example 10: In Vitro Effects of Fab8 on NCI-H82 Cell Viability

### <Methods>

Fab production: Fab3 through Fab10 were obtained via culturing of recombinant cells encoding such Fabs followed by purification. SEQ ID NOS assigned to the sequences used for Fab production are shown in FIG. 2.

Viability assay: NCI-H82 cells were incubated different amounts of Fab8 for 72 hours in complete DMEM F12 medium containing 10% FBS. Cells were then added with MTT (3-(4,5-dimethylthiazol-2-yl)-2,5-diphenyltetrazolium bromide) to a final concentration of 0.5 mg/mL and incubated for 4 hours. The resulting formazan product was solubilized overnight with 10% SDS in 0.01M HCJ. Absorbance was read on a SYNERGY™ HT Multi Detection Reader to (BioTek®) at 570 nm. Cell viability (%) was calculated as % of MTT staining signal on cells incubated without an anti-NMDAR2B Fab.

### <Results>

The viability results are provided in FIG. 9. A 50% decrease in cell viability was produced by 100 μg/ml or about 2 μM of Fab8 protein.

### Example 11: In Vivo Anti-Cancer Effect of the NMDA Receptor Antagonist, Ifenprodil

### <Materials>

Anti-NMDA receptor agent: ifenprodil was used as an exemplary NMDA receptor antagonist

### <Methods>

Mouse treatment: Male nu/nu mice were injected subcutaneously with NCI H82 cells (about 10⁷ cells/mouse). Tumors became obvious after 2-3 weeks and commenced growing rapidly. Animals were divided into two groups. One group was intraperitoneally treated with ifenprodil (2.5 mg/kg body weight) once daily (ED50 in mice is about 350 mg/kg body weight). The other group was treated with saline vehicle. Changes in the tumor size and body weight were recorded daily.

### <Results>

FIG. 10 provides changes in the tumor size (top) and body weight (bottom). As shown in FIG. 10 (top), while tumors in saline-treated control animals grew >6 times their size over the two-week treatment period, the tumors of ifenprodil-treated animals first shrank to about 80% of their original size in about a day and then slowly grew back to their original size in about 10 days of treatment, followed by further growth to <2 times their original size by two weeks of treatment (p<0.002). As shown in FIG. 10 (bottom), body weight measurements performed over 8 days of treatment showed no differences between groups.

### Example 12: Combination Treatment: Chemotherapy and Anti-NMDA Receptor Therapy

### <Materials>

Anti-cancer agent: Cyclophosphamide, an alkylating agent, was used as an exemplary anti-cancer agent.

Anti-NMDA receptor agent: ifenprodil was used as an exemplary NMDA receptor antagonist

### <Methods>

Mouse treatment: Male nu/nu mice were injected subcutaneously with NCI H82 cells (about 10⁷ cells/mouse). Tumors became obvious after 2-3 weeks and commenced growing rapidly. Animals were divided into two groups. One group was first injected daily with cyclophosphamide (75 mg/kg body weight) for 3 days (cyclophosphamide treatment does not inhibit tumor growth beyond the time of cyclophosphamide administration) and then intraperitoneally with ifenprodil (2.5 mg/kg body weight) once daily. The other group was treated with saline vehicle. Changes in the tumor size were recorded daily.

### <Results>

FIG. 11 provides changes in the tumor size. As shown in FIG. 11, cyclophosphamide treatment slowed but did not stop tumor growth. However, once the ifenprodil treatment was initiated, the tumors of animals receiving the combination therapy continued to slowly shrink throughout the course of treatment for at least 6 days, while tumors in saline-treated control animals grew >7 times their size over the 10-day treatment period (p<0.001). Since tumors in animals that only received ifenprodil but not cyclophosphamide continued to grow (after a day of decrease in size) in Example 11, the reduction in tumor size in FIG. 11 indicates synergy between the anti-cancer agent and NMDA receptor antagonist.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### APPENDIX TO THE SPECIFICATION

### Amino Acid and Nucleic Acid Sequences

NMDAR2B:
Human NMDAR2B (NCBI Reference Sequence: NP_000825.2)
Amino acid Sequence:
(SEQ ID NO: 1)
MKPRAECCSPKFWLVLAVLAVSGSRARSQKSPPSIGIAVILVGTSDEVAIKDAHEKDDF
HHLSVVPRVELVAMNETDPKSIITRICDLMSDRKIQGVVFADDTDQEAIAQILDFISAQ
TLTPILGIHGGSSMIMADKDESSMFFQFGPSIEQQASVMLNIMEEYDWYIFSIVTTYFPG
YQDFVNKIRSTIENSFVGWELEEVLLLDMSLDDGDSKIQNQLKKLQSPIILLYCTKEEA
TYIFEVANSVGLTGYGYTWIVPSLVAGDTDTVPAEFPTGLISVSYDEWDYGLPARVRD
GIAIITTAASDMLSEHSFIPEPKSSCYNTHEKRIYQSNMLNRYLINVTFEGRNLSFSEDG
YQMHPKLVIILLNKERKWERVGKWKDKSLQMKYYVWPRMCPETEEQEDDHLSIVTL
EEAPFVIVESVDPLSGTCMRNTVPCQKRIVTENKTDEEPGYIKKCCKGFCIDILKKISKS
VKFTYDLYLVTNGKHGKKINGTWNGMIGEVVMKRAYMAVGSLTINEERSEVVDFSVP
FIETGISVMVSRSNGTVSPSAFLEPFSADVWVMMFVMLLIVSAVAVFVFEYFSPVGYNR
CLADGREPGGPSFTIGKAIWLLWGLVFNNSVPVQNPKGTTSKIMVSVWAFFAVIFLAS
YTANLAAFMIQEEYVDQVSGLSDKKFQRPNDFSPPFRFGTVPNGSTERNIRNNYAEM
HAYMGKFNQRGVDDALLSLKTGKLDAFIYDAAVLNYMAGRDEGCKLVTIGSGKVFAS
TGYGIAIQKDSGWKRQVDLAILQLFGDGEMEELEALWLTGICHNEKNEVMSSQLDIDN
MAGVFYMLGAAMALSLITFICEHLFYWQFRHCFMGVCSGKPGMVFSISRGIYSCIHGVA
IEERQSVMNSPTATMNNTHSNILRLLRTAKNMANLSGVNGSPQSALDFIRRESSVYDIS
EHRRSFTHSDCKSYNNPPCEENLFSDYISEVERTFGNLQLKDSNVYQDHYHHHHRPHS
IGSASSIDGLYDCDNPPFTTQSRSISKKPLDIGLPSSKHSQLSDLYGKFSFKSDRYSGHDD
LIRSDVSDISTHTVTYGNIEGNAAKRRKQQYKDSLKKRPASAKSRREFDEIELAYRRRP
PRSPDHKRYFRDKEGLRDFYLDQFRTKENSPHWEHVDLTDIYKERSDDFKRDSVSGG
GPCTNRSHIKHGTGDKHGVVSGVPAPWEKNLTNVEWEDRSGGNFCRSCPSKLHNYST
TVTGQNSGRQACIRCEACKKAGNLYDISEDNSLQELDQPAAPVAVTSNASTTKYPQSP
TNSKAQKKNRNKLRRQHSYDTFVDLQKEEAALAPRSVSLKDKGRFMDGSPYAHMFE
MSAGESTFANNKSSVPTAGHHHHNNPGGGYMLSKSLYPDRVTQNPFIPTFGDDQCLL
HGSKSYFFRQPTVAGASKARPDFRALVTNKPVVSALHGAVPARFQKDICIGNQSNPCV
PNNKNPRAFNGSSNGHVYEKLSSIESDV
Nucleic acid Sequence:
(SEQ ID NO: 11)
ATGAAGCCCAGAGCGGAGTGCTGTTCTCCCAAGTTCTGGTTGGTGTTGGCCGTCCTGG
CCGTGTCAGGCAGCAGAGCTCGTTCTCAGAAGAGCCCCCCCAGCATTGGCATTGCTGT
CATCCTCGTGGGCACTTCCGACGAGGTGGCCATCAAGGATGCCCACGAGAAAGATGA
TTTCCACCATCTCTCCGTGGTACCCCGGGTGGAACTGGTAGCCATGAATGAGACCGAC
CCAAAGAGCATCATCACCCGCATCTGTGATCTCATGTCTGACCGGAAGATCCAGGGGG
TGGTGTTTGCTGATGACACAGACCAGGAAGCCATCGCCCAGATCCTCGATTTCATTTC
AGCACAGACTCTCACCCCCATCCTGGGCATCCACGGGGGCTCCTCTATGATAATGGCA
GATAAGGATGAATCCTCCATGTTCTTCCAGTTTGGCCCATCAATTGAACAGCAAGCT
TCCGTAATGCTCAACATCATGGAAGAATATGACTGGTACATCTTTTCTATCGTCACC
ACCTATTTCCCTGGCTACCAGGACTTTGTAAACAAGATCCGCAGCACCATTGAGAAT
AGCTTTGTGGGCTGGGAGCTAGAGGAGGTCCTCCTACTGGACATGTCCCTGGACGAT
GGAGATTCTAAGATCCAGAATCAGCTCAAGAAACTTCAAAGCCCCATCATTCTTCTT
TACTGTACCAAGGAAGAAGCCACCTACATCTTTGAAGTGGCCAACTCAGTAGGGCTG
ACTGGCTATGGCTACACGTGGATCGTGCCCAGTCTGGTGGCAGGGGATACAGACACA
GTGCCTGCGGAGTTCCCCACTGGGCTCATCTCTGTATCATATGATGAATGGGACTATG
GCCTCCCCGCCAGAGTGAGAGATGGAATTGCCATAATCACCACTGCTGCTTCTGACAT
GCTGTCTGAGCACAGCTTCATCCCTGAGCCCAAAAGCAGTTGTTACAACACCCACGAG
AAGAGAATCTACCAGTCCAATATGCTAAATAGGTATCTGATCAATGTCACTTTTGAG
GGGAGGAATTTGTCCTTCAGTGAAGATGGCTACCAGATGCACCCGAAACTGGTGATA
ATTCTTCTGAACAAGGAGAGGAAGTGGGAAAGGGTGGGGAAGTGGAAAGACAAGTC
CCTGCAGATGAAGTACTATGTGTGGCCCCGAATGTGTCCAGAGACTGAAGAGCAGGA
GGATGACCATCTGAGCATTGTGACCCTGGAGGAGGCACCATTTGTCATTGTGGAAAG
TGTGGACCCTCTGAGTGGAACCTGCATGAGGAACACAGTCCCCTGCCAAAAACGCAT
AGTCACTGAGAATAAAACAGACGAGGAGCCGGGTTACATCAAAAAATGCTGCAAGGG
GTTCTGTATTGACATCCTTAAGAAAATTTCTAAATCTGTGAAGTTCACCTATGACCT
TTACCTGGTTACCAATGGCAAGCATGGGAAGAAAATCAATGGAACCTGGAATGGTAT
GATTGGAGAGGTGGTCATGAAGAGGGCCTACATGGCAGTGGGCTCACTCACCATCAA
TGAGGAACGATCGGAGGTGGTCGACTTCTCTGTGCCCTTCATAGAGACAGGCATCAG
TGTCATGGTGTCACGCAGCAATGGGACTGTCTCACCTTCTGCCTTCTTAGAGCCATTC
AGCGCTGACGTATGGGTGATGATGTTTGTGATGCTGCTCATCGTCTCAGCCGTGGCT
GTCTTTGTCTTTGAGTACTTCAGCCCTGTGGGTTATAACAGGTGCCTCGCTGATGGCA
GAGAGCCTGGTGGACCCTCTTTCACCATCGGCAAAGCTATTTGGTTGCTCTGGGGTCT
GGTGTTTAACAACTCCGTACCTGTGCAGAACCCAAAGGGGACCACCTCCAAGATCAT
GGTGTCAGTGTGGGCCTTCTTTGCTGTCATCTTCCTGGCCAGCTACACTGCCAACTTA
GCTGCCTTCATGATCCAAGAGGAATATGTGGACCAGGTTTCTGGCCTGAGCGACAAA
AAGTTCCAGAGACCTAATGACTTCTCACCCCCTTTCCGCTTTGGGACCGTGCCCAACG
GCAGCACAGAGAGAAATATTCGCAATAACTATGCAGAAATGCATGCCTACATGGGAA
AGTTCAACCAGAGGGGTGTAGATGATGCATTGCTCTCCCTGAAAACAGGGAAACTGG
ATGCCTTCATCTATGATGCAGCAGTGCTGAACTATATGGCAGGCAGAGATGAAGGCT
GCAAGCTGGTGACCATTGGCAGTGGGAAGGTCTTTGCTTCCACTGGCTATGGCATTG
CCATCCAAAAAGATTCTGGGTGGAAGCGCCAGGTGGACCTTGCTATCCTGCAGCTCTT
TGGAGATGGGGAGATGGAAGAACTGGAAGCTCTCTGGCTCACTGGCATTTGTCACAA
TGAGAAGAATGAGGTCATGAGCAGCCAGCTGGACATTGACAACATGGCAGGGGTCTT
CTACATGTTGGGGGCGGCCATGGCTCTCAGCCTCATCACCTTCATCTGCGAACACCTT
TTCTATTGGCAGTTCCGACATTGCTTTATGGGTGTCTGTTCTGGCAAGCCTGGCATGG
TCTTCTCCATCAGCAGAGGTATCTACAGCTGCATCCATGGGGTGGCGATCGAGGAGC
GCCAGTCTGTAATGAACTCCCCCACCGCAACCATGAACAACACACACTCCAACATCCT
GCGCCTGCTGCGCACGGCCAAGAACATGGCTAACCTGTCTGGTGTGAATGGCTCACCG
CAGAGCGCCCTGGACTTCATCCGACGGGAGTCATCCGTCTATGACATCTCAGAGCACC
GCCGCAGCTTCACGCATTCTGACTGCAAATCCTACAACAACCCGCCCTGTGAGGAGAA
CCTCTTCAGTGACTACATCAGTGAGGTAGAGAGAACGTTCGGGAACCTGCAGCTGAA
GGACAGCAACGTGTACCAAGATCACTACCACCATCACCACCGGCCCCATAGTATTGGC
AGTGCCAGCTCCATCGATGGGCTCTACGACTGTGACAACCCACCCTTCACCACCCAGT
CCAGGTCCATCAGCAAGAAGCCCCTGGACATCGGCCTCCCCTCCTCCAAGCACAGCCA
GCTCAGTGACCTGTACGGCAAATTCTCCTTCAAGAGCGACCGCTACAGTGGCCACGAC
GACTTGATCCGCTCCGATGTCTCTGACATCTCAACCCACACCGTCACCTATGGGAACA
TCGAGGGCAATGCCGCCAAGAGGCGTAAGCAGCAATATAAGGACAGCCTGAAGAAGC
GGCCTGCCTCGGCCAAGTCCCGCAGGGAGTTTGACGAGATCGAGCTGGCCTACCGTCG
CCGACCGCCCCGCTCCCCTGACCACAAGCGCTACTTCAGGGACAAGGAAGGGCTACGG
GACTTCTACCTGGACCAGTTCCGAACAAAGGAGAACTCACCCCACTGGGAGCACGTAG
ACCTGACCGACATCTACAAGGAGCGGAGTGATGACTTTAAGCGCGACTCCGTCAGCG
GAGGAGGGCCCTGTACCAACAGGTCTCACATCAAGCACGGGACGGGCGACAAACACG
GCGTGGTCAGCGGGGTACCTGCACCTTGGGAGAAGAACCTGACCAACGTGGAGTGGG
AGGACCGGTCCGGGGGCAACTTCTGCCGCAGCTGTCCCTCCAAGCTGCACAACTACTC
CACGACGGTGACGGGTCAGAACTCGGGCAGGCAGGCGTGCATCCGGTGTGAGGCTTG
CAAGAAAGCAGGCAACCTGTATGACATCAGTGAGGACAACTCCCTGCAGGAACTGGA
CCAGCCGGCTGCCCCAGTGGCGGTGACGTCAAACGCCTCCACCACTAAGTACCCTCAG
AGCCCGACTAATTCCAAGGCCCAGAAGAAGAACCGGAACAAACTGCGCCGGCAGCAC
TCCTACGACACCTTCGTGGACCTGCAGAAGGAAGAAGCCGCCCTGGCCCCGCGCAGCG
TAAGCCTGAAAGACAAGGGCCGATTCATGGATGGGAGCCCCTACGCCCACATGTTTG
AGATGTCAGCTGGCGAGAGCACCTTTGCCAACAACAAGTCCTCAGTGCCCACTGCCGG
ACATCACCACCACAACAACCCCGGCGGCGGGTACATGCTCAGCAAGTCGCTCTACCCT
GACCGGGTCACGCAAAACCCTTTCATCCCCACTTTTGGGGACGACCAGTGCTTGCTCC
ATGGCAGCAAATCCTACTTCTTCAGGCAGCCCACGGTGGCGGGGGCGTCGAAAGCCAG
GCCGGACTTCCGGGCCCTTGTCACCAACAAGCCGGTGGTCTCGGCCCTTCATGGGGCC
GTGCCAGCCCGTTTCCAGAAGGACATCTGTATAGGGAACCAGTCCAACCCCTGTGTGC
CTAACAACAAAAACCCCAGGGCTTTCAATGGCTCCAGCAATGGGCATGTTTATGAGA
AACTTTCTAGTATTGAGTCTGATGTCTGA
Human NMDAR2B extracellular peptide
Amino acid Sequence:
(SEQ ID NO: 2)
KDAHEKDDF
Human NMDAR2B extracellular peptide with an added N-terminal tyrosine,
amidated
Amino acid Sequence:
(SEQ ID NO: 3)
YKDAHEKDDF-amide
Human NMDAR1 extracellular peptide
Amino acid Sequence:
(SEQ ID NO: 5)
MSIYSDKSIH-amide

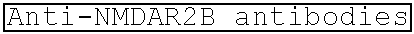

*Boxed sequences correspond to CDRs (CDR 1, CDR 2, and CDR 3, in the order of
appearance).
** Italics represent constant regions.
***
The C-terminal K (lysine) in heavy chain sequences may or may not be
present.
Anti-NMDAR2B Antibody 3 (Ab3):
Ab3 Heavy chain variable region (VH) CDR 1 (Ab3 CDR-H1)
Amino acid Sequence:
(SEQ ID NO: 32)

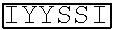

Nucleic acid Sequence:
(SEQ ID NO: 132)

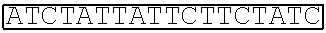

Ab3 VH CDR 3 (Ab3 CDR-H3)
Amino acid Sequence:
(SEQ ID NO: 34)

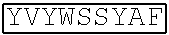

Nucleic acid Sequence:
(SEQ ID NO: 134)

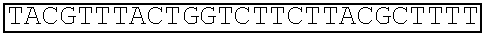

Antibody 3 Light chain (Ab3 LC)
Amino acid Sequence:
(SEQ ID NO: 35)

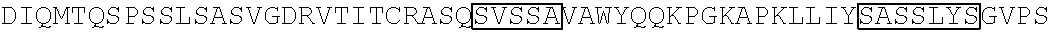

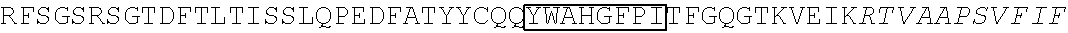

PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Nucleic acid Sequence:
(SEQ ID NO: 135)
GATATCCAGATGACCCAGTCCCCGAGCTCCCTGTCCGCCTCTGTGGGCGATAGGGTCA

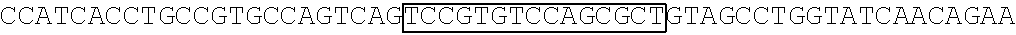

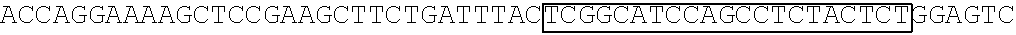

CCTTCTCGCTTCTCTGGTAGCCGTTCCGGGACGGATTTCACTCTGACCATCAGCAGTC

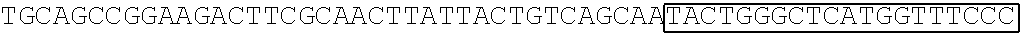

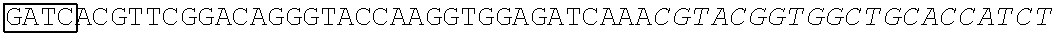

GTCTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGGAGAGTGT
Antibody 3 Light chain variable region (Ab3 VL)
Amino acid Sequence:
(SEQ ID NO: 36)

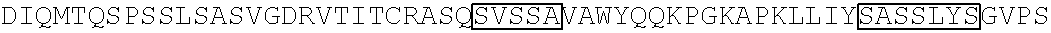

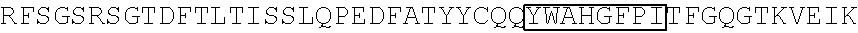

Nucleic acid Sequence:
(SEQ ID NO: 136)
GATATCCAGATGACCCAGTCCCCGAGCTCCCTGTCCGCCTCTGTGGGCGATAGGGTCA

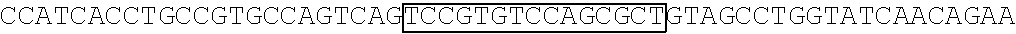

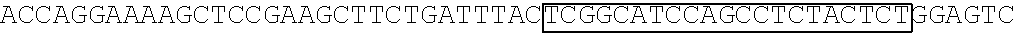

CCTTCTCGCTTCTCTGGTAGCCGTTCCGGGACGGATTTCACTCTGACCATCAGCAGTC

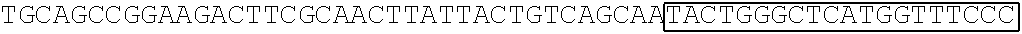

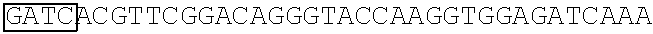

Ab3 VL CDR 1 (Ab3 CDR-L1)
Amino acid Sequence:
(SEQ ID NO: 37)

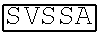

Nucleic acid Sequence:
(SEQ ID NO: 137)

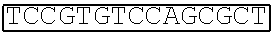

Ab3 VL CDR 2 (Ab3 CDR-L2)
Amino acid Sequence:
(SEQ ID NO: 38)

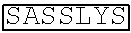

Nucleic acid Sequence:
(SEQ ID NO: 138)

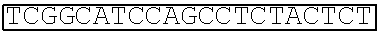

Ab3 VL CDR 3 (Ab3 CDR-L3)
Amino acid Sequence:
(SEQ ID NO: 39)

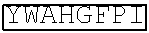

Nucleic acid Sequence:
(SEQ ID NO: 139)

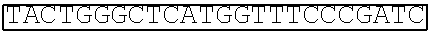

Anti-NMDAR2B Antibody 4 (Ab4):
Antibody 4 Heavy chain (Ab4 HC)
Amino acid Sequence:
(SEQ ID NO: 40)

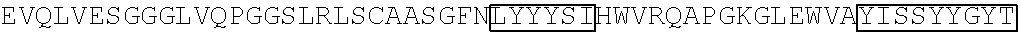

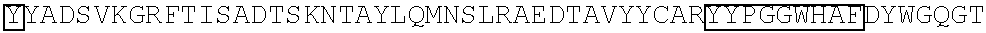

LVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
Nucleic acid Sequence:
(SEQ ID NO: 140)
GAGGTTCAGCTGGTGGAGTCTGGCGGTGGCCTGGTGCAGCCAGGGGGCTCACTCCGTT

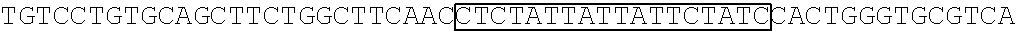

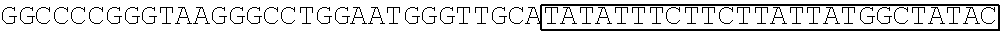

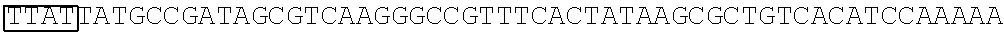

CACAGCCTACCTACAAATGAACAGCTTAAGAGCTGAGGACACTGCCGTCTATTATTG

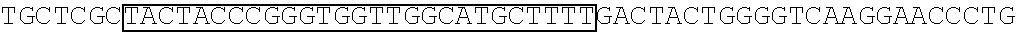

GTCACCGTCTCCTCGGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAA
GAAAGTTGAGCCCAAATCTTGTGACAAAACTCACACATGCCCACCGTGCCCAGCACCTGA
ACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATC
CCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACCGTGGACAA
GAGCAGGTGGCAGCAGGGGAACGTCTTCTCATGCTCCGTGATGCATGAGGCTCTGCACAA
CCACTACACGCAGAAGAGCCTCTCCCTGTCTCCGGGTAAA
Antibody 4 Heavy chain variable region (Ab4 VH)
Amino acid Sequence:
(SEQ ID NO: 41)

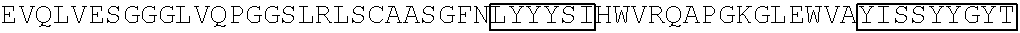

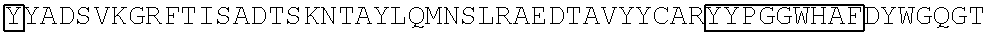

LVTVSS
Nucleic acid Sequence:
(SEQ ID NO: 141)
GAGGTTCAGCTGGTGGAGTCTGGCGGTGGCCTGGTGCAGCCAGGGGGCTCACTCCGTT

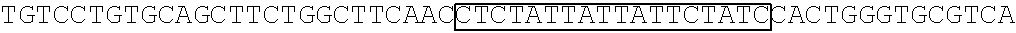

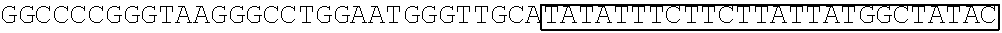

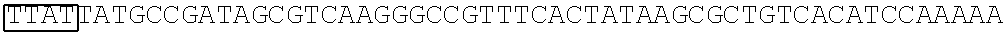

CACAGCCTACCTACAAATGAACAGCTTAAGAGCTGAGGACACTGCCGTCTATTATTG

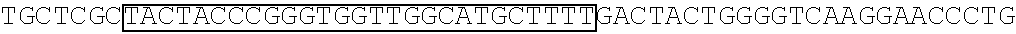

GTCACCGTCTCCTCG
Ab4 VH CDR 1 (Ab4 CDR-H1)
Amino acid Sequence:
(SEQ ID NO: 42)

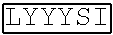

Nucleic acid Sequence:
(SEQ ID NO: 142)

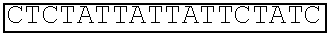

Ab4 VH CDR 2 (Ab4 CDR-H2)
Amino acid Sequence:
(SEQ ID NO: 43)

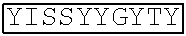

Nucleic acid Sequence:
(SEQ ID NO: 143)

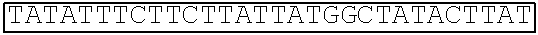

Ab4 VH CDR 3 (Ab4 CDR-H3)
Amino acid Sequence:
(SEQ ID NO: 44)

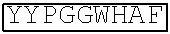

Nucleic acid Sequence:
(SEQ ID NO: 144)

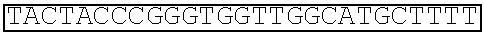

Antibody 4 Light chain (Ab4 LC)
Amino acid Sequence:
(SEQ ID NO: 45)

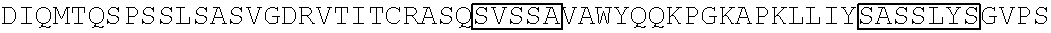

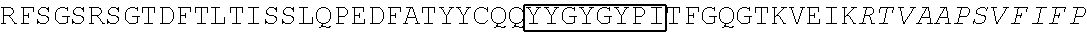

PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Nucleic acid Sequence:
(SEQ ID NO: 145)
GATATCCAGATGACCCAGTCCCCGAGCTCCCTGTCCGCCTCTGTGGGCGATAGGGTCA

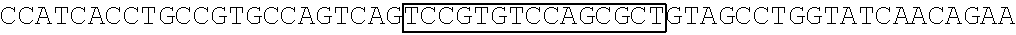

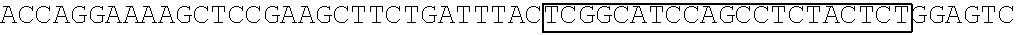

CCTTCTCGCTTCTCTGGTAGCCGTTCCGGGACGGATTTCACTCTGACCATCAGCAGTC

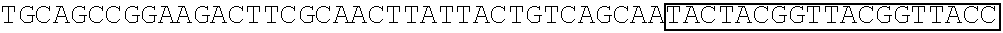

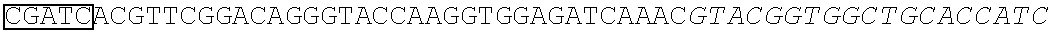

TGTCTTCATCTTCCCGCCATCTGATGAGCAGTTGAAATCTGGAACTGCCTCTGTTGTGTG
CCTGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTG
CGAAGTCACCCATCAGGGCCTGAGCTCGCCCGTCACAAAGAGCTTCAACAGGGGAGAGTG
T
Antibody 4 Light chain variable region (Ab4 VL)
Amino acid Sequence:
(SEQ ID NO: 46)

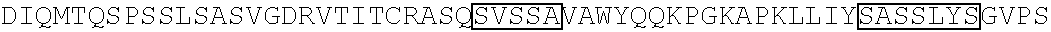

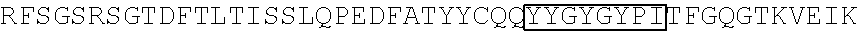

Nucleic acid Sequence:
(SEQ ID NO: 146)
GATATCCAGATGACCCAGTCCCCGAGCTCCCTGTCCGCCTCTGTGGGCGATAGGGTCA

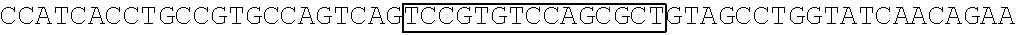

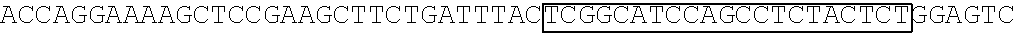

CCTTCTCGCTTCTCTGGTAGCCGTTCCGGGACGGATTTCACTCTGACCATCAGCAGTC

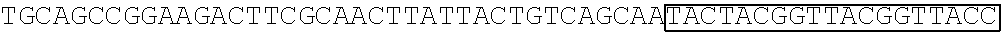

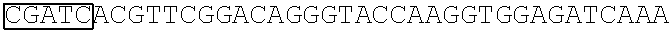

Ab4 VL CDR 1 (Ab4 CDR-L1)
Amino acid Sequence:
(SEQ ID NO: 47)

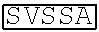

Nucleic acid Sequence:
(SEQ ID NO: 147)

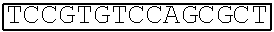

Ab4 VL CDR 2 (Ab4 CDR-L2)
Amino acid Sequence:
(SEQ ID NO: 48)

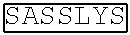

Nucleic acid Sequence:
(SEQ ID NO: 148)

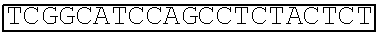

Ab4 VL CDR 3 (Ab4 CDR-L3)
Amino acid Sequence:
(SEQ ID NO: 49)

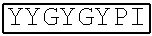

Nucleic acid Sequence:
(SEQ ID NO: 149)

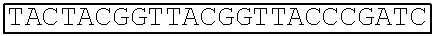

Anti-NMDAR2B Antibody 5 (Ab5):
Antibody 5 Heavy chain (Ab5 HC)
Amino acid Sequence:
(SEQ ID NO: 50)

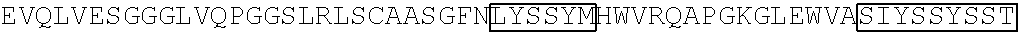

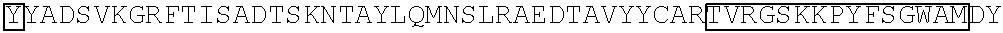

WGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Nucleic acid Sequence:
(SEQ ID NO: 150)
GAGGTTCAGCTGGTGGAGTCTGGCGGTGGCCTGGTGCAGCCAGGGGGCTCACTCCGTT

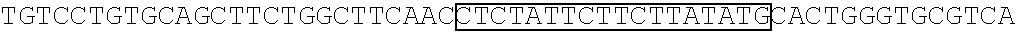

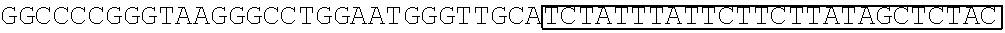

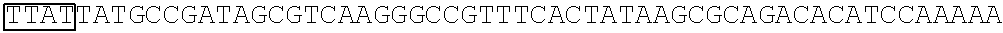

CACAGCCTACCTACAAATGAACAGCTTAAGAGCTGAGGACACTGCCGTCTATTATTG

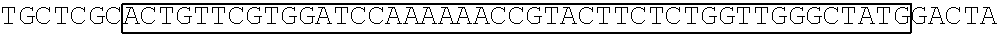

CTGGGGTCAAGGAACCCTGGTCACCGTCTCCTCGGCTAGCACCAAGGGCCCATCGGTCT
TCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAA
Antibody 5 Heavy chain variable region (Ab5 VH)
Amino acid Sequence:
(SEQ ID NO: 51)

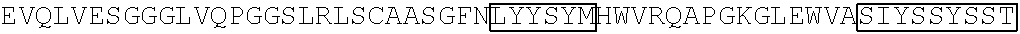

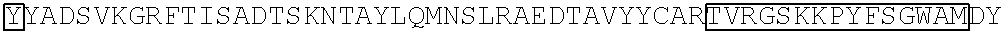

WGQGTLVTVSS
Nucleic acid Sequence:
(SEQ ID NO: 151)
GAGGTTCAGCTGGTGGAGTCTGGCGGTGGCCTGGTGCAGCCAGGGGGCTCACTCCGTT

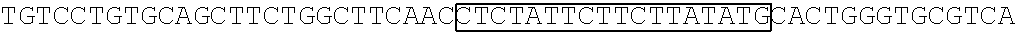

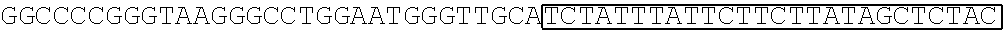

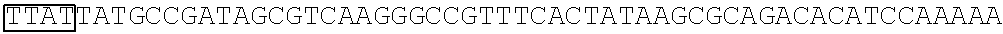

CACAGCCTACCTACAAATGAACAGCTTAAGAGCTGAGGACACTGCCGTCTATTATTG

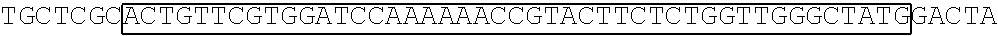

CTGGGGTCAAGGAACCCTGGTCACCGTCTCCTCG
Ab5 VH CDR 1 (Ab5 CDR-H1)
Amino acid Sequence:
(SEQ ID NO: 52)

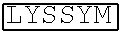

Nucleic acid Sequence:
(SEQ ID NO: 152)

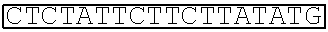

Ab5 VH CDR 2 (Ab5 CDR-H2)
Amino acid Sequence:
(SEQ ID NO: 53)

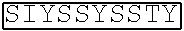

Nucleic acid Sequence:
(SEQ ID NO: 153)

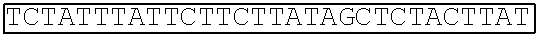

Ab5 VH CDR 3 (Ab5 CDR-H3)
Amino acid Sequence:
(SEQ ID NO: 54)

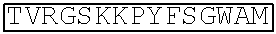

Nucleic acid Sequence:
(SEQ ID NO: 154)

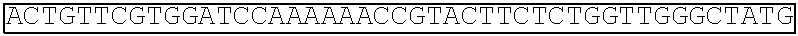

Antibody 5 Light chain (Ab5 LC)
Amino acid Sequence:
(SEQ ID NO: 55)

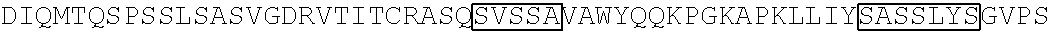

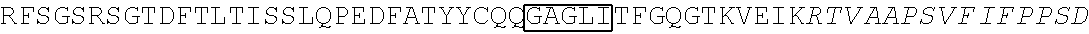

EQLKSGTASVVCLLNNFYPREAKVQWKVDNALQSGNSQESVTEQDSKDSTYSLSSTLTLS
KADYEKHKVYACEVTHQGLSSPVTKSFNRGEC
Nucleic acid Sequence:
(SEQ ID NO: 155)
GATATCCAGATGACCCAGTCCCCGAGCTCCCTGTCCGCCTCTGTGGGCGATAGGGTCA

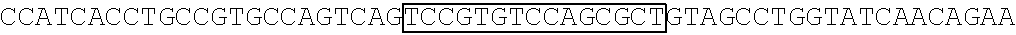

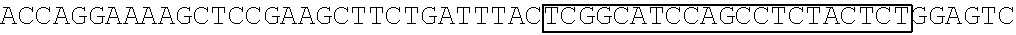

CCTTCTCGCTTCTCTGGTAGCCGTTCCGGGACGGATTTCACTCTGACCATCAGCAGTC

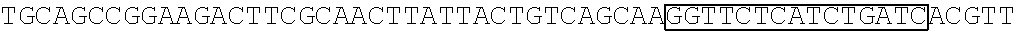

TCGGACAGGGTACCAAGGTGGAGATCAAACGTACGGTGGCTGCACCATCTGTCTTCATC
TTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
Antibody 5 Light chain variable region (Ab5 VL)
Amino acid Sequence:
(SEQ ID NO: 56)

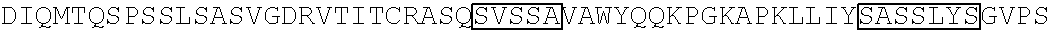

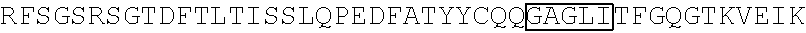

Nucleic acid Sequence:
(SEQ ID NO: 156)
GATATCCAGATGACCCAGTCCCCGAGCTCCCTGTCCGCCTCTGTGGGCGATAGGGTCA

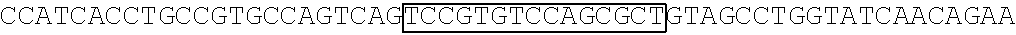

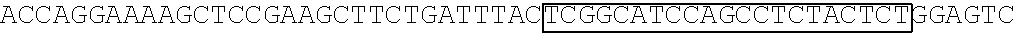

CCTTCTCGCTTCTCTGGTAGCCGTTCCGGGACGGATTTCACTCTGACCATCAGCAGTC

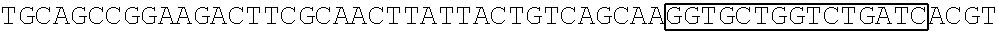

TCGGACAGGGTACCAAGGTGGAGATCAAA
Ab5 VL CDR 1 (Ab5 CDR-L1)
Amino acid Sequence:
(SEQ ID NO: 57)

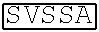

Nucleic acid Sequence:
(SEQ ID NO: 157)

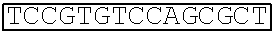

Ab5 VL CDR 2 (Ab5 CDR-L2)
Amino acid Sequence:
(SEQ ID NO: 58)

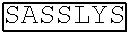

Nucleic acid Sequence:
(SEQ ID NO: 158)

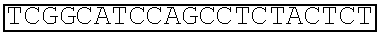

Ab5 VL CDR 3 (Ab5 CDR-L3)
Amino acid Sequence:
(SEQ ID NO: 59)

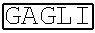

Nucleic acid Sequence:
(SEQ ID NO: 159)

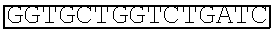

Anti-NMDAR2B Antibody 6 (Ab6):
Antibody 6 Heavy chain (Ab6 HC)
Amino acid Sequence:
(SEQ ID NO: 60)

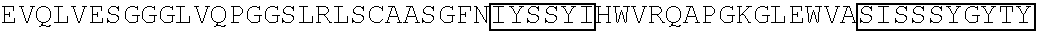

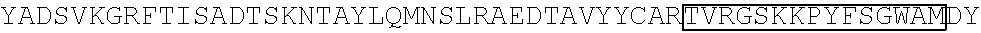

WGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Nucleic acid Sequence:
(SEQ ID NO: 160)
GAGGTTCAGCTGGTGGAGTCTGGCGGTGGCCTGGTGCAGCCAGGGGGCTCACTCCGTT

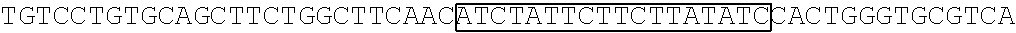

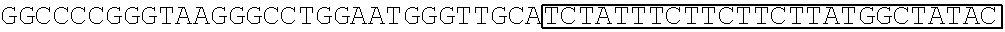

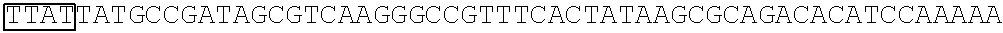

CACAGCCTACCTACAAATGAACAGCTTAAGAGCTGAGGACACTGCCGTCTATTATTG

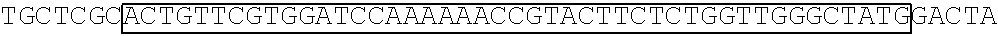

CTGGGGTCAAGGAACCCTGGTCACCGTCTCCTCGGCTAGCACCAAGGGCCCATCGGTCT
TCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAA
Antibody 6 Heavy chain variable region (Ab6 VH)
Amino acid Sequence:
(SEQ ID NO: 61)

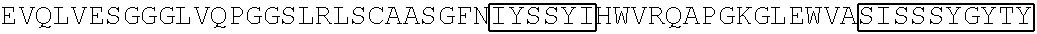

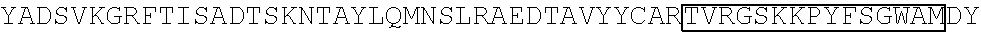

WGQGTLVTVSS
Nucleic acid Sequence:
(SEQ ID NO: 161)
GAGGTTCAGCTGGTGGAGTCTGGCGGTGGCCTGGTGCAGCCAGGGGGCTCACTCCGTT

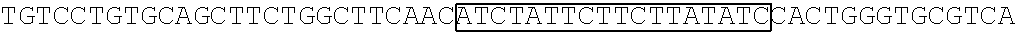

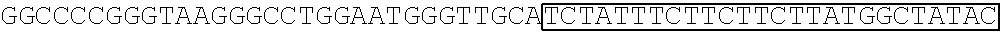

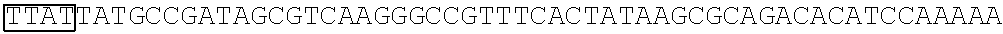

CACAGCCTACCTACAAATGAACAGCTTAAGAGCTGAGGACACTGCCGTCTATTATTG

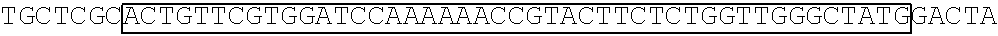

CTGGGGTCAAGGAACCCTGGTCACCGTCTCCTCG
Ab6 VH CDR 1 (Ab6 CDR-H1)
Amino acid Sequence:
(SEQ ID NO: 62)

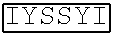

Nucleic acid Sequence:
(SEQ ID NO: 162)

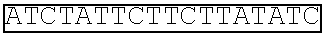

Ab6 VH CDR 2 (Ab6 CDR-H2)
Amino acid Sequence:
(SEQ ID NO: 63)

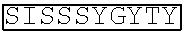

Nucleic acid Sequence:
(SEQ ID NO: 163)

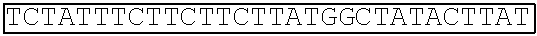

Ab6 VH CDR 3 (Ab6 CDR-H3)
Amino acid Sequence:
(SEQ ID NO: 64)

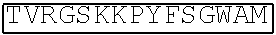

Nucleic acid Sequence:
(SEQ ID NO: 164)

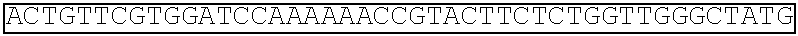

Antibody 6 Light chain (Ab6 LC)
Amino acid Sequence:
(SEQ ID NO: 65)

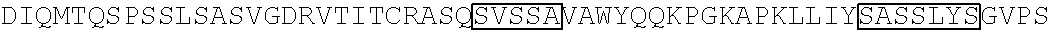

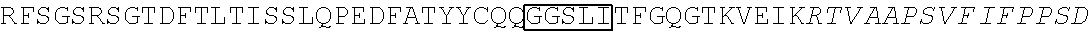

EQLKSGTASVVCLLNNFYPREAKVQWKVDNALQSGNSQESVTEQDSKDSTYSLSSTLTLS
KADYEKHKVYACEVTHQGLSSPVTKSFNRGEC
Nucleic acid Sequence:
(SEQ ID NO: 165)
GATATCCAGATGACCCAGTCCCCGAGCTCCCTGTCCGCCTCTGTGGGCGATAGGGTCA

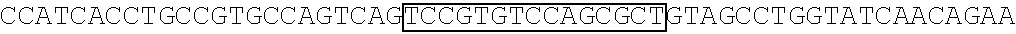

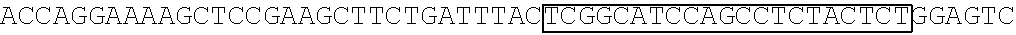

CCTTCTCGCTTCTCTGGTAGCCGTTCCGGGACGGATTTCACTCTGACCATCAGCAGTC

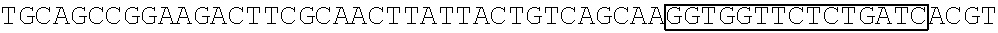

TCGGACAGGGTACCAAGGTGGAGATCAAACGTACGGTGGCTGCACCATCTGTCTTCATC
TTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
Antibody 6 Light chain variable region (Ab6 VL)
Amino acid Sequence:
(SEQ ID NO: 66)

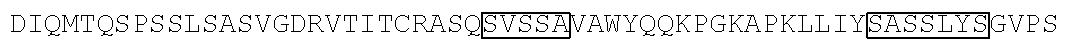

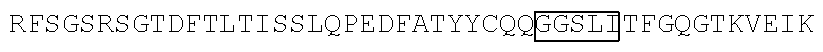

Nucleic acid Sequence:
(SEQ ID NO: 166)
GATATCCAGATGACCCAGTCCCCGAGCTCCCTGTCCGCCTCTGTGGGCGATAGGGTCA

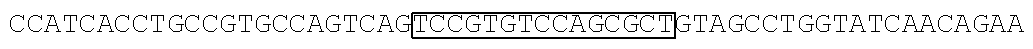

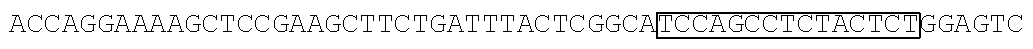

CCTTCTCGCTTCTCTGGTAGCCGTTCCGGGACGGATTTCACTCTGACCATCAGCAGTC

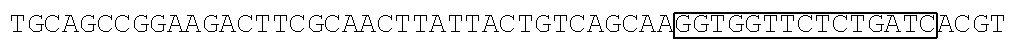

TCGGACAGGGTACCAAGGTGGAGATCAAA
Ab6 VL CDR 1 (Ab6 CDR-L1)
Amino acid Sequence:
(SEQ ID NO: 67)

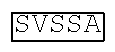

Nucleic acid Sequence:
(SEQ ID NO: 167)

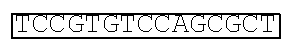

Ab6 VL CDR 2 (Ab6 CDR-L2)
Amino acid Sequence:
(SEQ ID NO: 68)

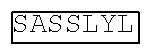

Nucleic acid Sequence:
(SEQ ID NO: 168)

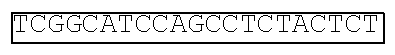

Ab6 VL CDR 3 (Ab6 CDR-L3)
Amino acid Sequence:
(SEQ ID NO: 69)

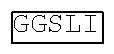

Nucleic acid Sequence:
(SEQ ID NO: 169)

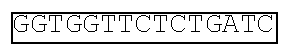

Anti-NMDAR2B Antibody 7 (Ab7):
Antibody 7 Heavy chain (Ab7 HC)
Amino acid Sequence:
(SEQ ID NO: 70)

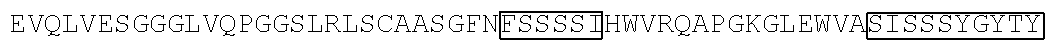

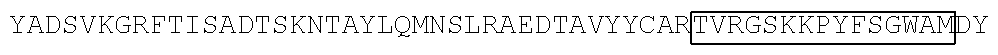

WGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Nucleic acid Sequence:
(SEQ ID NO: 170)
GAGGTTCAGCTGGTGGAGTCTGGCGGTGGCCTGGTGCAGCCAGGGGGCTCACTCCGTT

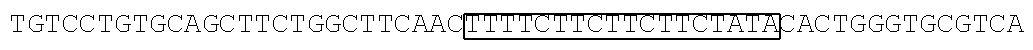

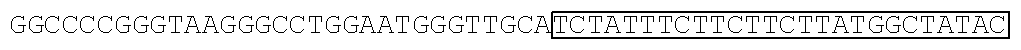

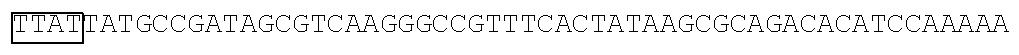

CACAGCCTACCTACAAATGAACAGCTTAAGAGCTGAGGACACTGCCGTCTATTATTG

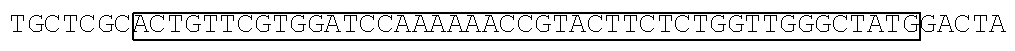

CTGGGGTCAAGGAACCCTGGTCACCGTCTCCTCGGCTAGCACCAAGGGCCCATCGGTCT
TCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAA
Antibody 7 Heavy chain variable region (Ab7 VH)
Amino acid Sequence:
(SEQ ID NO: 71)

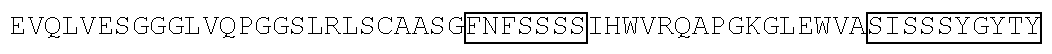

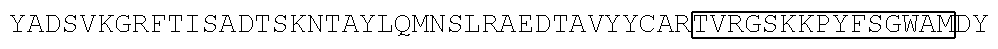

WGQGTLVTVSS
Nucleic acid Sequence:
(SEQ ID NO: 171)
GAGGTTCAGCTGGTGGAGTCTGGCGGTGGCCTGGTGCAGCCAGGGGGCTCACTCCGTT

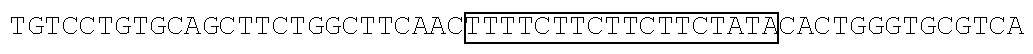

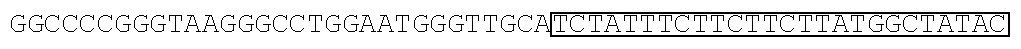

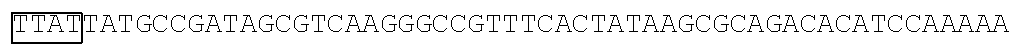

CACAGCCTACCTACAAATGAACAGCTTAAGAGCTGAGGACACTGCCGTCTATTATTG

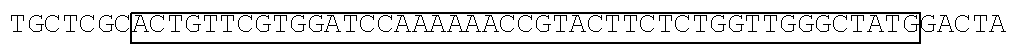

CTGGGGTCAAGGAACCCTGGTCACCGTCTCCTCG
Ab7 VH CDR 1 (Ab7 CDR-H1)
Amino acid Sequence:
(SEQ ID NO: 72)

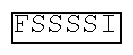

Nucleic acid Sequence:
(SEQ ID NO: 172)

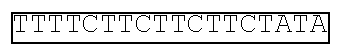

Ab7 VH CDR 2 (Ab7 CDR-H2)
Amino acid Sequence:
(SEQ ID NO: 73)

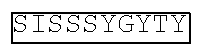

Nucleic acid Sequence:
(SEQ ID NO: 173)

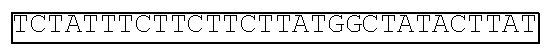

Ab7 VH CDR 3 (Ab7 CDR-H3)
Amino acid Sequence:
(SEQ ID NO: 74)

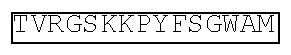

Nucleic acid Sequence:
(SEQ ID NO: 174)

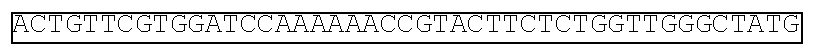

Antibody 7 Light chain (Ab7 LC)
Amino acid Sequence:
(SEQ ID NO: 75)

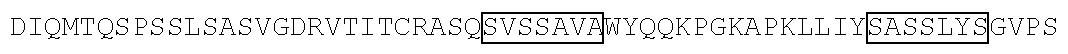

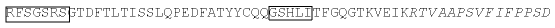

EQLKSGTASVVCLLNNFYPREAKVQWKVDNALQSGNSQESVTEQDSKDSTYSLSSTLTLS
KADYEKHKVYACEVTHQGLSSPVTKSFNRGEC
Nucleic acid Sequence:
(SEQ ID NO: 175)
GATATCCAGATGACCCAGTCCCCGAGCTCCCTGTCCGCCTCTGTGGGCGATAGGGTCA

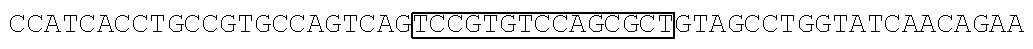

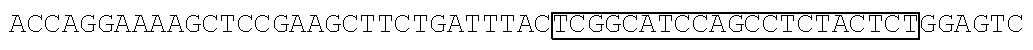

CCTTCTCGCTTCTCTGGTAGCCGTTCCGGGACGGATTTCACTCTGACCATCAGCAGTC

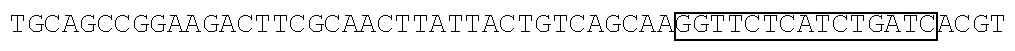

TCGGACAGGGTACCAAGGTGGAGATCAAACGTACGGTGGCTGCACCATCTGTCTTCATC
TTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
Antibody 7 Light chain variable region (Ab7 VL)
Amino acid Sequence:
(SEQ ID NO: 76)

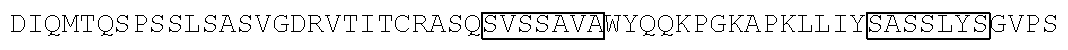

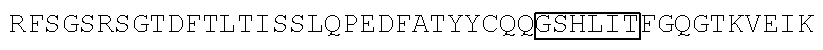

Nucleic acid Sequence:
(SEQ ID NO: 176)
GATATCCAGATGACCCAGTCCCCGAGCTCCCTGTCCGCCTCTGTGGGCGATAGGGTCA

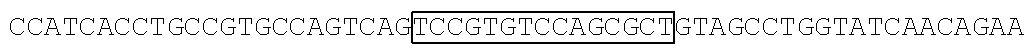

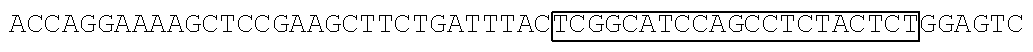

CCTTCTCGCTTCTCTGGTAGCCGTTCCGGGACGGATTTCACTCTGACCATCAGCAGTC

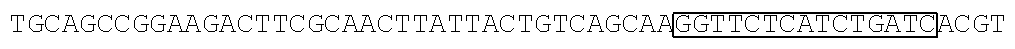

TCGGACAGGGTACCAAGGTGGAGATCAAA
Ab7 VL CDR 1 (Ab7 CDR-L1)
Amino acid Sequence:
(SEQ ID NO: 77)

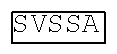

Nucleic acid Sequence:
(SEQ ID NO: 177)

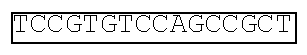

Ab7 VL CDR 2 (Ab7 CDR-L2)
Amino acid Sequence:
(SEQ ID NO: 78)

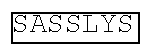

Nucleic acid Sequence:
(SEQ ID NO: 178)

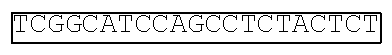

Ab7 VL CDR 3 (Ab7 CDR-L3)
Amino acid Sequence:
(SEQ ID NO: 79)

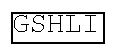

Nucleic acid Sequence:
(SEQ ID NO: 179)

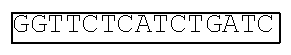

Anti-NMDAR2B Antibody 8 (Ab8):
Antibody 8 Heavy chain (Ab8 HC)
Amino acid Sequence:
(SEQ ID NO: 80)

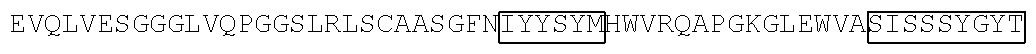

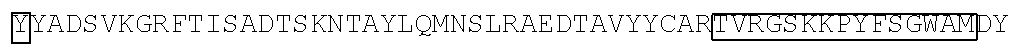

WGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Nucleic acid Sequence:
(SEQ ID NO: 180)
GAGGTTCAGCTGGTGGAGTCTGGCGGTGGCCTGGTGCAGCCAGGGGGCTCACTCCGTT

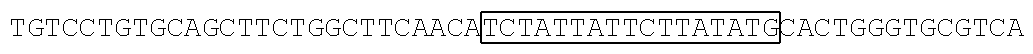

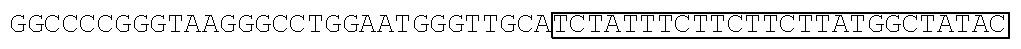

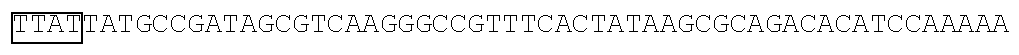

CACAGCCTACCTACAAATGAACAGCTTAAGAGCTGAGGACACTGCCGTCTATTATTG

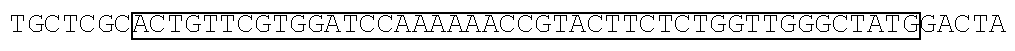

CTGGGGTCAAGGAACCCTGGTCACCGTCTCCTCGGCTAGCACCAAGGGCCCATCGGTCT
TCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAA
Antibody 8 Heavy chain variable region (Ab8 VH)
Amino acid Sequence:
(SEQ ID NO: 81)

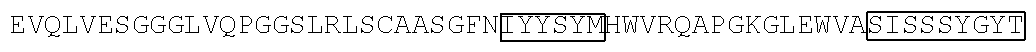

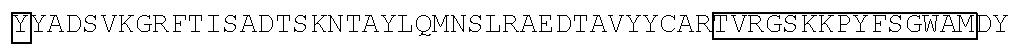

WGQGTLVTVSS
Nucleic acid Sequence:
(SEQ ID NO: 181)
GAGGTTCAGCTGGTGGAGTCTGGCGGTGGCCTGGTGCAGCCAGGGGGCTCACTCCGTT

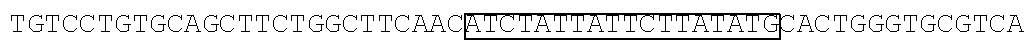

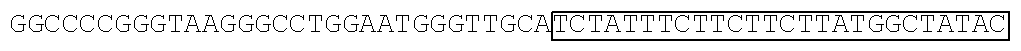

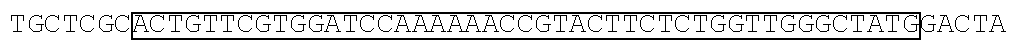

CACAGCCTACCTACAAATGAACAGCTTAAGAGCTGAGGACACTGCCGTCTATTATTG

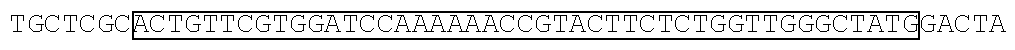

CTGGGGTCAAGGAACCCTGGTCACCGTCTCCTCG
Ab8 VH CDR 1 (Ab8 CDR-H1)
Amino acid Sequence:
(SEQ ID NO: 82)

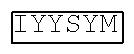

Nucleic acid Sequence:
(SEQ ID NO: 182)

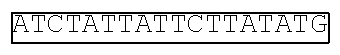

Ab8 VH CDR 2 (Ab8 CDR-H2)
Amino acid Sequence:
(SEQ ID NO: 83)

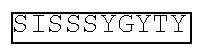

Nucleic acid Sequence:
(SEQ ID NO: 183)

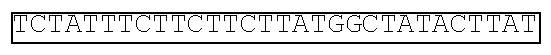

Ab8 VH CDR 3 (Ab8 CDR-H3)
Amino acid Sequence:
(SEQ ID NO: 84)

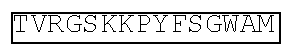

Nucleic acid Sequence:
(SEQ ID NO: 184)

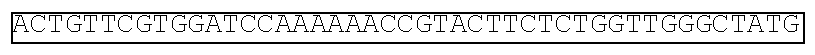

Antibody 8 Light chain (Ab8 LC)
Amino acid Sequence:
(SEQ ID NO: 85)

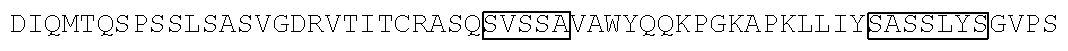

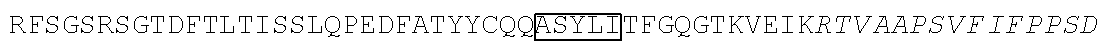

EQLKSGTASVVCLLNNFYPREAKVQWKVDNALQSGNSQESVTEQDSKDSTYSLSSTLTLS
KADYEKHKVYACEVTHQGLSSPVTKSFNRGEC
Nucleic acid Sequence:
(SEQ ID NO: 185)
GATATCCAGATGACCCAGTCCCCGAGCTCCCTGTCCGCCTCTGTGGGCGATAGGGTCA

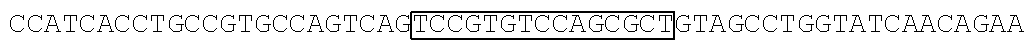

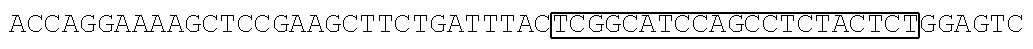

CCTTCTCGCTTCTCTGGTAGCCGTTCCGGGACGGATTTCACTCTGACCATCAGCAGTC

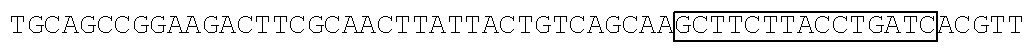

CGGACAGGGTACCAAGGTGGAGATCAAACGTACGGTGGCTGCACCATCTGTCTTCATCT
TCCCGCCATCTGATGAGCAGTTGAAATCTGGAACTGCCTCTGTTGTGTGCCTGCTGAATA
ACTTCTATCCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCTCAGCAGCA
CCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
Antibody 8 Light chain variable region (Ab8 VL)
Amino acid Sequence:
(SEQ ID NO: 86)

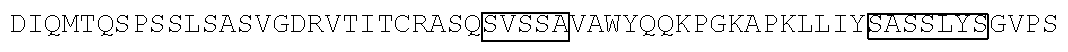

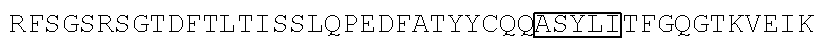

Nucleic acid Sequence:
(SEQ ID NO: 186)
GATATCCAGATGACCCAGTCCCCGAGCTCCCTGTCCGCCTCTGTGGGCGATAGGGTCA

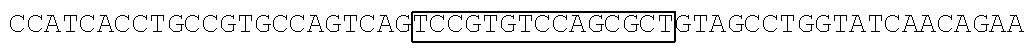

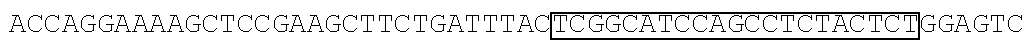

CCTTCTCGCTTCTCTGGTAGCCGTTCCGGGACGGATTTCACTCTGACCATCAGCAGTC

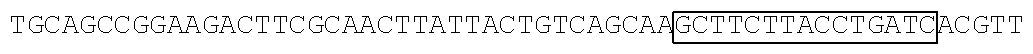

CGGACAGGGTACCAAGGTGGAGATCAAA
Ab8 VL CDR 1 (Ab8 CDR-L1)
Amino acid Sequence:
(SEQ ID NO: 87)

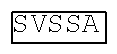

Nucleic acid Sequence:
(SEQ ID NO: 187)

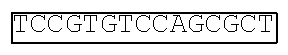

Ab8 VL CDR 2 (Ab8 CDR-L2)
Amino acid Sequence:
(SEQ ID NO: 88)

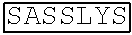

Nucleic acid Sequence:
(SEQ ID NO: 188)

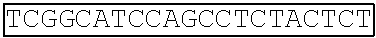

Ab8 VL CDR 3 (Ab8 CDR-L3)
Amino acid Sequence:
(SEQ ID NO: 89)

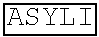

Nucleic acid Sequence:
(SEQ ID NO: 189)

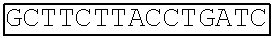

Anti-NMDAR2B Antibody 9 (Ab9):
Antibody 9 Heavy chain (Ab9 HC)
Amino acid Sequence:
(SEQ ID NO: 90)

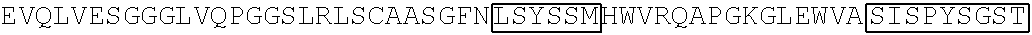

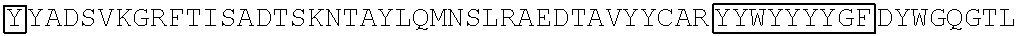

VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
Nucleic acid Sequence:
(SEQ ID NO: 190)
GAGGTTCAGCTGGTGGAGTCTGGCGGTGGCCTGGTGCAGCCAGGGGGCTCACTCCGTT

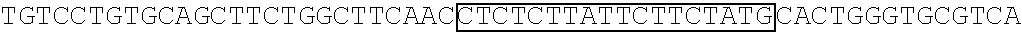

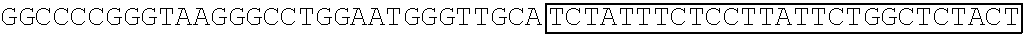

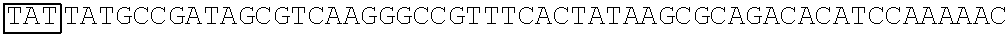

ACAGCCTACCTACAAATGAACAGCTTAAGAGCTGAGGACACTGCCGTCTATTATTGT

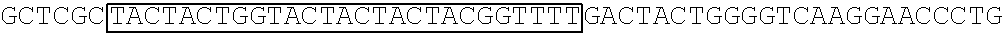

GTCACCGTCTCCTCGGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAA
GAAAGTTGAGCCCAAATCTTGTGACAAAACTCACACATGCCCACCGTGCCCAGCACCTGA
ACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATC
CCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACCGTGGACAA
GAGCAGGTGGCAGCAGGGGAACGTCTTCTCATGCTCCGTGATGCATGAGGCTCTGCACAA
CCACTACACGCAGAAGAGCCTCTCCCTGTCTCCGGGTAAA
Antibody 9 Heavy chain variable region (Ab9 VH)
Amino acid Sequence:
(SEQ ID NO: 91)

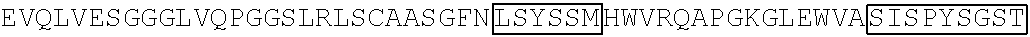

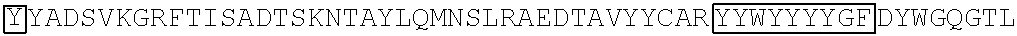

VTVSS
Nucleic acid Sequence:
(SEQ ID NO: 191)
GAGGTTCAGCTGGTGGAGTCTGGCGGTGGCCTGGTGCAGCCAGGGGGCTCACTCCGTT

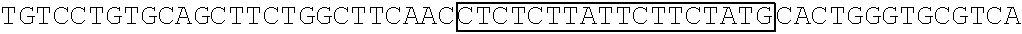

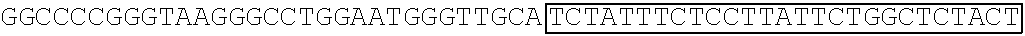

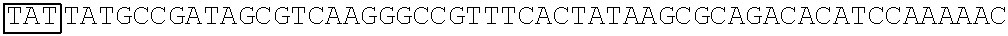

ACAGCCTACCTACAAATGAACAGCTTAAGAGCTGAGGACACTGCCGTCTATTATTGT

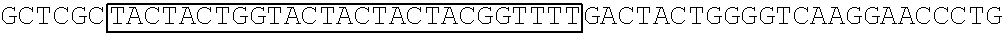

GTCACCGTCTCCTCG
Ab9 VH CDR 1 (Ab9 CDR-H1)
Amino acid Sequence:
(SEQ ID NO: 92)

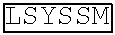

Nucleic acid Sequence:
(SEQ ID NO: 192)

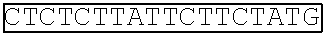

Ab9 VH CDR 2 (Ab9 CDR-H2)
Amino acid Sequence:
(SEQ ID NO: 93)

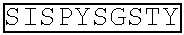

Nucleic acid Sequence:
(SEQ ID NO: 193)

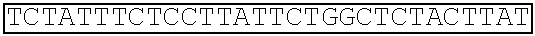

Ab9 VH CDR 3 (Ab9 CDR-H3)
Amino acid Sequence:
(SEQ ID NO: 94)

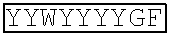

Nucleic acid Sequence:
(SEQ ID NO: 194)

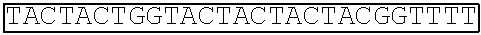

Antibody 9 Light chain (Ab9 LC)
Amino acid Sequence:
(SEQ ID NO: 95)

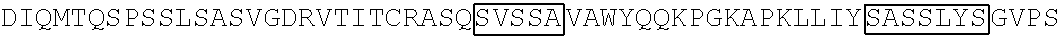

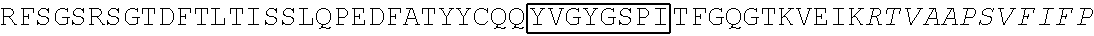

PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Nucleic acid Sequence:
(SEQ ID NO: 195)
GATATCCAGATGACCCAGTCCCCGAGCTCCCTGTCCGCCTCTGTGGGCGATAGGGTCA

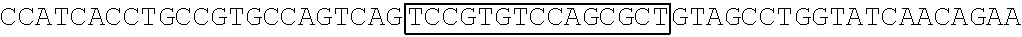

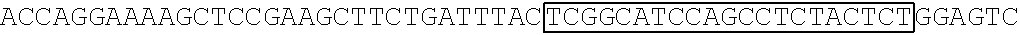

CCTTCTCGCTTCTCTGGTAGCCGTTCCGGGACGGATTTCACTCTGACCATCAGCAGTC

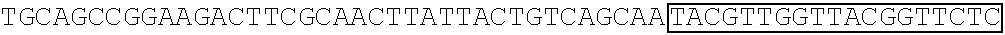

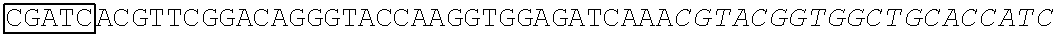

TGTCTTCATCTTCCCGCCATCTGATGAGCAGTTGAAATCTGGAACTGCCTCTGTTGTGTG
CCTGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTG
CGAAGTCACCCATCAGGGCCTGAGCTCGCCCGTCACAAAGAGCTTCAACAGGGGAGAGTG
T
Antibody 9 Light chain variable region (Ab9 VL)
Amino acid Sequence:
(SEQ ID NO: 96)

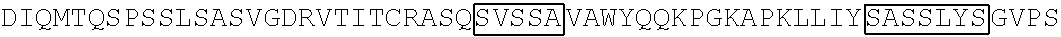

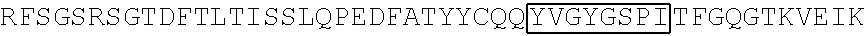

Nucleic acid Sequence:
(SEQ ID NO: 196)
GATATCCAGATGACCCAGTCCCCGAGCTCCCTGTCCGCCTCTGTGGGCGATAGGGTCA

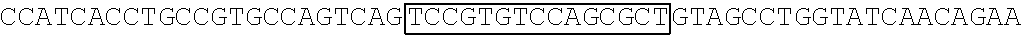

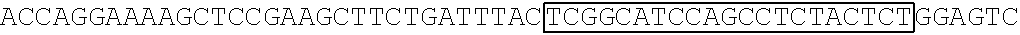

CCTTCTCGCTTCTCTGGTAGCCGTTCCGGGACGGATTTCACTCTGACCATCAGCAGTC

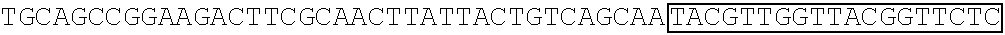

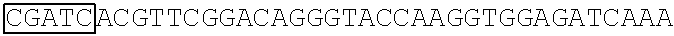

Ab9 VL CDR 1 (Ab9 CDR-L1)
Amino acid Sequence:
(SEQ ID NO: 97)

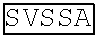

Nucleic acid Sequence:
(SEQ ID NO: 197)

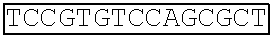

Ab9 VL CDR 2 (Ab9 CDR-L2)
Amino acid Sequence:
(SEQ ID NO: 98)

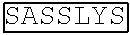

Nucleic acid Sequence:
(SEQ ID NO: 198)

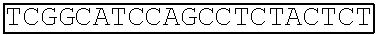

Ab9 VL CDR 3 (Ab9 CDR-L3)
Amino acid Sequence:
(SEQ ID NO: 99)

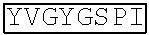

Nucleic acid Sequence:
(SEQ ID NO: 199)

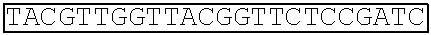

Anti-NMDAR2B Antibody 10 (Ab10):
Antibody 10 Heavy chain (Ab10 HC)
Amino acid Sequence:
(SEQ ID NO: 100)

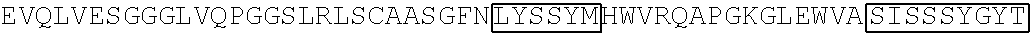

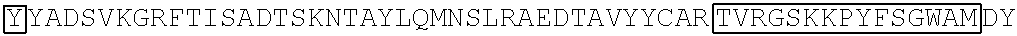

WGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Nucleic acid Sequence:
(SEQ ID NO: 200)
GAGGTTCAGCTGGTGGAGTCTGGCGGTGGCCTGGTGCAGCCAGGGGGCTCACTCCGTT

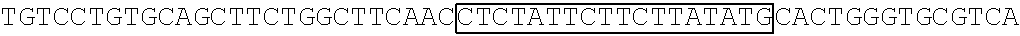

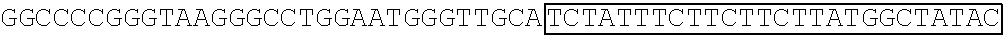

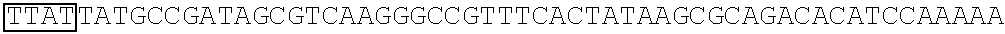

CACAGCCTACCTACAAATGAACAGCTTAAGAGCTGAGGACACTGCCGTCTATTATTG

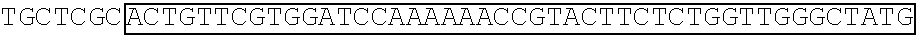

CTGGGGTCAAGGAACCCTGGTCACCGTCTCCTCGGCTAGCACCAAGGGCCCATCGGTCT
TCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAA
Antibody 10 Heavy chain variable region (Ab10 VH)
Amino acid Sequence:
(SEQ ID NO: 101)

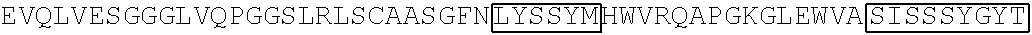

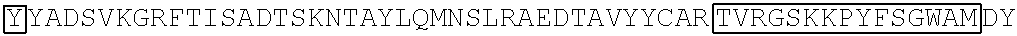

WGQGTLVTVSS
Nucleic acid Sequence:
(SEQ ID NO: 201)
GAGGTTCAGCTGGTGGAGTCTGGCGGTGGCCTGGTGCAGCCAGGGGGCTCACTCCGTT

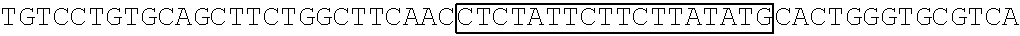

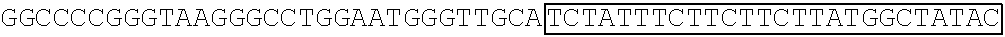

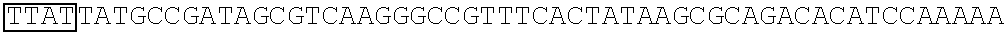

CACAGCCTACCTACAAATGAACAGCTTAAGAGCTGAGGACACTGCCGTCTATTATTG

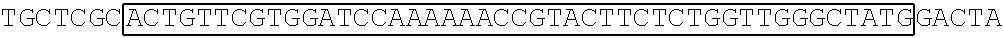

CTGGGGTCAAGGAACCCTGGTCACCGTCTCCTCG
Ab 10 VH CDR 1 (Ab10 CDR-H1)
Amino acid Sequence:
(SEQ ID NO: 102)

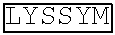

Nucleic acid Sequence:
(SEQ ID NO: 202)

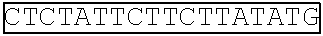

Ab 10 VH CDR 2 (Ab10 CDR-H2)
Amino acid Sequence:
(SEQ ID NO: 103)

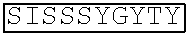

Nucleic acid Sequence:
(SEQ ID NO: 203)

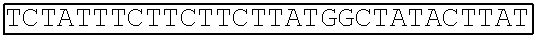

Ab 10 VH CDR 3 (Ab10 CDR-H3)
Amino acid Sequence:
(SEQ ID NO: 104)

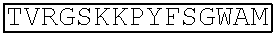

Nucleic acid Sequence:
(SEQ ID NO: 204)

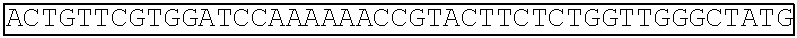

Antibody 10 Light chain (Ab10 LC)
Amino acid Sequence:
(SEQ ID NO: 105)

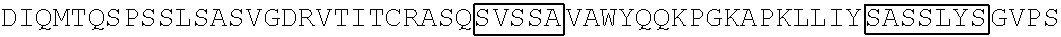

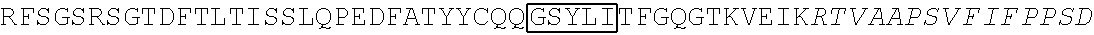

EQLKSGTASVVCLLNNFYPREAKVQWKVDNALQSGNSQESVTEQDSKDSTYSLSSTLTLS
KADYEKHKVYACEVTHQGLSSPVTKSFNRGEC
Nucleic acid Sequence:
(SEQ ID NO: 205)
GATATCCAGATGACCCAGTCCCCGAGCTCCCTGTCCGCCTCTGTGGGCGATAGGGTCA

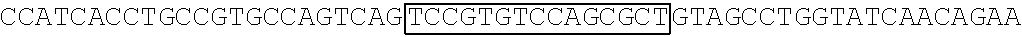

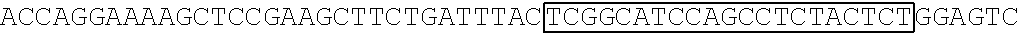

CCTTCTCGCTTCTCTGGTAGCCGTTCCGGGACGGATTTCACTCTGACCATCAGCAGTC

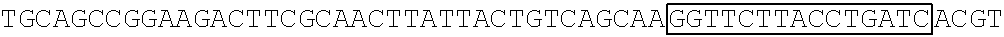

TCGGACAGGGTACCAAGGTGGAGATCAAACGTACGGTGGCTGCACCATCTGTCTTCATC
TTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
Antibody 10 Light chain variable region (Ab10 VL)
Amino acid Sequence:
(SEQ ID NO: 106)

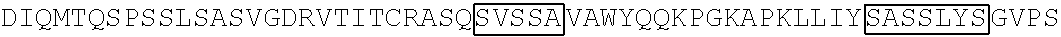

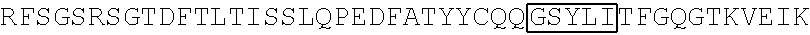

Nucleic acid Sequence:
(SEQ ID NO: 206)
GATATCCAGATGACCCAGTCCCCGAGCTCCCTGTCCGCCTCTGTGGGCGATAGGGTCA

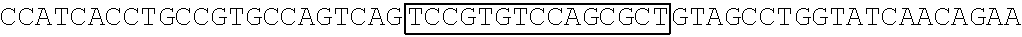

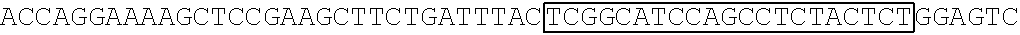

CCTTCTCGCTTCTCTGGTAGCCGTTCCGGGACGGATTTCACTCTGACCATCAGCAGTC

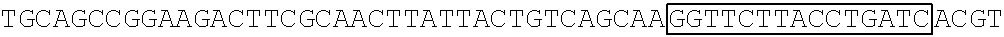

TCGGACAGGGTACCAAGGTGGAGATCAAA
Ab 10 VL CDR 1 (Ab10 CDR-L1)
Amino acid Sequence:
(SEQ ID NO: 107)

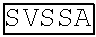

Nucleic acid Sequence:
(SEQ ID NO: 207)

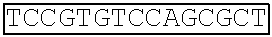

Ab 10 VL CDR 2 (Ab10 CDR-L2)
Amino acid Sequence:
(SEQ ID NO: 108)

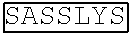

Nucleic acid Sequence:
(SEQ ID NO: 208)

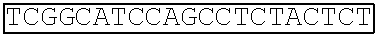

Ab 10 VL CDR 3 (Ab10 CDR-L3)
Amino acid Sequence:
(SEQ ID NO: 109)

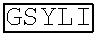

Nucleic acid Sequence:
(SEQ ID NO: 209)

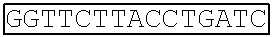

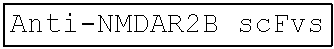

scFv subparts:
Glycine-Serine linker unit (Gly-Gly-Gly-Gly-Ser linker unit, or GS linker unit)
Amino acid Sequence:
GGGG(SEQ ID NO: 110)S
Nucleic acid Sequence:
(SEQ ID NO: 210)
GGTGGTGGTGGTTCT
(Gly-Gly-Gly-Gly-Ser)X3 linker ((Gly4Ser)3 linker, (G4S)3 linker, G4S X3 linker,
or G4SX3 linker)
Protein Sequence:
(SEQ ID NO: 111)
GGGGSGGGGSGGGGS
DNA Sequence:
(SEQ ID NO: 211)
GGTGGTGGTGGTTCTGGCGGCGGCGGCTCCGGTGGTGGTGGTTCC
Anti-NMDAR2B Antibody 4 scFv (Ab4 scFv):
Antibody 4 scFv VH-G4S3-VL (Ab4 scFv HL or Ab4scFvHL)
Amino acid Sequence:
(SEQ ID NO: 240)

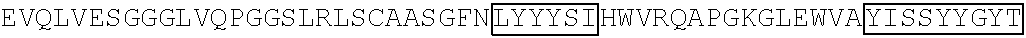

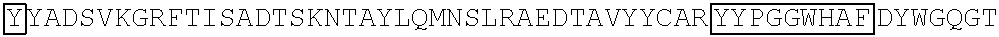

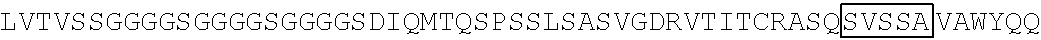

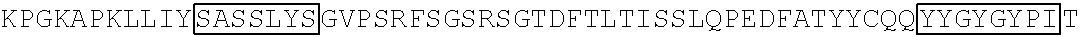

FGQGTKVEIK
Nucleic acid Sequence:
(SEQ ID NO: 340)
GAGGTTCAGCTGGTGGAGTCTGGCGGTGGCCTGGTGCAGCCAGGGGGCTCACTCCGTT

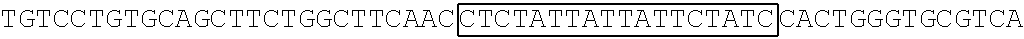

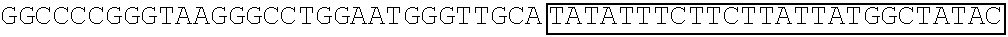

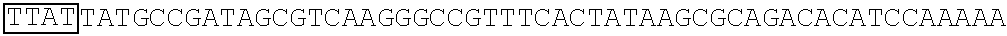

CACAGCCTACCTACAAATGAACAGCTTAAGAGCTGAGGACACTGCCGTCTATTATTG

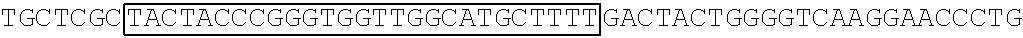

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

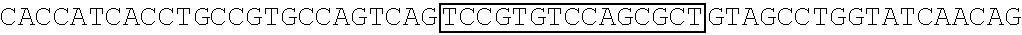

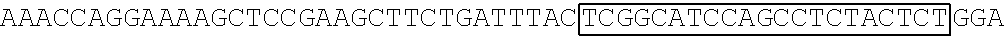

GTCCCTTCTCGCTTCTCTGGTAGCCGTTCCGGGACGGATTTCACTCTGACCATCAGCA

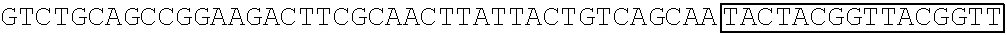

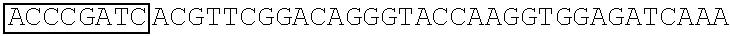

Antibody 4 scFv VL-G4S3-VH (Ab4 scFv LH or Ab4scFvLH)
Amino acid Sequence:
(SEQ ID NO: 245)

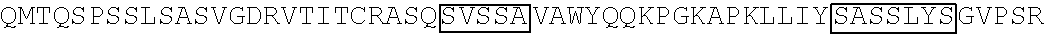

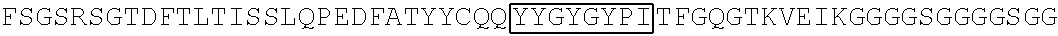

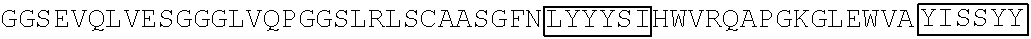

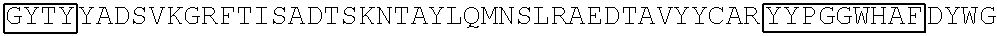

QGTLVTVSS
Nucleic acid Sequence:
(SEQ ID NO: 345)
GATATCCAGATGACCCAGTCCCCGAGCTCCCTGTCCGCCTCTGTGGGCGATAGGGTCA

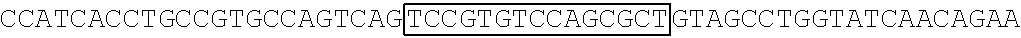

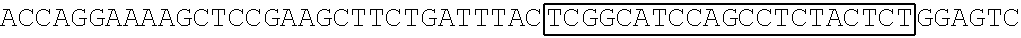

CCTTCTCGCTTCTCTGGTAGCCGTTCCGGGACGGATTTCACTCTGACCATCAGCAGTC

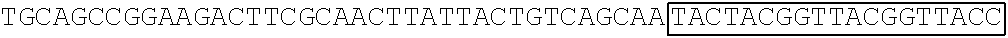

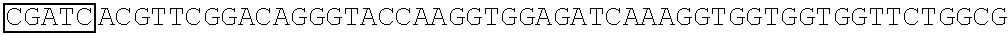

GCGGCGGCTCCGGTGGTGGTGGTTCCGAGGTTCAGCTGGTGGAGTCTGGCGGTGGCCT

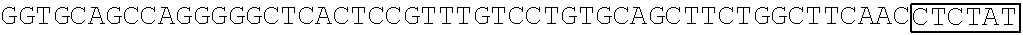

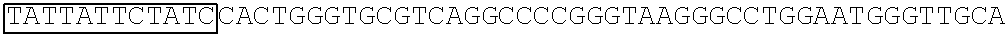

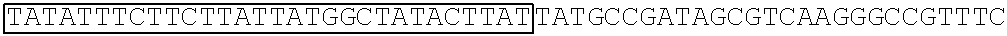

ACTATAAGCGCAGACACATCCAAAAACACAGCCTACCTACAAATGAACAGCTTAAGA

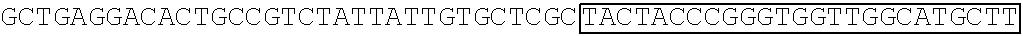

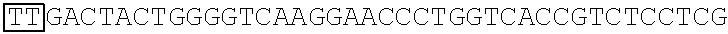

Anti-NMDAR2B Antibody 5 scFv (Ab5 scFv):
Antibody 5 scFv VH-G4S3-VL (Ab5 scFv HL or Ab5scFvHL)
Amino acid Sequence:
(SEQ ID NO: 250)

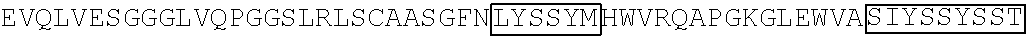

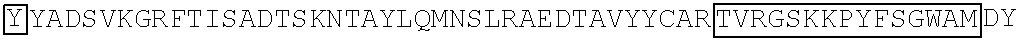

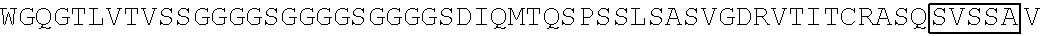

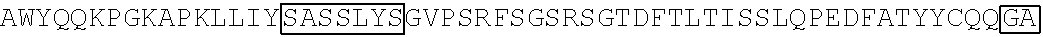

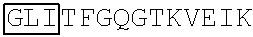

Nucleic acid Sequence:
(SEQ ID NO: 350)
GAGGTTCAGCTGGTGGAGTCTGGCGGTGGCCTGGTGCAGCCAGGGGGCTCACTCCGTT

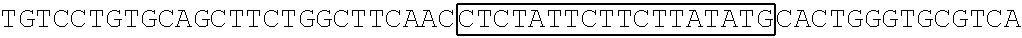

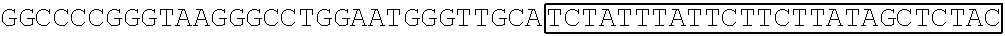

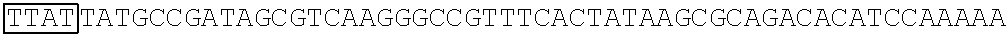

CACAGCCTACCTACAAATGAACAGCTTAAGAGCTGAGGACACTGCCGTCTATTATTG

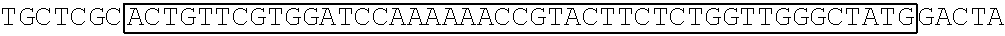

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

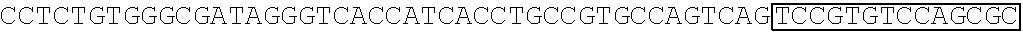

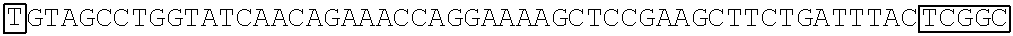

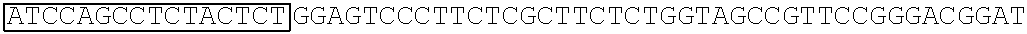

TTCACTCTGACCATCAGCAGTCTGCAGCCGGAAGACTTCGCAACTTATTACTGTCAGC

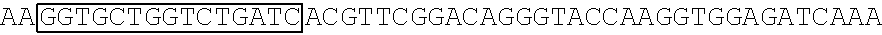

Antibody 5 scFv VL-G4S3-VH (Ab5 scFv LH or Ab5scFvLH)
Amino acid Sequence:
(SEQ ID NO: 255)

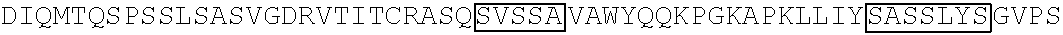

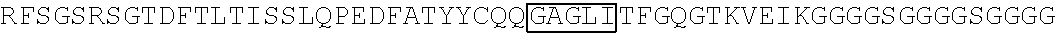

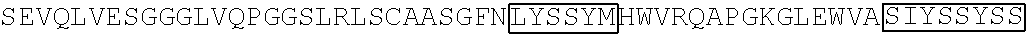

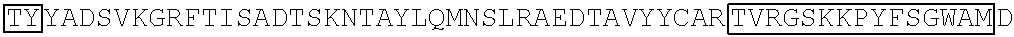

YWGQGTLVTVSS
Nucleic acid Sequence:
(SEQ ID NO: 355)
GATATCCAGATGACCCAGTCCCCGAGCTCCCTGTCCGCCTCTGTGGGCGATAGGGTCA

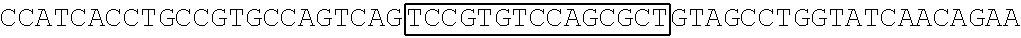

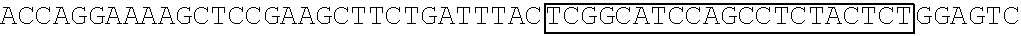

CCTTCTCGCTTCTCTGGTAGCCGTTCCGGGACGGATTTCACTCTGACCATCAGCAGTC

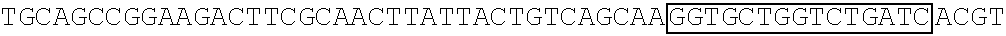

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

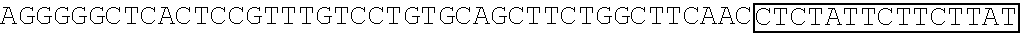

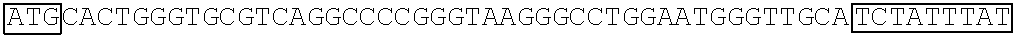

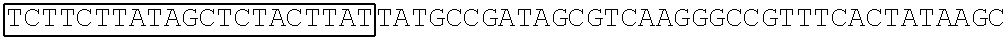

GCAGACACATCCAAAAACACAGCCTACCTACAAATGAACAGCTTAAGAGCTGAGGAC

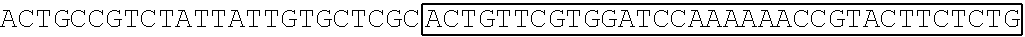

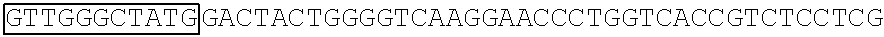

Anti-NMDAR2B Antibody 6 scFv (Ab6 scFv):
Antibody 6 scFv VH-G4S3-VL (Ab6 scFv HL or Ab6scFvHL)
Amino acid Sequence:
(SEQ ID NO: 260)

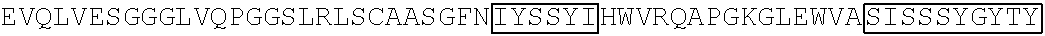

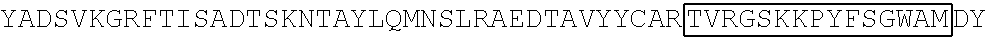

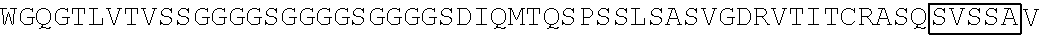

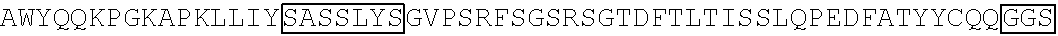

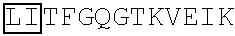

Nucleic acid Sequence:
(SEQ ID NO: 360)
GAGGTTCAGCTGGTGGAGTCTGGCGGTGGCCTGGTGCAGCCAGGGGGCTCACTCCGTT

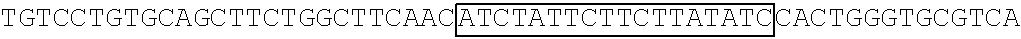

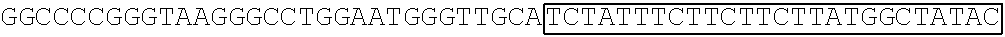

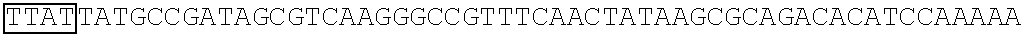

CACAGCCTACCTACAAATGAACAGCTTAAGAGCTGAGGACACTGCCGTCTATTATTG

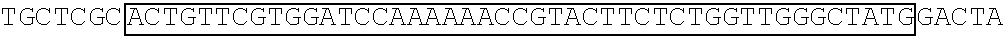

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

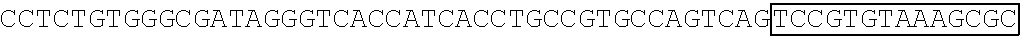

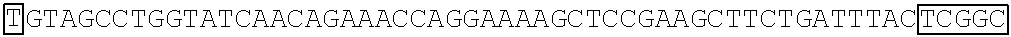

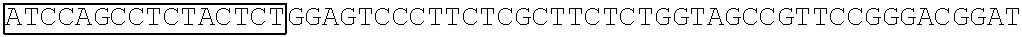

TTCACTCTGACCATCAGCAGTCTGCAGCCGGAAGACTTCGCAACTTATTACTGTCAGC

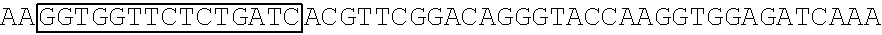

Antibody 6 scFv VL-G4S3-VH (Ab6 scFv LH or Ab6scFvLH)
Amino acid Sequence:
(SEQ ID NO: 265)

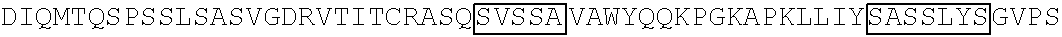

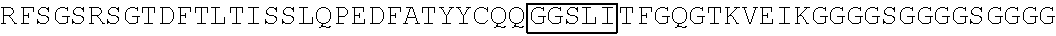

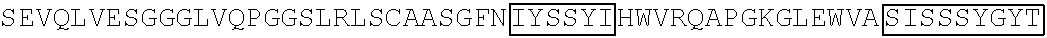

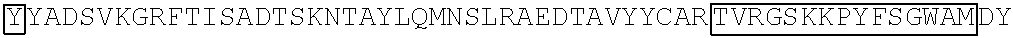

WGQGTLVTVSS
Nucleic acid Sequence:
(SEQ ID NO: 365)
GATATCCAGATGACCCAGTCCCCGAGCTCCCTGTCCGCCTCTGTGGGCGATAGGGTCA

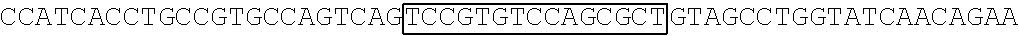

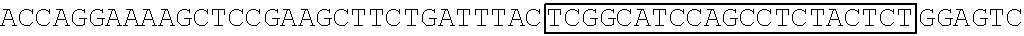

CCTTCTCGCTTCTCTGGTAGCCGTTCCGGGACGGATTTCACTCTGACCATCAGCAGTC

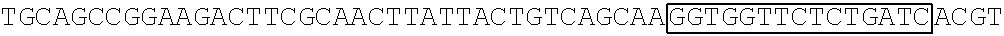

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

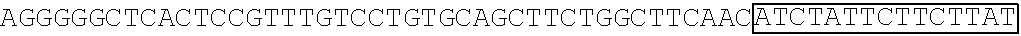

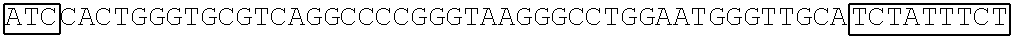

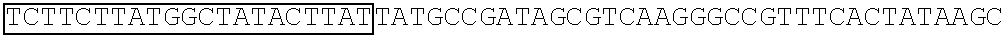

GCAGACACATCCAAAAACACAGCCTACCTACAAATGAACAGCTTAAGAGCTGAGGAC

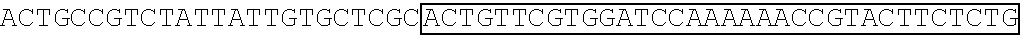

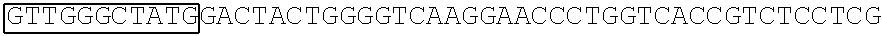

Anti-NMDAR2B Antibody 7 scFv (Ab7 scFv):
Antibody 7 scFv VH-G4S3-VL (Ab7 scFv HL or Ab 7scFvHL)
Amino acid Sequence:
(SEQ ID NO: 270)

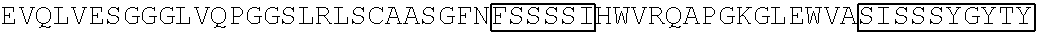

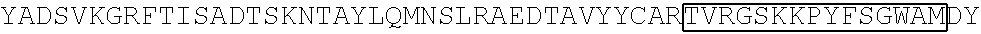

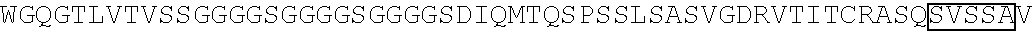

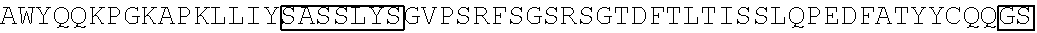

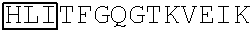

Nucleic acid Sequence:
(SEQ ID NO: 370)
GAGGTTCAGCTGGTGGAGTCTGGCGGTGGCCTGGTGCAGCCAGGGGGCTCACTCCGTT

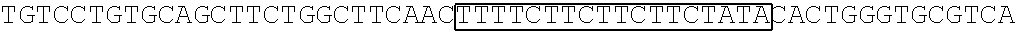

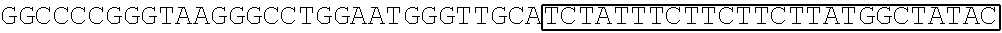

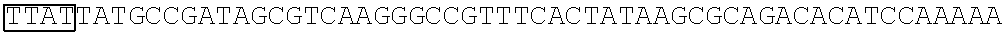

CACAGCCTACCTACAAATGAACAGCTTAAGAGCTGAGGACACTGCCGTCTATTATTG

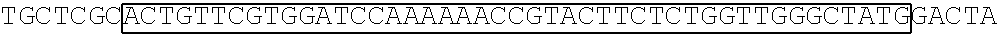

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

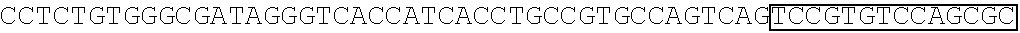

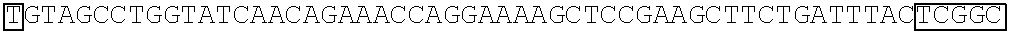

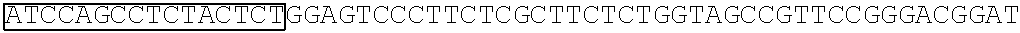

TTCACTCTGACCATCAGCAGTCTGCAGCCGGAAGACTTCGCAACTTATTACTGTCAGC

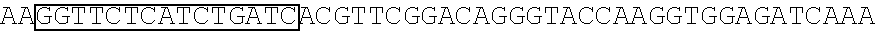

Antibody 7 scFv VL-G4S3-VH (Ab7 scFv LH or Ab7scFvLH)
Amino acid Sequence:
(SEQ ID NO: 275)

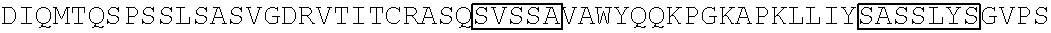

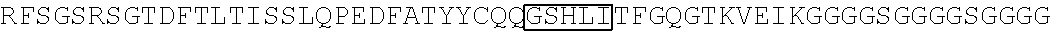

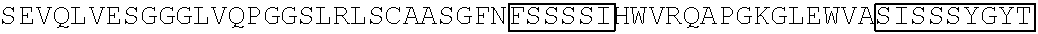

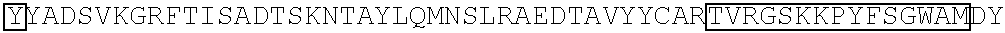

WGQGTLVTVSS
Nucleic acid Sequence:
(SEQ ID NO: 375)
GATATCCAGATGACCCAGTCCCCGAGCTCCCTGTCCGCCTCTGTGGGCGATAGGGTCA

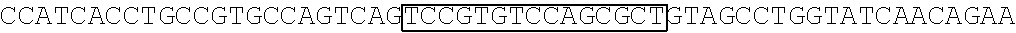

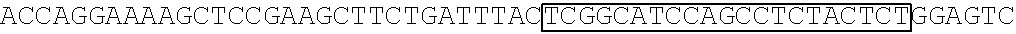

CCTTCTCGCTTCTCTGGTAGCCGTTCCGGGACGGATTTCACTCTGACCATCAGCAGTC

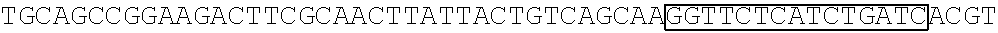

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

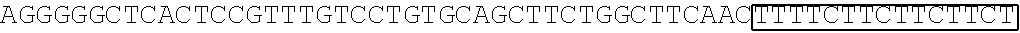

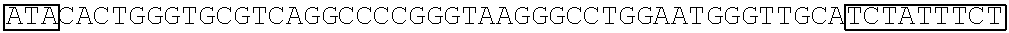

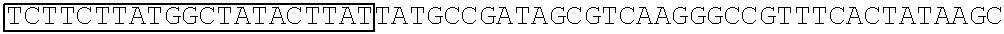

GCAGACACATCCAAAAACACAGCCTACCTACAAATGAACAGCTTAAGAGCTGAGGAC

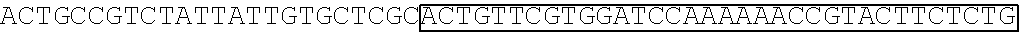

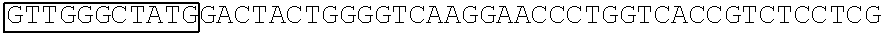

Anti-NMDAR2B Antibody 8 scFv (Ab8 scFv):
Antibody 8 scFv VH-G4S3-VL (Ab8 scFv HL or Ab8scFvHL)
Amino acid Sequence:
(SEQ ID NO: 280)

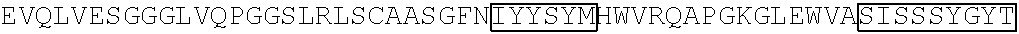

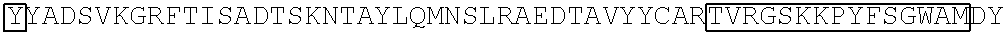

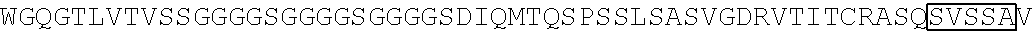

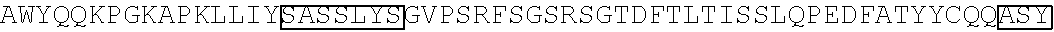

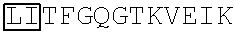

Nucleic acid Sequence:
(SEQ ID NO: 380)
GAGGTTCAGCTGGTGGAGTCTGGCGGTGGCCTGGTGCAGCCAGGGGGCTCACTCCGTT

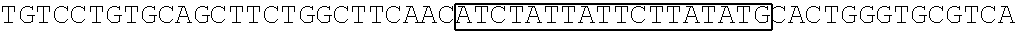

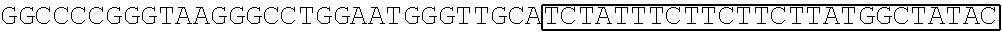

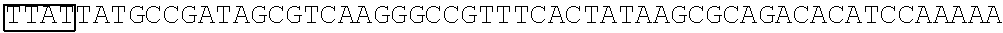

CACAGCCTACCTACAAATGAACAGCTTAAGAGCTGAGGACACTGCCGTCTATTATTG

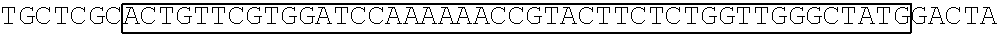

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

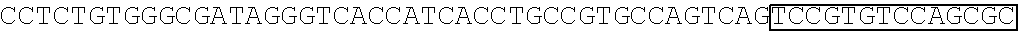

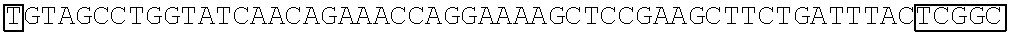

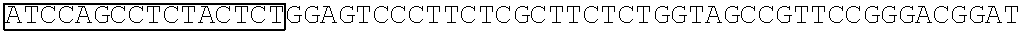

TTCACTCTGACCATCAGCAGTCTGCAGCCGGAAGACTTCGCAACTTATTACTGTCAGC

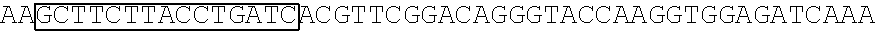

Antibody 8 scFv VL-G4S3-VH (Ab8 scFv LH or Ab8scFvLH)
Amino acid Sequence:
(SEQ ID NO: 285)

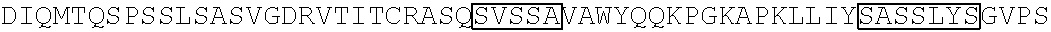

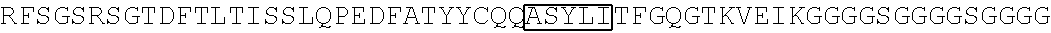

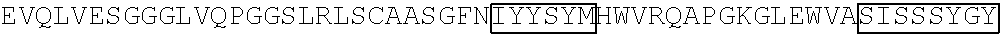

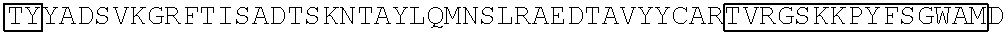

YWGQGTLVTVSS
Nucleic acid Sequence:
(SEQ ID NO: 385)
GATATCCAGATGACCCAGTCCCCGAGCTCCCTGTCCGCCTCTGTGGGCGATAGGGTCA

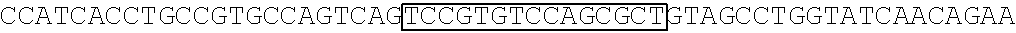

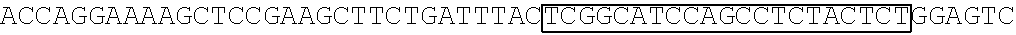

CCTTCTCGCTTCTCTGGTAGCCGTTCCGGGACGGATTTCACTCTGACCATCAGCAGTC

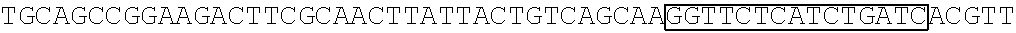

CGGACAGGGTACCAAGGTGGAGATCAAAGGTGGTGGTGGTTCTGGCGGCGGCGGCTC
CGGTGGTGGTGGTTCCGAGGTTCAGCTGGTGGAGTCTGGCGGTGGCCTGGTGCAGCC

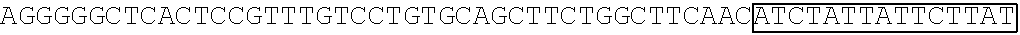

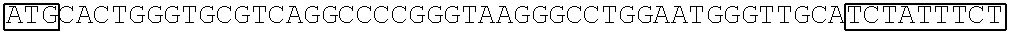

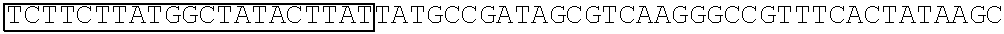

GCAGACACATCCAAAAACACAGCCTACCTACAAATGAACAGCTTAAGAGCTGAGGAC

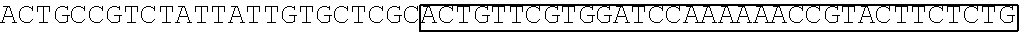

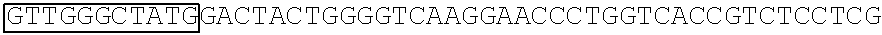

Anti-NMDAR2B Antibody 9 scFv (Ab9 scFv):
Antibody 9 scFv VH-G4S3-VL (Ab9 scFv HL or Ab9scFvHL)
Amino acid Sequence:
(SEQ ID NO: 290)

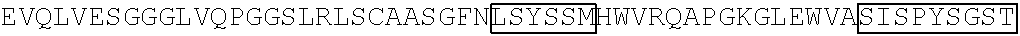

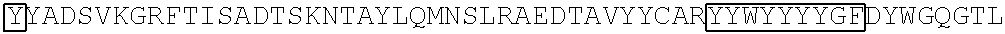

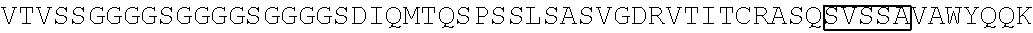

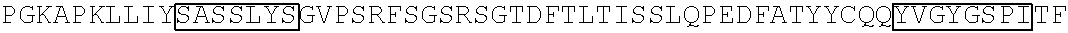

GQGTKVEIK
Nucleic acid Sequence:
(SEQ ID NO: 390)
GAGGTTCAGCTGGTGGAGTCTGGCGGTGGCCTGGTGCAGCCAGGGGGCTCACTCCGTT

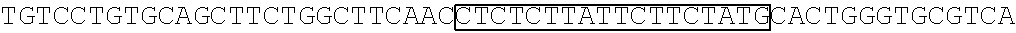

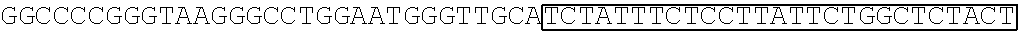

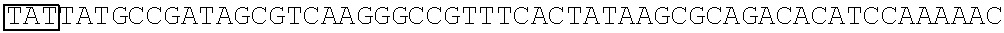

ACAGCCTACCTACAAATGAACAGCTTAAGAGCTGAGGACACTGCCGTCTATTATTGT

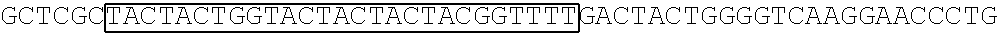

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

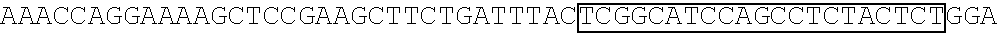

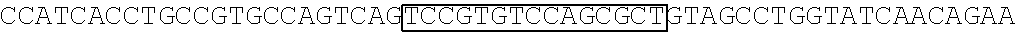

GTCCCTTCTCGCTTCTCTGGTAGCCGTTCCGGGACGGATTTCACTCTGACCATCAGCA

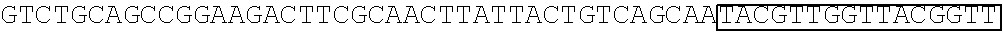

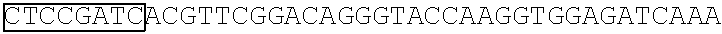

Antibody 9 scFv VL-G4S3-VH (Ab9 scFv LH or Ab9scFvLH)
Amino acid Sequence:
(SEQ ID NO: 295)

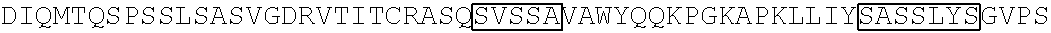

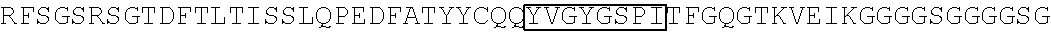

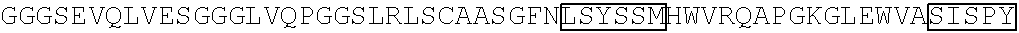

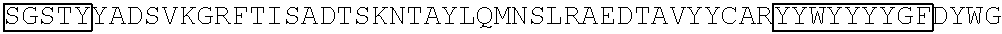

QGTLVTVSS
Nucleic acid Sequence:
(SEQ ID NO: 395)
GATATCCAGATGACCCAGTCCCCGAGCTCCCTGTCCGCCTCTGTGGGCGATAGGGTCA

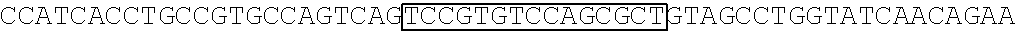

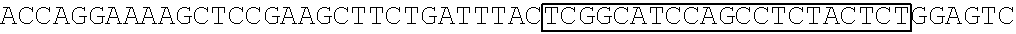

CCTTCTCGCTTCTCTGGTAGCCGTTCCGGGACGGATTTCACTCTGACCATCAGCAGTC

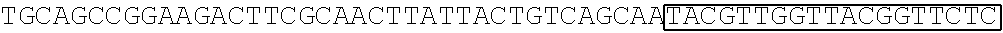

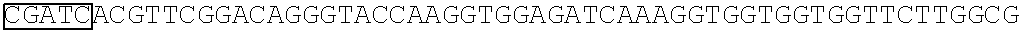

GCGGCGGCTCCGGTGGTGGTGGTTCCGAGGTTCAGCTGGTGGAGTCTGGCGGTGGCCT

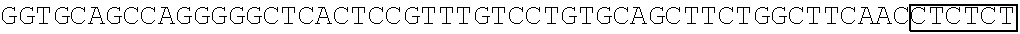

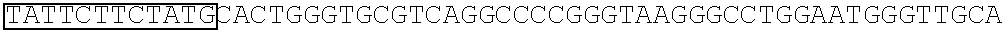

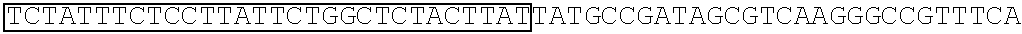

CTATAAGCGCAGACACATCCAAAAACACAGCCTACCTACAAATGAACAGCTTAAGAG

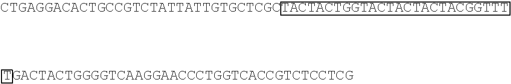

Anti-NMDAR2B Antibody 10 scFv (Ab10 scFv):
Antibody 10 scFv VH-G4S3-VL (Ab 10 scFv HL or Ab 10scFvHL)
Amino acid Sequence:
(SEQ ID NO: 300)

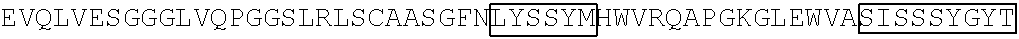

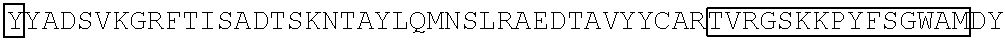

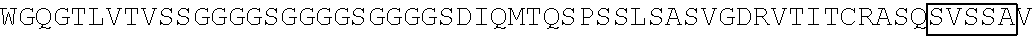

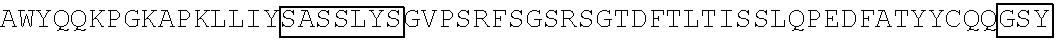

Nucleic acid Sequence:
(SEQ ID NO: 400)
GAGGTTCAGCTGGTGGAGTCTGGCGGTGGCCTGGTGCAGCCAGGGGGCTCACTCCGTT

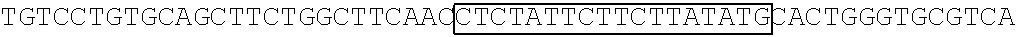

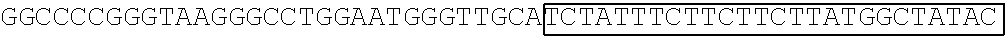

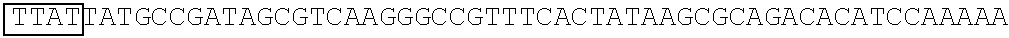

CACAGCCTACCTACAAATGAACAGCTTAAGAGCTGAGGACACTGCCGTCTATTATTG

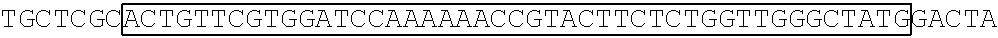

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

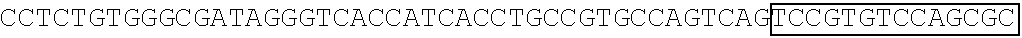

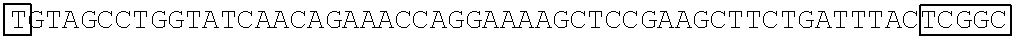

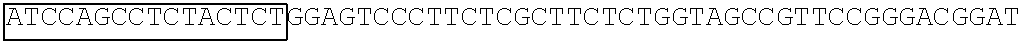

TTCACTCTGACCATCAGCAGTCTGCAGCCGGAAGACTTCGCAACTTATTACTGTCAGC

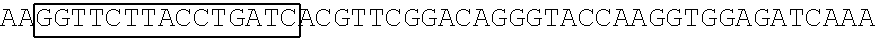

Antibody 10 scFv VL-G4S3-VH (Ab10 scFv LH or Ab10scFvLH)
Amino acid Sequence:
(SEQ ID NO: 305)

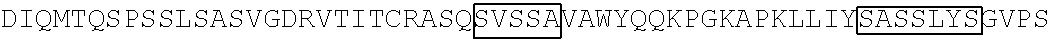

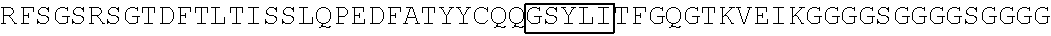

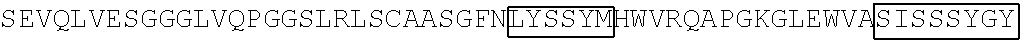

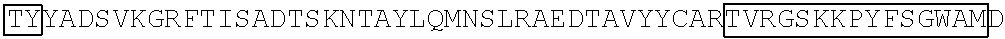

YWGQGTLVTVSS
Nucleic acid Sequence:
(SEQ ID NO: 405)
GATATCCAGATGACCCAGTCCCCGAGCTCCCTGTCCGCCTCTGTGGGCGATAGGGTCA

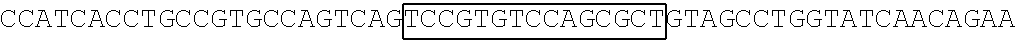

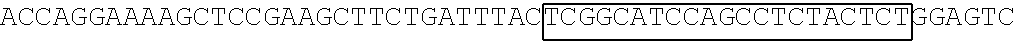

CCTTCTCGCTTCTCTGGTAGCCGTTCCGGGACGGATTTCACTCTGACCATCAGCAGTC

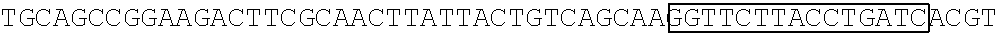

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

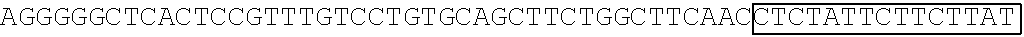

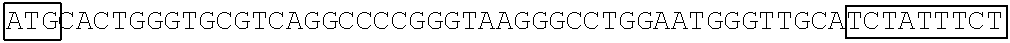

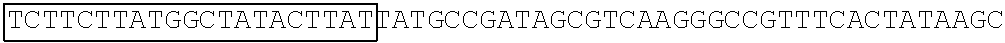

GCAGACACATCCAAAAACACAGCCTACCTACAAATGAACAGCTTAAGAGCTGAGGAC

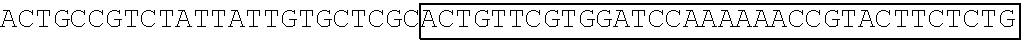

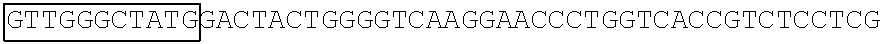

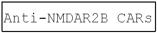

CAR subparts:
Lead sequence (LS)
)Amino acid Sequence:
(SEQ ID NO: 112
METPAQLLFLLLLWLPDTTG
Nucleic acid Sequence:
(SEQ ID NO: 212)
ATGGAAACCCCAGCGCAGCTTCTCTTCCTCCTGCTACTCTGGCTCCCAGATACCACCG
GA
Human CD28 hinge (CD28 hinge, or CD28H)
Amino acid Sequence:
(SEQ ID NO: 113)
LEVKGKHLCPSPLFPGPSKP
Nucleic acid Sequence:
(SEQ ID NO: 213)
CTCGAGGTGAAAGGGAAACACCTTTGTCCAAGTCCCCTATTTCCCGGACCTTCTAAGC
CC
Human CD28 transmembrane domain (CD28 TM domain, or CD28TM)
Amino acid Sequence:
(SEQ ID NO: 114)
FWVLVVVGGVLACYSLLVTVAFIIFWV
Nucleic acid Sequence:
(SEQ ID NO: 214)
TTTTGGGTGCTGGTGGTGGTTGGTGGAGTCCTGGCTTGCTATAGCTTGCTAGTAACA
GTGGCCTTTATTATTTTCTGGGTG
Human CD28 costimulatory domain (CD28 CS domain, or CD28CS)
Amino acid Sequence:
(SEQ ID NO: 115)
RSKRSRLLHSDYMNMTPRRPGPTRKHYQPYAPPRDFAAYRSKL
Nucleic acid Sequence:
(SEQ ID NO: 215)
AGGAGTAAGAGGAGCAGGCTCCTGCACAGTGACTACATGAACATGACTCCCCGCCGC
CCCGGGCCCACCCGCAAGCATTACCAGCCCTATGCCCCACCACGCGACTTCGCAGCCT
ATCGCTCCAAGCTT
Human 4-1BB costimulatory domain (4-1BB CS domain, 41BB CS domain, or
41BBCS)
Amino acid Sequence:
(SEQ ID NO: 116)
KRGRKKLLYIFKQPFMRPVQTTQEEDGCSCRFPEEEEGGCEL
Nucleic acid Sequence:
(SEQ ID NO: 216)
AAACGGGGCAGAAAGAAACTCCTGTATATATTCAAACAACCATTTATGAGACCAGTA
CAAACTACTCAAGAGGAAGATGGCTGTAGCTGCCGATTTCCAGAAGAAGAAGAAGGA
GGATGTGAACTG
Human DAP10 costimulatory domain (DAP10 CS domain, or DAP10CS)
Amino acid Sequence:
(SEQ ID NO: 117)
LCARPRRSPAQEDGKVYINMPGRG
Nucleic acid Sequence:
(SEQ ID NO: 217)
CTGTGCGCACGCCCACGCCGCAGCCCCGCCCAAGAAGATGGCAAAGTCTACATCAACA
TGCCAGGCAGGGGC
Human CD3 zeta intracellular signaling domain (CD3z ICS domain, CD3 Z ICS, or
CD3zICS)
Amino acid Sequence:
(SEQ ID NO: 118)
RVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPP
R
Nucleic acid Sequence:
(SEQ ID NO: 218)
AGAGTGAAGTTCAGCAGGAGCGCAGACGCCCCCGCGTACCAGCAGGGCCAGAACCAG
CTCTATAACGAGCTCAATCTAGGACGAAGAGAGGAGTACGATGTTTTGGACAAGAGA
CGTGGCCGGGACCCTGAGATGGGGGGAAAGCCGCAGAGAAGGAAGAACCCTCAGGAA
GGCCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCAGT
ACAGCCACCAAGGACACCTACGACGCCCTTCACATGCAGGCCCTGCCCCCTCGC
T2A ribosomal skip sequence (T2A)
Amino acid Sequence:
(SEQ ID NO: 119)
AGAKRSGSGEGRGSLLTCGDVEENPGPR
Nucleic acid Sequence:
(SEQ ID NO: 219)
GCCGGCGCCAAAAGGTCTGGCTCCGGTGAGGGCAGAGGAAGTCTTCTAACATGCGGT
GACGTGGAGGAGAATCCCGGCCCTAGA
truncated CD19 (trCD19)
Amino acid Sequence:
(SEQ ID NO: 120)
MPPPRLLFFLLFLTPMEVRPEEPLVVKVEEGDNAVLQCLKGTSDGPTQQLTWSRESPL
KPFLKLSLGLPGLGIHMRPLAIWLFIFNVSQQMGGFYLCQPGPPSEKAWQPGWTVNV
EGSGELFRWNVSDLGGLGCGLKNRSSEGPSSPSGKLMSPKLYVWAKDRPEIWEGEPPC
LPPRDSLNQSLSQDLTMAPGSTLWLSCGVPPDSVSRGPLSWTHVHPKGPKSLLSLELK
DDRPARDMWVMETGLLLPRATAQDAGKYYCHRGNLTMSFHLEITARPVLWHWLLR
TGGWKVSAVTLAYLIFCLCSLVGILHLQRALVLRRKRKRMT
Nucleic acid Sequence:
(SEQ ID NO: 220)
ATGCCACCTCCTCGCCTCCTCTTCTTCCTCCTCTTCCTCACCCCCATGGAAGTCAGGCC
CGAGGAACCTCTAGTGGTGAAGGTGGAAGAGGGAGATAACGCTGTGCTGCAGTGCCT
CAAGGGGACCTCAGATGGCCCCACTCAGCAGCTGACCTGGTCTCGGGAGTCCCCGCTT
AAACCCTTCTTAAAACTCAGCCTGGGGCTGCCAGGCCTGGGAATCCACATGAGGCCCC
TGGCCATCTGGCTTTTCATCTTCAACGTCTCTCAACAGATGGGGGGCTTCTACCTGTG
CCAGCCGGGGCCCCCCTCTGAGAAGGCCTGGCAGCCTGGCTGGACAGTCAATGTGGAG
GGCAGCGGGGAGCTGTTCCGGTGGAATGTTTCGGACCTAGGTGGCCTGGGCTGTGGC
CTGAAGAACAGGTCCTCAGAGGGCCCCAGCTCCCCTTCCGGGAAGCTCATGAGCCCCA
AGCTGTATGTGTGGGCCAAAGACCGCCCTGAGATCTGGGAGGGAGAGCCTCCGTGTC
TCCCACCGAGGGACAGCCTGAACCAGAGCCTCAGCCAGGACCTCACCATGGCCCCTGG
CTCCACACTCTGGCTGTCCTGTGGGGTACCCCCTGACTCTGTGTCCAGGGGCCCCCTC
TCCTGGACCCATGTGCACCCCAAGGGGCCTAAGTCATTGCTGAGCCTAGAGCTGAAGG
ACGATCGCCCGGCCAGAGATATGTGGGTAATGGAGACGGGTCTGTTGTTGCCCCGGG
CCACAGCTCAAGACGCTGGAAAGTATTATTGTCACCGTGGCAACCTGACCATGTCATT
CCACCTGGAGATCACTGCTCGGCCAGTACTATGGCACTGGCTGCTGAGGACTGGTGGC
TGGAAGGTCTCAGCTGTGACTTTGGCTTATCTGATCTTCTGCCTGTGTTCCCTTGTGG
GCATTCTTCATCTTCAAAGAGCCCTGGTCCTGAGGAGGAAAAGAAAGCGAATGACTT
AA

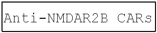

CARs comprising Ab4scFvHL:
Ab4scFvHL-CD28H-CD28TM-CD28CS-CD3zICS
Amino acid Sequence:
(SEQ ID NO: 241)

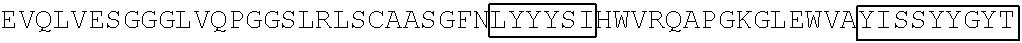

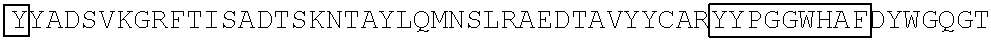

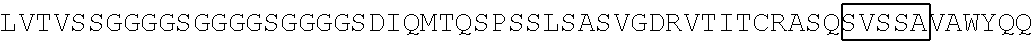

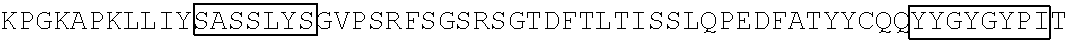

FGQGTKVEIKLEVKGKHLCPSPLFPGPSKPFWVLVVVGGVLACYSLLVTVAFIIFWVRS
KRSRLLHSDYMNMTPRRPGPTRKHYQPYAPPRDFAAYRSKLRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 341)
GAGGTTCAGCTGGTGGAGTCTGGCGGTGGCCTGGTGCAGCCAGGGGGCTCACTCCGTT

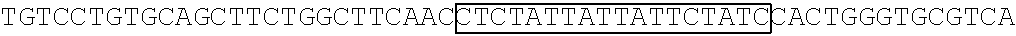

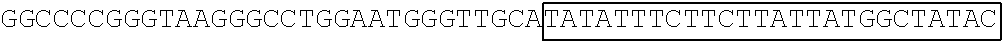

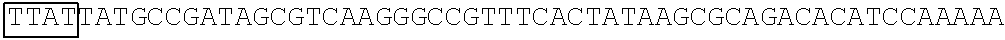

CACAGCCTACCTACAAATGAACAGCTTAAGAGCTGAGGACACTGCCGTCTATTATTG

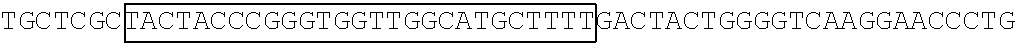

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

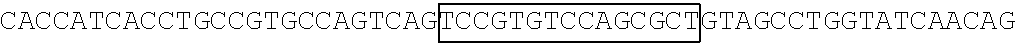

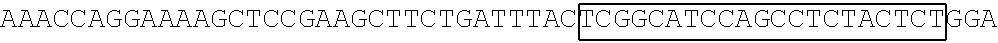

GTCCCTTCTCGCTTCTCTGGTAGCCGTTCCGGGACGGATTTCACTCTGACCATCAGCA

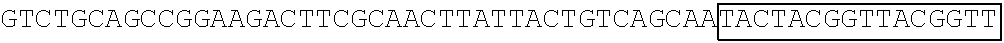

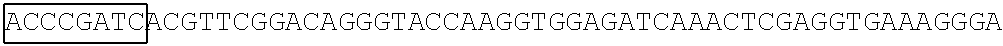

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACATGAC
TCCCCGCCGCCCCGGGCCCACCCGCAAGCATTACCAGCCCTATGCCCCACCACGCGACT
TCGCAGCCTATCGCTCCAAGCTTAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab4scFvHL-CD28H-CD28TM-41BBCS-CD3zICS
Amino acid Sequence:
(SEQ ID NO: 242)

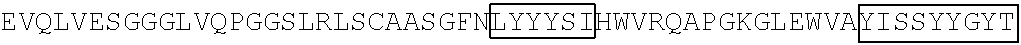

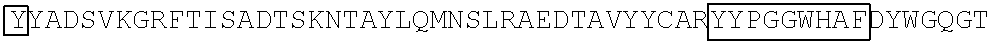

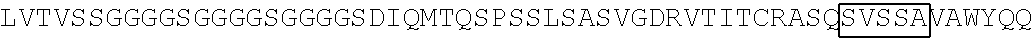

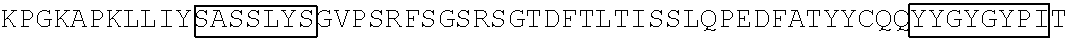

FGQGTKVEIKLEVKGKHLCPSPLFPGPSKPFWVLVVVGGVLACYSLLVTVAFIIFWVKR
GRKKLLYIFKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKMAEAYSE
IGMKGERRRGKGHDGLYQGLSTATKDTYDALHMQALPPR
Nucleic acid Sequence:
(SEQ ID NO: 342)
GAGGTTCAGCTGGTGGAGTCTGGCGGTGGCCTGGTGCAGCCAGGGGGCTCACTCCGTT

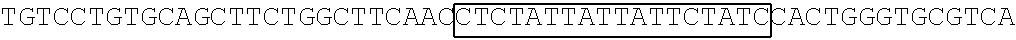

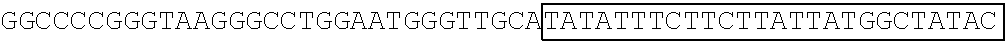

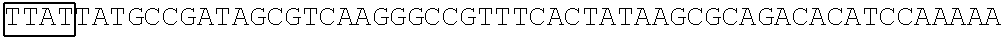

CACAGCCTACCTACAAATGAACAGCTTAAGAGCTGAGGACACTGCCGTCTATTATTG

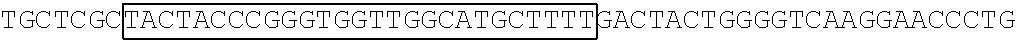

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

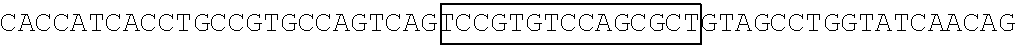

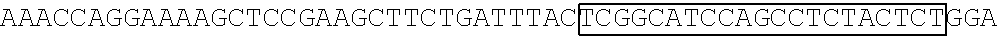

GTCCCTTCTCGCTTCTCTGGTAGCCGTTCCGGGACGGATTTCACTCTGACCATCAGCA

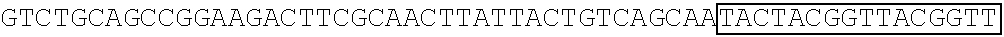

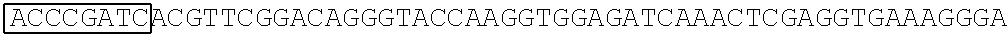

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAAACGGGGCAGAAAGAAACTCCTGTATATATTCAAACAACCATTTAT
GAGACCAGTACAAACTACTCAAGAGGAAGATGGCTGTAGCTGCCGATTTCCAGAAGA
AGAAGAAGGAGGATGTGAACTGAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab4scFvHL-CD28H-CD28TM-DAP10CS-CD3zICS
Amino acid Sequence:
(SEQ ID NO: 243)

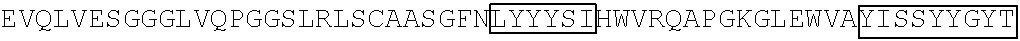

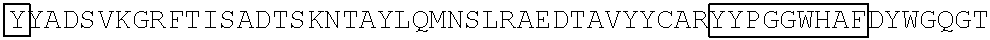

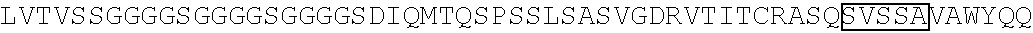

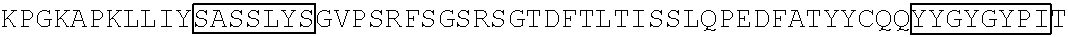

FGQGTKVEIKLEVKGKHLCPSPLFPGPSKPFWVLVVVGGVLACYSLLVTVAFIIFWVLC
ARPRRSPAQEDGKVYINMPGRGRVKFSRSADAPAYQQGQNQLYNELNLGRREEYDVL
DKRRGRDPEMGGKPQRRKNPQEGLYNELQKDKMAEAYSEIGMKGERRRGKGHDGLY
QGLSTATKDTYDALHMQALPPR
Nucleic acid Sequence:
(SEQ ID NO: 343)
GAGGTTCAGCTGGTGGAGTCTGGCGGTGGCCTGGTGCAGCCAGGGGGCTCACTCCGTT

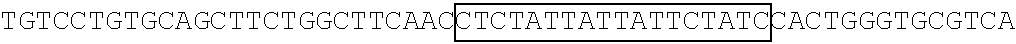

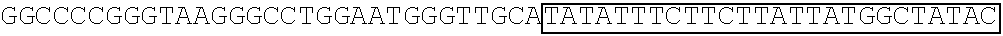

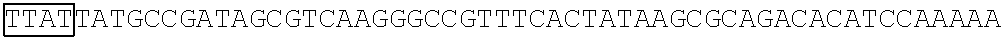

CACAGCCTACCTACAAATGAACAGCTTAAGAGCTGAGGACACTGCCGTCTATTATTG

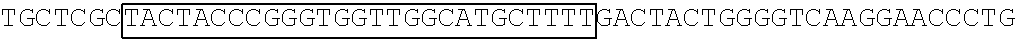

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

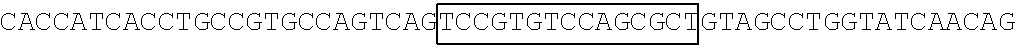

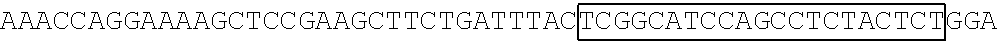

GTCCCTTCTCGCTTCTCTGGTAGCCGTTCCGGGACGGATTTCACTCTGACCATCAGCA

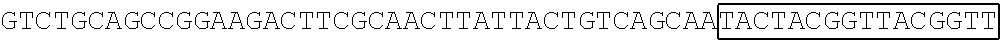

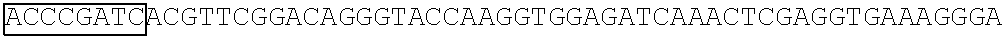

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGCTGTGCGCACGCCCACGCCGCAGCCCCGCCCAAGAAGATGGCAAAGTC
TACATCAACATGCCAGGCAGGGGCAGAGTGAAGTTCAGCAGGAGCGCAGACGCCCCC
GCGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACA
TGCAGGCCCTGCCCCCTCGC
CARs comprising Ab4scFvLH:
Ab4scFvLH-CD28H-CD28TM-CD28CS-CD3zICS
Amino acid Sequence:
(SEQ ID NO: 246)

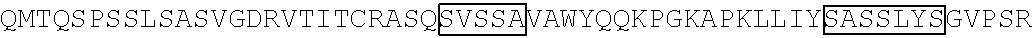

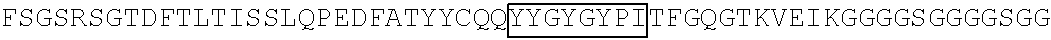

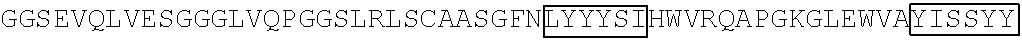

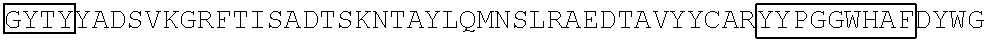

QGTLVTVSSLEVKGKHLCPSPLFPGPSKPFWVLVVVGGVLACYSLLVTVAFIIFWVRSK
RSRLLHSDYMNMTPRRPGPTRKHYQPYAPPRDFAAYRSKLRVKFSRSADAPAYQQGQ
NQLYNELNLGRREEYDVLDKRRGRDPEMGGKPQRRKNPQEGLYNELQKDKMAEAYS
EIGMKGERRRGKGHDGLYQGLSTATKDTYDALHMQALPPR
Nucleic acid Sequence:
(SEQ ID NO: 346)
GATATCCAGATGACCCAGTCCCCGAGCTCCCTGTCCGCCTCTGTGGGCGATAGGGTCA

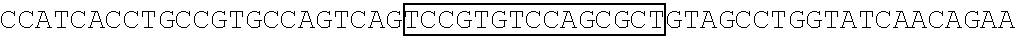

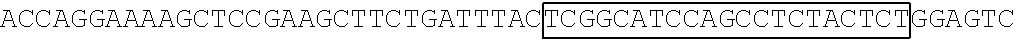

CCTTCTCGCTTCTCTGGTAGCCGTTCCGGGACGGATTTCACTCTGACCATCAGCAGTC

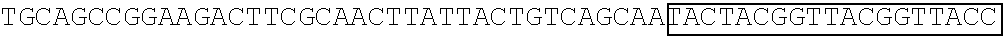

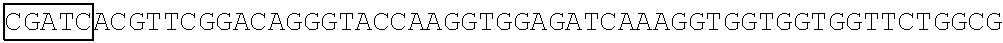

GCGGCGGCTCCGGTGGTGGTGGTTCCGAGGTTCAGCTGGTGGAGTCTGGCGGTGGCCT

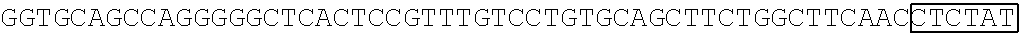

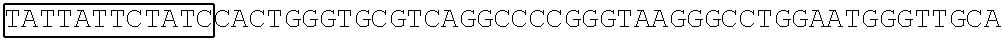

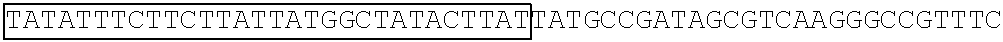

ACTATAAGCGCAGACACATCCAAAAACACAGCCTACCTACAAATGAACAGCTTAAGA

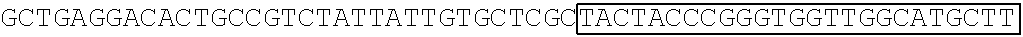

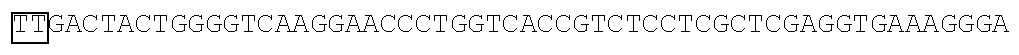

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACATGAC
TCCCCGCCGCCCCGGGCCCACCCGCAAGCATTACCAGCCCTATGCCCCACCACGCGACT
TCGCAGCCTATCGCTCCAAGCTTAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab4scFvLH-CD28H-CD28TM-41BBCS-CD3zICS
Amino acid Sequence:
(SEQ ID NO: 247)

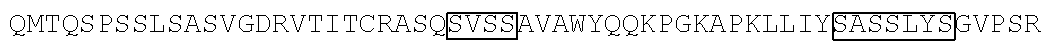

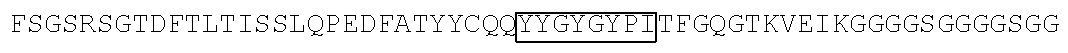

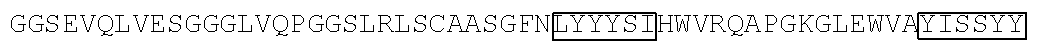

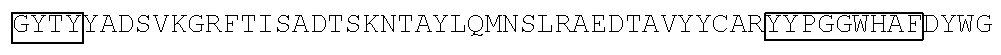

QGTLVTVSSLEVKGKHLCPSPLFPGPSKPFWVLVVVGGVLACYSLLVTVAFIIFWVKRG
RKKLLYIFKQPFMRPVQTTQEEDGCSCRFPEEEEGGCELRVKFSRSADAPAYQQGQNQ
LYNELNLGRREEYDVLDKRRGRDPEMGGKPQRRKNPQEGLYNELQKDKMAEAYSEIG
MKGERRRGKGHDGLYQGLSTATKDTYDALHMQALPPR
Nucleic acid Sequence:
(SEQ ID NO: 347)
GATATCCAGATGACCCAGTCCCCGAGCTCCCTGTCCGCCTCTGTGGGCGATAGGGTCA

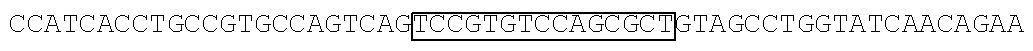

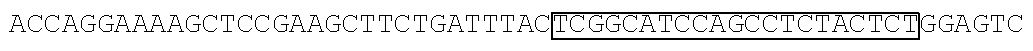

CCTTCTCGCTTCTCTGGTAGCCGTTCCGGGACGGATTTCACTCTGACCATCAGCAGTC

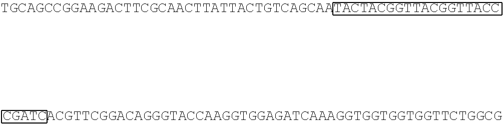

GCGGCGGCTCCGGTGGTGGTGGTTCCGAGGTTCAGCTGGTGGAGTCTGGCGGTGGCCT

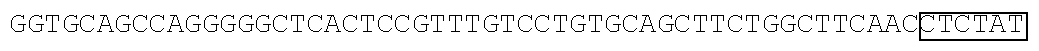

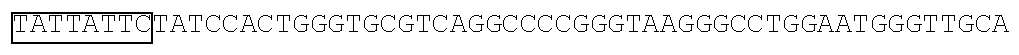

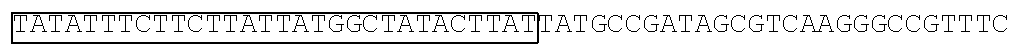

ACTATAAGCGCAGACACATCCAAAAACACAGCCTACCTACAAATGAACAGCTTAAGA

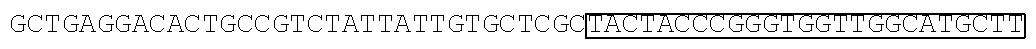

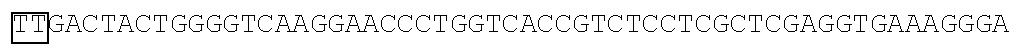

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAAACGGGGCAGAAAGAAACTCCTGTATATATTCAAACAACCATTTAT
GAGACCAGTACAAACTACTCAAGAGGAAGATGGCTGTAGCTGCCGATTTCCAGAAGA
AGAAGAAGGAGGATGTGAACTGAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab4scFvLH-CD28H-CD28TM-DAP10CS-CD3zICS
Amino acid Sequence:
(SEQ ID NO: 248)

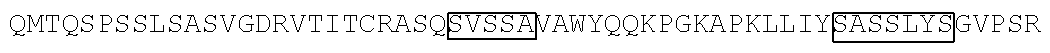

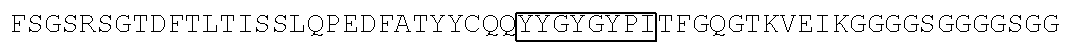

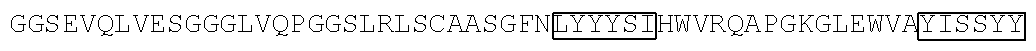

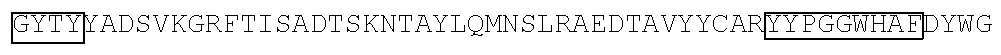

QGTLVTVSSLEVKGKHLCPSPLFPGPSKPFWVLVVVGGVLACYSLLVTVAFIIFWVLCA
RPRRSPAQEDGKVYINMPGRGRVKFSRSADAPAYQQGQNQLYNELNLGRREEYDVLD
KRRGRDPEMGGKPQRRKNPQEGLYNELQKDKMAEAYSEIGMKGERRRGKGHDGLYQ
GLSTATKDTYDALHMQALPPR
Nucleic acid Sequence:
(SEQ ID NO: 348)
GATATCCAGATGACCCAGTCCCCGAGCTCCCTGTCCGCCTCTGTGGGCGATAGGGTCA

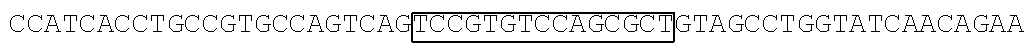

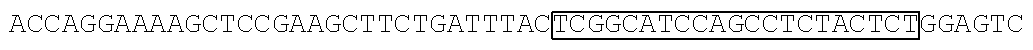

CCTTCTCGCTTCTCTGGTAGCCGTTCCGGGACGGATTTCACTCTGACCATCAGCAGTC

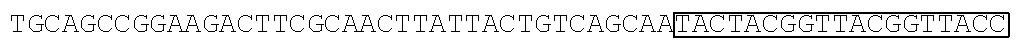

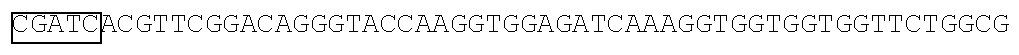

GCGGCGGCTCCGGTGGTGGTGGTTCCGAGGTTCAGCTGGTGGAGTCTGGCGGTGGCCT

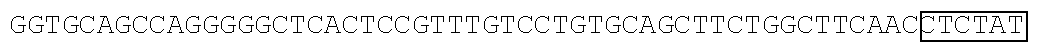

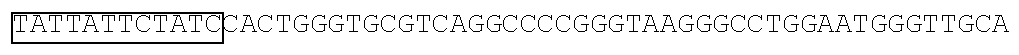

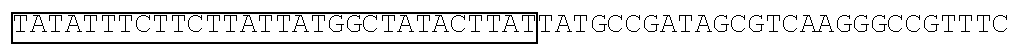

ACTATAAGCGCAGACACATCCAAAAACACAGCCTACCTACAAATGAACAGCTTAAGA

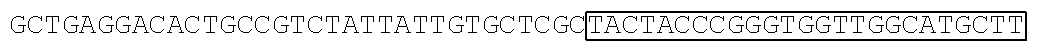

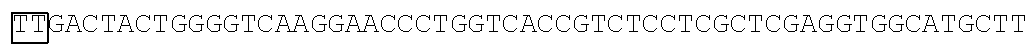

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGCTGTGCGCACGCCCACGCCGCAGCCCCGCCCAAGAAGATGGCAAAGTC
TACATCAACATGCCAGGCAGGGGCAGAGTGAAGTTCAGCAGGAGCGCAGACGCCCCC
GCGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACA
TGCAGGCCCTGCCCCCTCGC
CARs comprising Ab5scFvHL:
Ab5scFvHL-CD28H-CD28TM-CD28CS-CD3zICS
Amino acid Sequence:
(SEQ ID NO: 251)

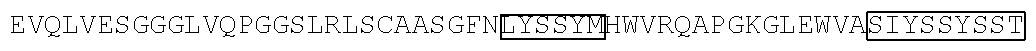

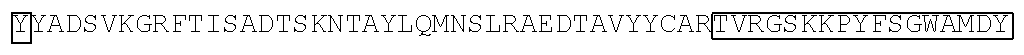

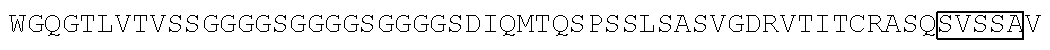

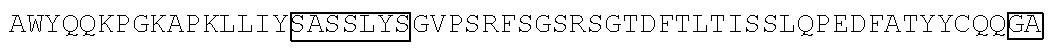

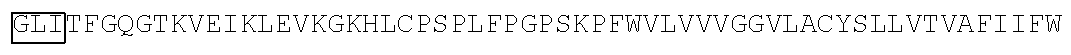

VRSKRSRLLHSDYMNMTPRRPGPTRKHYQPYAPPRDFAAYRSKLRVKFSRSADAPAY
QQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
Nucleic acid Sequence:
(SEQ ID NO: 351)
GAGGTTCAGCTGGTGGAGTCTGGCGGTGGCCTGGTGCAGCCAGGGGGCTCACTCCGTT

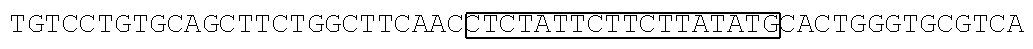

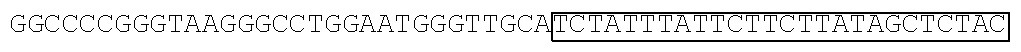

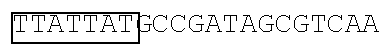

CACAGCCTACCTACAAATGAACAGCTTAAGAGCTGAGGACACTGCCGTCTATTATTG

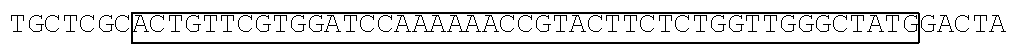

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

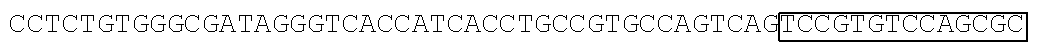

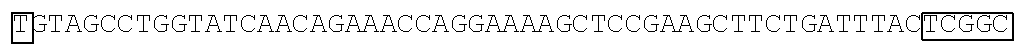

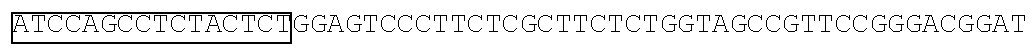

TTCACTCTGACCATCAGCAGTCTGCAGCCGGAAGACTTCGCAACTTATTACTGTCAGC

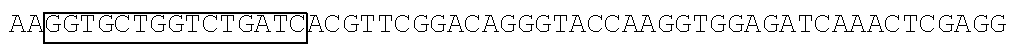

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCCA
CCACGCGACTTCGCAGCCTATCGCTCCAAGCTTAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab5scFvHL-CD28H-CD28TM-41BBCS-CD3zICS
Amino acid Sequence:
(SEQ ID NO: 252)

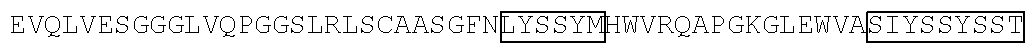

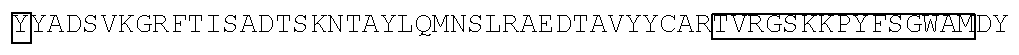

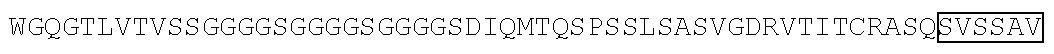

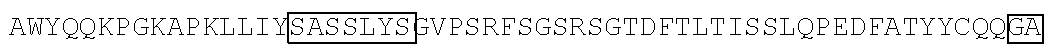

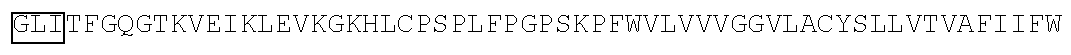

VKRGRKKLLYIFKQPFMRPVQTTQEEDGCSCRFPEEEEGGCELRVKFSRSADAPAYQQ
GQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPR
Nucleic acid Sequence:
(SEQ ID NO: 352)
GAGGTTCAGCTGGTGGAGTCTGGCGGTGGCCTGGTGCAGCCAGGGGGCTCACTCCGTT

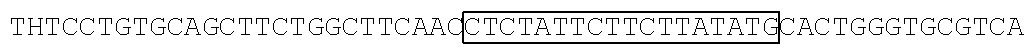

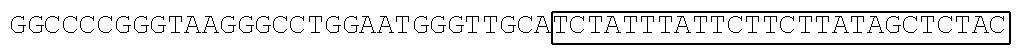

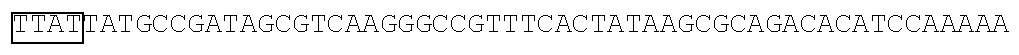

CACAGCCTACCTACAAATGAACAGCTTAAGAGCTGAGGACACTGCCGTCTATTATTG

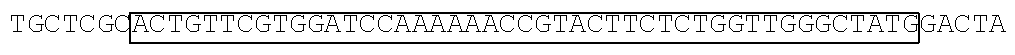

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

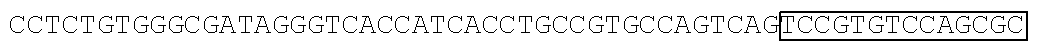

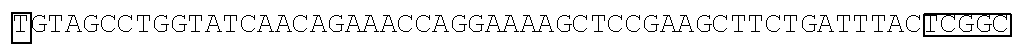

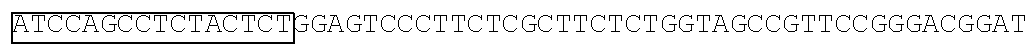

TTCACTCTGACCATCAGCAGTCTGCAGCCGGAAGACTTCGCAACTTATTACTGTCAGC

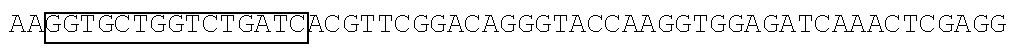

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAAACGGGGCAGAAAGAAACTCCTGTATATATTCAAACA
ACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab5scFvHL-CD28H-CD28TM-DAP10CS-CD3zICS
Amino acid Sequence:
(SEQ ID NO: 253)

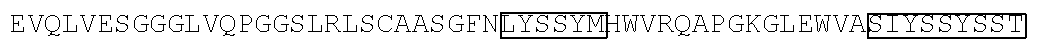

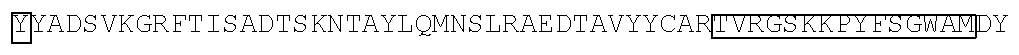

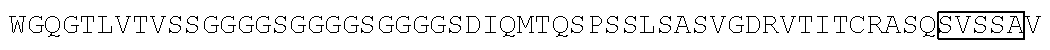

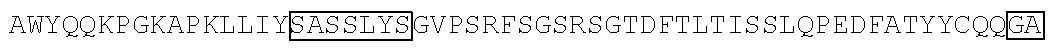

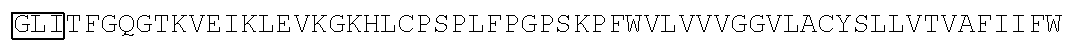

VLCARPRRSPAQEDGKVYINMPGRGRVKFSRSADAPAYQQGQNQLYNELNLGRREEY
DVLDKRRGRDPEMGGKPQRRKNPQEGLYNELQKDKMAEAYSEIGMKGERRRGKGHD
GLYQGLSTATKDTYDALHMQALPPR
Nucleic acid Sequence:
(SEQ ID NO: 353)
GAGGTTCAGCTGGTGGAGTCTGGCGGTGGCCTGGTGCAGCCAGGGGGCTCACTCCGTT

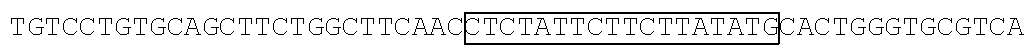

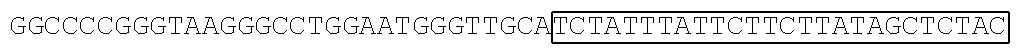

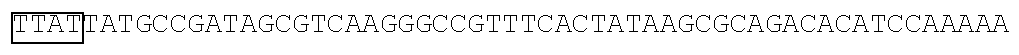

CACAGCCTACCTACAAATGAACAGCTTAAGAGCTGAGGACACTGCCGTCTATTATTG

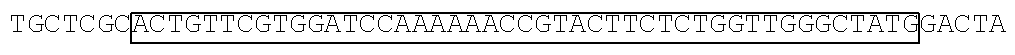

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

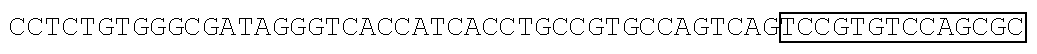

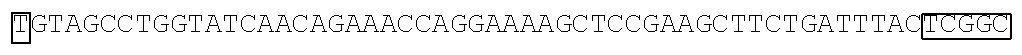

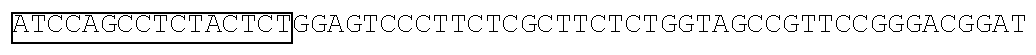

TTCACTCTGACCATCAGCAGTCTGCAGCCGGAAGACTTCGCAACTTATTACTGTCAGC

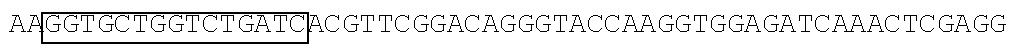

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGCTGTGCGCACGCCCACGCCGCAGCCCCGCCCAAGAAGAT
GGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGG
GAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGC
CCTTCACATGCAGGCCCTGCCCCCTCGC
CARs comprising Ab5scFvLH:
Ab5scFvLH-CD28H-CD28TM-CD28CS-CD3zICS
Amino acid Sequence:
(SEQ ID NO: 256)

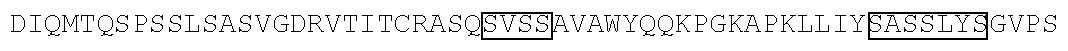

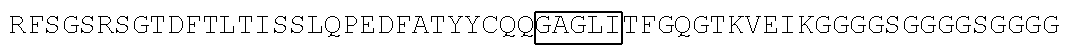

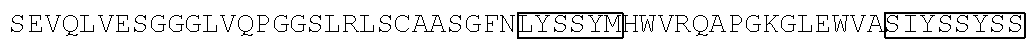

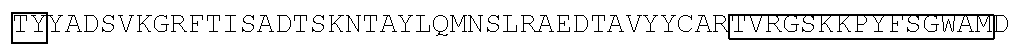

YWGQGTLVTVSSLEVKGKHLCPSPLFPGPSKPFWVLVVVGGVLACYSLLVTVAFIIFWV
RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 356)
GATATCCAGATGACCCAGTCCCCGAGCTCCCTGTCCGCCTCTGTGGGCGATAGGGTCA

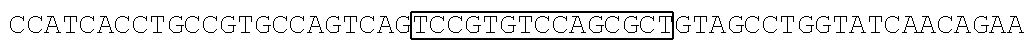

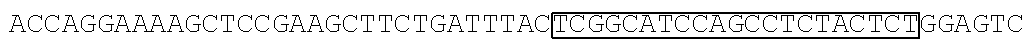

CCTTCTCGCTTCTCTGGTAGCCGTTCCGGGACGGATTTCACTCTGACCATCAGCAGTC

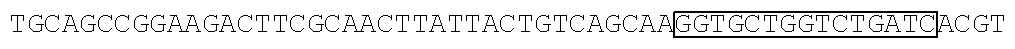

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

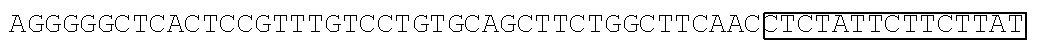

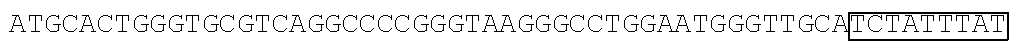

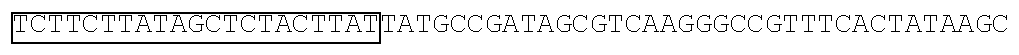

GCAGACACATCCAAAAACACAGCCTACCTACAAATGAACAGCTTAAGAGCTGAGGAC

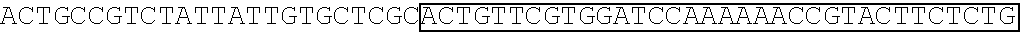

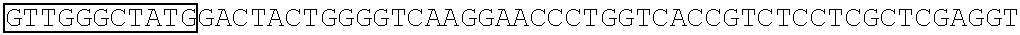

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACC
ACGCGACTTCGCAGCCTATCGCTCCAAGCTTAGAGTGAAGTTCAGCAGGAGCGCAGA
CGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCC
TTCACATGCAGGCCCTGCCCCCTCGC
Ab5scFvLH-CD28H-CD28TM-41BBCS-CD3zICS
Amino acid Sequence:
(SEQ ID NO: 257)

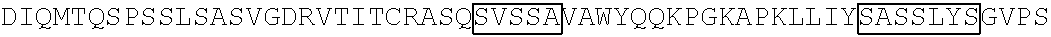

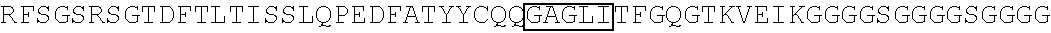

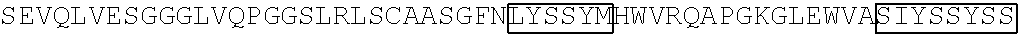

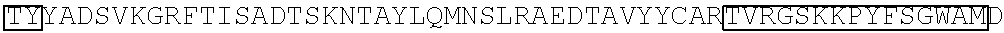

YWGQGTLVTVSSLEVKGKHLCPSPLFPGPSKPFWVLVVVGGVLACYSLLVTVAFIIFWV
KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 357)
GATATCCAGATGACCCAGTCCCCGAGCTCCCTGTCCGCCTCTGTGGGCGATAGGGTCA

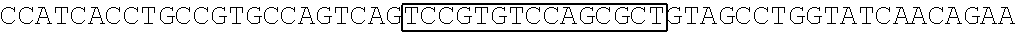

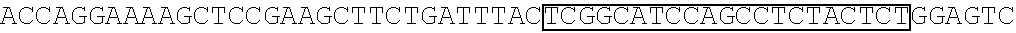

CCTTCTCGCTTCTCTGGTAGCCGTTCCGGGACGGATTTCACTCTGACCATCAGCAGTC

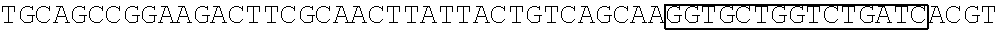

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

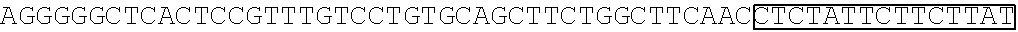

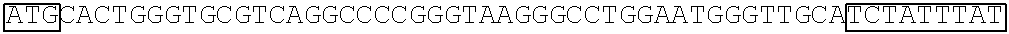

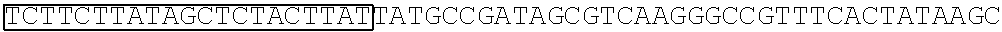

GCAGACACATCCAAAAACACAGCCTACCTACAAATGAACAGCTTAAGAGCTGAGGAC

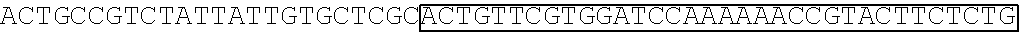

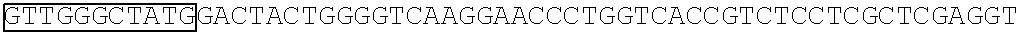

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAA
CCATTTATGAGACCAGTACAAACTACTCAAGAGGAAGATGGCTGTAGCTGCCGATTT
CCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAA
AGCCGCAGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCT
TCACATGCAGGCCCTGCCCCCTCGC
Ab5scFvLH-CD28H-CD28TM-DAP10CS-CD3zICS
Amino acid Sequence:
(SEQ ID NO: 258)

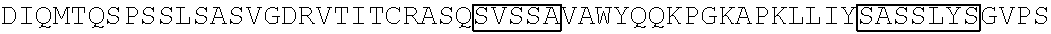

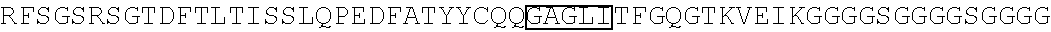

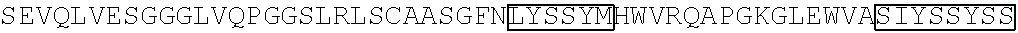

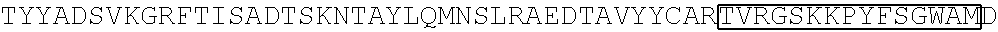

YWGQGTLVTVSSLEVKGKHLCPSPLFPGPSKPFWVLVVVGGVLACYSLLVTVAFIIFWV
LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 358)
GATATCCAGATGACCCAGTCCCCGAGCTCCCTGTCCGCCTCTGTGGGCGATAGGGTCA

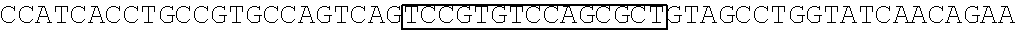

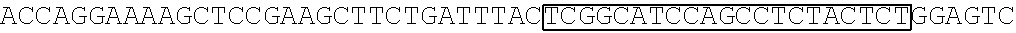

CCTTCTCGCTTCTCTGGTAGCCGTTCCGGGACGGATTTCACTCTGACCATCAGCAGTC

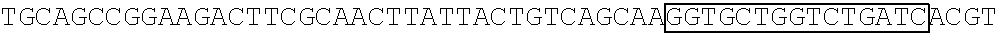

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

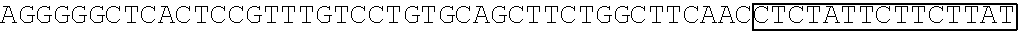

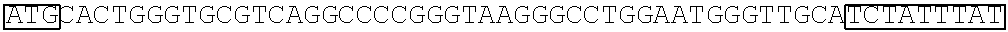

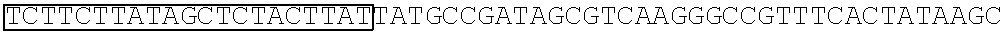

GCAGACACATCCAAAAACACAGCCTACCTACAAATGAACAGCTTAAGAGCTGAGGAC

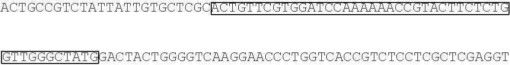

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGCTGTGCGCACGCCCACGCCGCAGCCCCGCCCAAGAAGATG
GCAAAGTCTACATCAACATGCCAGGCAGGGGCAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
CARs comprising Ab6scFvHL:
Ab6scFvHL-CD28H-CD28TM-CD28CS-CD3zICS
Amino acid Sequence:
(SEQ ID NO: 261)

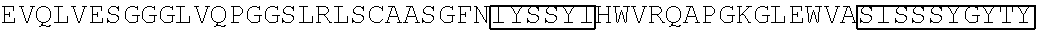

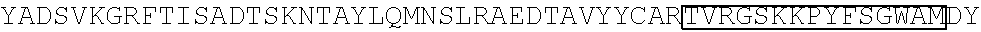

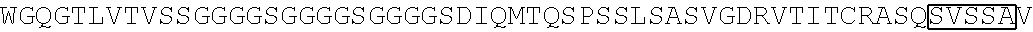

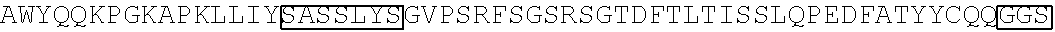

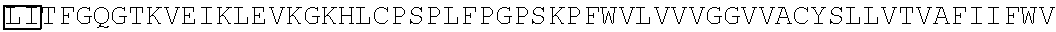

RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 361)
GAGGTTCAGCTGGTGGAGTCTGGCGGTGGCCTGGTGCAGCCAGGGGGCTCACTCCGTT

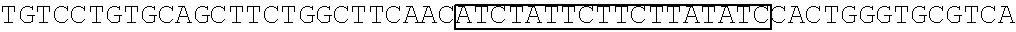

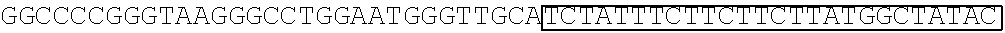

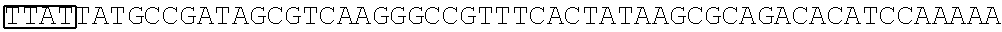

CACAGCCTACCTACAAATGAACAGCTTAAGAGCTGAGGACACTGCCGTCTATTATTG

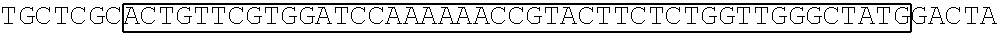

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

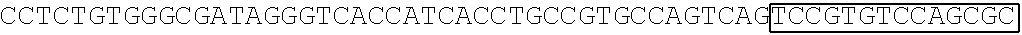

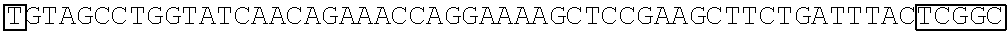

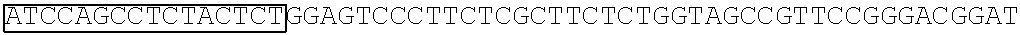

TTCACTCTGACCATCAGCAGTCTGCAGCCGGAAGACTTCGCAACTTATTACTGTCAGC

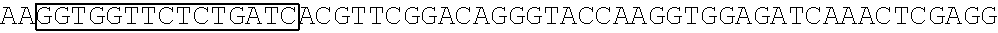

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCCA
CCACGCGACTTCGCAGCCTATCGCTCCAAGCTTAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab6scFvHL-CD28H-CD28TM-41BBCS-CD3zICS
Amino acid Sequence:
(SEQ ID NO: 262)

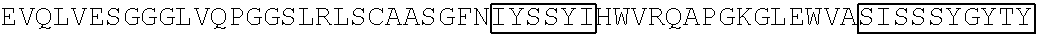

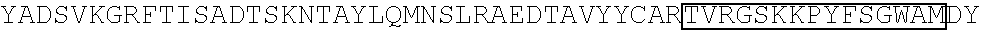

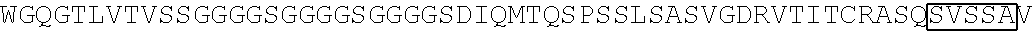

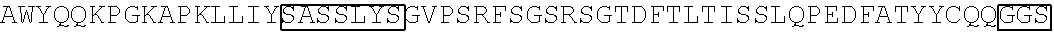

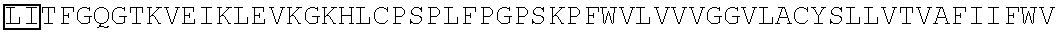

KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 362)
GAGGTTCAGCTGGTGGAGTCTGGCGGTGGCCTGGTGCAGCCAGGGGGCTCACTCCGTT

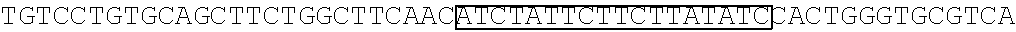

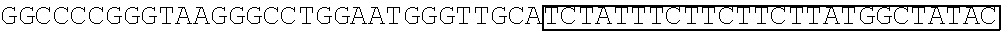

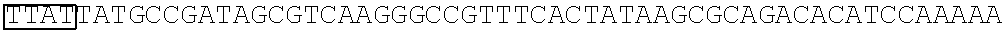

CACAGCCTACCTACAAATGAACAGCTTAAGAGCTGAGGACACTGCCGTCTATTATTG

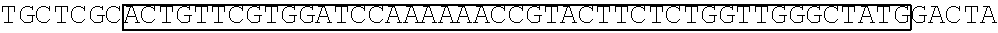

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

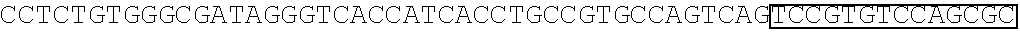

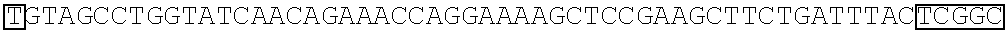

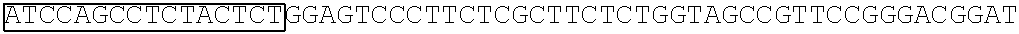

TTCACTCTGACCATCAGCAGTCTGCAGCCGGAAGACTTCGCAACTTATTACTGTCAGC

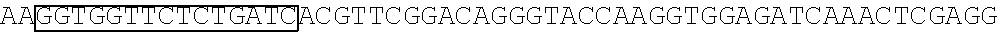

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAAACGGGGCAGAAAGAAACTCCTGTATATATTCAAACA
ACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab6scFvHL-CD28H-CD28TM-DAP10CS-CD3zICS
Amino acid Sequence:
(SEQ ID NO: 263)

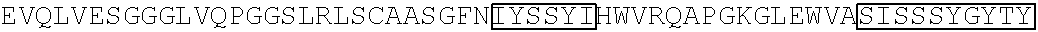

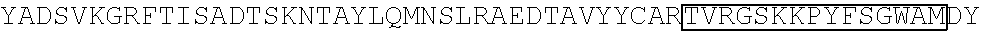

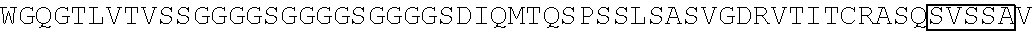

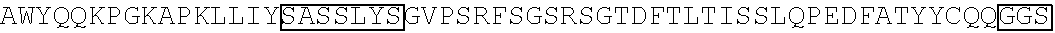

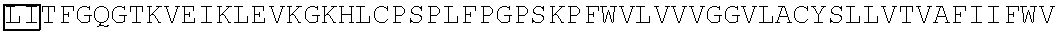

LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 363)
GAGGTTCAGCTGGTGGAGTCTGGCGGTGGCCTGGTGCAGCCAGGGGGCTCACTCCGTT

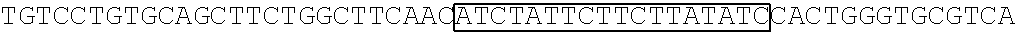

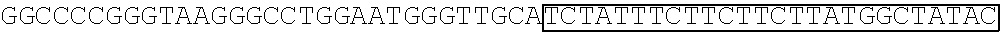

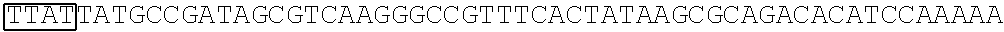

CACAGCCTACCTACAAATGAACAGCTTAAGAGCTGAGGACACTGCCGTCTATTATTG

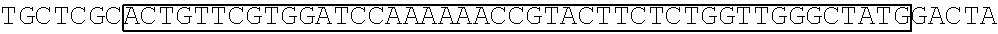

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

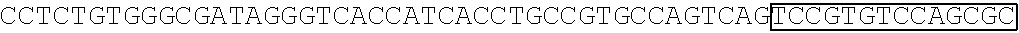

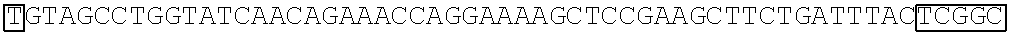

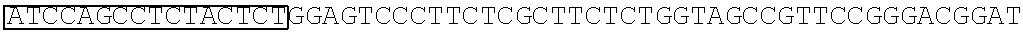

TTCACTCTGACCATCAGCAGTCTGCAGCCGGAAGACTTCGCAACTTATTACTGTCAGC

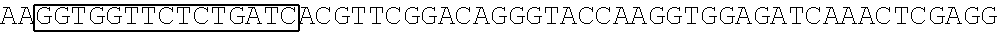

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGCTGTGCGCACGCCCACGCCGCAGCCCCGCCCAAGAAGAT
GGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGG
GAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGC
CCTTCACATGCAGGCCCTGCCCCCTCGC
CARs comprising Ab6scFvLH:
Ab6scFvLH-CD28H-CD28TM-CD28CS-CD3zICS
Amino acid Sequence:
(SEQ ID NO: 266)

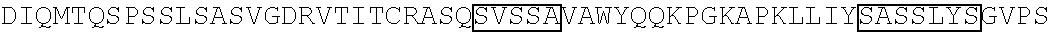

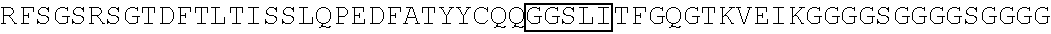

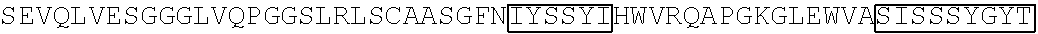

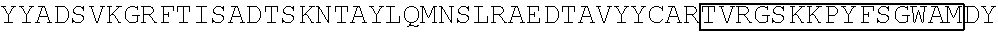

WGQGTLVTVSSLEVKGKHLCPSPLFPGPSKPFWVLVVVGGVLACYSLLVTVAFIIFWV
RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 366)
GATATCCAGATGACCCAGTCCCCGAGCTCCCTGTCCGCCTCTGTGGGCGATAGGGTCA

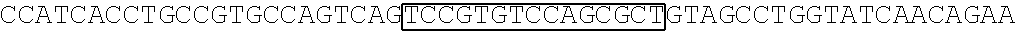

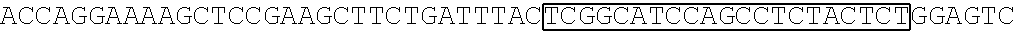

CCTTCTCGCTTCTCTGGTAGCCGTTCCGGGACGGATTTCACTCTGACCATCAGCAGTC

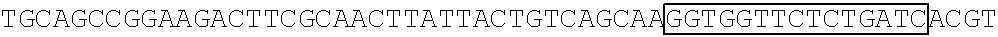

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

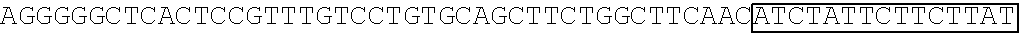

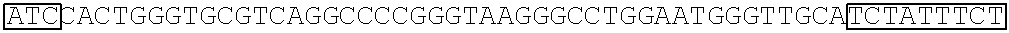

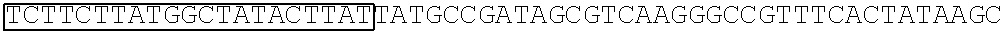

GCAGACACATCCAAAAACACAGCCTACCTACAAATGAACAGCTTAAGAGCTGAGGAC

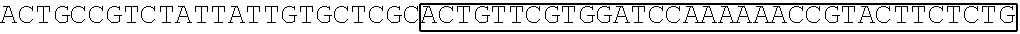

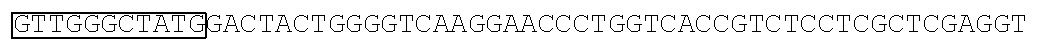

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACC
ACGCGACTTCGCAGCCTATCGCTCCAAGCTTAGAGTGAAGTTCAGCAGGAGCGCAGA
CGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCC
TTCACATGCAGGCCCTGCCCCCTCGC
Ab6scFvLH-CD28H-CD28TM-41BBCS-CD3zICS
Amino acid Sequence:
(SEQ ID NO: 267)

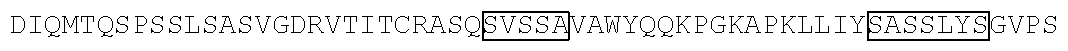

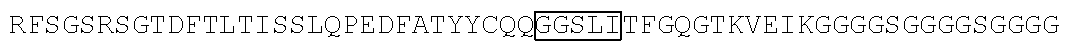

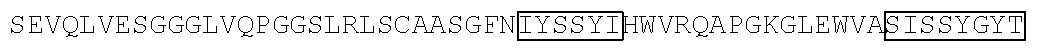

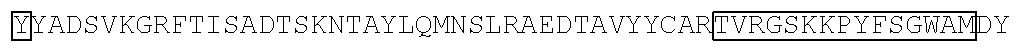

WGQGTLVTVSSLEVKGKHLCPSPLFPGPSKPFWVLVVVGGVLACYSLLVTVAFIIFWV
KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 367)
GATATCCAGATGACCCAGTCCCCGAGCTCCCTGTCCGCCTCTGTGGGCGATAGGGTCA

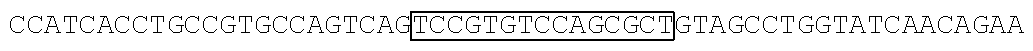

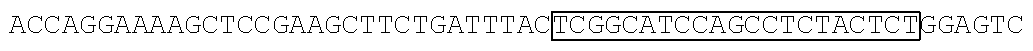

CCTTCTCGCTTCTCTGGTAGCCGTTCCGGGACGGATTTCACTCTGACCATCAGCAGTC

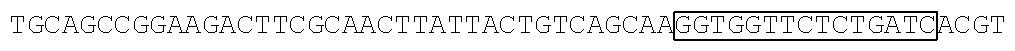

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

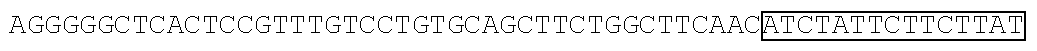

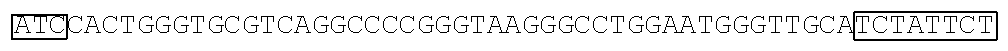

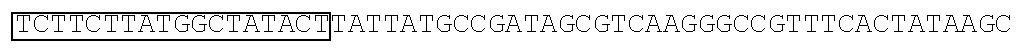

GCAGACACATCCAAAAACACAGCCTACCTACAAATGAACAGCTTAAGAGCTGAGGAC

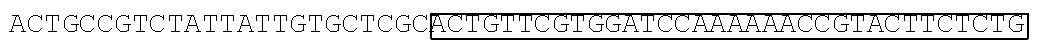

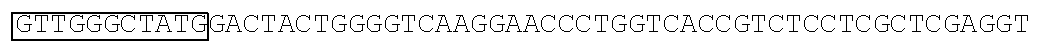

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAA
CCATTTATGAGACCAGTACAAACTACTCAAGAGGAAGATGGCTGTAGCTGCCGATTT
CCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAA
AGCCGCAGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCT
TCACATGCAGGCCCTGCCCCCTCGC
Ab6scFvLH-CD28H-CD28TM-DAP10CS-CD3zICS
Amino acid Sequence:
(SEQ ID NO: 268)

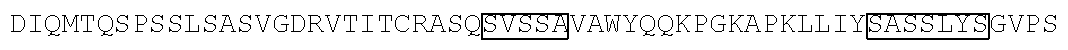

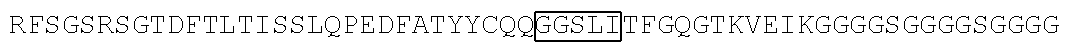

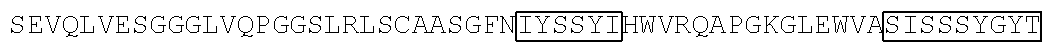

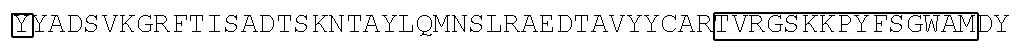

WGQGTLVTVSSLEVKGKHLCPSPLFPGPSKPFWVLVVVGGVLACYSLLVTVAFIIFWVL
CARPRRSPAQEDGKVYINMPGRGRVKFSRSADAPAYQQGQNQLYNELNLGRREEYDV
LDKRRGRDPEMGGKPQRRKNPQEGLYNELQKDKMAEAYSEIGMKGERRRGKGHDGL
YQGLSTATKDTYDALHMQALPPR
Nucleic acid Sequence:
(SEQ ID NO: 368)
GATATCCAGATGACCCAGTCCCCGAGCTCCCTGTCCGCCTCTGTGGGCGATAGGGTCA

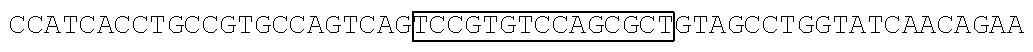

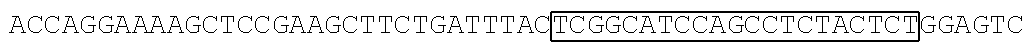

CCTTCTCGCTTCTCTGGTAGCCGTTCCGGGACGGATTTCACTCTGACCATCAGCAGTC

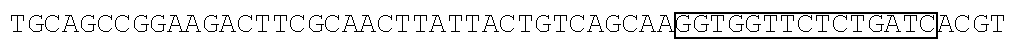

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

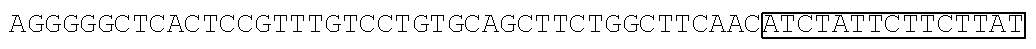

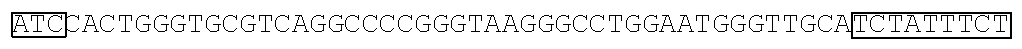

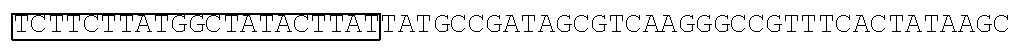

GCAGACACATCCAAAAACACAGCCTACCTACAAATGAACAGCTTAAGAGCTGAGGAC

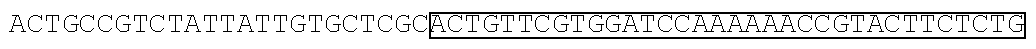

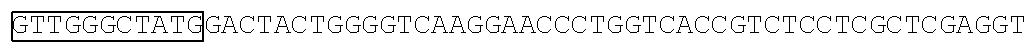

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGCTGTGCGCACGCCCACGCCGCAGCCCCGCCCAAGAAGATG
GCAAAGTCTACATCAACATGCCAGGCAGGGGCAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
CARs comprising Ab7scFvHL:
Ab7scFvHL-CD28H-CD28TM-CD28CS-CD3zICS
Amino acid Sequence:
(SEQ ID NO: 271)

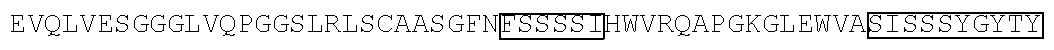

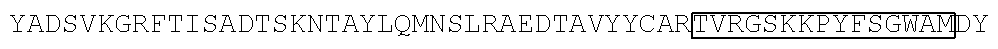

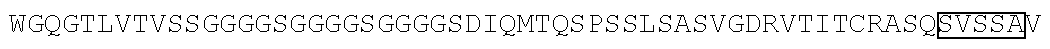

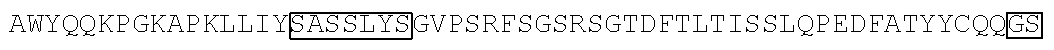

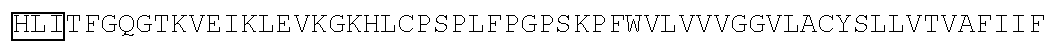

WVRSKRSRLLHSDYMNMTPRRPGPTRKHYQPYAPPRDFAAYRSKLRVKFSRSADAP
AYQQGQNQLYNELNLGRREEYDVLDKRRGRDPEMGGKPQRRKNPQEGLYNELQKDK
MAEAYSEIGMKGERRRGKGHDGLYQGLSTATKDTYDALHMQALPPR
Nucleic acid Sequence:
(SEQ ID NO: 371)
GAGGTTCAGCTGGTGGAGTCTGGCGGTGGCCTGGTGCAGCCAGGGGGCTCACTCCGTT

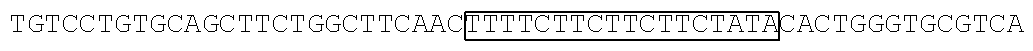

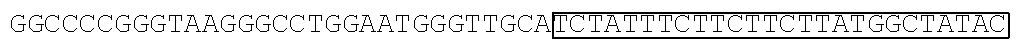

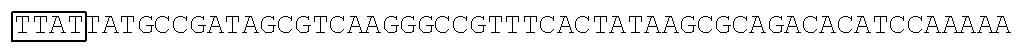

CACAGCCTACCTACAAATGAACAGCTTAAGAGCTGAGGACACTGCCGTCTATTATTG

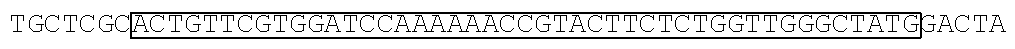

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

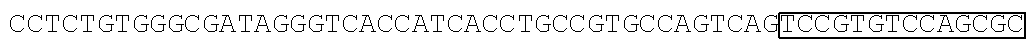

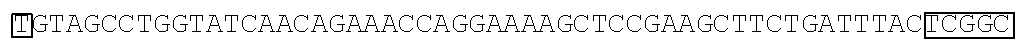

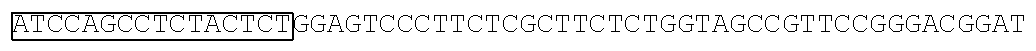

TTCACTCTGACCATCAGCAGTCTGCAGCCGGAAGACTTCGCAACTTATTACTGTCAGC

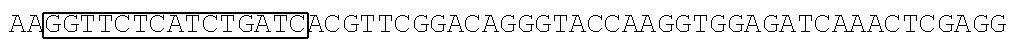

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCCA
CCACGCGACTTCGCAGCCTATCGCTCCAAGCTTAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab7scFvHL-CD28H-CD28TM-41BBCS-CD3zICS
Amino acid Sequence:
(SEQ ID NO: 272)

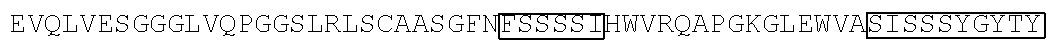

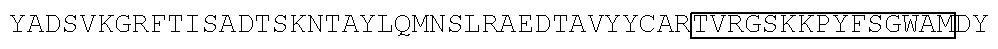

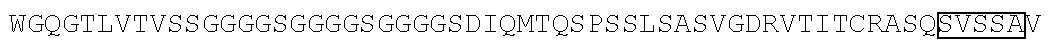

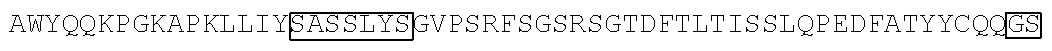

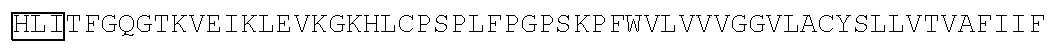

WVKRGRKKLLYIFKQPFMRPVQTTQEEDGCSCRFPEEEEGGCELRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 372)
GAGGTTCAGCTGGTGGAGTCTGGCGGTGGCCTGGTGCAGCCAGGGGGCTCACTCCGTT

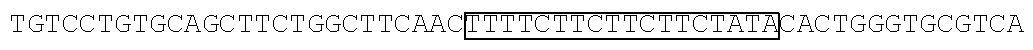

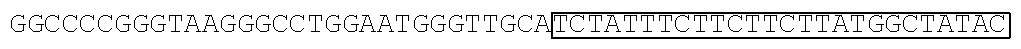

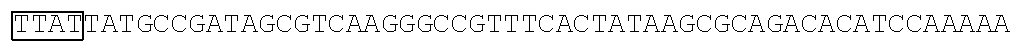

CACAGCCTACCTACAAATGAACAGCTTAAGAGCTGAGGACACTGCCGTCTATTATTG

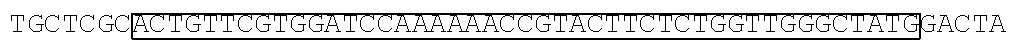

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

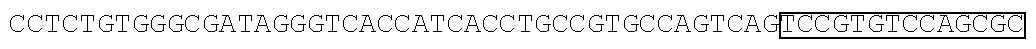

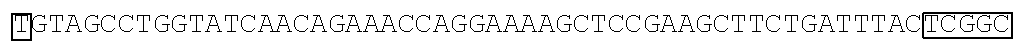

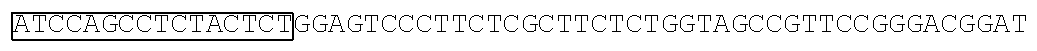

TTCACTCTGACCATCAGCAGTCTGCAGCCGGAAGACTTCGCAACTTATTACTGTCAGC

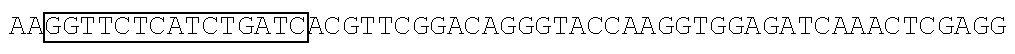

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAAACGGGGCAGAAAGAAACTCCTGTATATATTCAAACA
ACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab7scFvHL-CD28H-CD28TM-DAP10CS-CD3zICS
Amino acid Sequence:
(SEQ ID NO: 273)

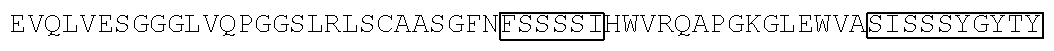

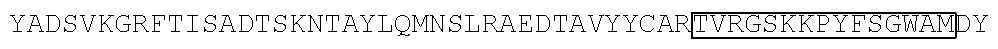

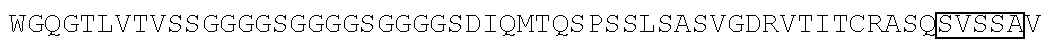

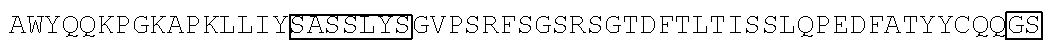

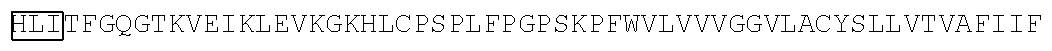

WVLCARPRRSPAQEDGKVYINMPGRGRVKFSRSADAPAYQQGQNQLYNELNLGRREE
YDVLDKRRGRDPEMGGKPQRRKNPQEGLYNELQKDKMAEAYSEIGMKGERRRGKGH
DGLYQGLSTATKDTYDALHMQALPPR
Nucleic acid Sequence:
(SEQ ID NO: 373)
GAGGTTCAGCTGGTGGAGTCTGGCGGTGGCCTGGTGCAGCCAGGGGGCTCACTCCGTT

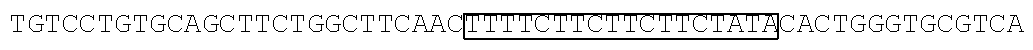

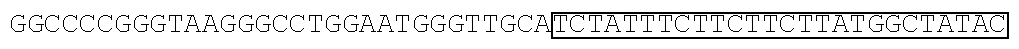

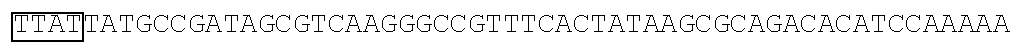

CACAGCCTACCTACAAATGAACAGCTTAAGAGCTGAGGACACTGCCGTCTATTATTG

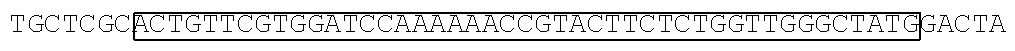

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

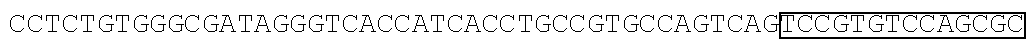

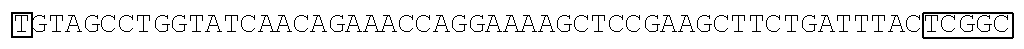

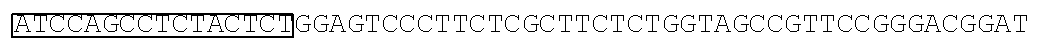

TTCACTCTGACCATCAGCAGTCTGCAGCCGGAAGACTTCGCAACTTATTACTGTCAGC

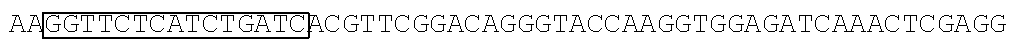

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGCTGTGCGCACGCCCACGCCGCAGCCCCGCCCAAGAAGAT
GGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGG
GAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGC
CCTTCACATGCAGGCCCTGCCCCCTCGC
CARs comprising Ab7scFvLH:
Ab7scFvLH-CD28H-CD28TM-CD28CS-CD3zICS
Amino acid Sequence:
(SEQ ID NO: 276)

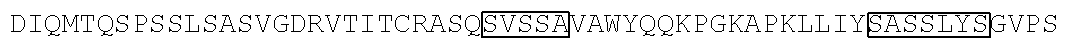

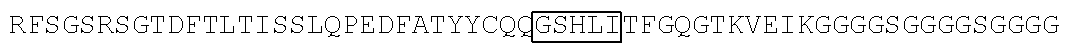

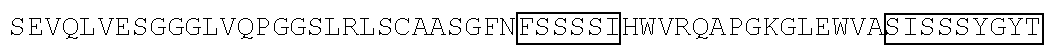

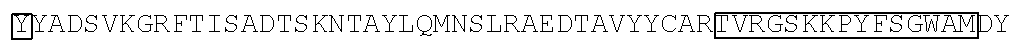

WGQGTLVTVSSLEVKGKHLCPSPLFPGPSKPFWVLVVVGGVLACYSLLVTVAFIIFWV
RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 376)
GATATCCAGATGACCCAGTCCCCGAGCTCCCTGTCCGCCTCTGTGGGCGATAGGGTCA

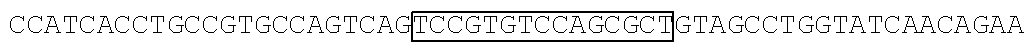

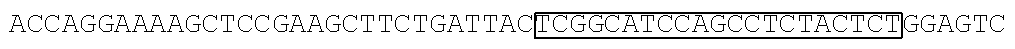

CCTTCTCGCTTCTCTGGTAGCCGTTCCGGGACGGATTTCACTCTGACCATCAGCAGTC

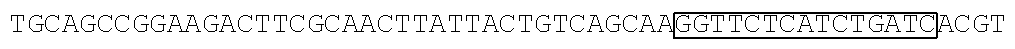

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

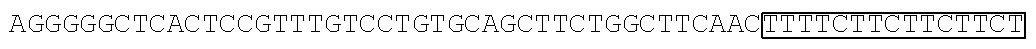

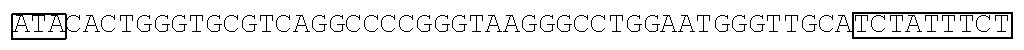

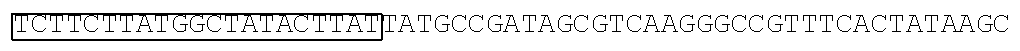

GCAGACACATCCAAAAACACAGCCTACCTACAAATGAACAGCTTAAGAGCTGAGGAC

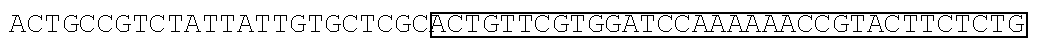

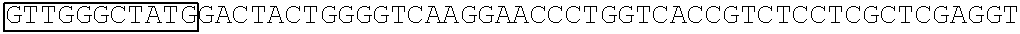

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACC
ACGCGACTTCGCAGCCTATCGCTCCAAGCTTAGAGTGAAGTTCAGCAGGAGCGCAGA
CGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCC
TTCACATGCAGGCCCTGCCCCCTCGC
Ab7scFvLH-CD28H-CD28TM-41BBCS-CD3zICS
Amino acid Sequence:
(SEQ ID NO: 277)

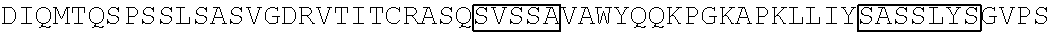

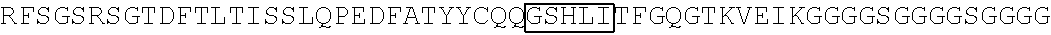

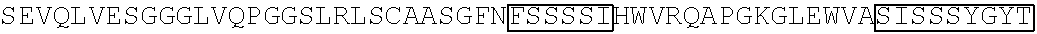

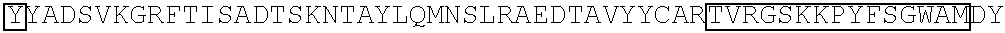

WGQGTLVTVSSLEVKGKHLCPSPLFPGPSKPFWVLVVVGGVLACYSLLVTVAFIIFWV
KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 377)
GATATCCAGATGACCCAGTCCCCGAGCTCCCTGTCCGCCTCTGTGGGCGATAGGGTCA

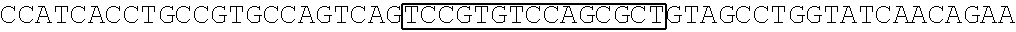

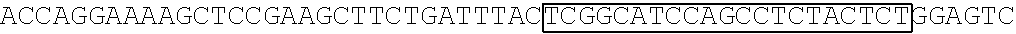

CCTTCTCGCTTCTCTGGTAGCCGTTCCGGGACGGATTTCACTCTGACCATCAGCAGTC

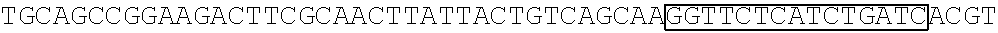

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

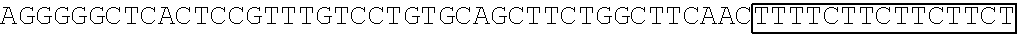

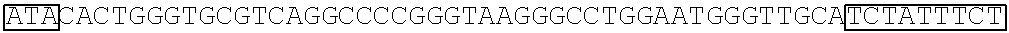

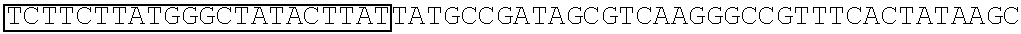

GCAGACACATCCAAAAACACAGCCTACCTACAAATGAACAGCTTAAGAGCTGAGGAC

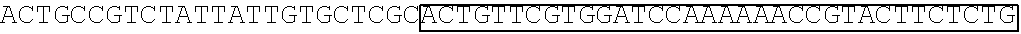

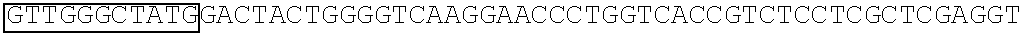

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAA
CCATTTATGAGACCAGTACAAACTACTCAAGAGGAAGATGGCTGTAGCTGCCGATTT
CCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAA
AGCCGCAGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCT
TCACATGCAGGCCCTGCCCCCTCGC
Ab7scFvLH-CD28H-CD28TM-DAP10CS-CD3zICS
Amino acid Sequence:
(SEQ ID NO: 278)

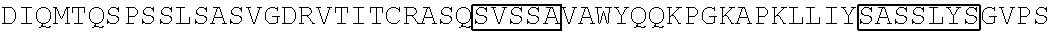

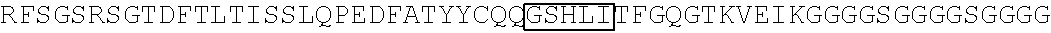

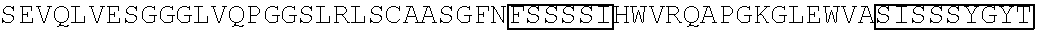

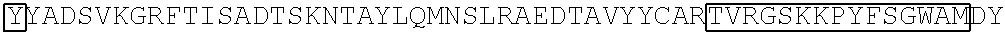

WGQGTLVTVSSLEVKGKHLCPSPLFPGPSKPFWVLVVVGGVLACYSLLVTVAFIIFWVL
CARPRRSPAQEDGKVYINMPGRGRVKFSRSADAPAYQQGQNQLYNELNLGRREEYDV
LDKRRGRDPEMGGKPQRRKNPQEGLYNELQKDKMAEAYSEIGMKGERRRGKGHDGL
YQGLSTATKDTYDALHMQALPPR
Nucleic acid Sequence:
(SEQ ID NO: 378)
GATATCCAGATGACCCAGTCCCCGAGCTCCCTGTCCGCCTCTGTGGGCGATAGGGTCA

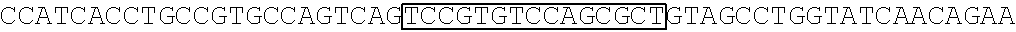

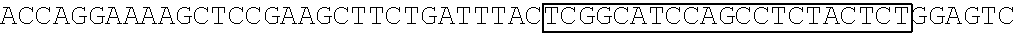

CCTTCTCGCTTCTCTGGTAGCCGTTCCGGGACGGATTTCACTCTGACCATCAGCAGTC

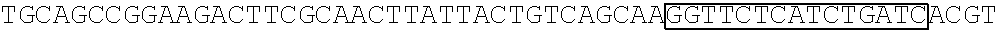

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

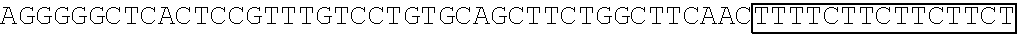

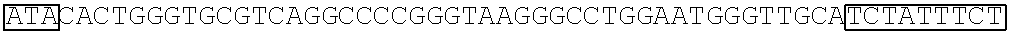

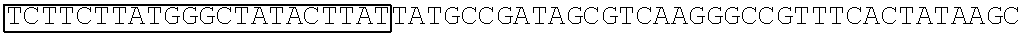

GCAGACACATCCAAAAACACAGCCTACCTACAAATGAACAGCTTAAGAGCTGAGGAC

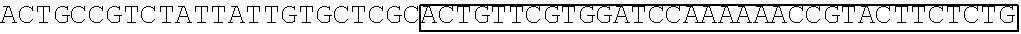

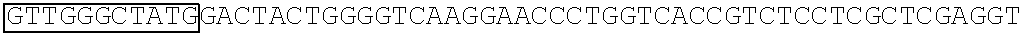

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGCTGTGCGCACGCCCACGCCGCAGCCCCGCCCAAGAAGATG
GCAAAGTCTACATCAACATGCCAGGCAGGGGCAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
CARs comprising Ab8scFvHL:
Ab8scFvHL-CD28H-CD28TM-CD28CS-CD3zICS
Amino acid Sequence:
(SEQ ID NO: 281)

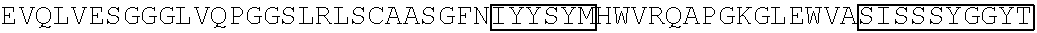

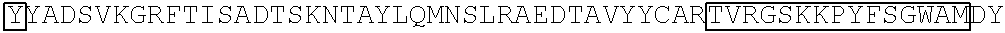

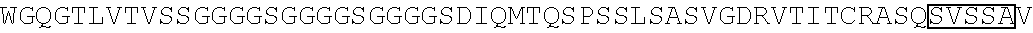

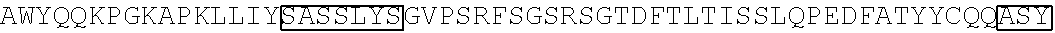

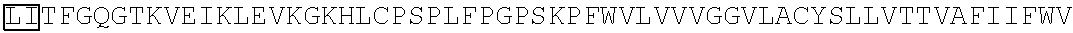

RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 381)
GAGGTTCAGCTGGTGGAGTCTGGCGGTGGCCTGGTGCAGCCAGGGGGCTCACTCCGTT

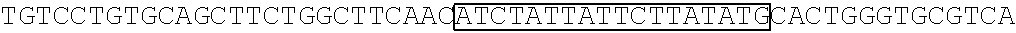

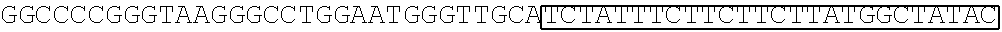

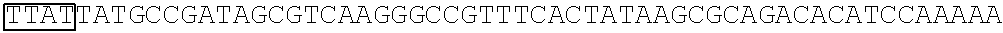

CACAGCCTACCTACAAATGAACAGCTTAAGAGCTGAGGACACTGCCGTCTATTATTG

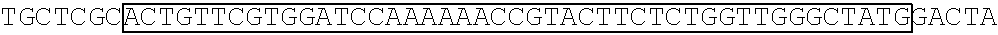

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

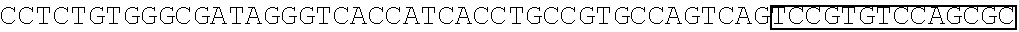

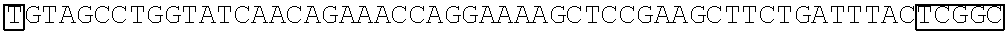

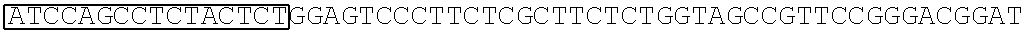

TTCACTCTGACCATCAGCAGTCTGCAGCCGGAAGACTTCGCAACTTATTACTGTCAGC

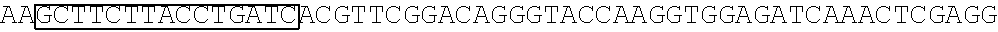

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCCA
CCACGCGACTTCGCAGCCTATCGCTCCAAGCTTAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab8scFvHL-CD28H-CD28TM-41BBCS-CD3zICS
Amino acid Sequence:
(SEQ ID NO: 282)

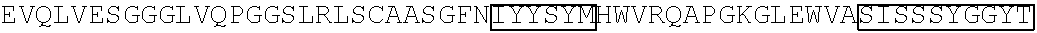

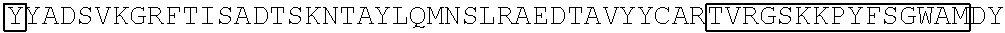

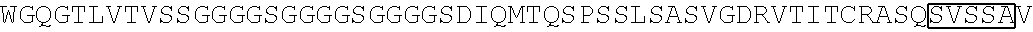

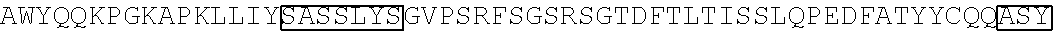

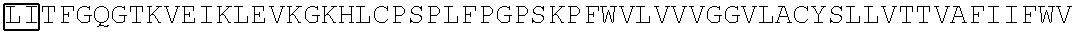

KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 382)
GAGGTTCAGCTGGTGGAGTCTGGCGGTGGCCTGGTGCAGCCAGGGGGCTCACTCCGTT

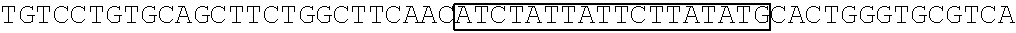

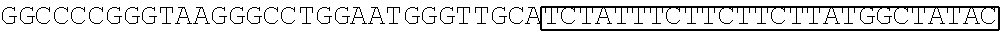

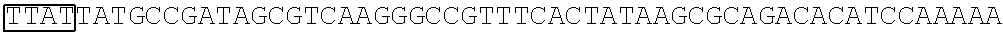

CACAGCCTACCTACAAATGAACAGCTTAAGAGCTGAGGACACTGCCGTCTATTATTG

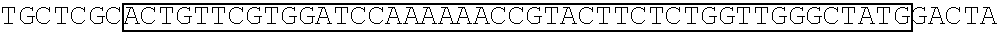

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

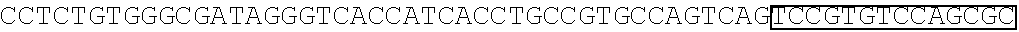

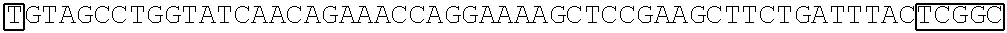

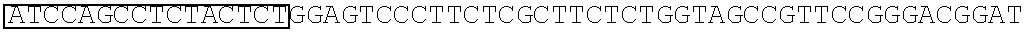

TTCACTCTGACCATCAGCAGTCTGCAGCCGGAAGACTTCGCAACTTATTACTGTCAGC

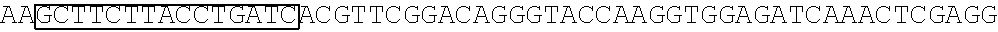

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAAACGGGGCAGAAAGAAACTCCTGTATATATTCAAACA
ACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab8scFvHL-CD28H-CD28TM-DAP10CS-CD3zICS
Amino acid Sequence:
(SEQ ID NO: 283)

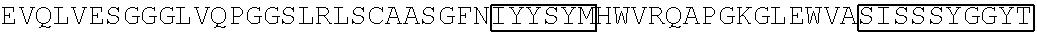

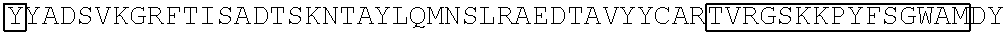

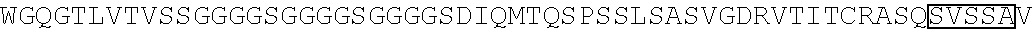

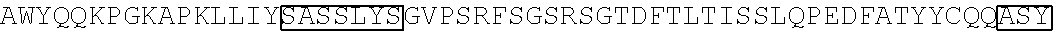

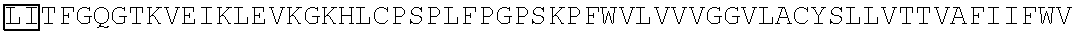

LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 383)
GAGGTTCAGCTGGTGGAGTCTGGCGGTGGCCTGGTGCAGCCAGGGGGCTCACTCCGTT

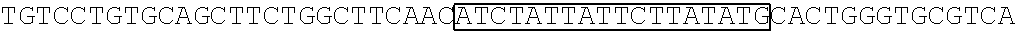

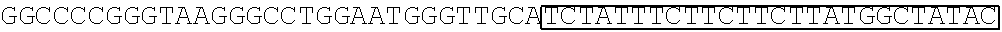

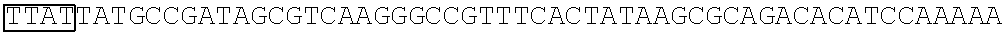

CACAGCCTACCTACAAATGAACAGCTTAAGAGCTGAGGACACTGCCGTCTATTATTG

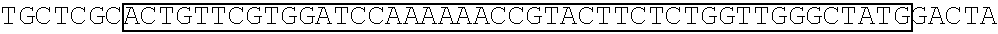

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

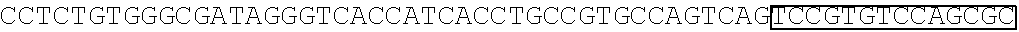

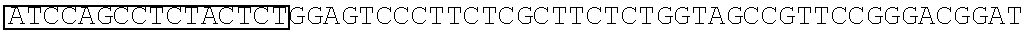

TTCACTCTGACCATCAGCAGTCTGCAGCCGGAAGACTTCGCAACTTATTACTGTCAGC

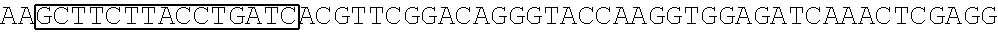

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGCTGTGCGCACGCCCACGCCGCAGCCCCGCCCAAGAAGAT
GGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGG
GAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGC
CCTTCACATGCAGGCCCTGCCCCCTCGC
CARs comprising Ab8scFvLH:
Ab8scFvLH-CD28H-CD28TM-CD28CS-CD3zICS
(SEQ ID NO: 286) Amino acid Sequence:

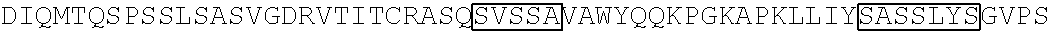

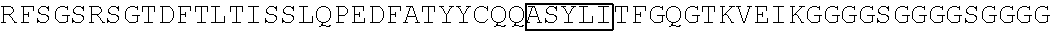

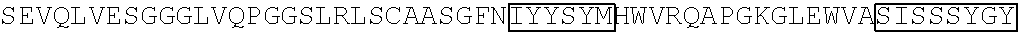

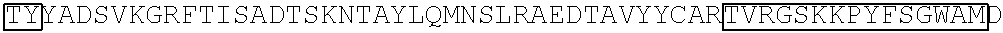

YWGQGTLVTVSSLEVKGKHLCPSPLFPGPSKPFWVLVVVGGVLACYSLLVTVAFIIFWV
RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 386)
GATATCCAGATGACCCAGTCCCCGAGCTCCCTGTCCGCCTCTGTGGGCGATAGGGTCA

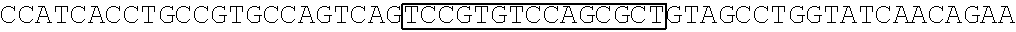

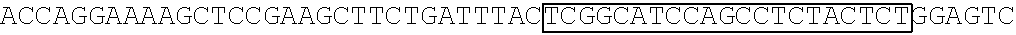

CCTTCTCGCTTCTCTGGTAGCCGTTCCGGGACGGATTTCACTCTGACCATCAGCAGTC

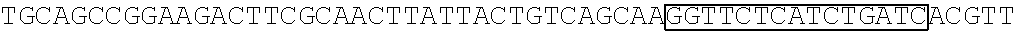

CGGACAGGGTACCAAGGTGGAGATCAAAGGTGGTGGTGGTTCTGGCGGCGGCGGCTC
CGGTGGTGGTGGTTCCGAGGTTCAGCTGGTGGAGTCTGGCGGTGGCCTGGTGCAGCC

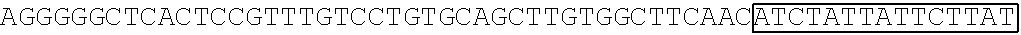

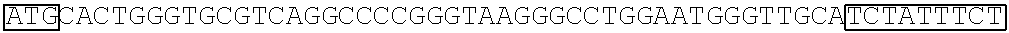

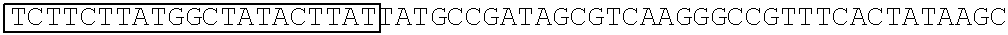

GCAGACACATCCAAAAACACAGCCTACCTACAAATGAACAGCTTAAGAGCTGAGGAC

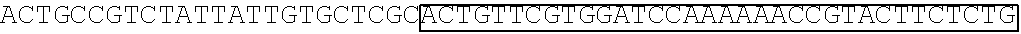

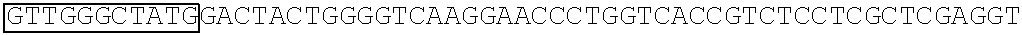

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACC
ACGCGACTTCGCAGCCTATCGCTCCAAGCTTAGAGTGAAGTTCAGCAGGAGCGCAGA
CGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCC
TTCACATGCAGGCCCTGCCCCCTCGC
Ab8scFvLH-CD28H-CD28TM-41BBCS-CD3zICS
)Amino acid Sequence:
(SEQ ID NO: 287)

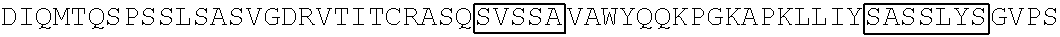

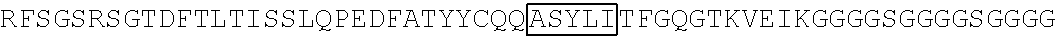

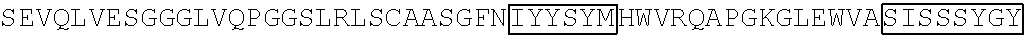

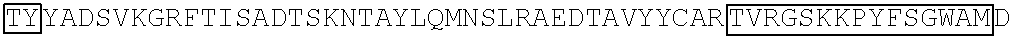

YWGQGTLVTVSSLEVKGKHLCPSPLFPGPSKPFWVLVVVGGVLACYSLLVTVAFIIFWV
KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 387)
GATATCCAGATGACCCAGTCCCCGAGCTCCCTGTCCGCCTCTGTGGGCGATAGGGTCA

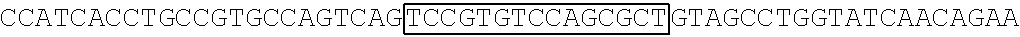

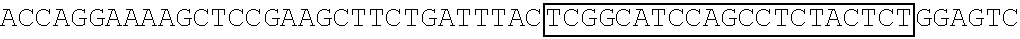

CCTTCTCGCTTCTCTGGTAGCCGTTCCGGGACGGATTTCACTCTGACCATCAGCAGTC

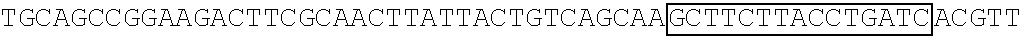

CGGACAGGGTACCAAGGTGGAGATCAAAGGTGGTGGTGGTTCTGGCGGCGGCGGCTC
CGGTGGTGGTGGTTCCGAGGTTCAGCTGGTGGAGTCTGGCGGTGGCCTGGTGCAGCC

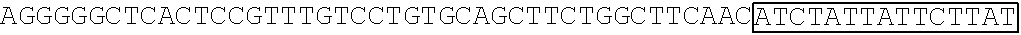

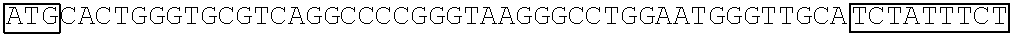

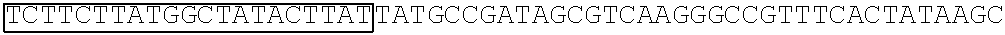

GCAGACACATCCAAAAACACAGCCTACCTACAAATGAACAGCTTAAGAGCTGAGGAC

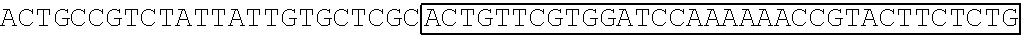

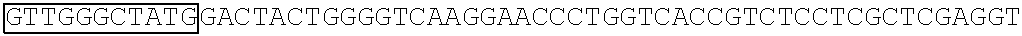

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAA
CCATTTATGAGACCAGTACAAACTACTCAAGAGGAAGATGGCTGTAGCTGCCGATTT
CCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAA
AGCCGCAGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCT
TCACATGCAGGCCCTGCCCCCTCGC
Ab8scFvLH-CD28H-CD28TM-DAP10CS-CD3zICS
Amino acid Sequence:
(SEQ ID NO: 288)

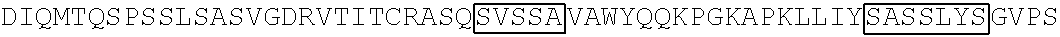

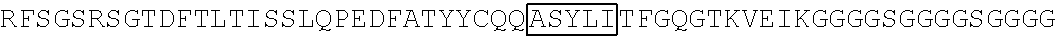

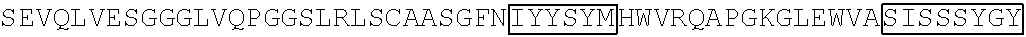

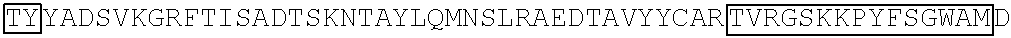

YWGQGTLVTVSSLEVKGKHLCPSPLFPGPSKPFWVLVVVGGVLACYSLLVTVAFIIFWV
LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 388)
GATATCCAGATGACCCAGTCCCCGAGCTCCCTGTCCGCCTCTGTGGGCGATAGGGTCA

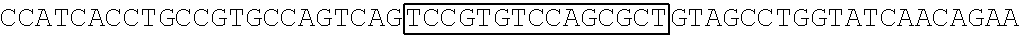

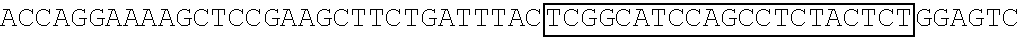

CCTTCTCGCTTCTCTGGTAGCCGTTCCGGGACGGATTTCACTCTGACCATCAGCAGTC

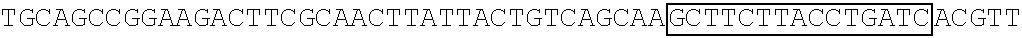

CGGACAGGGTACCAAGGTGGAGATCAAAGGTGGTGGTGGTTCTGGCGGCGGCGGCTC
CGGTGGTGGTGGTTCCGAGGTTCAGCTGGTGGAGTCTGGCGGTGGCCTGGTGCAGCC

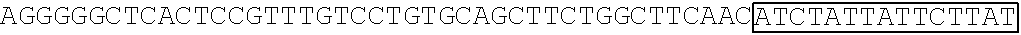

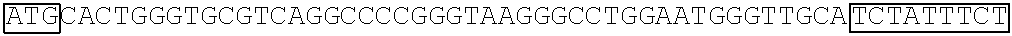

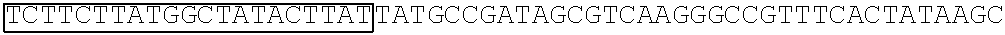

GCAGACACATCCAAAAACACAGCCTACCTACAAATGAACAGCTTAAGAGCTGAGGAC

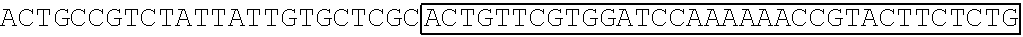

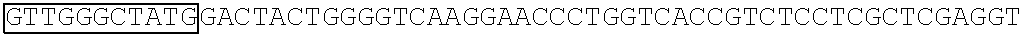

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGCTGTGCGCACGCCCACGCCGCAGCCCCGCCCAAGAAGATG
GCAAAGTCTACATCAACATGCCAGGCAGGGGCAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
CARs comprising Ab9scFvHL:
Ab9scFvHL-CD28H-CD28TM-CD28CS-CD3zICS
Amino acid Sequence:
(SEQ ID NO: 291)

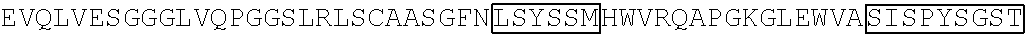

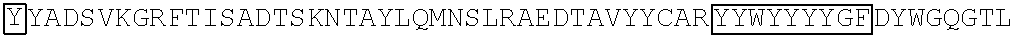

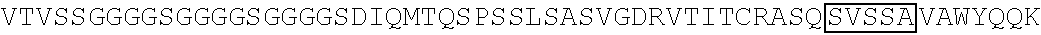

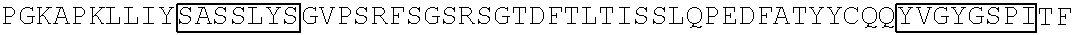

GQGTKVEIKLEVKGKHLCPSPLFPGPSKPFWVLVVVGGVLACYSLLVTVAFIIFWVRSK
RSRLLHSDYMNMTPRRPGPTRKHYQPYAPPRDFAAYRSKLRVKFSRSADAPAYQQGQ
NQLYNELNLGRREEYDVLDKRRGRDPEMGGKPQRRKNPQEGLYNELQKDKMAEAYS
EIGMKGERRRGKGHDGLYQGLSTATKDTYDALHMQALPPR
Nucleic acid Sequence:
(SEQ ID NO: 391)
GAGGTTCAGCTGGTGGAGTCTGGCGGTGGCCTGGTGCAGCCAGGGGGCTCACTCCGTT

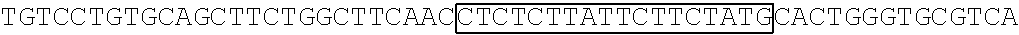

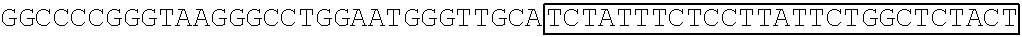

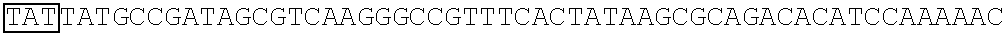

ACAGCCTACCTACAAATGAACAGCTTAAGAGCTGAGGACACTGCCGTCTATTATTGT

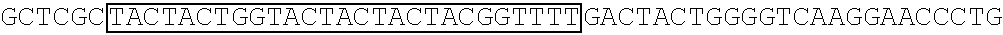

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

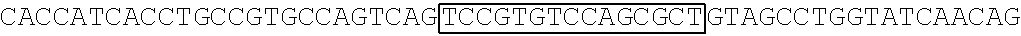

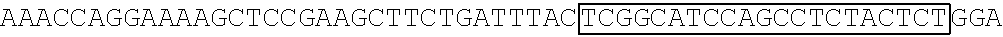

GTCCCTTCTCGCTTCTCTGGTAGCCGTTCCGGGACGGATTTCACTCTGACCATCAGCA

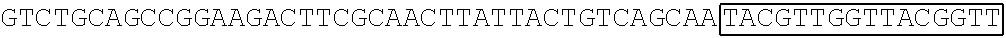

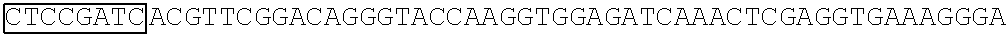

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACATGAC
TCCCCGCCGCCCCGGGCCCACCCGCAAGCATTACCAGCCCTATGCCCCACCACGCGACT
TCGCAGCCTATCGCTCCAAGCTTAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab9scFvHL-CD28H-CD28TM-41BBCS-CD3zICS
Amino acid Sequence:
(SEQ ID NO: 292)

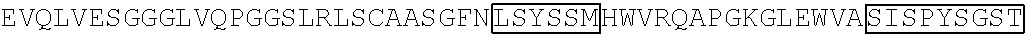

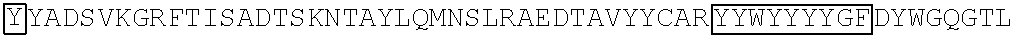

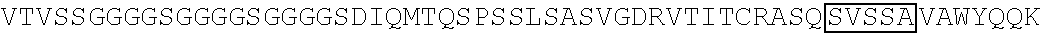

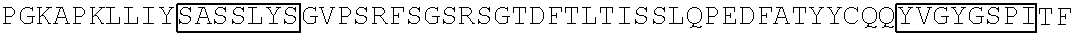

GQGTKVEIKLEVKGKHLCPSPLFPGPSKPFWVLVVVGGVLACYSLLVTVAFIIFWVKRG
RKKLLYIFKQPFMRPVQTTQEEDGCSCRFPEEEEGGCELRVKFSRSADAPAYQQGQNQ
LYNELNLGRREEYDVLDKRRGRDPEMGGKPQRRKNPQEGLYNELQKDKMAEAYSEIG
MKGERRRGKGHDGLYQGLSTATKDTYDALHMQALPPR
Nucleic acid Sequence:
(SEQ ID NO: 392)
GAGGTTCAGCTGGTGGAGTCTGGCGGTGGCCTGGTGCAGCCAGGGGGCTCACTCCGTT

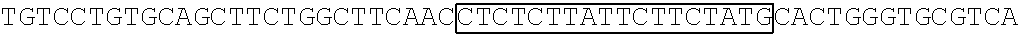

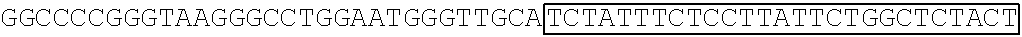

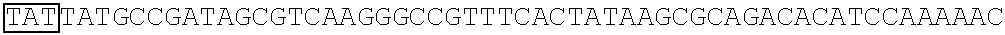

ACAGCCTACCTACAAATGAACAGCTTAAGAGCTGAGGACACTGCCGTCTATTATTGT

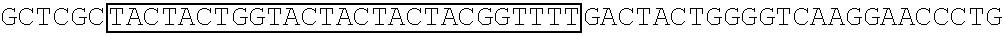

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

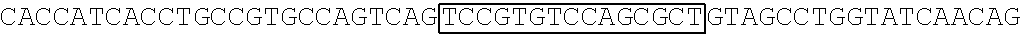

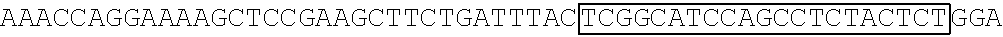

GTCCCTTCTCGCTTCTCTGGTAGCCGTTCCGGGACGGATTTCACTCTGACCATCAGCA

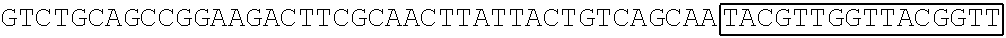

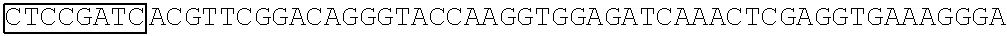

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGAAACGGGGCAGAAAGAAACTCCTGTATATATTCAAACAACCATTTAT
GAGACCAGTACAAACTACTCAAGAGGAAGATGGCTGTAGCTGCCGATTTCCAGAAGA
AGAAGAAGGAGGATGTGAACTGAGAGTGAAGTTCAGCAGGAGCGCAGACGCCCCCGC
GTACCAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGCA
GAGAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATG
CAGGCCCTGCCCCCTCGC
Ab9scFvHL-CD28H-CD28TM-DAP10CS-CD3zICS
Amino acid Sequence:
(SEQ ID NO: 293)

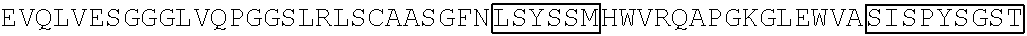

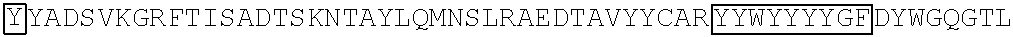

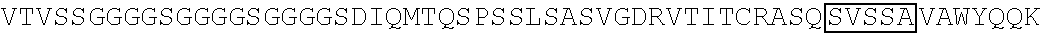

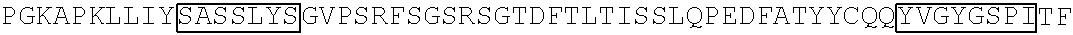

GQGTKVEIKLEVKGKHLCPSPLFPGPSKPFWVLVVVGGVLACYSLLVTVAFIIFWVLCA
RPRRSPAQEDGKVYINMPGRGRVKFSRSADAPAYQQGQNQLYNELNLGRREEYDVLD
KRRGRDPEMGGKPQRRKNPQEGLYNELQKDKMAEAYSEIGMKGERRRGKGHDGLYQ
GLSTATKDTYDALHMQALPPR
Nucleic acid Sequence:
(SEQ ID NO: 393)
GAGGTTCAGCTGGTGGAGTCTGGCGGTGGCCTGGTGCAGCCAGGGGGCTCACTCCGTT

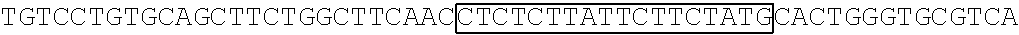

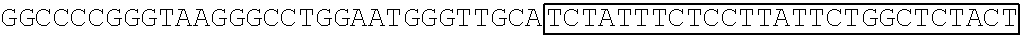

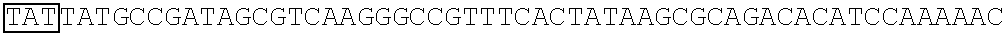

ACAGCCTACCTACAAATGAACAGCTTAAGAGCTGAGGACACTGCCGTCTATTATTGT

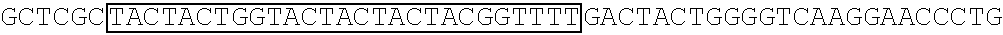

GTCACCGTCTCCTCGGGTGGTGGTGGTTCTGGCGGCGGCGGCTCCGGTGGTGGTGGTT
CCGATATCCAGATGACCCAGTCCCCGAGCTCCCTGTCCGCCTCTGTGGGCGATAGGGT

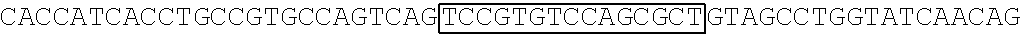

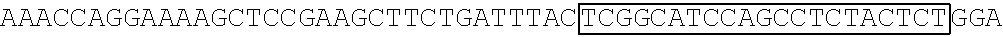

GTCCCTTCTCGCTTCTCTGGTAGCCGTTCCGGGACGGATTTCACTCTGACCATCAGCA

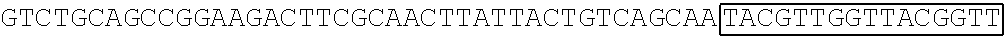

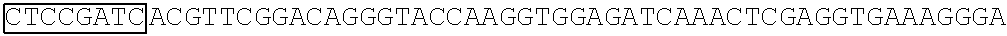

AACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTAT
TTTCTGGGTGCTGTGCGCACGCCCACGCCGCAGCCCCGCCCAAGAAGATGGCAAAGTC
TACATCAACATGCCAGGCAGGGGCAGAGTGAAGTTCAGCAGGAGCGCAGACGCCCCC
GCGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACA
TGCAGGCCCTGCCCCCTCGC
CARs comprising Ab9scFvLH:
Ab9scFvLH-CD28H-CD28TM-CD28CS-CD3zICS
Amino acid Sequence:
(SEQ ID NO: 296)

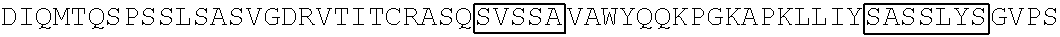

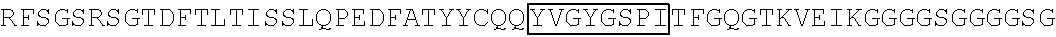

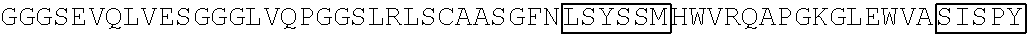

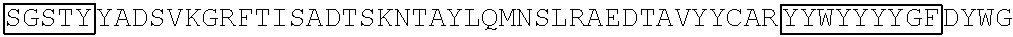

QGTLVTVSSLEVKGKHLCPSPLFPGPSKPFWVLVVVGGVLACYSLLVTVAFIIFWVRSK
RSRLLHSDYMNMTPRRPGPTRKHYQPYAPPRDFAAYRSKLRVKFSRSADAPAYQQGQ
NQLYNELNLGRREEYDVLDKRRGRDPEMGGKPQRRKNPQEGLYNELQKDKMAEAYS
EIGMKGERRRGKGHDGLYQGLSTATKDTYDALHMQALPPR
Nucleic acid Sequence:
(SEQ ID NO: 396)
GATATCCAGATGACCCAGTCCCCGAGCTCCCTGTCCGCCTCTGTGGGCGATAGGGTCA

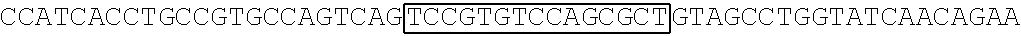

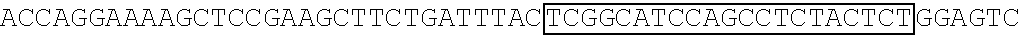

CCTTCTCGCTTCTCTGGTAGCCGTTCCGGGACGGATTTCACTCTGACCATCAGCAGTC

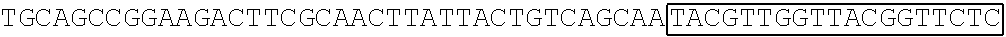

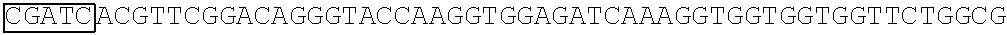

GCGGCGGCTCCGGTGGTGGTGGTTCCGAGGTTCAGCTGGTGGAGTCTGGCGGTGGCCT

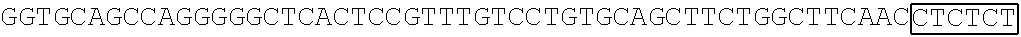

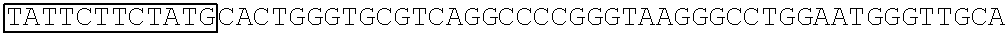

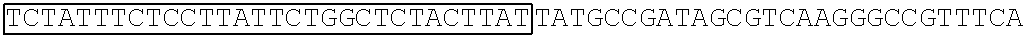

CTATAAGCGCAGACACATCCAAAAACACAGCCTACCTACAAATGAACAGCTTAAGAG

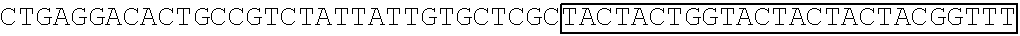

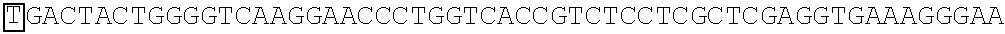

ACACCTTTGTCCAAGTCCCCTATTTCCCGGACCTTCTAAGCCCTTTTGGGTGCTGGTG
GTGGTTGGTGGAGTCCTGGCTTGCTATAGCTTGCTAGTAACAGTGGCCTTTATTATT
TTCTGGGTGAGGAGTAAGAGGAGCAGGCTCCTGCACAGTGACTACATGAACATGACT
CCCCGCCGCCCCGGGCCCACCCGCAAGCATTACCAGCCCTATGCCCCACCACGCGACTT
CGCAGCCTATCGCTCCAAGCTTAGAGTGAAGTTCAGCAGGAGCGCAGACGCCCCCGCG
TACCAGCAGGGCCAGAACCAGCTCTATAACGAGCTCAATCTAGGACGAAGAGAGGAG
TACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTTCACATGC
AGGCCCTGCCCCCTCGC
Ab9scFvLH-CD28H-CD28TM-41BBCS-CD3zICS
Amino acid Sequence:
(SEQ ID NO: 297)

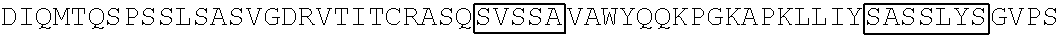

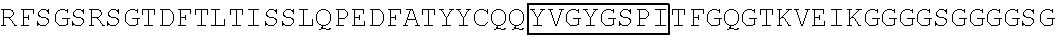

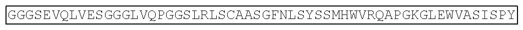

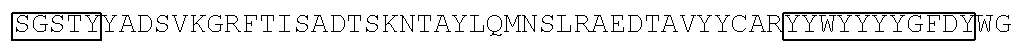

QGTLVTVSSLEVKGKHLCPSPLFPGPSKPFWVLVVVGGVLACYSLLVTVAFIIFWVKRG
RKKLLYIFKQPFMRPVQTTQEEDGCSCRFPEEEEGGCELRVKFSRSADAPAYQQGQNQ
LYNELNLGRREEYDVLDKRRGRDPEMGGKPQRRKNPQEGLYNELQKDKMAEAYSEIG
MKGERRRGKGHDGLYQGLSTATKDTYDALHMQALPPR
Nucleic acid Sequence:
(SEQ ID NO: 397)
GATATCCAGATGACCCAGTCCCCGAGCTCCCTGTCCGCCTCTGTGGGCGATAGGGTCA

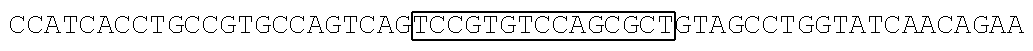

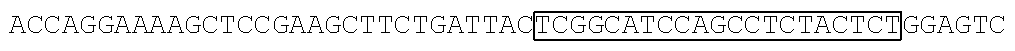

CCTTCTCGCTTCTCTGGTAGCCGTTCCGGGACGGATTTCACTCTGACCATCAGCAGTC

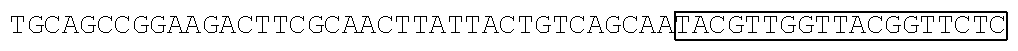

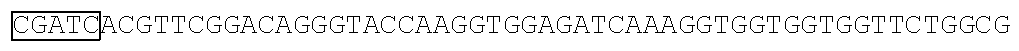

GCGGCGGCTCCGGTGGTGGTGGTTCCGAGGTTCAGCTGGTGGAGTCTGGCGGTGGCCT

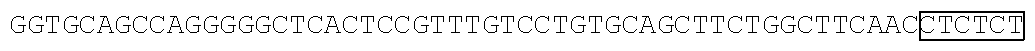

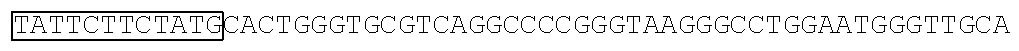

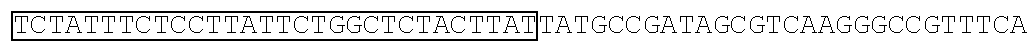

CTATAAGCGCAGACACATCCAAAAACACAGCCTACCTACAAATGAACAGCTTAAGAG

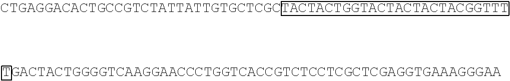

ACACCTTTGTCCAAGTCCCCTATTTCCCGGACCTTCTAAGCCCTTTTGGGTGCTGGTG
GTGGTTGGTGGAGTCCTGGCTTGCTATAGCTTGCTAGTAACAGTGGCCTTTATTATT
TTCTGGGTGAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAA
GAAGAAGGAGGATGTGAACTGAGAGTGAAGTTCAGCAGGAGCGCAGACGCCCCCGCG
TACCAGCAGGGCCAGAACCAGCTCTATAACGAGCTCAATCTAGGACGAAGAGAGGAG
TACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTTCACATGC
AGGCCCTGCCCCCTCGC
Ab9scFvLH-CD28H-CD28TM-DAP10CS-CD3zICS
Amino acid Sequence:
(SEQ ID NO: 298)

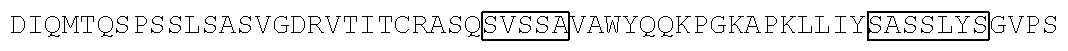

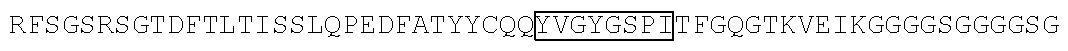

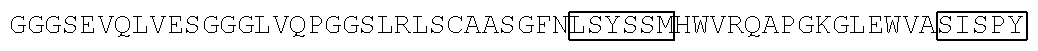

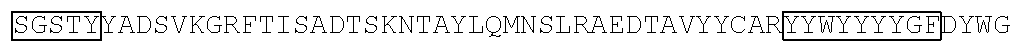

QGTLVTVSSLEVKGKHLCPSPLFPGPSKPFWVLVVVGGVLACYSLLVTVAFIIFWVLCA
RPRRSPAQEDGKVYINMPGRGRVKFSRSADAPAYQQGQNQLYNELNLGRREEYDVLD
KRRGRDPEMGGKPQRRKNPQEGLYNELQKDKMAEAYSEIGMKGERRRGKGHDGLYQ
GLSTATKDTYDALHMQALPPR
Nucleic acid Sequence:
(SEQ ID NO: 398)
GATATCCAGATGACCCAGTCCCCGAGCTCCCTGTCCGCCTCTGTGGGCGATAGGGTCA

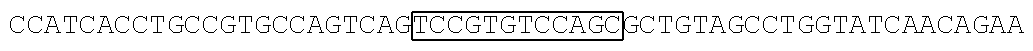

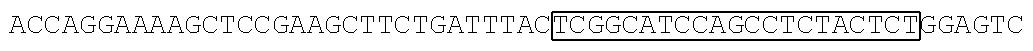

CCTTCTCGCTTCTCTGGTAGCCGTTCCGGGACGGATTTCACTCTGACCATCAGCAGTC

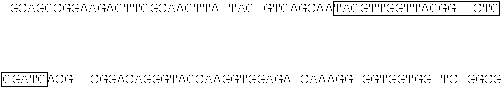

GCGGCGGCTCCGGTGGTGGTGGTTCCGAGGTTCAGCTGGTGGAGTCTGGCGGTGGCCT

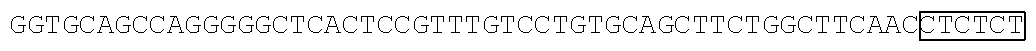

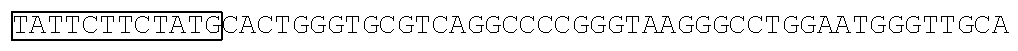

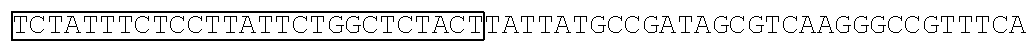

CTATAAGCGCAGACACATCCAAAAACACAGCCTACCTACAAATGAACAGCTTAAGAG

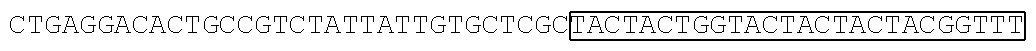

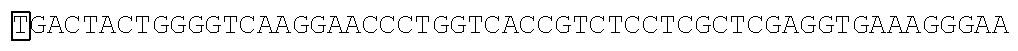

ACACCTTTGTCCAAGTCCCCTATTTCCCGGACCTTCTAAGCCCTTTTGGGTGCTGGTG
GTGGTTGGTGGAGTCCTGGCTTGCTATAGCTTGCTAGTAACAGTGGCCTTTATTATT
TTCTGGGTGCTGTGCGCACGCCCACGCCGCAGCCCCGCCCAAGAAGATGGCAAAGTCT
ACATCAACATGCCAGGCAGGGGCAGAGTGAAGTTCAGCAGGAGCGCAGACGCCCCCG
CGTACCAGCAGGGCCAGAACCAGCTCTATAACGAGCTCAATCTAGGACGAAGAGAGG
AGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCACAT
GCAGGCCCTGCCCCCTCGC
CARs comprising Ab10scFvHL:
Ab 10scFvHL-CD28H-CD28TM-CD28CS-CD3zICS
Amino acid Sequence:
(SEQ ID NO: 301)

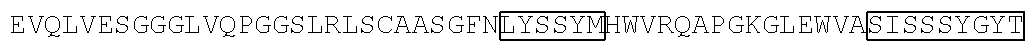

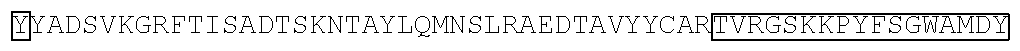

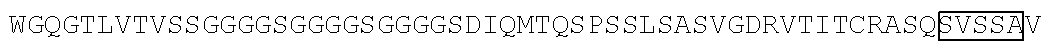

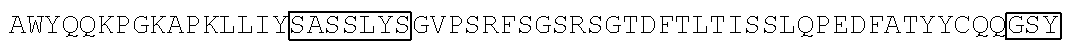

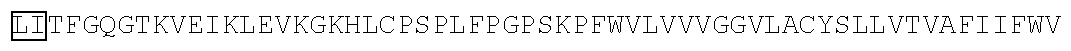

RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 401)
GAGGTTCAGCTGGTGGAGTCTGGCGGTGGCCTGGTGCAGCCAGGGGGCTCACTCCGTT

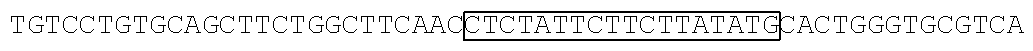

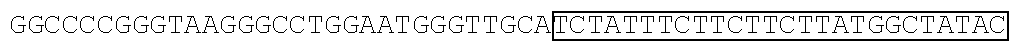

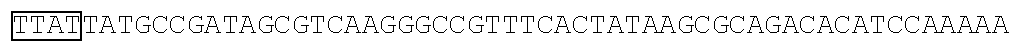

CACAGCCTACCTACAAATGAACAGCTTAAGAGCTGAGGACACTGCCGTCTATTATTG

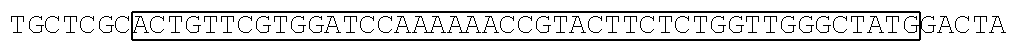

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

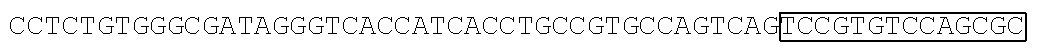

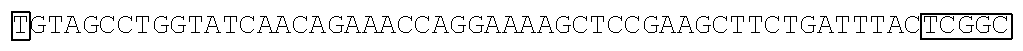

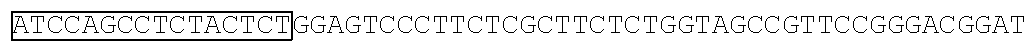

TTCACTCTGACCATCAGCAGTCTGCAGCCGGAAGACTTCGCAACTTATTACTGTCAGC

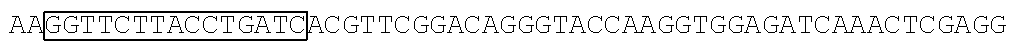

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCCA
CCACGCGACTTCGCAGCCTATCGCTCCAAGCTTAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab 10scFvHL-CD28H-CD28TM-41BBCS-CD3zICS
Amino acid Sequence:
(SEQ ID NO: 302)

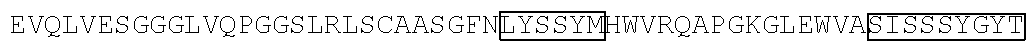

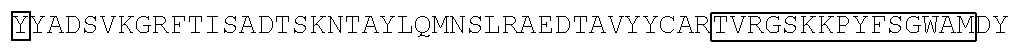

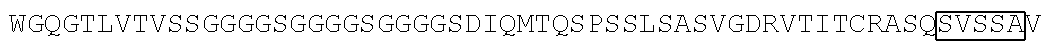

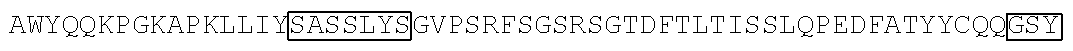

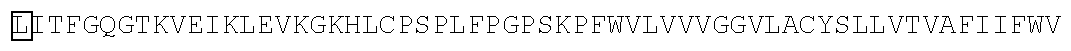

KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 402)
GAGGTTCAGCTGGTGGAGTCTGGCGGTGGCCTGGTGCAGCCAGGGGGCTCACTCCGTT

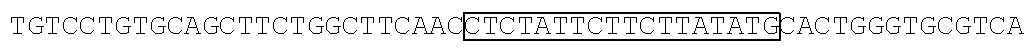

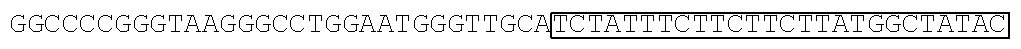

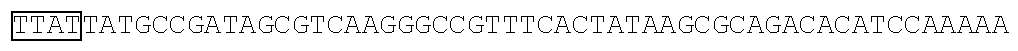

CACAGCCTACCTACAAATGAACAGCTTAAGAGCTGAGGACACTGCCGTCTATTATTG

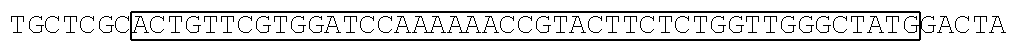

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

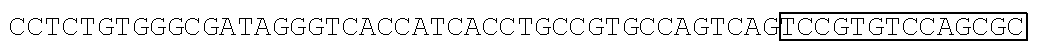

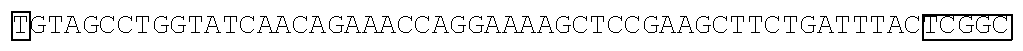

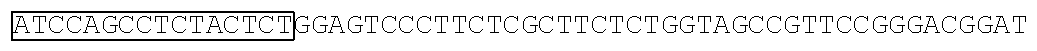

TTCACTCTGACCATCAGCAGTCTGCAGCCGGAAGACTTCGCAACTTATTACTGTCAGC

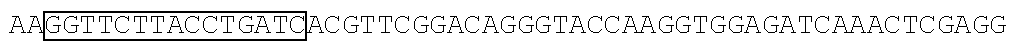

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAAACGGGGCAGAAAGAAACTCCTGTATATATTCAAACA
ACCATTTATGAGACCAGTACAAACTACTCAAGAGGAAGATGGCTGTAGCTGCCGATT
TCCAGAAGAAGAAGAAGGAGGATGTGAACTGAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
Ab 10scFvHL-CD28H-CD28TM-DAP10CS-CD3zICS
Amino acid Sequence:
(SEQ ID NO: 303)

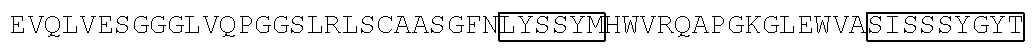

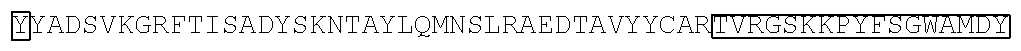

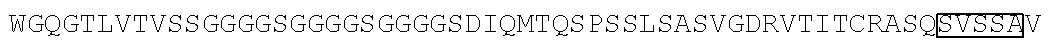

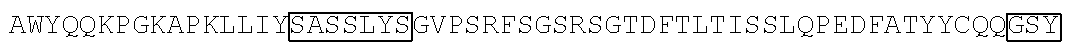

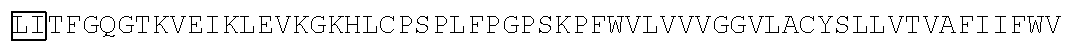

LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 403)
GAGGTTCAGCTGGTGGAGTCTGGCGGTGGCCTGGTGCAGCCAGGGGGCTCACTCCGTT

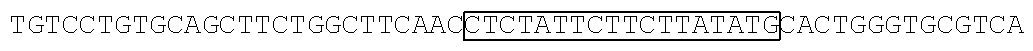

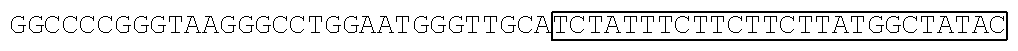

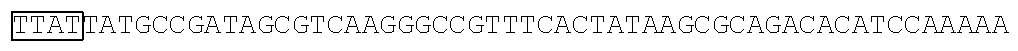

CACAGCCTACCTACAAATGAACAGCTTAAGAGCTGAGGACACTGCCGTCTATTATTG

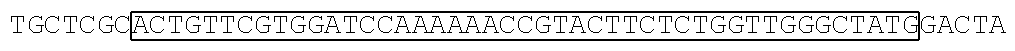

CTGGGGTCAAGGAACCCTGGTCACCGTCTCCTCGGGTGGTGGTGGTTCTGGCGGCGGC
GGCTCCGGTGGTGGTGGTTCCGATATCCAGATGACCCAGTCCCCGAGCTCCCTGTCCG

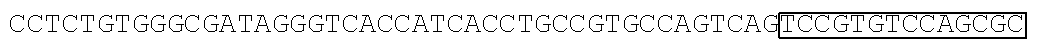

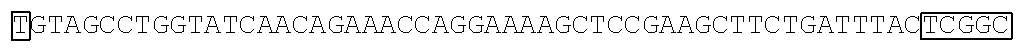

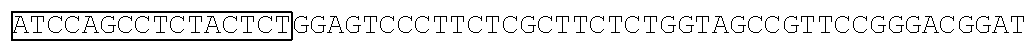

TTCACTCTGACCATCAGCAGTCTGCAGCCGGAAGACTTCGCAACTTATTACTGTCAGC

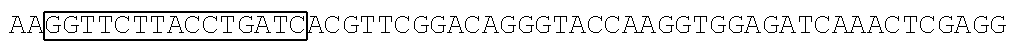

TGAAAGGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGCTGTGCGCACGCCCACGCCGCAGCCCCGCCCAAGAAGAT
GGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGG
GAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGC
CCTTCACATGCAGGCCCTGCCCCCTCGC
CARs comprising Ab10scFvLH:
Ab9scFvLH-CD28H-CD28TM-CD28CS-CD3zICS
Amino acid Sequence:
(SEQ ID NO: 306)

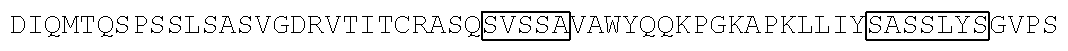

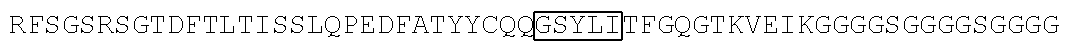

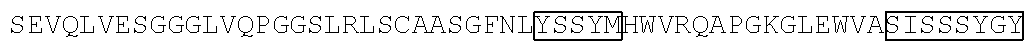

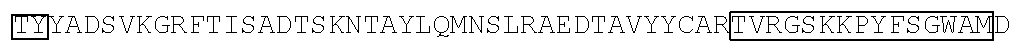

YWGQGTLVTVSSLEVKGKHLCPSPLFPGPSKPFWVLVVVGGVLACYSLLVTVAFIIFWV
RSKRSRLLHSDYMNMTPRRPGPTRKHYQPYAPPRDFAAYRSKLRVKFSRSADAPAYQ
QGQNQLYNELNLGRREEYDVLDKRRGRDPEMGGKPQRRKNPQEGLYNELQKDKMA
EAYSEIGMKGERRRGKGHDGLYQGLSTATKDTYDALHMQALPPR
Nucleic acid Sequence:
(SEQ ID NO: 406)
GATATCCAGATGACCCAGTCCCCGAGCTCCCTGTCCGCCTCTGTGGGCGATAGGGTCA

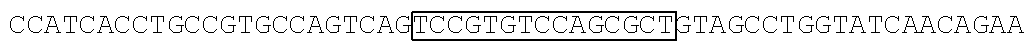

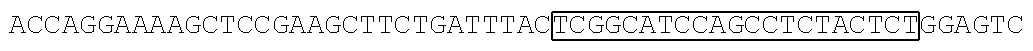

CCTTCTCGCTTCTCTGGTAGCCGTTCCGGGACGGATTTCACTCTGACCATCAGCAGTC

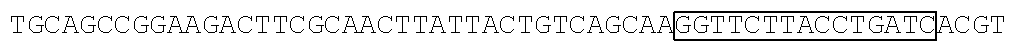

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

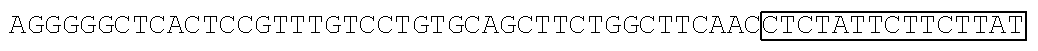

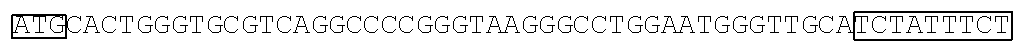

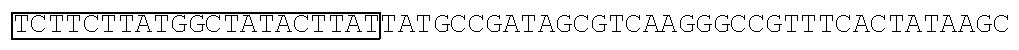

GCAGACACATCCAAAAACACAGCCTACCTACAAATGAACAGCTTAAGAGCTGAGGAC

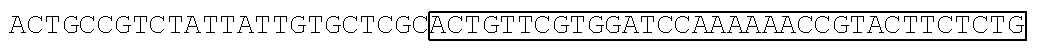

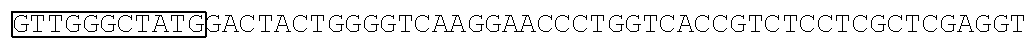

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACC
ACGCGACTTCGCAGCCTATCGCTCCAAGCTTAGAGTGAAGTTCAGCAGGAGCGCAGA
CGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCC
TTCACATGCAGGCCCTGCCCCCTCGC
Ab 10scFvLH-CD28H-CD28TM-41BBCS-CD3zICS
Amino acid Sequence:
(SEQ ID NO: 307)

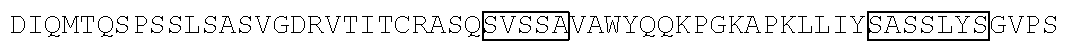

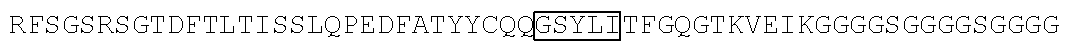

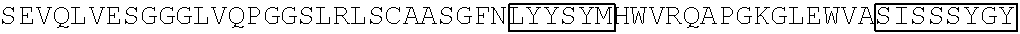

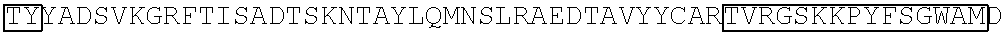

YWGQGTLVTVSSLEVKGKHLCPSPLFPGPSKPFWVLVVVGGVLACYSLLVTVAFIIFWV
KRGRKKLLYIFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQKDKMAEAY
SEIGMKGERRRGKGHDGLYQGLSTATKDTYDALHMQALPPR
Nucleic acid Sequence:
(SEQ ID NO: 407)
GATATCCAGATGACCCAGTCCCCGAGCTCCCTGTCCGCCTCTGTGGGCGATAGGGTCA

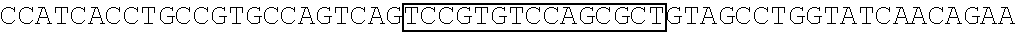

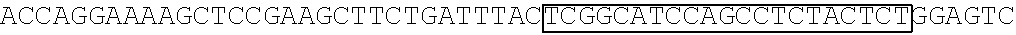

CCTTCTCGCTTCTCTGGTAGCCGTTCCGGGACGGATTTCACTCTGACCATCAGCAGTC

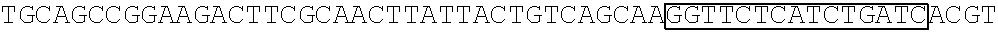

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

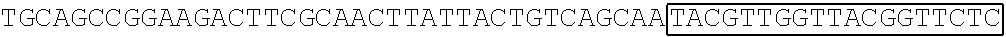

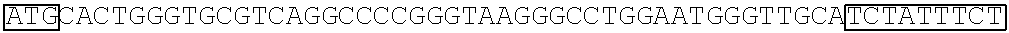

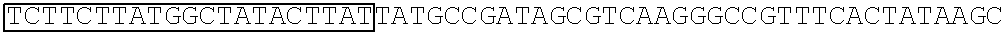

GCAGACACATCCAAAAACACAGCCTACCTACAAATGAACAGCTTAAGAGCTGAGGAC

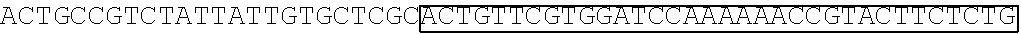

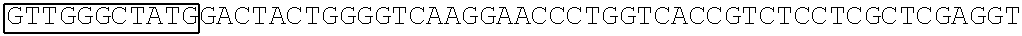

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAA
CCATTTATGAGACCAGTACAAACTACTCAAGAGGAAGATGGCTGTAGCTGCCGATTT
CCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAA
AGCCGCAGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCT
TCACATGCAGGCCCTGCCCCCTCGC
Ab 10scFvLH-CD28H-CD28TM-DAP10CS-CD3zICS
Amino acid Sequence:
(SEQ ID NO: 308)

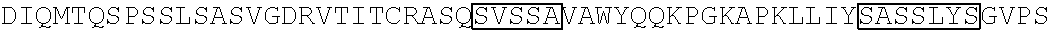

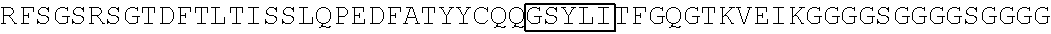

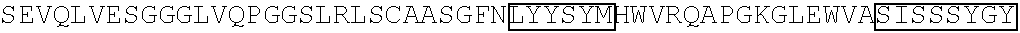

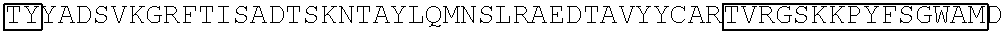

YWGQGTLVTVSSLEVKGKHLCPSPLFPGPSKPFWVLVVVGGVLACYSLLVTVAFIIFWV
LCARPRRSPAQEDGKVYINMPGRGRVKFSRSADAPAYQQGQNQLYNELNLGRREEYD
VLDKRRGRDPEMGGKPQRRKNPQEGLYNELQKDKMAEAYSEIGMKGERRRGKGHDG
LYQGLSTATKDTYDALHMQALPPR
Nucleic acid Sequence:
(SEQ ID NO: 408)
GATATCCAGATGACCCAGTCCCCGAGCTCCCTGTCCGCCTCTGTGGGCGATAGGGTCA

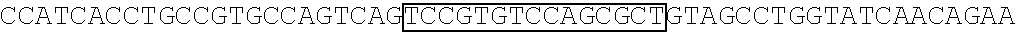

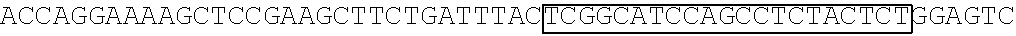

CCTTCTCGCTTCTCTGGTAGCCGTTCCGGGACGGATTTCACTCTGACCATCAGCAGTC

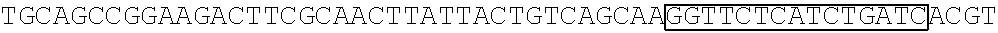

TCGGACAGGGTACCAAGGTGGAGATCAAAGGTGGTGGTGGTTCTGGCGGCGGCGGCT
CCGGTGGTGGTGGTTCCGAGGTTCAGCTGGTGGAGTCTGGCGGTGGCCTGGTGCAGCC

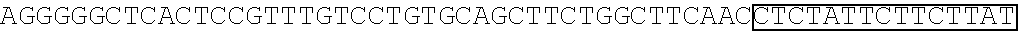

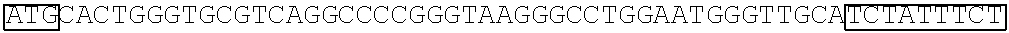

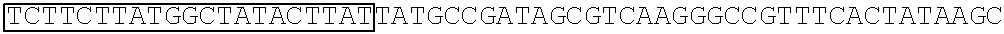

GCAGACACATCCAAAAACACAGCCTACCTACAAATGAACAGCTTAAGAGCTGAGGAC

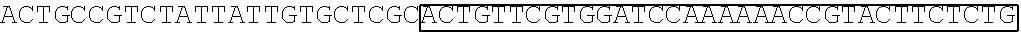

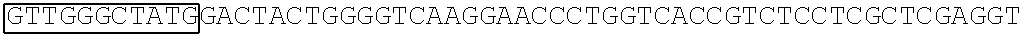

GAAAGGGAAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACAGTGGCC
TTTATTATTTTCTGGGTGCTGTGCGCACGCCCACGCCGCAGCCCCGCCCAAGAAGATG
GCAAAGTCTACATCAACATGCCAGGCAGGGGCAGAGTGAAGTTCAGCAGGAGCGCAG
ACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGG
AAAGCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGACGCC
CTTCACATGCAGGCCCTGCCCCCTCGC
CARs comprising Ab8scFvHL with LS, T2A, and trCD19:
LS-Ab8scFvHL-CD28H-CD28TM-CD28CS-CD3zICS-T2A-trCD19
Amino acid Sequence:
(SEQ ID NO: 284)

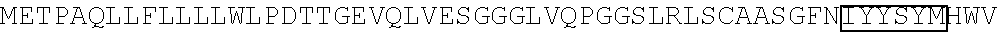

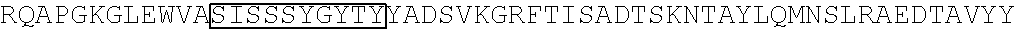

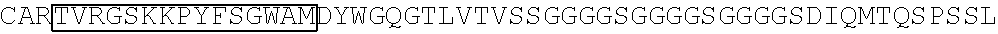

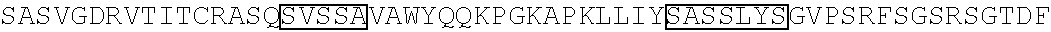

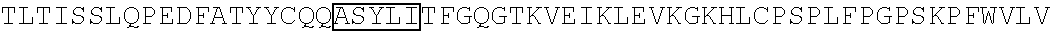

VVGGVLACYSLLVTVAFIIFWVRSKRSRLLHSDYMNMTPRRPGPTRKHYQPYAPPRDF
AAYRSKLRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDAL
HMQALPPRAGAKRSGSGEGRGSLLTCGDVEENPGPRMPPPRLLFFLLFLTPMEVRPEE
PLVVKVEEGDNAVLQCLKGTSDGPTQQLTWSRESPLKPFLKLSLGLPGLGIHMRPLAI
WLFIFNVSQQMGGFYLCQPGPPSEKAWQPGWTVNVEGSGELFRWNVSDLGGLGCGL
KNRSSEGPSSPSGKLMSPKLYVWAKDRPEIWEGEPPCLPPRDSLNQSLSQDLTMAPGS
TLWLSCGVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYLIFCLCSL
VGILHLQRALVLRRKRKRMT
Nucleic acid Sequence:
(SEQ ID NO: 384)
ATGGAAACCCCAGCGCAGCTTCTCTTCCTCCTGCTACTCTGGCTCCCAGATACCACCG
GAGAGGTTCAGCTGGTGGAGTCTGGCGGTGGCCTGGTGCAGCCAGGGGGCTCACTCC

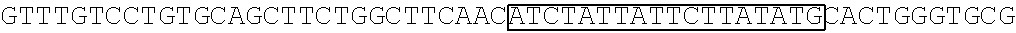

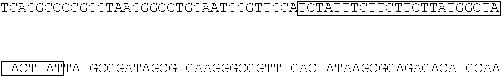

AAACACAGCCTACCTACAAATGAACAGCTTAAGAGCTGAGGACACTGCCGTCTATTA

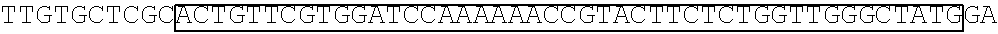

CTACTGGGGTCAAGGAACCCTGGTCACCGTCTCCTCGGGTGGTGGTGGTTCTGGCGGC
GGCGGCTCCGGTGGTGGTGGTTCCGATATCCAGATGACCCAGTCCCCGAGCTCCCTGT

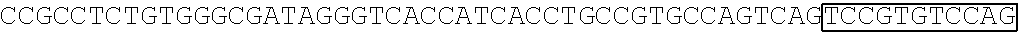

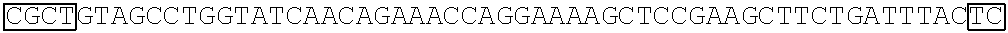

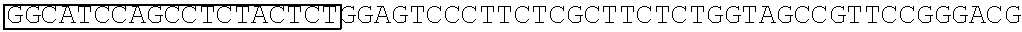

GATTTCACTCTGACCATCAGCAGTCTGCAGCCGGAAGACTTCGCAACTTATTACTGTC

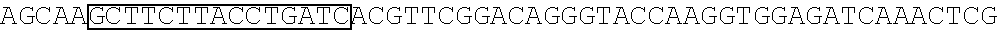

AGGTGAAAGGGAAACACCTTTGTCCAAGTCCCCTATTTCCCGGACCTTCTAAGCCCTT
TTGGGTGCTGGTGGTGGTTGGTGGAGTCCTGGCTTGCTATAGCTTGCTAGTAACAGT
GGCCTTTATTATTTTCTGGGTGAGGAGTAAGAGGAGCAGGCTCCTGCACAGTGACTA
CATGAACATGACTCCCCGCCGCCCCGGGCCCACCCGCAAGCATTACCAGCCCTATGCC
CCACCACGCGACTTCGCAGCCTATCGCTCCAAGCTTAGAGTGAAGTTCAGCAGGAGCG
CAGACGCCCCCGCGTACCAGCAGGGCCAGAACCAGCTCTATAACGAGCTCAATCTAGG
ACGAAGAGAGGAGTACGATGTTTTGGACAAGAGACGTGGCCGGGACCCTGAGATGGG
GGGAAAGCCGCAGAGAAGGAAGAACCCTCAGGAAGGCCTGTACAATGAACTGCAGAA
AGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGAC
GCCCTTCACATGCAGGCCCTGCCCCCTCGCGCCGGCGCCAAAAGGTCTGGCTCCGGTG
AGGGCAGAGGAAGTCTTCTAACATGCGGTGACGTGGAGGAGAATCCCGGCCCTAGAA
TGCCACCTCCTCGCCTCCTCTTCTTCCTCCTCTTCCTCACCCCCATGGAAGTCAGGCCC
GAGGAACCTCTAGTGGTGAAGGTGGAAGAGGGAGATAACGCTGTGCTGCAGTGCCTC
AAGGGGACCTCAGATGGCCCCACTCAGCAGCTGACCTGGTCTCGGGAGTCCCCGCTTA
AACCCTTCTTAAAACTCAGCCTGGGGCTGCCAGGCCTGGGAATCCACATGAGGCCCCT
GGCCATCTGGCTTTTCATCTTCAACGTCTCTCAACAGATGGGGGGCTTCTACCTGTGC
CAGCCGGGGCCCCCCTCTGAGAAGGCCTGGCAGCCTGGCTGGACAGTCAATGTGGAGG
GCAGCGGGGAGCTGTTCCGGTGGAATGTTTCGGACCTAGGTGGCCTGGGCTGTGGCC
TGAAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTCT
CCCACCGAGGGACAGCCTGAACCAGAGCCTCAGCCAGGACCTCACCATGGCCCCTGGC
TCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGA
CGATCGCCCGGCCAGAGATATGTGGGTAATGGAGACGGGTCTGTTGTTGCCCCGGGCC
ACAGCTCAAGACGCTGGAAAGTATTATTGTCACCGTGGCAACCTGACCATGTCATTCC
ACCTGGAGATCACTGCTCGGCCAGTACTATGGCACTGGCTGCTGAGGACTGGTGGCTG
GAAGGTCTCAGCTGTGACTTTGGCTTATCTGATCTTCTGCCTGTGTTCCCTTGTGGGC
ATTCTTCATCTTCAAAGAGCCCTGGTCCTGAGGAGGAAAAGAAAGCGAATGACTTAA
CARs comprising Ab8scFvLH with LS, T2A, and trCD19:
LS-Ab8scFvLH-CD28H-CD28TM-CD28CS-CD3zICS-T2A-trCD19
Amino acid Sequence:
(SEQ ID NO: 289)

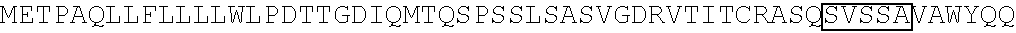

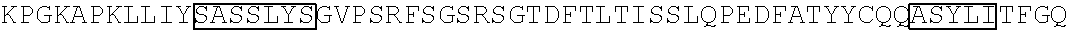

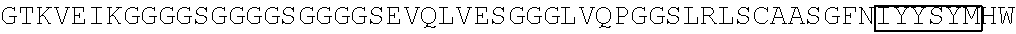

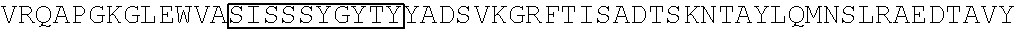

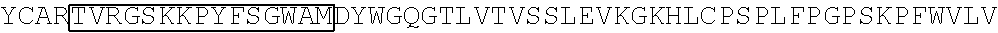

VVGGVLACYSLLVTVAFIIFWVRSKRSRLLHSDYMNMTPRRPGPTRKHYQPYAPPRDF
AAYRSKLRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDAL
HMQALPPRAGAKRSGSGEGRGSLLTCGDVEENPGPRMPPPRLLFFLLFLTPMEVRPEE
PLVVKVEEGDNAVLQCLKGTSDGPTQQLTWSRESPLKPFLKLSLGLPGLGIHMRPLAI
WLFIFNVSQQMGGFYLCQPGPPSEKAWQPGWTVNVEGSGELFRWNVSDLGGLGCGL
KNRSSEGPSSPSGKLMSPKLYVWAKDRPEIWEGEPPCLPPRDSLNQSLSQDLTMAPGS
TLWLSCGVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYLIFCLCSL
VGILHLQRALVLRRKRKRMT
Nucleic acid Sequence:
(SEQ ID NO: 389)
ATGGAAACCCCAGCGCAGCTTCTCTTCCTCCTGCTACTCTGGCTCCCAGATACCACCG
GAGATATCCAGATGACCCAGTCCCCGAGCTCCCTGTCCGCCTCTGTGGGCGATAGGGT

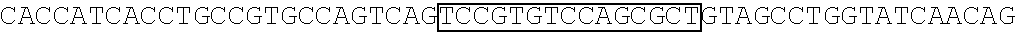

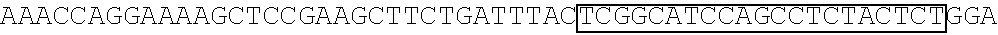

GTCCCTTCTCGCTTCTCTGGTAGCCGTTCCGGGACGGATTTCACTCTGACCATCAGCA

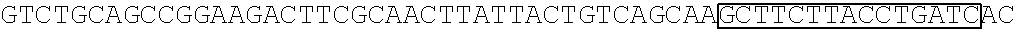

GTTCGGACAGGGTACCAAGGTGGAGATCAAAGGTGGTGGTGGTTCTGGCGGCGGCGG
CTCCGGTGGTGGTGGTTCCGAGGTTCAGCTGGTGGAGTCTGGCGGTGGCCTGGTGCA

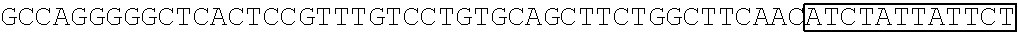

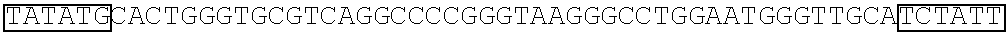

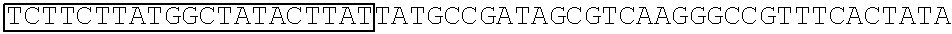

AGCGCAGACACATCCAAAAACACAGCCTACCTACAAATGAACAGCTTAAGAGCTGAG

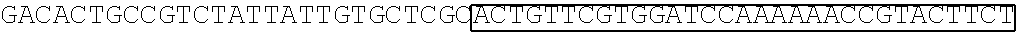

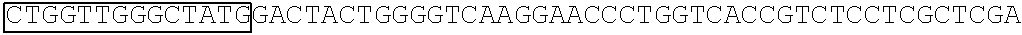

GGTGAAAGGGAAACACCTTTGTCCAAGTCCCCTATTTCCCGGACCTTCTAAGCCCTTT
TGGGTGCTGGTGGTGGTTGGTGGAGTCCTGGCTTGCTATAGCTTGCTAGTAACAGTG
GCCTTTATTATTTTCTGGGTGAGGAGTAAGAGGAGCAGGCTCCTGCACAGTGACTAC
ATGAACATGACTCCCCGCCGCCCCGGGCCCACCCGCAAGCATTACCAGCCCTATGCCC
CACCACGCGACTTCGCAGCCTATCGCTCCAAGCTTAGAGTGAAGTTCAGCAGGAGCGC
AGACGCCCCCGCGTACCAGCAGGGCCAGAACCAGCTCTATAACGAGCTCAATCTAGGA
CGAAGAGAGGAGTACGATGTTTTGGACAAGAGACGTGGCCGGGACCCTGAGATGGGG
GGAAAGCCGCAGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTACGAC
GCCCTTCACATGCAGGCCCTGCCCCCTCGCGCCGGCGCCAAAAGGTCTGGCTCCGGTG
AGGGCAGAGGAAGTCTTCTAACATGCGGTGACGTGGAGGAGAATCCCGGCCCTAGAA
TGCCACCTCCTCGCCTCCTCTTCTTCCTCCTCTTCCTCACCCCCATGGAAGTCAGGCCC
GAGGAACCTCTAGTGGTGAAGGTGGAAGAGGGAGATAACGCTGTGCTGCAGTGCCTC
AAGGGGACCTCAGATGGCCCCACTCAGCAGCTGACCTGGTCTCGGGAGTCCCCGCTTA
AACCCTTCTTAAAACTCAGCCTGGGGCTGCCAGGCCTGGGAATCCACATGAGGCCCCT
GGCCATCTGGCTTTTCATCTTCAACGTCTCTCAACAGATGGGGGGCTTCTACCTGTGC
CAGCCGGGGCCCCCCTCTGAGAAGGCCTGGCAGCCTGGCTGGACAGTCAATGTGGAGG
GCAGCGGGGAGCTGTTCCGGTGGAATGTTTCGGACCTAGGTGGCCTGGGCTGTGGCC
TGAAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTCT
CCCACCGAGGGACAGCCTGAACCAGAGCCTCAGCCAGGACCTCACCATGGCCCCTGGC
TCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGA
CGATCGCCCGGCCAGAGATATGTGGGTAATGGAGACGGGTCTGTTGTTGCCCCGGGCC
ACAGCTCAAGACGCTGGAAAGTATTATTGTCACCGTGGCAACCTGACCATGTCATTCC
ACCTGGAGATCACTGCTCGGCCAGTACTATGGCACTGGCTGCTGAGGACTGGTGGCTG
GAAGGTCTCAGCTGTGACTTTGGCTTATCTGATCTTCTGCCTGTGTTCCCTTGTGGGC
ATTCTTCATCTTCAAAGAGCCCTGGTCCTGAGGAGGAAAAGAAAGCGAATGACTTAA
CARs comprising Ab9scFvHL with LS, T2A, and trCD19:
LS-Ab9scFvHL-CD28H-CD28TM-CD28CS-CD3zICS-T2A-trCD19
Amino acid Sequence:
(SEQ ID NO: 294)

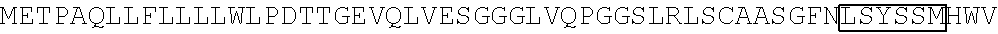

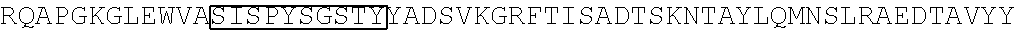

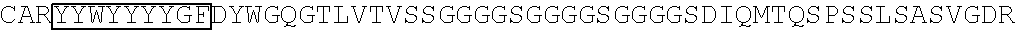

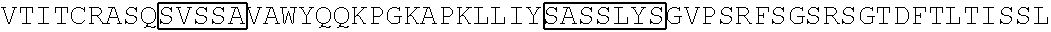

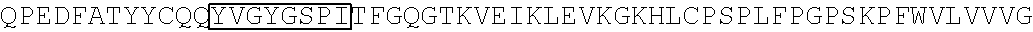

GVLACYSLLVTVAFIIFWVRSKRSRLLHSDYMNMTPRRPGPTRKHYQPYAPPRDFAAY
RSKLRVKFSRSADAPAYQQGQNQLYNELNLGRREEYDVLDKRRGRDPEMGGKPQRR
KNPQEGLYNELQKDKMAEAYSEIGMKGERRRGKGHDGLYQGLSTATKDTYDALHMQ
ALPPRAGAKRSGSGEGRGSLLTCGDVEENPGPRMPPPRLLFFLLFLTPMEVRPEEPLV
VKVEEGDNAVLQCLKGTSDGPTQQLTWSRESPLKPFLKLSLGLPGLGIHMRPLAIWLF
IFNVSQQMGGFYLCQPGPPSEKAWQPGWTVNVEGSGELFRWNVSDLGGLGCGLKNRS
SEGPSSPSGKLMSPKLYVWAKDRPEIWEGEPPCLPPRDSLNQSLSQDLTMAPGSTLWL
SCGVPPDSVSRGPLSWTHVHPKGPKSLLSLELKDDRPARDMWVMETGLLLPRATAQ
DAGKYYCHRGNLTMSFHLEITARPVLWHWLLRTGGWKVSAVTLAYLIFCLCSLVGILH
LQRALVLRRKRKRMT
Nucleic acid Sequence:
(SEQ ID NO: 394)
ATGGAAACCCCAGCGCAGCTTCTCTTCCTCCTGCTACTCTGGCTCCCAGATACCACCG
GAGAGGTTCAGCTGGTGGAGTCTGGCGGTGGCCTGGTGCAGCCAGGGGGCTCACTCC

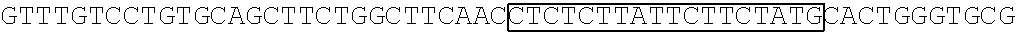

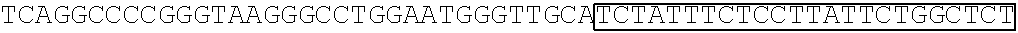

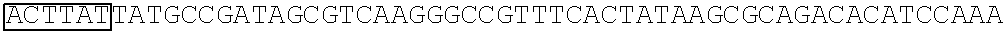

AACACAGCCTACCTACAAATGAACAGCTTAAGAGCTGAGGACACTGCCGTCTATTAT

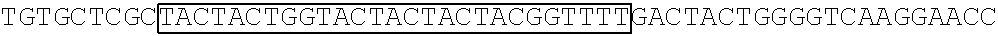

CTGGTCACCGTCTCCTCGGGTGGTGGTGGTTCTGGCGGCGGCGGCTCCGGTGGTGGTG
GTTCCGATATCCAGATGACCCAGTCCCCGAGCTCCCTGTCCGCCTCTGTGGGCGATAG

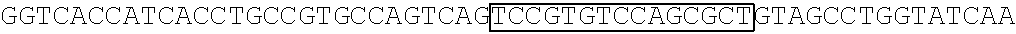

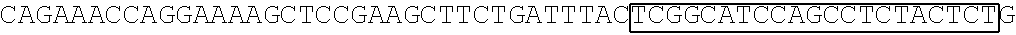

GAGTCCCTTCTCGCTTCTCTGGTAGCCGTTCCGGGACGGATTTCACTCTGACCATCAG

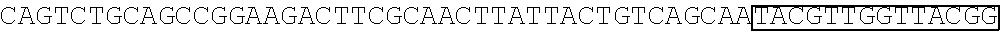

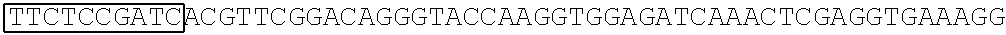

GAAACACCTTTGTCCAAGTCCCCTATTTCCCGGACCTTCTAAGCCCTTTTGGGTGCTG
GTGGTGGTTGGTGGAGTCCTGGCTTGCTATAGCTTGCTAGTAACAGTGGCCTTTATT
ATTTTCTGGGTGAGGAGTAAGAGGAGCAGGCTCCTGCACAGTGACTACATGAACATG
ACTCCCCGCCGCCCCGGGCCCACCCGCAAGCATTACCAGCCCTATGCCCCACCACGCGA
CTTCGCAGCCTATCGCTCCAAGCTTAGAGTGAAGTTCAGCAGGAGCGCAGACGCCCCC
GCGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACA
TGCAGGCCCTGCCCCCTCGCGCCGGCGCCAAAAGGTCTGGCTCCGGTGAGGGCAGAGG
AAGTCTTCTAACATGCGGTGACGTGGAGGAGAATCCCGGCCCTAGAATGCCACCTCCT
CGCCTCCTCTTCTTCCTCCTCTTCCTCACCCCCATGGAAGTCAGGCCCGAGGAACCTCT
AGTGGTGAAGGTGGAAGAGGGAGATAACGCTGTGCTGCAGTGCCTCAAGGGGACCTC
AGATGGCCCCACTCAGCAGCTGACCTGGTCTCGGGAGTCCCCGCTTAAACCCTTCTTA
AAACTCAGCCTGGGGCTGCCAGGCCTGGGAATCCACATGAGGCCCCTGGCCATCTGGC
TTTTCATCTTCAACGTCTCTCAACAGATGGGGGGCTTCTACCTGTGCCAGCCGGGGCC
CCCCTCTGAGAAGGCCTGGCAGCCTGGCTGGACAGTCAATGTGGAGGGCAGCGGGGA
GCTGTTCCGGTGGAATGTTTCGGACCTAGGTGGCCTGGGCTGTGGCCTGAAGAACAG
GTCCTCAGAGGGCCCCAGCTCCCCTTCCGGGAAGCTCATGAGCCCCAAGCTGTATGTG
TGGGCCAAAGACCGCCCTGAGATCTGGGAGGGAGAGCCTCCGTGTCTCCCACCGAGGG
ACAGCCTGAACCAGAGCCTCAGCCAGGACCTCACCATGGCCCCTGGCTCCACACTCTG
GCTGTCCTGTGGGGTACCCCCTGACTCTGTGTCCAGGGGCCCCCTCTCCTGGACCCAT
GTGCACCCCAAGGGGCCTAAGTCATTGCTGAGCCTAGAGCTGAAGGACGATCGCCCGG
CCAGAGATATGTGGGTAATGGAGACGGGTCTGTTGTTGCCCCGGGCCACAGCTCAAG
ACGCTGGAAAGTATTATTGTCACCGTGGCAACCTGACCATGTCATTCCACCTGGAGA
TCACTGCTCGGCCAGTACTATGGCACTGGCTGCTGAGGACTGGTGGCTGGAAGGTCTC
AGCTGTGACTTTGGCTTATCTGATCTTCTGCCTGTGTTCCCTTGTGGGCATTCTTCAT
CTTCAAAGAGCCCTGGTCCTGAGGAGGAAAAGAAAGCGAATGACTTAA
CARs comprising Ab9scFvLH with LS, T2A, and trCD19:
LS-Ab9scFvLH-CD28H-CD28TM-CD28CS-CD3zICS-T2A-trCD19
Amino acid Sequence:
(SEQ ID NO: 299)

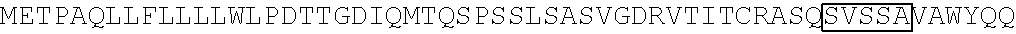

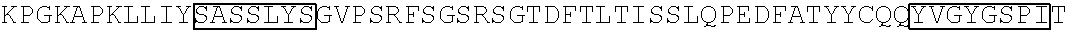

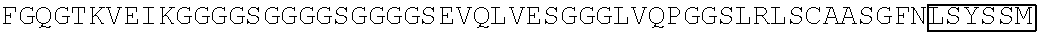

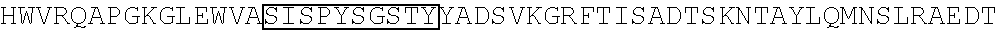

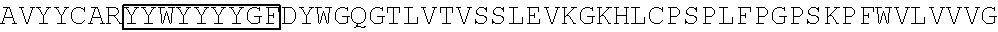

GVLACYSLLVTVAFIIFWVRSKRSRLLHSDYMNMTPRRPGPTRKHYQPYAPPRDFAAY
RSKLRVKFSRSADAPAYQQGQNQLYNELNLGRREEYDVLDKRRGRDPEMGGKPQRR
KNPQEGLYNELQKDKMAEAYSEIGMKGERRRGKGHDGLYQGLSTATKDTYDALHMQ
ALPPRAGAKRSGSGEGRGSLLTCGDVEENPGPRMPPPRLLFFLLFLTPMEVRPEEPLV
VKVEEGDNAVLQCLKGTSDGPTQQLTWSRESPLKPFLKLSLGLPGLGIHMRPLAIWLF
IFNVSQQMGGFYLCQPGPPSEKAWQPGWTVNVEGSGELFRWNVSDLGGLGCGLKNRS
SEGPSSPSGKLMSPKLYVWAKDRPEIWEGEPPCLPPRDSLNQSLSQDLTMAPGSTLWL
SCGVPPDSVSRGPLSWTHVHPKGPKSLLSLELKDDRPARDMWVMETGLLLPRATAQ
DAGKYYCHRGNLTMSFHLEITARPVLWHWLLRTGGWKVSAVTLAYLIFCLCSLVGILH
LQRALVLRRKRKRMT
Nucleic acid Sequence:
(SEQ ID NO: 399)
ATGGAAACCCCAGCGCAGCTTCTCTTCCTCCTGCTACTCTGGCTCCCAGATACCACCG
GAGATATCCAGATGACCCAGTCCCCGAGCTCCCTGTCCGCCTCTGTGGGCGATAGGGT

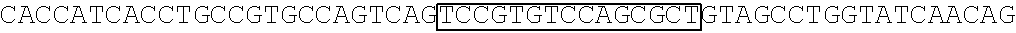

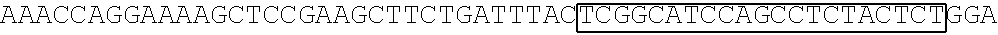

GTCCCTTCTCGCTTCTCTGGTAGCCGTTCCGGGACGGATTTCACTCTGACCATCAGCA

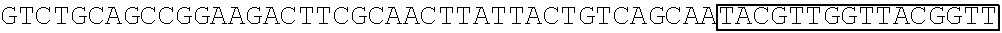

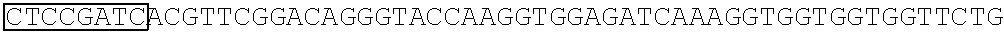

GCGGCGGCGGCTCCGGTGGTGGTGGTTCCGAGGTTCAGCTGGTGGAGTCTGGCGGTG

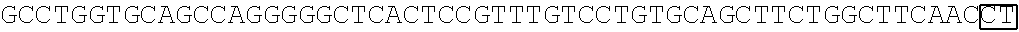

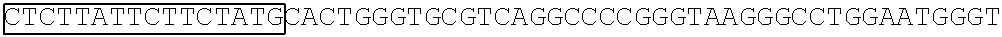

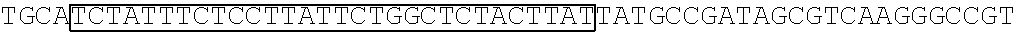

TTCACTATAAGCGCAGACACATCCAAAAACACAGCCTACCTACAAATGAACAGCTTA

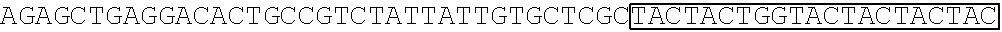

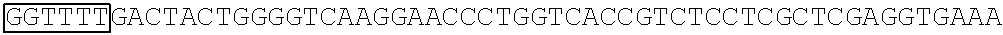

GGGAAACACCTTTGTCCAAGTCCCCTATTTCCCGGACCTTCTAAGCCCTTTTGGGTGC
TGGTGGTGGTTGGTGGAGTCCTGGCTTGCTATAGCTTGCTAGTAACAGTGGCCTTTA
TTATTTTCTGGGTGAGGAGTAAGAGGAGCAGGCTCCTGCACAGTGACTACATGAACA
TGACTCCCCGCCGCCCCGGGCCCACCCGCAAGCATTACCAGCCCTATGCCCCACCACGC
GACTTCGCAGCCTATCGCTCCAAGCTTAGAGTGAAGTTCAGCAGGAGCGCAGACGCCC
CCGCGTACCAGCAGGGCCAGAACCAGCTCTATAACGAGCTCAATCTAGGACGAAGAG
AGGAGTACGATGTTTTGGACAAGAGACGTGGCCGGGACCCTGAGATGGGGGGAAAGC
CGCAGAGAAGGAAGAACCCTCAGGAAGGCCTGTACAATGAACTGCAGAAAGATAAGA
TGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCA
CATGCAGGCCCTGCCCCCTCGCGCCGGCGCCAAAAGGTCTGGCTCCGGTGAGGGCAGA
GGAAGTCTTCTAACATGCGGTGACGTGGAGGAGAATCCCGGCCCTAGAATGCCACCT
CCTCGCCTCCTCTTCTTCCTCCTCTTCCTCACCCCCATGGAAGTCAGGCCCGAGGAACC
TCTAGTGGTGAAGGTGGAAGAGGGAGATAACGCTGTGCTGCAGTGCCTCAAGGGGAC
CTCAGATGGCCCCACTCAGCAGCTGACCTGGTCTCGGGAGTCCCCGCTTAAACCCTTC
TTAAAACTCAGCCTGGGGCTGCCAGGCCTGGGAATCCACATGAGGCCCCTGGCCATCT
GGCTTTTCATCTTCAACGTCTCTCAACAGATGGGGGGCTTCTACCTGTGCCAGCCGGG
GCCCCCCTCTGAGAAGGCCTGGCAGCCTGGCTGGACAGTCAATGTGGAGGGCAGCGGG
GAGCTGTTCCGGTGGAATGTTTCGGACCTAGGTGGCCTGGGCTGTGGCCTGAAGAAC
AGGTCCTCAGAGGGCCCCAGCTCCCCTTCCGGGAAGCTCATGAGCCCCAAGCTGTATG
TGTGGGCCAAAGACCGCCCTGAGATCTGGGAGGGAGAGCCTCCGTGTCTCCCACCGAG
GGACAGCCTGAACCAGAGCCTCAGCCAGGACCTCACCATGGCCCCTGGCTCCACACTC
TGGCTGTCCTGTGGGGTACCCCCTGACTCTGTGTCCAGGGGCCCCCTCTCCTGGACCC
ATGTGCACCCCAAGGGGCCTAAGTCATTGCTGAGCCTAGAGCTGAAGGACGATCGCCC
GGCCAGAGATATGTGGGTAATGGAGACGGGTCTGTTGTTGCCCCGGGCCACAGCTCA
AGACGCTGGAAAGTATTATTGTCACCGTGGCAACCTGACCATGTCATTCCACCTGGA
GATCACTGCTCGGCCAGTACTATGGCACTGGCTGCTGAGGACTGGTGGCTGGAAGGT
CTCAGCTGTGACTTTGGCTTATCTGATCTTCTGCCTGTGTTCCCTTGTGGGCATTCTT
CATCTTCAAAGAGCCCTGGTCCTGAGGAGGAAAAGAAAGCGAATGACTTAA

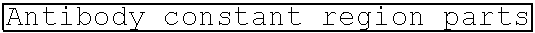

Human IgG constant region domains:
Human IgG1 CH1 domain
Amino acid Sequence:
(SEQ ID NO: 310)
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKV
Nucleic acid Sequence:
(SEQ ID NO: 410)
GCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTT
Human IgG1 hinge
Amino acid Sequence:
(SEQ ID NO: 311)
EPKSCDKTHTCPPCP
Nucleic acid Sequence:
(SEQ ID NO: 411)
GAGCCCAAATCTTGTGACAAAACTCACACATGCCCACCGTGCCCA
Human IgG1 CH2 domain
Amino acid Sequence:
(SEQ ID NO: 312)
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
Nucleic acid Sequence:
(SEQ ID NO: 412)
GCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCC
CCATCGAGAAAACCATCTCCAAAGCCAAA
Human IgG1 CH3 domain (356E and 358M; with C-terminal K)
Amino acid Sequence:
(SEQ ID NO: 313)
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
Nucleic acid Sequence:
(SEQ ID NO: 413)
GGGCAGCCCCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGC
CTCTCCCTGTCTCCGGGTAAA
Human IgG1 CH3 domain (356E and 358M; without C-terminal K)
Amino acid Sequence:
(SEQ ID NO: 314)
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
Nucleic acid Sequence:
(SEQ ID NO: 414)
GGGCAGCCCCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGC
CTCTCCCTGTCTCCGGGT
Human IgG1 CH3 domain (356D and 358L; with C-terminal K)
Amino acid Sequence:
(SEQ ID NO: 315)
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Nucleic acid Sequence:
(SEQ ID NO: 415)
GGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGC
CTCTCCCTGTCTCCGGGTAAA
Human IgG1 CH3 domain (356D and 358L; without C-terminal K)
Amino acid Sequence:
(SEQ ID NO: 316)
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
Nucleic acid Sequence:
(SEQ ID NO: 416)
GGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGC
CTCTCCCTGTCTCCGGGT
Human IgG1 Fc (356E and 358M; with C-terminal K)
Amino acid Sequence:
(SEQ ID NO: 317)
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
Nucleic acid Sequence:
(SEQ ID NO: 417)
GCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTGGTCAAAG
GCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTAAA
Human IgG1 Fc (356E and 358M; without C-terminal K)
Amino acid Sequence:
(SEQ ID NO: 318)
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPG
Nucleic acid Sequence:
(SEQ ID NO: 418)
GCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTGGTCAAAG
GCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
Human IgG1 Fc (356D and 358L; with C-terminal K)
Amino acid Sequence:
(SEQ ID NO: 319)
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
Nucleic acid Sequence:
(SEQ ID NO: 419)
GCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCC
TGCCCCCATCCCGGGATGAGCTGACCAAGAACCAGGTCAGCCTGACCTGCCTGGTCAAAG
GCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTAAA
Human IgG1 Fc (356D and 358L; without C-terminal K)
Amino acid Sequence:
(SEQ ID NO: 320)
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPG
Nucleic acid Sequence:
(SEQ ID NO: 420)
GCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCC
TGCCCCCATCCCGGGATGAGCTGACCAAGAACCAGGTCAGCCTGACCTGCCTGGTCAAAG
GCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
Human IgG1 heavy chain constant region (356E and 358M; with C-terminal K)
Amino acid Sequence:
(SEQ ID NO: 321)
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRW
QQGNVFSCSVMHEALHNHYTQKSLSLSPGK
Nucleic acid Sequence:
(SEQ ID NO: 421)
GCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGT
CAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAG
AGCCTCTCCCTGTCTCCGGGTAAA
Human IgG1 heavy chain constant region (356E and 358M; without C-terminal
K)
Amino acid Sequence:
(SEQ ID NO: 322)
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRW
QQGNVFSCSVMHEALHNHYTQKSLSLSPG
Nucleic acid Sequence:
(SEQ ID NO: 422)
GCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGT
CAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAG
AGCCTCTCCCTGTCTCCGGGT
Human IgG1 heavy chain constant region (356D and 358L; with C-terminal K)
Amino acid Sequence:
(SEQ ID NO: 323)
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRW
QQGNVFSCSVMHEALHNHYTQKSLSLSPGK
Nucleic acid Sequence:
(SEQ ID NO: 423)
GCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGT
CAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAG
AGCCTCTCCCTGTCTCCGGGTAAA
Human IgG1 heavy chain constant region (356D and 358L; without C-terminal K)
Amino acid Sequence:
(SEQ ID NO: 324)
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRW
QQGNVFSCSVMHEALHNHYTQKSLSLSPG
Nucleic acid Sequence:
(SEQ ID NO: 424)
GCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGT
CAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAG
AGCCTCTCCCTGTCTCCGGGT
Human Ig kappa light chain constant domain
Amino acid Sequence:
(SEQ ID NO: 325)
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
Nucleic acid Sequence:
(SEQ ID NO: 425)
CGTACGGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGAC
AGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAG
AGCTTCAACAGGGGAGAGTGT
Human Ig lambda light chain constant domain
Amino acid Sequence:
(SEQ ID NO: 326)
GQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
Nucleic acid Sequence:
(SEQ ID NO: 426)
GGTCAGCCCAAGGCTGCCCCCTCGGTCACTCTGTTCCCGCCCTCCTCTGAGGAGCTTCAAG
CCAACAAGGCCACACTGGTGTGTCTCATAAGTGACTTCTACCCGGGAGCCGTGACAGTGG
CCTGGAAGGCAGATAGCAGCCCCGTCAAGGCGGGAGTGGAGACCACCACACCCTCCAAAC
AAAGCAACAACAAGTACGCGGCCAGCAGCTATCTGAGCCTGACGCCTGAGCAGTGGAAGT
CCCACAGAAGCTACAGCTGCCAGGTCACGCATGAAGGGAGCACCGTGGAGAAGACAGTGG
CCCCTACAGAATGTTCATAG

What is claimed is:

1-44. (canceled)

45. An antibody (Ab) or antigen-binding Ab fragment thereof, wherein the Ab or Ab fragment binds to N-methyl D-aspartate (NMDA) receptor subtype 2B (NMDAR2B), optionally having SEQ ID NO: 1, and which antibody (Ab) or antigen-binding Ab fragment comprises:
(a) a heavy chain variable region (VH) comprising
a VH complementarity determining region 1 (CDR-H1),
a VH complementarity determining region 2 (CDR-H2), and a
VH complementarity determining region 3 (CDR-H3); and
(b) a light chain variable region (VL) comprising
a VL complementarity determining region 1 (CDR-L1),
a VL complementarity determining region 2 (CDR-L2), and, a
VL complementarity determining region 3 (CDR-L3),
wherein:
(A) the CDR-H1 comprises or consists of the CDR-H1 sequence contained in SEQ ID NO: 81, 91, 41, 51, 61, 71, or 101 or comprises or consists of the amino acid sequence of SEQ ID NO: 82, 92, 42, 52, 62, 72, 102, or 32;
(B) the CDR-H2 comprises or consists of the CDR-H2 sequence contained in SEQ ID NO: 81, 91, 41, 51, 61, 71, or 101 or comprises or consists of the amino acid sequence of SEQ ID NO: 83, 93, 43, 53, 63, 73, or 103;
(C) the CDR-H3 comprises or consists of the CDR-H3 sequence contained in SEQ ID NO: 81, 91, 41, 51, 61, 71, or 101 or comprises or consists of the amino acid sequence of SEQ ID NO: 84, 94, 44, 54, 64, 74, 104, or 34;
(D) the CDR-L1 comprises or consists of the CDR-L1 sequence contained in SEQ ID NO: 86, 96, 46, 56, 66, 76, 106, or 36 or comprises or consists of the amino acid sequence of SEQ ID NO: 87, 97, 47, 57, 67, 77, 107, or 37;
(E) the CDR-L2 comprises or consists of the CDR-L2 sequence contained in SEQ ID NO: 86, 96, 46, 56, 66, 76, 106, or 36 or comprises or consists of the amino acid sequence of SEQ ID NO: 88, 98, 48, 58, 68, 78, 108, or 38; and/or
(F) the CDR-L3 comprises or consists of the CDR-L3 sequence contained in SEQ ID NO: 86, 96, 46, 56, 66, 76, 106, or 36 or comprises or consists of the amino acid sequence of SEQ ID NO: 89, 99, 49, 59, 69, 79, 109, or 39,
optionally wherein: (I)
(i) the CDR-H1, CDR-H2, and CDR-H3 comprise or consist of the amino acid sequences set forth in SEQ ID NOs: 82, 83, and 84, respectively, and/or the CDR-L1,
(ii) the CDR-L2, and CDR-L3 comprise or consist of the amino acid sequence set forth in SEQ ID NOs: 87, 88, and 89, respectively,
(iii) the CDR-H1, CDR-H2, and CDR-H3 comprise or consist of the amino acid sequences set forth in SEQ ID NOs: 92, 93, and 94, respectively, and/or the CDR-L1, CDR-L2, and CDR-L3 comprise or consist of the amino acid sequence set forth in SEQ ID NOs: 97, 98, and 99, respectively,
(iv) the CDR-H1, CDR-H2, and CDR-H3 comprise or consist of the amino acid sequences set forth in SEQ ID NOs: 42, 43, and 44, respectively, and/or the CDR-L1, CDR-L2, and CDR-L3 comprise or consist of the amino acid sequence set forth in SEQ ID NOs: 47, 48, and 49, respectively,
(v) the CDR-H1, CDR-H2, and CDR-H3 comprise or consist of the amino acid sequences set forth in SEQ ID NOs: 52, 53, and 54, respectively, and/or the CDR-L1, CDR-L2, and CDR-L3 comprise or consist of the amino acid sequence set forth in SEQ ID NOs: 57, 58, and 59, respectively,
(vi) the CDR-H1, CDR-H2, and CDR-H3 comprise or consist of the amino acid sequences set forth in SEQ ID NOs: 62, 63, and 64, respectively, and/or the CDR-L1, CDR-L2, and CDR-L3 comprise or consist of the amino acid sequence set forth in SEQ ID NOs: 67, 68, and 69, respectively,
(vii) the CDR-H1, CDR-H2, and CDR-H3 comprise or consist of the amino acid sequences set forth in SEQ ID NOs: 72, 73, and 74, respectively, and/or the CDR-L1, CDR-L2, and CDR-L3 comprise or consist of the amino acid sequence set forth in SEQ ID NOs: 77, 78, and 79, respectively,
(viii) the CDR-H1, CDR-H2, and CDR-H3 comprise or consist of the amino acid sequences set forth in SEQ ID NOs: 102, 103, and 104, respectively, and/or the CDR-L1, CDR-L2, and CDR-L3 comprise or consist of the amino acid sequence set forth in SEQ ID NOs: 107, 108, and 109, respectively, or
(ix) the CDR-H1 and CDR-H3 comprise or consist of the amino acid sequences set forth in SEQ ID NOs: 32 and 34, respectively, and/or the CDR-L1, CDR-L2, and CDR-L3 comprise or consist of the amino acid sequence set forth in SEQ ID NOs: 37, 38, and 39, respectively; or
(II) an affinity matured variant of any of the foregoing.

46. A chimeric antigen receptor (CAR) comprising:
a. an antigen-binding domain that binds to human NMDAR2B;
b. a transmembrane (TM) domain;
c. an intracellular signaling (ICS) domain;
d. optionally a hinge that joins said antigen-binding domain and said TM domain; and
e. optionally one or more costimulatory (CS) domains;
optionally wherein the antigen-binding domain is an Ab or Ab fragment according to claim 45,
further optionally wherein the antigen-binding domain
i. comprises an scFv;
ii. comprises an amino acid sequence which is at least 80%, at least 85%, at least 90%, at least 92%, at least 94%, at least 95%, at least 96%, at least 97%, at least 98%, at least 99%, or 100% identical to SEQ ID NOs: 280, 285, 290, 295, 240, 245, 250, 255, 260, 265, 270, 275, 300, or 305 and comprises the same CDRs as those contained in said respective SEQ ID NOs; and/or
iii. competes for binding to NMDAR2B with a scFv comprising an amino acid sequence which is at least 80%, at least 85%, at least 90%, at least 92%, at least 94%, at least 95%, at least 96%, at least 97%, at least 98%, at least 99%, or 100% identical to SEQ ID NOs: 280, 285, 290, 295, 240, 245, 250, 255, 260, 265, 270, 275, 300, or 305;
further optionally wherein the TM domain is derived from:
(i) the TM region, or a membrane-spanning portion thereof, of a protein selected from the group consisting of CD28, CD3e, CD4, CDS, CDS, CD9, CD16, CD22, CD33, CD37, CD45, CD64, CD80, CD86, CD134, CD137, CD154, TCRa, TCRb, and CD3z; and/or
(ii) the TM region of CD28, or a membrane-spanning portion thereof, optionally comprising an amino acid sequence which is at least 80%, at least 85%, at least 90%, at least 95%, at least 98% at least 99%, or 100% identical to the amino acid sequence of SEQ ID NO: 114;
further optionally wherein the ICS domain is derived from:
(i) a cytoplasmic signaling sequence, or a functional fragment thereof, of a protein selected from the group consisting of CD3z, a lymphocyte receptor chain, a TCR/CD3 complex protein, an Fe receptor (FcR) subunit, an IL-2 receptor subunit, FcRg, FcRb, CD3g, CD3d, CD3e, CDS, CD22, CD66d, CD79a, CD79b, CD278 (ICOS), FceRI, DAP10, and DAP12; and/or
(ii) a cytoplasmic signaling sequence of CD3z, or a functional fragment thereof, optionally comprising an amino acid sequence which is at least 80%, at least 85%, at least 90%, at least 95%, at least 98% at least 99%, or 100% identical to the amino acid sequence of SEQ ID NO: 118;
further optionally wherein the hinge is derived from CD28, optionally comprising an amino sequence which is at least 80%, at least 85%, at least 90%, at least 95%, at least 98% at least 99%, or 100% identical to the amino acid sequence of SEQ ID NO: 113;
further optionally wherein the one or more CS domains is derived from:
(i) a cytoplasmic signaling sequence, or functional fragment thereof, of a protein selected from the group consisting of CD28, DAP10, 4-1BB (CD137), CD2, CD4, CD5, CD8, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL2Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RAN KL, VLA1, VLA-6, and CD83 ligand; and/or
(ii) a cytoplasmic signaling sequence of CD28, 4-1BB, or DAP10, or functional fragment thereof, optionally comprising an amino sequence which is at least 80%, at least 85%, at least 90%, at least 95%, at least 98% at least 99%, or 100% identical to the amino acid sequence of SEQ ID NO: 115, 116, or 117;
further optionally comprising an amino acid sequence which is at least 80%, at least 85%, at least 90%, at least 95%, at least 98% at least 99%, or 100% identical to the amino acid sequence of
(i) Ab8scFvHL-CD28H-CD28TM-CD28CS-CD3zICS (SEQ ID NO: 281),
(ii) Ab8scFvHL-CD28H-CD28TM-41BBCS-CD3zICS (SEQ ID NO: 282),
(iii) Ab8scFvHL-CD28H-CD28TM-DAP10CS-CD3zICS (SEQ ID NO: 283),
(iv) Ab8scFvLH-CD28H-CD28TM-CD28CS-CD3zICS (SEQ ID NO: 286),
(v) Ab8scFvLH-CD28H-CD28TM-41BBCS-CD3zICS (SEQ ID NO: 287),
(vi) Ab8scFvLH-CD28H-CD28TM-DAP10CS-CD3zICS (SEQ ID NO: 288),
(vii) Ab9scFvHL-CD28H-CD28TM-CD28CS-CD3zICS (SEQ ID NO: 291),
(viii) Ab9scFvHL-CD28H-CD28TM-41BBCS-CD3zICS (SEQ ID NO: 292),
(ix) Ab9scFvHL-CD28H-CD28TM-DAP10CS-CD3zICS (SEQ ID NO: 293),
(x) Ab9scFvLH-CD28H-CD28TM-CD28CS-CD3zICS (SEQ ID NO: 296),
(xi) Ab9scFvLH-CD28H-CD28TM-41BBCS-CD3zICS (SEQ ID NO: 297),
(xii) Ab9scFvLH-CD28H-CD28TM-DAP10CS-CD3zICS (SEQ ID NO: 298),
(xiii) Ab4scFvHL-CD28H-CD28TM-CD28CS-CD3zICS (SEQ ID NO: 241),
(xiv) Ab4scFvHL-CD28H-CD28TM-41BBCS-CD3zICS (SEQ ID NO: 242),
(xv) Ab4scFvHL-CD28H-CD28TM-DAP10CS-CD3zICS (SEQ ID NO: 243),
(xvi) Ab4scFvLH-CD28H-CD28TM-CD28CS-CD3zICS (SEQ ID NO: 246),
(xvii) Ab4scFvLH-CD28H-CD28TM-41BBCS-CD3zICS (SEQ ID NO: 247),
(xviii) Ab4scFvLH-CD28H-CD28TM-DAP10CS-CD3zICS (SEQ ID NO: 248),
(xix) Ab5scFvHL-CD28H-CD28TM-CD28CS-CD3zICS (SEQ ID NO: 251),
(xx) Ab5scFvHL-CD28H-CD28TM-41BBCS-CD3zICS (SEQ ID NO: 252),
(xxi) Ab5scFvHL-CD28H-CD28TM-DAP10CS-CD3zICS (SEQ ID NO: 253),
(xxii) Ab5scFvLH-CD28H-CD28TM-CD28CS-CD3zICS (SEQ ID NO: 256),
(xxiii) Ab5scFvLH-CD28H-CD28TM-41BBCS-CD3zICS (SEQ ID NO: 257),
(xiv) Ab5scFvLH-CD28H-CD28TM-DAP10CS-CD3zICS (SEQ ID NO: 258),
(xv) Ab6scFvHL-CD28H-CD28TM-CD28CS-CD3zICS (SEQ ID NO: 261),
(xvi) Ab6scFvHL-CD28H-CD28TM-41BBCS-CD3zICS (SEQ ID NO: 262),
(xvii) Ab6scFvHL-CD28H-CD28TM-DAP10CS-CD3zICS (SEQ ID NO: 263),
(xviii) Ab6scFvLH-CD28H-CD28TM-CD28CS-CD3zICS (SEQ ID NO: 266),
(xix) Ab6scFvLH-CD28H-CD28TM-41BBCS-CD3zICS (SEQ ID NO: 267),
(xxx) Ab6scFvLH-CD28H-CD28TM-DAP10CS-CD3zICS (SEQ ID NO: 268),
(xxxi) Ab7scFvHL-CD28H-CD28TM-CD28CS-CD3zICS (SEQ ID NO: 271),
(xxxii) Ab7scFvHL-CD28H-CD28TM-41BBCS-CD3zICS (SEQ ID NO: 272),
(xxxiii) Ab7scFvHL-CD28H-CD28TM-DAP10CS-CD3zICS (SEQ ID NO: 273),
(xxxiv) Ab7scFvLH-CD28H-CD28TM-CD28CS-CD3zICS (SEQ ID NO: 276),
(xxxv) Ab7scFvLH-CD28H-CD28TM-41BBCS-CD3zICS (SEQ ID NO: 277),
(xxxvi) Ab7scFvLH-CD28H-CD28TM-DAP10CS-CD3zICS (SEQ ID NO: 278),
(xxxvii) Ab10scFvHL-CD28H-CD28TM-CD28CS-CD3zICS (SEQ ID NO: 301),
(xxxviii) Ab10scFvHL-CD28H-CD28TM-41BBCS-CD3zICS (SEQ ID NO: 302),
(xxxix) Ab10scFvHL-CD28H-CD28TM-DAP10CS-CD3zICS (SEQ ID NO: 303),
(xi) Ab10scFvLH-CD28H-CD28TM-CD28CS-CD3zICS (SEQ ID NO: 306),
(xii) Ab10scFvLH-CD28H-CD28TM-41BBCS-CD3zICS (SEQ ID NO: 307), or
(xiii) Ab10scFvLH-CD28H-CD28TM-DAP10CS-CD3zICS (SEQ ID NO: 308).

47. An isolated polynucleotide or a combination of isolated polynucleotides encoding the Ab or Ab fragment of claim 45, optionally wherein:
(i) the VH-encoding polynucleotide comprises the CDR-H1-, CDR-H2-, and CDR-H3-encoding nucleic acid sequences of SEQ ID NOs: 182, 183, and 184, respectively, and the VL-encoding polynucleotide comprises the CDR-L1-, CDR-L2-, and CDR-L3-encoding nucleic acid sequences of SEQ ID NOs: 187, 188, and 189, respectively;
(ii) the VH-encoding polynucleotide comprises the CDR-H1-, CDR-H2-, and CDR-H3-encoding nucleic acid sequences of SEQ ID NOs: 192, 193, and 194, respectively, and the VL-encoding polynucleotide comprises the CDR-L1-, CDR-L2-, and CDR-L3-encoding nucleic acid sequences of SEQ ID NOs: 197, 198, and 199, respectively;
(iii) the VH-encoding polynucleotide comprises the CDR-H1-, CDR-H2-, and CDR-H3-encoding nucleic acid sequences of SEQ ID NOs: 142, 143, and 144, respectively, and the VL-encoding polynucleotide comprises the CDR-L1-, CDR-L2-, and CDR-L3-encoding nucleic acid sequences of SEQ ID NOs: 147, 148, and 149, respectively;
(iv) the VH-encoding polynucleotide comprises the CDR-H1-, CDR-H2-, and CDR-H3-encoding nucleic acid sequences of SEQ ID NOs: 152, 153, and 154, respectively, and the VL-encoding polynucleotide comprises the CDR-L1-, CDR-L2-, and CDR-L3-encoding nucleic acid sequences of SEQ ID NOs: 157, 158, and 159, respectively;
(v) the VH-encoding polynucleotide comprises the CDR-H1-, CDR-H2-, and CDR-H3-encoding nucleic acid sequences of SEQ ID NOs: 162, 163, and 164, respectively, and the VL-encoding polynucleotide comprises the CDR-L1-, CDR-L2-, and CDR-L3-encoding nucleic acid sequences of SEQ ID NOs: 167, 168, and 169, respectively;
(vi) the VH-encoding polynucleotide comprises the CDR-H1-, CDR-H2-, and CDR-H3-encoding nucleic acid sequences of SEQ ID NOs: 172, 173, and 174, respectively, and the VL-encoding polynucleotide comprises the CDR-L1-, CDR-L2-, and CDR-L3-encoding nucleic acid sequences of SEQ ID NOs: 177, 178, and 179, respectively;
(vii) the VH-encoding polynucleotide comprises the CDR-H1-, CDR-H2-, and CDR-H3-encoding nucleic acid sequences of SEQ ID NOs: 202, 203, and 204, respectively, and the VL-encoding polynucleotide comprises the CDR-L1-, CDR-L2-, and CDR-L3-encoding nucleic acid sequences of SEQ ID NOs: 207, 208, and 209, respectively; or
(viii) the VH-encoding polynucleotide comprises the CDR-H1- and CDR-H3-encoding nucleic acid sequences of SEQ ID NOs: 132 and 134, respectively, and the VL-encoding polynucleotide comprises the CDR-L1-, CDR-L2-, and CDR-L3-encoding nucleic acid sequences of SEQ ID NOs: 137, 138, and 139, respectively;
optionally wherein the isolated polynucleotide or the combination of isolated polynucleotides comprise:
(i) the VH-encoding nucleic acid sequence is at least 85%, at least 90%, at least 92%, at least 94%, at least 95%, at least 96%, at least 97%, at least 98%, at least 99%, or 100% identical to SEQ ID NO: 181 and encodes the same CDRs as those contained in SEQ ID NO: 81, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 186 and encodes the same CDRs as those contained in SEQ ID NO: 86;
(ii) the VH-encoding nucleic acid sequence is at least 85%, at least 90%, at least 92%, at least 94%, at least 95%, at least 96%, at least 97%, at least 98%, at least 99%, or 100% identical to SEQ ID NO: 191 and encodes the same CDRs as those contained in SEQ ID NO: 91, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 196 and encodes the same CDRs as those contained in SEQ ID NO: 96;
(iii) the VH-encoding nucleic acid sequence is at least 85%, at least 90%, at least 92%, at least 94%, at least 95%, at least 96%, at least 97%, at least 98%, at least 99%, or 100% identical to SEQ ID NO: 141 and encodes the same CDRs as those contained in SEQ ID NO: 41, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 146 and encodes the same CDRs as those contained in SEQ ID NO: 46;
(iv) the VH-encoding nucleic acid sequence is at least 85%, at least 90%, at least 92%, at least 94%, at least 95%, at least 96%, at least 97%, at least 98%, at least 99%, or 100% identical to SEQ ID NO: 151 and encodes the same CDRs as those contained in SEQ ID NO: 51, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 156 and encodes the same CDRs as those contained in SEQ ID NO: 56;
(v) the VH-encoding nucleic acid sequence is at least 85%, at least 90%, at least 92%, at least 94%, at least 95%, at least 96%, at least 97%, at least 98%, at least 99%, or 100% identical to SEQ ID NO: 161 and encodes the same CDRs as those contained in SEQ ID NO: 61, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 166 and encodes the same CDRs as those contained in SEQ ID NO: 66;
(vi) the VH-encoding nucleic acid sequence is at least 85%, at least 90%, at least 92%, at least 94%, at least 95%, at least 96%, at least 97%, at least 98%, at least 99%, or 100% identical to SEQ ID NO: 171 and encodes the same CDRs as those contained in SEQ ID NO: 71, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 176 and encodes the same CDRs as those contained in SEQ ID NO: 76;
(vii) the VH-encoding nucleic acid sequence is at least 85%, at least 90%, at least 92%, at least 94%, at least 95%, at least 96%, at least 97%, at least 98%, at least 99%, or 100% identical to SEQ ID NO: 201 and encodes the same CDRs as those contained in SEQ ID N& 101, and/or the VL-encoding nucleic acid sequence is at least 85%, at least 90%, at least 92%, at least 94%, at least 95%, at least 96%, at least 97%, at least 98%, at least 99%, or 100% identical to SEQ ID NO: 206 and encodes the same CDRs as those contained in SEQ ID NO: 106; or
(viii) the VL-encoding nucleic acid sequence is at least 85%, at least 90%, at least 92%, at least 94%, at least 95%, at least 96%, at least 97%, at least 98%, at least 99%, or 100% identical to SEQ ID NO: 136 and encodes the same CDRs as those contained in SEQ ID NO: 36.

48. A recombinant or isolated cell comprising:
(i) the Ab or Ab fragment of claim 45, or
(ii) a CAR comprising the Ab or Ab fragment of claim 45.

49. A pharmaceutical composition comprising:
(i) the Ab or Ab fragment of claim 45,
(ii) a CAR comprising the Ab or Ab fragment of claim 45,
(iii) a polynucleotide or combination of polynucleotides encoding the Ab or Ab fragment of claim 45, or
(iv) a cell which expresses the Ab or Ab fragment of claim 45, and
a pharmaceutically acceptable excipient or carrier;
optionally further comprising another therapeutic agent, optionally wherein the other therapeutic agent is:
(i) an anti-cancer agent, an anti-proliferative drug, a cytotoxic drug, an anti-angiogenic drug, an apoptotic drug, an immunostimulatory drug, an NMDA receptor antagonist, an NMDA receptor signaling inhibitor, an NMDAR1 inhibitor, an NMDAR2B inhibitor;
(ii) an enzyme, a hormone, a toxin, a radio isotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, or an oligonucleotide;
(iii) a chemotherapeutic agent, optionally one or more selected from alkylating agents, antimetabolites, plant alkaloids, and anti-cancer antibiotics, further optionally one or more selected from cyclophosphamide, cisplatin, carboplatin, oxaliplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof;
(iv) an immunotherapeutic agent, optionally an immune checkpoint inhibitor or a growth factor or growth factor receptor inhibitor, further optionally an inhibitor of PO-L1, PD-1, CTLA-4, VISTA, EGF, EGFR, VEGF, and/or VEGFR, or an antibody or antibody fragment against PD-L1, PD-1, CTLA-4, VISTA, EGF, EGFR, VEGF, and/or VEGFR, or an antibody or antibody fragment against a cancer antigen other than NMDAR2B; and/or
(v) an anti-emetic agent, optionally one or more selected from a neurokinin-1 receptor antagonist (NK1 RA), serotonin receptor antagonist (5-HT3 RA), dexamethasone, olanzapine, and palonosetron.

50. A method of treating a subject, the method comprising administering to the subject in need thereof a therapeutically effective amount of:
(i) the Ab or Ab fragment of claim 45,
(ii) a CAR comprising the Ab or Ab fragment of claim 45,
(iii) a polynucleotide or combination of polynucleotides encoding the Ab or Ab fragment of claim 45,
(iv) a cell which expresses the Ab or Ab fragment of claim 45, or
(v) a pharmaceutical composition comprising any of (i) to (iv),
optionally wherein the method is for the treatment of cancer, further optionally wherein the cancer is selected from pancreatic cancer, prostate cancer, ovarian cancer, small cell lung cancer, and breast cancer.


SEQUENCE LISTING (426 sequences)

SEQ ID NO: 1 - protein, 1484 aa, synthetic construct [sequence omitted]

SEQ ID NO: 2 - protein, 9 aa, synthetic construct
  source 1..9: note = Human NMDAR2B extracellular peptide
KDAHEKDDF

SEQ ID NO: 3 - protein, 10 aa, synthetic construct
  source 1..10: note = Human NMDAR2B extracellular peptide with an added N-terminal tyrosine, amidated
YKDAHEKDDF

SEQ ID NO: 4 - skipped

SEQ ID NO: 5 - protein, 10 aa, synthetic construct
  source 1..10: note = Human NMDAR1 extracellular peptide
MSIYSDKSIH

SEQ ID NO: 6-10 - skipped (5 entries)

SEQ ID NO: 11 - DNA, 4455 bp, synthetic construct
  source 1..4455: mol_type = other DNA; note = Human NMDAR2B (NCBI Reference Sequence: NP_000825.2)
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccgtcagcggaggagggccctgtaccaacaggtctcacatcaagcacgggacgggcgacaaacacggcgtggtcagcggggtacctgcaccttgggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaactgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctga

SEQ ID NO: 12-31 - skipped (20 entries)

SEQ ID NO: 32 - protein, 6 aa, synthetic construct
  source 1..6: note = Ab3 Heavy chain variable region (VH) CDR 1 (Ab3 CDR-H1)
IYYSSI

SEQ ID NO: 33 - skipped

SEQ ID NO: 34 - protein, 9 aa, synthetic construct
  source 1..9: note = Ab3 VH CDR 3 (Ab3 CDR-H3)
YVYWSSYAF

SEQ ID NO: 35 - protein, 216 aa, synthetic construct
  source 1..216: note = Antibody 3 Light chain (Ab3 LC)
DIQMTQSPSSLSASVGDRVTITCRASQSVSSAVAWYQQKPGKAPKLLIYSASSLYSGVPSRFSGSRSGTDFTLTISSLQPEDFATYYCQQYWAHGFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 109 aa, synthetic construct
  source 1..109: note = Antibody 3 Light chain variable region (Ab3 VL)
DIQMTQSPSSLSASVGDRVTITCRASQSVSSAVAWYQQKPGKAPKLLIYSASSLYSGVPSRFSGSRSGTDFTLTISSLQPEDFATYYCQQYWAHGFPITFGQGTKVEIK

SEQ ID NO: 37 - protein, 5 aa, synthetic construct
  source 1..5: note = Ab3 VL CDR 1 (Ab3 CDR-L1)
SVSSA

SEQ ID NO: 38 - protein, 7 aa, synthetic construct
  source 1..7: note = Ab3 VL CDR 2 (Ab3 CDR-L2)
SASSLYS

SEQ ID NO: 39 - protein, 8 aa, synthetic construct
  source 1..8: note = Ab3 VL CDR 3 (Ab3 CDR-L3)
YWAHGFPI

SEQ ID NO: 40 - protein, 450 aa, synthetic construct
  source 1..450: note = Antibody 4 Heavy chain (Ab4 HC)
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 120 aa, synthetic construct
  source 1..120: note = Antibody 4 Heavy chain variable region (Ab4 VH)
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSS

SEQ ID NO: 42 - protein, 6 aa, synthetic construct
  source 1..6: note = Ab4 VH CDR 1 (Ab4 CDR-H1)
LYYYSI

SEQ ID NO: 43 - protein, 10 aa, synthetic construct
  source 1..10: note = Ab4 VH CDR 2 (Ab4 CDR-H2)
YISSYYGYTY

SEQ ID NO: 44 - protein, 9 aa, synthetic construct
  source 1..9: note = Ab4 VH CDR 3 (Ab4 CDR-H3)
YYPGGWHAF

SEQ ID NO: 45 - protein, 216 aa, synthetic construct
  source 1..216: note = Antibody 4 Light chain (Ab4 LC)
DIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 109 aa, synthetic construct
  source 1..109: note = Antibody 4 Light chain variable region (Ab4 VL)
DIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIK

SEQ ID NO: 47 - protein, 5 aa, synthetic construct
  source 1..5: note = Ab4 VL CDR 1 (Ab4 CDR-L1)
SVSSA

SEQ ID NO: 48 - protein, 7 aa, synthetic construct
  source 1..7: note = Ab4 VL CDR 2 (Ab4 CDR-L2)
SASSLYS

SEQ ID NO: 49 - protein, 8 aa, synthetic construct
  source 1..8: note = Ab4 VL CDR 3 (Ab4 CDR-L3)
YYGYGYPI

SEQ ID NO: 50 - protein, 456 aa, synthetic construct
  source 1..456: note = Antibody 5 Heavy chain (Ab5 HC)
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 126 aa, synthetic construct
  source 1..126: note = Antibody 5 Heavy chain variable region (Ab5 VH)
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSS

SEQ ID NO: 52 - protein, 6 aa, synthetic construct
  source 1..6: note = Ab5 VH CDR 1 (Ab5 CDR-H1)
LYSSYM

SEQ ID NO: 53 - protein, 10 aa, synthetic construct
  source 1..10: note = Ab5 VH CDR 2 (Ab5 CDR-H2)
SIYSSYSSTY

SEQ ID NO: 54 - protein, 15 aa, synthetic construct
  source 1..15: note = Ab5 VH CDR 3 (Ab5 CDR-H3)
TVRGSKKPYFSGWAM

SEQ ID NO: 55 - protein, 213 aa, synthetic construct
  source 1..213: note = Antibody 5 Light chain (Ab5 LC)
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 106 aa, synthetic construct
  source 1..106: note = Antibody 5 Light chain variable region (Ab5 VL)
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIK

SEQ ID NO: 57 - protein, 5 aa, synthetic construct
  source 1..5: note = Ab5 VL CDR 1 (Ab5 CDR-L1)
SVSSA

SEQ ID NO: 58 - protein, 7 aa, synthetic construct
  source 1..7: note = Ab5 VL CDR 2 (Ab5 CDR-L2)
SASSLYS

SEQ ID NO: 59 - protein, 5 aa, synthetic construct
  source 1..5: note = Ab5 VL CDR 3 (Ab5 CDR-L3)
GAGLI

SEQ ID NO: 60 - protein, 456 aa, synthetic construct
  source 1..456: note = Antibody 6 Heavy chain (Ab6 HC)
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 126 aa, synthetic construct
  source 1..126: note = Antibody 6 Heavy chain variable region (Ab6 VH)
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 62 - protein, 6 aa, synthetic construct
  source 1..6: note = Ab6 VH CDR 1 (Ab6 CDR-H1)
IYSSYI

SEQ ID NO: 63 - protein, 10 aa, synthetic construct
  source 1..10: note = Ab6 VH CDR 2 (Ab6 CDR-H2)
SISSSYGYTY

SEQ ID NO: 64 - protein, 15 aa, synthetic construct
  source 1..15: note = Ab6 VH CDR 3 (Ab6 CDR-H3)
TVRGSKKPYFSGWAM

SEQ ID NO: 65 - protein, 213 aa, synthetic construct
  source 1..213: note = Antibody 6 Light chain (Ab6 LC)
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 106 aa, synthetic construct
  source 1..106: note = Antibody 6 Light chain variable region (Ab6 VL)
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIK

SEQ ID NO: 67 - protein, 5 aa, synthetic construct
  source 1..5: note = Ab6 VL CDR 1 (Ab6 CDR-L1)
SVSSA

SEQ ID NO: 68 - protein, 7 aa, synthetic construct
  source 1..7: note = Ab6 VL CDR 2 (Ab6 CDR-L2)
SASSLYS

SEQ ID NO: 69 - protein, 5 aa, synthetic construct
  source 1..5: note = Ab6 VL CDR 3 (Ab6 CDR-L3)
GGSLI

SEQ ID NO: 70 - protein, 456 aa, synthetic construct
  source 1..456: note = Antibody 7 Heavy chain (Ab7 HC)
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 126 aa, synthetic construct
  source 1..126: note = Antibody 7 Heavy chain variable region (Ab7 VH)
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 72 - protein, 6 aa, synthetic construct
  source 1..6: note = Ab7 VH CDR 1 (Ab7 CDR-H1)
FSSSSI

SEQ ID NO: 73 - protein, 10 aa, synthetic construct
  source 1..10: note = Ab7 VH CDR 2 (Ab7 CDR-H2)
SISSSYGYTY

SEQ ID NO: 74 - protein, 15 aa, synthetic construct
  source 1..15: note = Ab7 VH CDR 3 (Ab7 CDR-H3)
TVRGSKKPYFSGWAM

SEQ ID NO: 75 - protein, 213 aa, synthetic construct
  source 1..213: note = Antibody 7 Light chain (Ab7 LC)
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 106 aa, synthetic construct
  source 1..106: note = Antibody 7 Light chain variable region (Ab7 VL)
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIK

SEQ ID NO: 77 - protein, 5 aa, synthetic construct
  source 1..5: note = Ab7 VL CDR 1 (Ab7 CDR-L1)
SVSSA

SEQ ID NO: 78 - protein, 7 aa, synthetic construct
  source 1..7: note = Ab7 VL CDR 2 (Ab7 CDR-L2)
SASSLYS

SEQ ID NO: 79 - protein, 5 aa, synthetic construct
  source 1..5: note = Ab7 VL CDR 3 (Ab7 CDR-L3)
GSHLI

SEQ ID NO: 80 - protein, 456 aa, synthetic construct
  source 1..456: note = Antibody 8 Heavy chain (Ab8 HC)
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 126 aa, synthetic construct
  source 1..126: note = Antibody 8 Heavy chain variable region (Ab8 VH)
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 82 - protein, 6 aa, synthetic construct
  source 1..6: note = Ab8 VH CDR 1 (Ab8 CDR-H1)
IYYSYM

SEQ ID NO: 83 - protein, 10 aa, synthetic construct
  source 1..10: note = Ab8 VH CDR 2 (Ab8 CDR-H2)
SISSSYGYTY

SEQ ID NO: 84 - protein, 15 aa, synthetic construct
  source 1..15: note = Ab8 VH CDR 3 (Ab8 CDR-H3)
TVRGSKKPYFSGWAM

SEQ ID NO: 85 - protein, 213 aa, synthetic construct
  source 1..213: note = Antibody 8 Light chain (Ab8 LC)
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 106 aa, synthetic construct
  source 1..106: note = Antibody 8 Light chain variable region (Ab8 VL)
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIK

SEQ ID NO: 87 - protein, 5 aa, synthetic construct
  source 1..5: note = Ab8 VL CDR 1 (Ab8 CDR-L1)
SVSSA

SEQ ID NO: 88 - protein, 7 aa, synthetic construct
  source 1..7: note = Ab8 VL CDR 2 (Ab8 CDR-L2)
SASSLYS

SEQ ID NO: 89 - protein, 5 aa, synthetic construct
  source 1..5: note = Ab8 VL CDR 3 (Ab8 CDR-L3)
ASYLI

SEQ ID NO: 90 - protein, 450 aa, synthetic construct
  source 1..450: note = Antibody 9 Heavy chain (Ab9 HC)
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 120 aa, synthetic construct
  source 1..120: note = Antibody 9 Heavy chain variable region (Ab9 VH)
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSS

SEQ ID NO: 92 - protein, 6 aa, synthetic construct
  source 1..6: note = Ab9 VH CDR 1 (Ab9 CDR-H1)
LSYSSM

SEQ ID NO: 93 - protein, 10 aa, synthetic construct
  source 1..10: note = Ab9 VH CDR 2 (Ab9 CDR-H2)
SISPYSGSTY

SEQ ID NO: 94 - protein, 9 aa, synthetic construct
  source 1..9: note = Ab9 VH CDR 3 (Ab9 CDR-H3)
YYWYYYYGF

SEQ ID NO: 95 - protein, 216 aa, synthetic construct
  source 1..216: note = Antibody 9 Light chain (Ab9 LC)
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 109 aa, synthetic construct
  source 1..109: note = Antibody 9 Light chain variable region (Ab9 VL)
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIK

SEQ ID NO: 97 - protein, 5 aa, synthetic construct
  source 1..5: note = Ab9 VL CDR 1 (Ab9 CDR-L1)
SVSSA

SEQ ID NO: 98 - protein, 7 aa, synthetic construct
  source 1..7: note = Ab9 VL CDR 2 (Ab9 CDR-L2)
SASSLYS

SEQ ID NO: 99 - protein, 8 aa, synthetic construct
  source 1..8: note = Ab9 VL CDR 3 (Ab9 CDR-L3)
YVGYGSPI

SEQ ID NO: 100 - protein, 456 aa, synthetic construct
  source 1..456: note = Antibody 10 Heavy chain (Ab10 HC)
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 126 aa, synthetic construct
  source 1..126: note = Antibody 10 Heavy chain variable region (Ab10 VH)
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 102 - protein, 6 aa, synthetic construct
  source 1..6: note = Ab10 VH CDR 1 (Ab10 CDR-H1)
LYSSYM

SEQ ID NO: 103 - protein, 10 aa, synthetic construct
  source 1..10: note = Ab10 VH CDR 2 (Ab10 CDR-H2)
SISSSYGYTY

SEQ ID NO: 104 - protein, 15 aa, synthetic construct
  source 1..15: note = Ab10 VH CDR 3 (Ab10 CDR-H3)
TVRGSKKPYFSGWAM

SEQ ID NO: 105 - protein, 213 aa, synthetic construct
  source 1..213: note = Antibody 10 Light chain (Ab10 LC)
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 106 aa, synthetic construct
  source 1..106: note = Antibody 10 Light chain variable region (Ab10 VL)
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIK

SEQ ID NO: 107 - protein, 5 aa, synthetic construct
  source 1..5: note = Ab10 VL CDR 1 (Ab10 CDR-L1)
SVSSA

SEQ ID NO: 108 - protein, 7 aa, synthetic construct
  source 1..7: note = Ab10 VL CDR 2 (Ab10 CDR-L2)
SASSLYS

SEQ ID NO: 109 - protein, 5 aa, synthetic construct
  source 1..5: note = Ab10 VL CDR 3 (Ab10 CDR-L3)
GSYLI

SEQ ID NO: 110 - protein, 5 aa, synthetic construct
  source 1..5: note = Glycine-Serine linker unit (Gly-Gly-Gly-Gly-Ser linker unit, or GS linker unit)
GGGGS

SEQ ID NO: 111 - protein, 15 aa, synthetic construct
  source 1..15: note = (Gly-Gly-Gly-Gly-Ser)X3 linker ((Gly4Ser)3 linker, (G4S)3 linker, G4S X3 linker, or G4SX3 linker)
GGGGSGGGGSGGGGS

SEQ ID NO: 112 - protein, 20 aa, synthetic construct
  source 1..20: note = Lead sequence (LS)
METPAQLLFLLLLWLPDTTG

SEQ ID NO: 113 - protein, 20 aa, synthetic construct
  source 1..20: note = Human CD28 hinge (CD28 hinge, or CD28H)
LEVKGKHLCPSPLFPGPSKP

SEQ ID NO: 114 - protein, 27 aa, synthetic construct
  source 1..27: note = Human CD28 transmembrane domain (CD28 TM domain, or CD28TM)
FWVLVVVGGVLACYSLLVTVAFIIFWV

SEQ ID NO: 115 - protein, 43 aa, synthetic construct
  source 1..43: note = Human CD28 costimulatory domain (CD28 CS domain, or CD28CS)
RSKRSRLLHSDYMNMTPRRPGPTRKHYQPYAPPRDFAAYRSKL

SEQ ID NO: 116 - protein, 42 aa, synthetic construct
  source 1..42: note = Human 4-1BB costimulatory domain (4-1BB CS domain, 41BB CS domain, or 41BBCS)
KRGRKKLLYIFKQPFMRPVQTTQEEDGCSCRFPEEEEGGCEL

SEQ ID NO: 117 - protein, 24 aa, synthetic construct
  source 1..24: note = Human DAP10 costimulatory domain (DAP10 CS domain, or DAP10CS)
LCARPRRSPAQEDGKVYINMPGRG

SEQ ID NO: 118 - protein, 113 aa, synthetic construct
  source 1..113: note = Human CD3 zeta intracellular signaling domain (CD3z ICS domain, CD3 z ICS, or CD3zICS)
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 119 - protein, 28 aa, synthetic construct
  source 1..28: note = T2A ribosomal skip sequence (T2A)
AGAKRSGSGEGRGSLLTCGDVEENPGPR

SEQ ID NO: 120 - protein, 327 aa, synthetic construct
  source 1..327: note = truncated CD19 (trCD19)
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 121-131 - skipped (11 entries)

SEQ ID NO: 132 - DNA, 18 bp, synthetic construct
  source 1..18: mol_type = other DNA; note = Ab3 Heavy chain variable region (VH) CDR 1 (Ab3 CDR-H1)
atctattattcttctatc

SEQ ID NO: 133 - skipped

SEQ ID NO: 134 - DNA, 27 bp, synthetic construct
  source 1..27: mol_type = other DNA; note = Ab3 VH CDR 3 (Ab3 CDR-H3)
tacgtttactggtcttcttacgctttt

SEQ ID NO: 135 - DNA, 648 bp, synthetic construct
  source 1..648: mol_type = other DNA; note = Antibody 3 Light chain (Ab3 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactgggctcatggtttcccgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - DNA, 327 bp, synthetic construct
  source 1..327: mol_type = other DNA; note = Antibody 3 Light chain variable region (Ab3 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactgggctcatggtttcccgatcacgttcggacagggtaccaaggtggagatcaaa

SEQ ID NO: 137 - DNA, 15 bp, synthetic construct
  source 1..15: mol_type = other DNA; note = Ab3 VL CDR 1 (Ab3 CDR-L1)
tccgtgtccagcgct

SEQ ID NO: 138 - DNA, 21 bp, synthetic construct
  source 1..21: mol_type = other DNA; note = Ab3 VL CDR 2 (Ab3 CDR-L2)
tcggcatccagcctctactct

SEQ ID NO: 139 - DNA, 24 bp, synthetic construct
  source 1..24: mol_type = other DNA; note = Ab3 VL CDR 3 (Ab3 CDR-L3)
tactgggctcatggtttcccgatc

SEQ ID NO: 140 - DNA, 1350 bp, synthetic construct
  source 1..1350: mol_type = other DNA; note = Antibody 4 Heavy chain (Ab4 HC)
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 141 - DNA, 360 bp, synthetic construct
  source 1..360: mol_type = other DNA; note = Antibody 4 Heavy chain variable region (Ab4 VH)
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcg

SEQ ID NO: 142 - DNA, 18 bp, synthetic construct
  source 1..18: mol_type = other DNA; note = Ab4 VH CDR 1 (Ab4 CDR-H1)
ctctattattattctatc

SEQ ID NO: 143 - DNA, 30 bp, synthetic construct
  source 1..30: mol_type = other DNA; note = Ab4 VH CDR 2 (Ab4 CDR-H2)
tatatttcttcttattatggctatacttat

SEQ ID NO: 144 - DNA, 27 bp, synthetic construct
  source 1..27: mol_type = other DNA; note = Ab4 VH CDR 3 (Ab4 CDR-H3)
tactacccgggtggttggcatgctttt

SEQ ID NO: 145 - DNA, 648 bp, synthetic construct
  source 1..648: mol_type = other DNA; note = Antibody 4 Light chain (Ab4 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 146 - DNA, 327 bp, synthetic construct
  source 1..327: mol_type = other DNA; note = Antibody 4 Light chain variable region (Ab4 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaa

SEQ ID NO: 147 - DNA, 15 bp, synthetic construct
  source 1..15: mol_type = other DNA; note = Ab4 VL CDR 1 (Ab4 CDR-L1)
tccgtgtccagcgct

SEQ ID NO: 148 - DNA, 21 bp, synthetic construct
  source 1..21: mol_type = other DNA; note = Ab4 VL CDR 2 (Ab4 CDR-L2)
tcggcatccagcctctactct

SEQ ID NO: 149 - DNA, 24 bp, synthetic construct
  source 1..24: mol_type = other DNA; note = Ab4 VL CDR 3 (Ab4 CDR-L3)
tactacggttacggttacccgatc

SEQ ID NO: 150 - DNA, 1368 bp, synthetic construct
  source 1..1368: mol_type = other DNA; note = Antibody 5 Heavy chain (Ab5 HC)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 151 - DNA, 378 bp, synthetic construct
  source 1..378: mol_type = other DNA; note = Antibody 5 Heavy chain variable region (Ab5 VH)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 152 - DNA, 18 bp, synthetic construct
  source 1..18: mol_type = other DNA; note = Ab5 VH CDR 1 (Ab5 CDR-H1)
ctctattcttcttatatg

SEQ ID NO: 153 - DNA, 30 bp, synthetic construct
  source 1..30: mol_type = other DNA; note = Ab5 VH CDR 2 (Ab5 CDR-H2)
tctatttattcttcttatagctctacttat

SEQ ID NO: 154 - DNA, 45 bp, synthetic construct
  source 1..45: mol_type = other DNA; note = Ab5 VH CDR 3 (Ab5 CDR-H3)
actgttcgtggatccaaaaaaccgtacttctctggttgggctatg

SEQ ID NO: 155 - DNA, 639 bp, synthetic construct
  source 1..639: mol_type = other DNA; note = Antibody 5 Light chain (Ab5 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 156 - DNA, 318 bp, synthetic construct
  source 1..318: mol_type = other DNA; note = Antibody 5 Light chain variable region (Ab5 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaa

SEQ ID NO: 157 - DNA, 15 bp, synthetic construct
  source 1..15: mol_type = other DNA; note = Ab5 VL CDR 1 (Ab5 CDR-L1)
tccgtgtccagcgct

SEQ ID NO: 158 - DNA, 21 bp, synthetic construct
  source 1..21: mol_type = other DNA; note = Ab5 VL CDR 2 (Ab5 CDR-L2)
tcggcatccagcctctactct

SEQ ID NO: 159 - DNA, 15 bp, synthetic construct
  source 1..15: mol_type = other DNA; note = Ab5 VL CDR 3 (Ab5 CDR-L3)
ggtgctggtctgatc

SEQ ID NO: 160 - DNA, 1368 bp, synthetic construct
  source 1..1368: mol_type = other DNA; note = Antibody 6 Heavy chain (Ab6 HC)
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - DNA, 378 bp, synthetic construct
  source 1..378: mol_type = other DNA; note = Antibody 6 Heavy chain variable region (Ab6 VH)
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 162 - DNA, 18 bp, synthetic construct
  source 1..18: mol_type = other DNA; note = Ab6 VH CDR 1 (Ab6 CDR-H1)
atctattcttcttatatc

SEQ ID NO: 163 - DNA, 30 bp, synthetic construct
  source 1..30: mol_type = other DNA; note = Ab6 VH CDR 2 (Ab6 CDR-H2)
tctatttcttcttcttatggctatacttat

SEQ ID NO: 164 - DNA, 45 bp, synthetic construct; identical to SEQ ID NO: 154
  source 1..45: mol_type = other DNA; note = Ab6 VH CDR 3 (Ab6 CDR-H3)

SEQ ID NO: 165 - DNA, 639 bp, synthetic construct
  source 1..639: mol_type = other DNA; note = Antibody 6 Light chain (Ab6 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 166 - DNA, 318 bp, synthetic construct
  source 1..318: mol_type = other DNA; note = Antibody 6 Light chain variable region (Ab6 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaa

SEQ ID NO: 167 - DNA, 15 bp, synthetic construct
  source 1..15: mol_type = other DNA; note = Ab6 VL CDR 1 (Ab6 CDR-L1)
tccgtgtccagcgct

SEQ ID NO: 168 - DNA, 21 bp, synthetic construct
  source 1..21: mol_type = other DNA; note = Ab6 VL CDR 2 (Ab6 CDR-L2)
tcggcatccagcctctactct

SEQ ID NO: 169 - DNA, 15 bp, synthetic construct
  source 1..15: mol_type = other DNA; note = Ab6 VL CDR 3 (Ab6 CDR-L3)
ggtggttctctgatc

SEQ ID NO: 170 - DNA, 1368 bp, synthetic construct
  source 1..1368: mol_type = other DNA; note = Antibody 7 Heavy chain (Ab7 HC)
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 171 - DNA, 378 bp, synthetic construct
  source 1..378: mol_type = other DNA; note = Antibody 7 Heavy chain variable region (Ab7 VH)
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 172 - DNA, 18 bp, synthetic construct
  source 1..18: mol_type = other DNA; note = Ab7 VH CDR 1 (Ab7 CDR-H1)
ttttcttcttcttctata

SEQ ID NO: 173 - DNA, 30 bp, synthetic construct; identical to SEQ ID NO: 163
  source 1..30: mol_type = other DNA; note = Ab7 VH CDR 2 (Ab7 CDR-H2)

SEQ ID NO: 174 - DNA, 45 bp, synthetic construct; identical to SEQ ID NO: 154
  source 1..45: mol_type = other DNA; note = Ab7 VH CDR 3 (Ab7 CDR-H3)

SEQ ID NO: 175 - DNA, 639 bp, synthetic construct
  source 1..639: mol_type = other DNA; note = Antibody 7 Light chain (Ab7 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 176 - DNA, 318 bp, synthetic construct
  source 1..318: mol_type = other DNA; note = Antibody 7 Light chain variable region (Ab7 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaa

SEQ ID NO: 177 - DNA, 15 bp, synthetic construct
  source 1..15: mol_type = other DNA; note = Ab7 VL CDR 1 (Ab7 CDR-L1)
tccgtgtccagcgct

SEQ ID NO: 178 - DNA, 21 bp, synthetic construct
  source 1..21: mol_type = other DNA; note = Ab7 VL CDR 2 (Ab7 CDR-L2)
tcggcatccagcctctactct

SEQ ID NO: 179 - DNA, 15 bp, synthetic construct
  source 1..15: mol_type = other DNA; note = Ab7 VL CDR 3 (Ab7 CDR-L3)
ggttctcatctgatc

SEQ ID NO: 180 - DNA, 1368 bp, synthetic construct
  source 1..1368: mol_type = other DNA; note = Antibody 8 Heavy chain (Ab8 HC)
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 181 - DNA, 378 bp, synthetic construct
  source 1..378: mol_type = other DNA; note = Antibody 8 Heavy chain variable region (Ab8 VH)
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 182 - DNA, 18 bp, synthetic construct
  source 1..18: mol_type = other DNA; note = Ab8 VH CDR 1 (Ab8 CDR-H1)
atctattattcttatatg

SEQ ID NO: 183 - DNA, 30 bp, synthetic construct; identical to SEQ ID NO: 163
  source 1..30: mol_type = other DNA; note = Ab8 VH CDR 2 (Ab8 CDR-H2)

SEQ ID NO: 184 - DNA, 45 bp, synthetic construct; identical to SEQ ID NO: 154
  source 1..45: mol_type = other DNA; note = Ab8 VH CDR 3 (Ab8 CDR-H3)

SEQ ID NO: 185 - DNA, 639 bp, synthetic construct
  source 1..639: mol_type = other DNA; note = Antibody 8 Light chain (Ab8 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 186 - DNA, 318 bp, synthetic construct
  source 1..318: mol_type = other DNA; note = Antibody 8 Light chain variable region (Ab8 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaa

SEQ ID NO: 187 - DNA, 15 bp, synthetic construct
  source 1..15: mol_type = other DNA; note = Ab8 VL CDR 1 (Ab8 CDR-L1)
tccgtgtccagcgct

SEQ ID NO: 188 - DNA, 21 bp, synthetic construct
  source 1..21: mol_type = other DNA; note = Ab8 VL CDR 2 (Ab8 CDR-L2)
tcggcatccagcctctactct

SEQ ID NO: 189 - DNA, 15 bp, synthetic construct
  source 1..15: mol_type = other DNA; note = Ab8 VL CDR 3 (Ab8 CDR-L3)
gcttcttacctgatc

SEQ ID NO: 190 - DNA, 1350 bp, synthetic construct
  source 1..1350: mol_type = other DNA; note = Antibody 9 Heavy chain (Ab9 HC)
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - DNA, 360 bp, synthetic construct
  source 1..360: mol_type = other DNA; note = Antibody 9 Heavy chain variable region (Ab9 VH)
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcg

SEQ ID NO: 192 - DNA, 18 bp, synthetic construct
  source 1..18: mol_type = other DNA; note = Ab9 VH CDR 1 (Ab9 CDR-H1)
ctctcttattcttctatg

SEQ ID NO: 193 - DNA, 30 bp, synthetic construct
  source 1..30: mol_type = other DNA; note = Ab9 VH CDR 2 (Ab9 CDR-H2)
tctatttctccttattctggctctacttat

SEQ ID NO: 194 - DNA, 27 bp, synthetic construct
  source 1..27: mol_type = other DNA; note = Ab9 VH CDR 3 (Ab9 CDR-H3)
tactactggtactactactacggtttt

SEQ ID NO: 195 - DNA, 648 bp, synthetic construct
  source 1..648: mol_type = other DNA; note = Antibody 9 Light chain (Ab9 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 196 - DNA, 327 bp, synthetic construct
  source 1..327: mol_type = other DNA; note = Antibody 9 Light chain variable region (Ab9 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaa

SEQ ID NO: 197 - DNA, 15 bp, synthetic construct
  source 1..15: mol_type = other DNA; note = Ab9 VL CDR 1 (Ab9 CDR-L1)
tccgtgtccagcgct

SEQ ID NO: 198 - DNA, 21 bp, synthetic construct
  source 1..21: mol_type = other DNA; note = Ab9 VL CDR 2 (Ab9 CDR-L2)
tcggcatccagcctctactct

SEQ ID NO: 199 - DNA, 24 bp, synthetic construct
  source 1..24: mol_type = other DNA; note = Ab9 VL CDR 3 (Ab9 CDR-L3)
tacgttggttacggttctccgatc

SEQ ID NO: 200 - DNA, 1368 bp, synthetic construct
  source 1..1368: mol_type = other DNA; note = Antibody 10 Heavy chain (Ab10 HC)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 201 - DNA, 378 bp, synthetic construct
  source 1..378: mol_type = other DNA; note = Antibody 10 Heavy chain variable region (Ab10 VH)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 202 - DNA, 18 bp, synthetic construct
  source 1..18: mol_type = other DNA; note = Ab10 VH CDR 1 (Ab10 CDR-H1)
ctctattcttcttatatg

SEQ ID NO: 203 - DNA, 30 bp, synthetic construct; identical to SEQ ID NO: 163
  source 1..30: mol_type = other DNA; note = Ab10 VH CDR 2 (Ab10 CDR-H2)

SEQ ID NO: 204 - DNA, 45 bp, synthetic construct; identical to SEQ ID NO: 154
  source 1..45: mol_type = other DNA; note = Ab10 VH CDR 3 (Ab10 CDR-H3)

SEQ ID NO: 205 - DNA, 639 bp, synthetic construct
  source 1..639: mol_type = other DNA; note = Antibody 10 Light chain (Ab10 LC)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 206 - DNA, 318 bp, synthetic construct
  source 1..318: mol_type = other DNA; note = Antibody 10 Light chain variable region (Ab10 VL)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaa

SEQ ID NO: 207 - DNA, 15 bp, synthetic construct
  source 1..15: mol_type = other DNA; note = Ab10 VL CDR 1 (Ab10 CDR-L1)
tccgtgtccagcgct

SEQ ID NO: 208 - DNA, 21 bp, synthetic construct
  source 1..21: mol_type = other DNA; note = Ab10 VL CDR 2 (Ab10 CDR-L2)
tcggcatccagcctctactct

SEQ ID NO: 209 - DNA, 15 bp, synthetic construct
  source 1..15: mol_type = other DNA; note = Ab10 VL CDR 3 (Ab10 CDR-L3)
ggttcttacctgatc

SEQ ID NO: 210 - DNA, 15 bp, synthetic construct
  source 1..15: mol_type = other DNA; note = Glycine-Serine linker unit (Gly-Gly-Gly-Gly-Ser linker unit, or GS linker unit)
ggtggtggtggttct

SEQ ID NO: 211 - DNA, 45 bp, synthetic construct
  source 1..45: mol_type = other DNA; note = (Gly-Gly-Gly-Gly-Ser)X3 linker ((Gly4Ser)3 linker, (G4S)3 linker, G4S X3 linker, or G4SX3 linker)
ggtggtggtggttctggcggcggcggctccggtggtggtggttcc

SEQ ID NO: 212 - DNA, 60 bp, synthetic construct
  source 1..60: mol_type = other DNA; note = Lead sequence (LS)
atggaaaccccagcgcagcttctcttcctcctgctactctggctcccagataccaccgga

SEQ ID NO: 213 - DNA, 60 bp, synthetic construct
  source 1..60: mol_type = other DNA; note = Human CD28 hinge (CD28 hinge, or CD28H)
ctcgaggtgaaagggaaacacctttgtccaagtcccctatttcccggaccttctaagccc

SEQ ID NO: 214 - DNA, 81 bp, synthetic construct
  source 1..81: mol_type = other DNA; note = Human CD28 transmembrane domain (CD28 TM domain, or CD28TM)
ttttgggtgctggtggtggttggtggagtcctggcttgctatagcttgctagtaacagtggcctttattattttctgggtg

SEQ ID NO: 215 - DNA, 129 bp, synthetic construct
  source 1..129: mol_type = other DNA; note = Human CD28 costimulatory domain (CD28 CS domain, or CD28CS)
aggagtaagaggagcaggctcctgcacagtgactacatgaacatgactccccgccgccccgggcccacccgcaagcattaccagccctatgccccaccacgcgacttcgcagcctatcgctccaagctt

SEQ ID NO: 216 - DNA, 126 bp, synthetic construct
  source 1..126: mol_type = other DNA; note = Human 4-1BB costimulatory domain (4-1BB CS domain, 41BB CS domain, or 41BBCS)
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 217 - DNA, 72 bp, synthetic construct
  source 1..72: mol_type = other DNA; note = Human DAP10 costimulatory domain (DAP10 CS domain, or DAP10CS)
ctgtgcgcacgcccacgccgcagccccgcccaagaagatggcaaagtctacatcaacatgccaggcaggggc

SEQ ID NO: 218 - DNA, 339 bp, synthetic construct
  source 1..339: mol_type = other DNA; note = Human CD3 zeta intracellular signaling domain (CD3z ICS domain, CD3z ICS, or CD3zICS)
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 219 - DNA, 84 bp, synthetic construct
  source 1..84: mol_type = other DNA; note = T2A ribosomal skip sequence (T2A)
gccggcgccaaaaggtctggctccggtgagggcagaggaagtcttctaacatgcggtgacgtggaggagaatcccggccctaga

SEQ ID NO: 220 - DNA, 984 bp, synthetic construct
  source 1..984: mol_type = other DNA; note = truncated CD19 (trCD19)
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 221-239 - skipped (19 entries)

SEQ ID NO: 240 - protein, 244 aa, synthetic construct
  source 1..244: note = Antibody 4 scFv VH-G4S3-VL (Ab4 scFv HL or Ab4scFvHL)
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIK

SEQ ID NO: 241 - protein, 447 aa, synthetic construct
  source 1..447: note = Ab4scFvHL-CD28H-CD28TM-CD28CS-CD3zICS
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 242 - protein, 446 aa, synthetic construct
  source 1..446: note = Ab4scFvHL-CD28H-CD28TM-41BBCS-CD3zICS
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 243 - protein, 428 aa, synthetic construct
  source 1..428: note = Ab4scFvHL-CD28H-CD28TM-DAP10CS-CD3zICS
EVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 244 - skipped

SEQ ID NO: 245 - protein, 242 aa, synthetic construct
  source 1..242: note = Antibody 4 scFv VL-G4S3-VH (Ab4 scFv LH or Ab4scFvLH)
QMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKGGGGSGGGGSGGGGSEVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSS

SEQ ID NO: 246 - protein, 445 aa, synthetic construct
  source 1..445: note = Ab4scFvLH-CD28H-CD28TM-CD28CS-CD3zICS
QMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKGGGGSGGGGSGGGGSEVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 247 - protein, 444 aa, synthetic construct
  source 1..444: note = Ab4scFvLH-CD28H-CD28TM-41BBCS-CD3zICS
QMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKGGGGSGGGGSGGGGSEVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 248 - protein, 426 aa, synthetic construct
  source 1..426: note = Ab4scFvLH-CD28H-CD28TM-DAP10CS-CD3zICS
QMTQSPSSLSASVGDRVTITCRASQSVSSAVAWYQQKPGKAPKLLIYSASSLYSGVPSRFSGSRSGTDFTLTISSLQPEDFATYYCQQYYGYGYPITFGQGTKVEIKGGGGSGGGGSGGGGSEVQLVESGGGLVQPGGSLRLSCAASGFNLYYYSIHWVRQAPGKGLEWVAYISSYYGYTYYADSVKGRFTISADTSKNTAYLQMNSLRAEDTAVYYCARYYPGGWHAFDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 249 - skipped

SEQ ID NO: 250 - protein, 247 aa, synthetic construct
  source 1..247: note = Antibody 5 scFv VH-G4S3-VL (Ab5 scFv HL or Ab5scFvHL)
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIK

SEQ ID NO: 251 - protein, 450 aa, synthetic construct
  source 1..450: note = Ab5scFvHL-CD28H-CD28TM-CD28CS-CD3zICS
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 252 - protein, 449 aa, synthetic construct
  source 1..449: note = Ab5scFvHL-CD28H-CD28TM-41BBCS-CD3zICS
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 253 - protein, 431 aa, synthetic construct
  source 1..431: note = Ab5scFvHL-CD28H-CD28TM-DAP10CS-CD3zICS
EVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 254 - skipped

SEQ ID NO: 255 - protein, 247 aa, synthetic construct
  source 1..247: note = Antibody 5 scFv VL-G4S3-VH (Ab5 scFv LH or Ab5scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKGGGGSGGGGSGGGGSEVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSS

SEQ ID NO: 256 - protein, 450 aa, synthetic construct
  source 1..450: note = Ab5scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKGGGGSGGGGSGGGGSEVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 257 - protein, 449 aa, synthetic construct
  source 1..449: note = Ab5scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKGGGGSGGGGSGGGGSEVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 258 - protein, 431 aa, synthetic construct
  source 1..431: note = Ab5scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGAGLITFGQGTKVEIKGGGGSGGGGSGGGGSEVQLVESGGGLVQPGGSLRLSCAASGFNLYSSYMHWVRQAPGKGLEWVASIYSSYSSTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 259 - skipped

SEQ ID NO: 260 - protein, 247 aa, synthetic construct
  source 1..247: note = Antibody 6 scFv VH-G4S3-VL (Ab6 scFv HL or Ab6scFvHL)
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIK

SEQ ID NO: 261 - protein, 450 aa, synthetic construct
  source 1..450: note = Ab6scFvHL-CD28H-CD28TM-CD28CS-CD3zICS
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 262 - protein, 449 aa, synthetic construct
  source 1..449: note = Ab6scFvHL-CD28H-CD28TM-41BBCS-CD3zICS
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 263 - protein, 431 aa, synthetic construct
  source 1..431: note = Ab6scFvHL-CD28H-CD28TM-DAP10CS-CD3zICS
EVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 264 - skipped

SEQ ID NO: 265 - protein, 247 aa, synthetic construct
  source 1..247: note = Antibody 6 scFv VL-G4S3-VH (Ab6 scFv LH or Ab6scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKGGGGSGGGGSGGGGSEVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 266 - protein, 450 aa, synthetic construct
  source 1..450: note = Ab6scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKGGGGSGGGGSGGGGSEVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 267 - protein, 449 aa, synthetic construct
  source 1..449: note = Ab6scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKGGGGSGGGGSGGGGSEVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 268 - protein, 431 aa, synthetic construct
  source 1..431: note = Ab6scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGGSLITFGQGTKVEIKGGGGSGGGGSGGGGSEVQLVESGGGLVQPGGSLRLSCAASGFNIYSSYIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 269 - skipped

SEQ ID NO: 270 - protein, 247 aa, synthetic construct
  source 1..247: note = Antibody 7 scFv VH-G4S3-VL (Ab7 scFv HL or Ab7scFvHL)
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIK

SEQ ID NO: 271 - protein, 450 aa, synthetic construct
  source 1..450: note = Ab7scFvHL-CD28H-CD28TM-CD28CS-CD3zICS
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 272 - protein, 449 aa, synthetic construct
  source 1..449: note = Ab7scFvHL-CD28H-CD28TM-41BBCS-CD3zICS
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 273 - protein, 431 aa, synthetic construct
  source 1..431: note = Ab7scFvHL-CD28H-CD28TM-DAP10CS-CD3zICS
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 274 - skipped

SEQ ID NO: 275 - protein, 247 aa, synthetic construct
  source 1..247: note = Antibody 7 scFv VL-G4S3-VH (Ab7 scFv LH or Ab7scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKGGGGSGGGGSGGGGSEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 276 - protein, 450 aa, synthetic construct
  source 1..450: note = Ab7scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKGGGGSGGGGSGGGGSEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 277 - protein, 449 aa, synthetic construct
  source 1..449: note = Ab7scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKGGGGSGGGGSGGGGSEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 278 - protein, 431 aa, synthetic construct
  source 1..431: note = Ab7scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSHLITFGQGTKVEIKGGGGSGGGGSGGGGSEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 279 - skipped

SEQ ID NO: 280 - protein, 247 aa, synthetic construct
  source 1..247: note = Antibody 8 scFv VH-G4S3-VL (Ab8 scFv HL or Ab8scFvHL)
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIK

SEQ ID NO: 281 - protein, 450 aa, synthetic construct
  source 1..450: note = Ab8scFvHL-CD28H-CD28TM-CD28CS-CD3zICS
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 282 - protein, 449 aa, synthetic construct
  source 1..449: note = Ab8scFvHL-CD28H-CD28TM-41BBCS-CD3zICS
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 283 - protein, 431 aa, synthetic construct
  source 1..431: note = Ab8scFvHL-CD28H-CD28TM-DAP10CS-CD3zICS
EVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 284 - protein, 825 aa, synthetic construct
  source 1..825: note = LS-Ab8scFvHL-CD28H-CD28TM-CD28CS-CD3zICS-T2A-trCD19
METPAQLLFLLLLWLPDTTGEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 285 - protein, 247 aa, synthetic construct
  source 1..247: note = Antibody 8 scFv VL-G4S3-VH (Ab8 scFv LH or Ab8scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKGGGGSGGGGSGGGGSEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 286 - protein, 450 aa, synthetic construct
  source 1..450: note = Ab8scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKGGGGSGGGGSGGGGSEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 287 - protein, 449 aa, synthetic construct
  source 1..449: note = Ab8scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKGGGGSGGGGSGGGGSEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 288 - protein, 431 aa, synthetic construct
  source 1..431: note = Ab8scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKGGGGSGGGGSGGGGSEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 289 - protein, 825 aa, synthetic construct
  source 1..825: note = LS-Ab8scFvLH-CD28H-CD28TM-CD28CS-CD3zICS-T2A-trCD19
METPAQLLFLLLLWLPDTTGDIQMTQSPSSLSASVGDRVTITCRASQSVSSAVAWYQQKPGKAPKLLIYSASSLYSGVPSRFSGSRSGTDFTLTISSLQPEDFATYYCQQASYLITFGQGTKVEIKGGGGSGGGGSGGGGSEVQLVESGGGLVQPGGSLRLSCAASGFNIYYSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 290 - protein, 244 aa, synthetic construct
  source 1..244: note = Antibody 9 scFv VH-G4S3-VL (Ab9 scFv HL or Ab9scFvHL)
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIK

SEQ ID NO: 291 - protein, 447 aa, synthetic construct
  source 1..447: note = Ab9scFvHL-CD28H-CD28TM-CD28CS-CD3zICS
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 292 - protein, 446 aa, synthetic construct
  source 1..446: note = Ab9scFvHL-CD28H-CD28TM-41BBCS-CD3zICS
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 293 - protein, 428 aa, synthetic construct
  source 1..428: note = Ab9scFvHL-CD28H-CD28TM-DAP10CS-CD3zICS
EVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 294 - protein, 822 aa, synthetic construct
  source 1..822: note = LS-Ab9scFvHL-CD28H-CD28TM-CD28CS-CD3zICS-T2A-trCD19
METPAQLLFLLLLWLPDTTGEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 295 - protein, 244 aa, synthetic construct
  source 1..244: note = Antibody 9 scFv VL-G4S3-VH (Ab9 scFv LH or Ab9scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKGGGGSGGGGSGGGGSEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSS

SEQ ID NO: 296 - protein, 447 aa, synthetic construct
  source 1..447: note = Ab9scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKGGGGSGGGGSGGGGSEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 297 - protein, 446 aa, synthetic construct
  source 1..446: note = Ab9scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKGGGGSGGGGSGGGGSEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 298 - protein, 428 aa, synthetic construct
  source 1..428: note = Ab9scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKGGGGSGGGGSGGGGSEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 299 - protein, 822 aa, synthetic construct
  source 1..822: note = LS-Ab9scFvLH-CD28H-CD28TM-CD28CS-CD3zICS-T2A-trCD19
METPAQLLFLLLLWLPDTTGDIQMTQSPSSLSASVGDRVTITCRASQSVSSAVAWYQQKPGKAPKLLIYSASSLYSGVPSRFSGSRSGTDFTLTISSLQPEDFATYYCQQYVGYGSPITFGQGTKVEIKGGGGSGGGGSGGGGSEVQLVESGGGLVQPGGSLRLSCAASGFNLSYSSMHWVRQAPGKGLEWVASISPYSGSTYYADSVKGRFTISADTSKNTAYLQMNSLRAEDTAVYYCARYYWYYYYGFDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 300 - protein, 247 aa, synthetic construct
  source 1..247: note = Antibody 10 scFv VH-G4S3-VL (Ab10 scFv HL or Ab10scFvHL)
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIK

SEQ ID NO: 301 - protein, 450 aa, synthetic construct
  source 1..450: note = Ab10scFvHL-CD28H-CD28TM-CD28CS-CD3zICS
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 302 - protein, 449 aa, synthetic construct
  source 1..449: note = Ab10scFvHL-CD28H-CD28TM-41BBCS-CD3zICS
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 303 - protein, 431 aa, synthetic construct
  source 1..431: note = Ab10scFvHL-CD28H-CD28TM-DAP10CS-CD3zICS
EVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 304 - skipped

SEQ ID NO: 305 - protein, 247 aa, synthetic construct
  source 1..247: note = Antibody 10 scFv VL-G4S3-VH (Ab10 scFv LH or Ab10scFvLH)
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKGGGGSGGGGSGGGGSEVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 306 - protein, 450 aa, synthetic construct
  source 1..450: note = Ab9scFvLH-CD28H-CD28TM-CD28CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKGGGGSGGGGSGGGGSEVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 307 - protein, 449 aa, synthetic construct
  source 1..449: note = Ab10scFvLH-CD28H-CD28TM-41BBCS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKGGGGSGGGGSGGGGSEVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 308 - protein, 431 aa, synthetic construct
  source 1..431: note = Ab10scFvLH-CD28H-CD28TM-DAP10CS-CD3zICS
DIQMTQSPSSLSASVGDRVTITCRASQSVSSAVAWYQQKPGKAPKLLIYSASSLYSGVPSRFSGSRSGTDFTLTISSLQPEDFATYYCQQGSYLITFGQGTKVEIKGGGGSGGGGSGGGGSEVQLVESGGGLVQPGGSLRLSCAASGFNLYSSYMHWVRQAPGKGLEWVASISSSYGYTYYADSVKGRFTISADTSKNTAYLQMNSLRAEDTAVYYCARTVRGSKKPYFSGWAMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 309 - skipped

SEQ ID NO: 310 - protein, 98 aa, synthetic construct
  source 1..98: note = Human IgG1 CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 311 - protein, 15 aa, synthetic construct
  source 1..15: note = Human IgG1 hinge
EPKSCDKTHTCPPCP

SEQ ID NO: 312 - protein, 110 aa, synthetic construct
  source 1..110: note = Human IgG1 CH2 domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 313 - protein, 107 aa, synthetic construct
  source 1..107: note = Human IgG1 CH3 domain (356E and 358M; with C-terminal K)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 106 aa, synthetic construct
  source 1..106: note = Human IgG1 CH3 domain (356E and 358M; without C-terminal K)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 315 - protein, 107 aa, synthetic construct
  source 1..107: note = Human IgG1 CH3 domain (356D and 358L; with C-terminal K)
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 106 aa, synthetic construct [sequence omitted]

SEQ ID NO: 317 - protein, 217 aa, synthetic construct
  source 1..217: note = Human IgG1 Fc (356E and 358M; with C-terminal K)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 216 aa, synthetic construct
  source 1..216: note = Human IgG1 Fc (356E and 358M; without C-terminal K)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 319 - protein, 217 aa, synthetic construct
  source 1..217: note = Human IgG1 Fc (356D and 358L; with C-terminal K)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 216 aa, synthetic construct
  source 1..216: note = Human IgG1 Fc (356D and 358L; without C-terminal K)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 330 aa, synthetic construct
  source 1..330: note = Human IgG1 heavy chain constant region (356E and 358M; with C-terminal K)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 329 aa, synthetic construct
  source 1..329: note = Human IgG1 heavy chain constant region (356E and 358M; without C-terminal K)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 330 aa, synthetic construct [sequence omitted]

SEQ ID NO: 324 - protein, 329 aa, synthetic construct
  source 1..329: note = Human IgG1 heavy chain constant region (356D and 358L; without C-terminal K)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 107 aa, synthetic construct [sequence omitted]

SEQ ID NO: 326 - protein, 106 aa, synthetic construct
  source 1..106: note = Human Ig lambda light chain constant domain
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 327-339 - skipped (13 entries)

SEQ ID NO: 340 - DNA, 732 bp, synthetic construct
  source 1..732: mol_type = other DNA; note = Antibody 4 scFv VH-G4S3-VL (Ab4 scFv HL or Ab4scFvHL)
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaa

SEQ ID NO: 341 - DNA, 1341 bp, synthetic construct
  source 1..1341: mol_type = other DNA; note = Ab4scFvHL-CD28H-CD28TM-CD28CS-CD3zICS
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 342 - DNA, 1338 bp, synthetic construct
  source 1..1338: mol_type = other DNA; note = Ab4scFvHL-CD28H-CD28TM-41BBCS-CD3zICS
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 343 - DNA, 1284 bp, synthetic construct
  source 1..1284: mol_type = other DNA; note = Ab4scFvHL-CD28H-CD28TM-DAP10CS-CD3zICS
gaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 344 - skipped

SEQ ID NO: 345 - DNA, 732 bp, synthetic construct
  source 1..732: mol_type = other DNA; note = Antibody 4 scFv VL-G4S3-VH (Ab4 scFv LH or Ab4scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcg

SEQ ID NO: 346 - DNA, 1341 bp, synthetic construct
  source 1..1341: mol_type = other DNA; note = Ab4scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 347 - DNA, 1338 bp, synthetic construct
  source 1..1338: mol_type = other DNA; note = Ab4scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 348 - DNA, 1284 bp, synthetic construct
  source 1..1284: mol_type = other DNA; note = Ab4scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactacggttacggttacccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattattattctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgctactacccgggtggttggcatgcttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 349 - skipped

SEQ ID NO: 350 - DNA, 741 bp, synthetic construct
  source 1..741: mol_type = other DNA; note = Antibody 5 scFv VH-G4S3-VL (Ab5 scFv HL or Ab5scFvHL)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaa

SEQ ID NO: 351 - DNA, 1350 bp, synthetic construct
  source 1..1350: mol_type = other DNA; note = Ab5scFvHL-CD28H-CD28TM-CD28CS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 352 - DNA, 1347 bp, synthetic construct
  source 1..1347: mol_type = other DNA; note = Ab5scFvHL-CD28H-CD28TM-41BBCS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 353 - DNA, 1293 bp, synthetic construct
  source 1..1293: mol_type = other DNA; note = Ab5scFvHL-CD28H-CD28TM-DAP10CS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 354 - skipped

SEQ ID NO: 355 - DNA, 741 bp, synthetic construct
  source 1..741: mol_type = other DNA; note = Antibody 5 scFv VL-G4S3-VH (Ab5 scFv LH or Ab5scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 356 - DNA, 1350 bp, synthetic construct
  source 1..1350: mol_type = other DNA; note = Ab5scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 357 - DNA, 1347 bp, synthetic construct
  source 1..1347: mol_type = other DNA; note = Ab5scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 358 - DNA, 1293 bp, synthetic construct
  source 1..1293: mol_type = other DNA; note = Ab5scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgctggtctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttattcttcttatagctctacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 359 - skipped

SEQ ID NO: 360 - DNA, 741 bp, synthetic construct
  source 1..741: mol_type = other DNA; note = Antibody 6 scFv VH-G4S3-VL (Ab6 scFv HL or Ab6scFvHL)
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaa

SEQ ID NO: 361 - DNA, 1350 bp, synthetic construct
  source 1..1350: mol_type = other DNA; note = Ab6scFvHL-CD28H-CD28TM-CD28CS-CD3zICS
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 362 - DNA, 1347 bp, synthetic construct
  source 1..1347: mol_type = other DNA; note = Ab6scFvHL-CD28H-CD28TM-41BBCS-CD3zICS
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 363 - DNA, 1293 bp, synthetic construct
  source 1..1293: mol_type = other DNA; note = Ab6scFvHL-CD28H-CD28TM-DAP10CS-CD3zICS
gaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 364 - skipped

SEQ ID NO: 365 - DNA, 741 bp, synthetic construct
  source 1..741: mol_type = other DNA; note = Antibody 6 scFv VL-G4S3-VH (Ab6 scFv LH or Ab6scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 366 - DNA, 1350 bp, synthetic construct
  source 1..1350: mol_type = other DNA; note = Ab6scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 367 - DNA, 1347 bp, synthetic construct
  source 1..1347: mol_type = other DNA; note = Ab6scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 368 - DNA, 1293 bp, synthetic construct
  source 1..1293: mol_type = other DNA; note = Ab6scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttctctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattcttcttatatccactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 369 - skipped

SEQ ID NO: 370 - DNA, 741 bp, synthetic construct
  source 1..741: mol_type = other DNA; note = Antibody 7 scFv VH-G4S3-VL (Ab7 scFv HL or Ab7scFvHL)
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaa

SEQ ID NO: 371 - DNA, 1350 bp, synthetic construct
  source 1..1350: mol_type = other DNA; note = Ab7scFvHL-CD28H-CD28TM-CD28CS-CD3zICS
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 372 - DNA, 1347 bp, synthetic construct
  source 1..1347: mol_type = other DNA; note = Ab7scFvHL-CD28H-CD28TM-41BBCS-CD3zICS
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 373 - DNA, 1293 bp, synthetic construct
  source 1..1293: mol_type = other DNA; note = Ab7scFvHL-CD28H-CD28TM-DAP10CS-CD3zICS
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 374 - skipped

SEQ ID NO: 375 - DNA, 741 bp, synthetic construct
  source 1..741: mol_type = other DNA; note = Antibody 7 scFv VL-G4S3-VH (Ab7 scFv LH or Ab7scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 376 - DNA, 1350 bp, synthetic construct
  source 1..1350: mol_type = other DNA; note = Ab7scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 377 - DNA, 1347 bp, synthetic construct
  source 1..1347: mol_type = other DNA; note = Ab7scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 378 - DNA, 1293 bp, synthetic construct
  source 1..1293: mol_type = other DNA; note = Ab7scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttctcatctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 379 - skipped

SEQ ID NO: 380 - DNA, 741 bp, synthetic construct
  source 1..741: mol_type = other DNA; note = Antibody 8 scFv VH-G4S3-VL (Ab8 scFv HL or Ab8scFvHL)
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaa

SEQ ID NO: 381 - DNA, 1350 bp, synthetic construct
  source 1..1350: mol_type = other DNA; note = Ab8scFvHL-CD28H-CD28TM-CD28CS-CD3zICS
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 382 - DNA, 1347 bp, synthetic construct
  source 1..1347: mol_type = other DNA; note = Ab8scFvHL-CD28H-CD28TM-41BBCS-CD3zICS
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 383 - DNA, 1293 bp, synthetic construct
  source 1..1293: mol_type = other DNA; note = Ab8scFvHL-CD28H-CD28TM-DAP10CS-CD3zICS
gaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 384 - DNA, 2478 bp, synthetic construct
  source 1..2478: mol_type = other DNA; note = LS-Ab8scFvHL-CD28H-CD28TM-CD28CS-CD3zICS-T2A-trCD19
atggaaaccccagcgcagcttctcttcctcctgctactctggctcccagataccaccggagaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 385 - DNA, 741 bp, synthetic construct
  source 1..741: mol_type = other DNA; note = Antibody 8 scFv VL-G4S3-VH (Ab8 scFv LH or Ab8scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 386 - DNA, 1350 bp, synthetic construct
  source 1..1350: mol_type = other DNA; note = Ab8scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 387 - DNA, 1347 bp, synthetic construct
  source 1..1347: mol_type = other DNA; note = Ab8scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 388 - DNA, 1293 bp, synthetic construct
  source 1..1293: mol_type = other DNA; note = Ab8scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 389 - DNA, 2478 bp, synthetic construct
  source 1..2478: mol_type = other DNA; note = LS-Ab8scFvLH-CD28H-CD28TM-CD28CS-CD3zICS-T2A-trCD19
atggaaaccccagcgcagcttctcttcctcctgctactctggctcccagataccaccggagatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacatctattattcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 390 - DNA, 732 bp, synthetic construct
  source 1..732: mol_type = other DNA; note = Antibody 9 scFv VH-G4S3-VL (Ab9 scFv HL or Ab9scFvHL)
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaa

SEQ ID NO: 391 - DNA, 1341 bp, synthetic construct
  source 1..1341: mol_type = other DNA; note = Ab9scFvHL-CD28H-CD28TM-CD28CS-CD3zICS
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 392 - DNA, 1338 bp, synthetic construct
  source 1..1338: mol_type = other DNA; note = Ab9scFvHL-CD28H-CD28TM-41BBCS-CD3zICS
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 393 - DNA, 1284 bp, synthetic construct
  source 1..1284: mol_type = other DNA; note = Ab9scFvHL-CD28H-CD28TM-DAP10CS-CD3zICS
gaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 394 - DNA, 2469 bp, synthetic construct
  source 1..2469: mol_type = other DNA; note = LS-Ab9scFvHL-CD28H-CD28TM-CD28CS-CD3zICS-T2A-trCD19
atggaaaccccagcgcagcttctcttcctcctgctactctggctcccagataccaccggagaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 395 - DNA, 732 bp, synthetic construct
  source 1..732: mol_type = other DNA; note = Antibody 9 scFv VL-G4S3-VH (Ab9 scFv LH or Ab9scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcg

SEQ ID NO: 396 - DNA, 1341 bp, synthetic construct
  source 1..1341: mol_type = other DNA; note = Ab9scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 397 - DNA, 1338 bp, synthetic construct
  source 1..1338: mol_type = other DNA; note = Ab9scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 398 - DNA, 1284 bp, synthetic construct
  source 1..1284: mol_type = other DNA; note = Ab9scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 399 - DNA, 2469 bp, synthetic construct
  source 1..2469: mol_type = other DNA; note = LS-Ab9scFvLH-CD28H-CD28TM-CD28CS-CD3zICS-T2A-trCD19
atggaaaccccagcgcagcttctcttcctcctgctactctggctcccagataccaccggagatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatacgttggttacggttctccgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctcttattcttctatgcactgggtgcgtcaggccccgggtaagggcctggaatgggttgcatctatttctccttattctggctctacttattatgccgatagcgtcaagggccgtttcactataagcgcagacacatccaaaaacacagcctacctacaaatgaacagcttaagagctgaggacactgccgtctattattgtgctcgctactactggtactactactacggttttgactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 400 - DNA, 741 bp, synthetic construct
  source 1..741: mol_type = other DNA; note = Antibody 10 scFv VH-G4S3-VL (Ab10 scFv HL or Ab10scFvHL)
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaa

SEQ ID NO: 401 - DNA, 1350 bp, synthetic construct
  source 1..1350: mol_type = other DNA; note = Ab10scFvHL-CD28H-CD28TM-CD28CS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 402 - DNA, 1347 bp, synthetic construct
  source 1..1347: mol_type = other DNA; note = Ab10scFvHL-CD28H-CD28TM-41BBCS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 403 - DNA, 1293 bp, synthetic construct
  source 1..1293: mol_type = other DNA; note = Ab10scFvHL-CD28H-CD28TM-DAP10CS-CD3zICS
gaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggtggtggttctggcggcggcggctccggtggtggtggttccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaactcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 404 - skipped

SEQ ID NO: 405 - DNA, 741 bp, synthetic construct
  source 1..741: mol_type = other DNA; note = Antibody 10 scFv VL-G4S3-VH (Ab10 scFv LH or Ab10scFvLH)
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 406 - DNA, 1350 bp, synthetic construct
  source 1..1350: mol_type = other DNA; note = Ab9scFvLH-CD28H-CD28TM-CD28CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 407 - DNA, 1347 bp, synthetic construct
  source 1..1347: mol_type = other DNA; note = Ab10scFvLH-CD28H-CD28TM-41BBCS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 408 - DNA, 1293 bp, synthetic construct
  source 1..1293: mol_type = other DNA; note = Ab10scFvLH-CD28H-CD28TM-DAP10CS-CD3zICS
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgatcacgttcggacagggtaccaaggtggagatcaaaggtggtggtggttctggcggcggcggctccggtggtggtggttccgaggttcagctggtggagtctggcggtggcctggtgcagccagggggctcactccgtttgtcctgtgcagcttctggcttcaacctctattcttcttatatgcactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 409 - skipped

SEQ ID NO: 410 - DNA, 294 bp, synthetic construct
  source 1..294: mol_type = other DNA; note = Human IgG1 CH1 domain
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 411 - DNA, 45 bp, synthetic construct
  source 1..45: mol_type = other DNA; note = Human IgG1 hinge
gagcccaaatcttgtgacaaaactcacacatgcccaccgtgccca

SEQ ID NO: 412 - DNA, 330 bp, synthetic construct
  source 1..330: mol_type = other DNA; note = Human IgG1 CH2 domain
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 413 - DNA, 321 bp, synthetic construct
  source 1..321: mol_type = other DNA; note = Human IgG1 CH3 domain (356E and 358M; with C-terminal K)
gggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 414 - DNA, 318 bp, synthetic construct
  source 1..318: mol_type = other DNA; note = Human IgG1 CH3 domain (356E and 358M; without C-terminal K)
gggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 415 - DNA, 321 bp, synthetic construct
  source 1..321: mol_type = other DNA; note = Human IgG1 CH3 domain (356D and 358L; with C-terminal K)
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 416 - DNA, 318 bp, synthetic construct
  source 1..318: mol_type = other DNA; note = Human IgG1 CH3 domain (356D and 358L; without C-terminal K)
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 417 - DNA, 651 bp, synthetic construct
  source 1..651: mol_type = other DNA; note = Human IgG1 Fc (356E and 358M; with C-terminal K)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 418 - DNA, 648 bp, synthetic construct
  source 1..648: mol_type = other DNA; note = Human IgG1 Fc (356E and 358M; without C-terminal K)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 419 - DNA, 651 bp, synthetic construct
  source 1..651: mol_type = other DNA; note = Human IgG1 Fc (356D and 358L; with C-terminal K)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 420 - DNA, 648 bp, synthetic construct
  source 1..648: mol_type = other DNA; note = Human IgG1 Fc (356D and 358L; without C-terminal K)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 421 - DNA, 990 bp, synthetic construct
  source 1..990: mol_type = other DNA; note = Human IgG1 heavy chain constant region (356E and 358M; with C-terminal K)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 422 - DNA, 987 bp, synthetic construct
  source 1..987: mol_type = other DNA; note = Human IgG1 heavy chain constant region (356E and 358M; without C-terminal K)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 423 - DNA, 990 bp, synthetic construct
  source 1..990: mol_type = other DNA; note = Human IgG1 heavy chain constant region (356D and 358L; with C-terminal K)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 424 - DNA, 987 bp, synthetic construct
  source 1..987: mol_type = other DNA; note = Human IgG1 heavy chain constant region (356D and 358L; without C-terminal K)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 425 - DNA, 321 bp, synthetic construct
  source 1..321: mol_type = other DNA; note = Human Ig kappa light chain constant domain
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 426 - DNA, 321 bp, synthetic construct
  source 1..321: mol_type = other DNA; note = Human Ig lambda light chain constant domain
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag